SEQUENCE LISTING (51 sequences)

SEQ ID NO: 1 - DNA, 1080 bp, synthetic construct
  misc_feature 1..1080: note = Synthetic sequence hu Oct4
  source 1..1080: mol_type = other DNA
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaac

SEQ ID NO: 2 - DNA, 951 bp, synthetic construct
  misc_feature 1..951: note = Synthetic Sequence hu Sox2
  source 1..951: mol_type = other DNA
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatg

SEQ ID NO: 3 - DNA, 1413 bp, synthetic construct
  misc_feature 1..1413: note = hu KLF4
  source 1..1413: mol_type = other DNA
atggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 4 - DNA, 1320 bp, synthetic construct
  misc_feature 1..1320: note = hMyc gene (from AAV-TRE3G-hMcy-Ef1a-rTta)
  source 1..1320: mol_type = other DNA
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 5 - DNA, 3522 bp, synthetic construct
  misc_feature 1..3522: note = hu L-Myc
  source 1..3522: mol_type = other DNA
gagtgcgggccgcgctctcggcggcgcgcatgtgcgtgtgtgctggctgccgggctgccccgagccggcggggagccggtccgctccaggtggcgggcggctggagcgaggtgaggctgcgggtggccagggcacgggcgcgggtcccgcggtgcgggctggctgcaggctgccttctgggcacggcgcgcccccgcccggccccgccgggccctgggagctgcgctccgggcggcgctggcaaagtttgctttgaactcgctgcccacagtcgggtccgcgcgctgcgattggcttcccctaccactctgacccggggcccggcttcccgggacgcgaggactgggcgcaggctgcaagctggtggggttggggaggaacgagagcccggcagccgactgtgccgagggacccggggacacctccttcgcccggccggcacccggtcagcacgtccccccttccctcccgcagggagcggacatggactacgactcgtaccagcactatttctacgactatgactgcggggaggatttctaccgctccacggcgcccagcgaggacatctggaagaaattcgagctggtgccatcgccccccacgtcgccgccctggggcttgggtcccggcgcaggggacccggcccccgggattggtcccccggagccgtggcccggagggtgcaccggagacgaagcggaatcccggggccactcgaaaggctggggcaggaactacgcctccatcatacgccgtgactgcatgtggagcggcttctcggcccgggaacggctggagagagctgtgagcgaccggctcgctcctggcgcgccccgggggaacccgcccaaggcgtccgccgccccggactgcactcccagcctcgaagccggcaacccggcgcccgccgccccctgtccgctgggcgaacccaagacccaggcctgctccgggtccgagagcccaagcgactcggagaatgaagaaattgatgttgtgacagtagagaagaggcagtctctgggtattcggaagccggtcaccatcacggtgcgagcagaccccctggatccctgcatgaagcatttccacatctccatccatcagcaacagcacaactatgctgcccgttttcctccagaaagctgctcccaagaagaggcttcagagaggggtccccaagaagaggttctggagagagatgctgcaggggaaaaggaagatgaggaggatgaagagattgtgagtcccccacctgtagaaagtgaggctgcccagtcctgccaccccaaacctgtcagttctgatactgaggatgtgaccaagaggaagaatcacaacttcctggagcgcaagaggcggaatgacctgcgttcgcgattcttggcgctgagggaccaggtgcccaccctggccagctgctccaaggcccccaaagtagtgatcctaagcaaggccttggaatacttgcaagccctggtgggggctgagaagaggatggctacagagaaaagacagctccgatgccggcagcagcagttgcagaaaagaattgcatacctcactggctactaactgaccaaaaagcctgacagttctgtcttacgaagacacaagtttattttttaacctccctctcccctttagtaatttgcacattttggttatggtgggacagtctggacagtagatcccagaatgcattgcagccggtgcacacacaataaaggcttgcattcttggaaaccttgaaacccagctctccctcttccctgactcatgggagtgctgtatgttctctggcgcctttggcttcccagcaggcagctgactgaggagccttggggtctgcctagctcactagctctgaagaaaaggctgacagatgctatgcaacaggtggtggatgttgtcaggggctccagcctgcatgaaatctcacactctgcatgagctttaggctaggaaaggatgctcccaactggtgtctctggggtgatgcaaggacagctgggcctggatgctctccctgaggctcctttttccagaagacacacgagctgtcttgggtgaagacaagcttgcagacttgatcaacattgaccattacctcactgtcagacactttacagtagccaaggagttggaaacctttatatattatgatgttagctgacccccttcctcccactcccaatgctgcgaccctgggaacacttaaaaagcttggcctctagattctttgtctcagagccctctgggctctctcctctgagggagggacctttctttcctcacaagggacttttttgttccattatgccttgttatgcaatgggctctacagcaccctttcccacaggtcagaaatatttccccaagacacagggaaatcggtcctagcctggggcctggggatagcttggagtcctggcccatgaacttgatccctgcccaggtgttttccgaggggcacttgaggcccagtcttttctcaaggcaggtgtaagacacctcagagggagaactgtactgctgcctctttcccacctgcctcatctcaatccttgagcggcaagtttgaagttcttctggaaccatgcaaatctgtcctcctcatgcaattccaaggagcttgctggctctgcagccacccttgggccccttccagcctgccatgaatcagatatctttcccagaatctgggcgtttctgaagttttggggagagctgttgggactcatccagtgctccagaaggtggacttgcttctggtgggttttaaaggagcctccaggagatatgcttagccaaccatgatggattttaccccagctggactcggcagctccaagtggaatccacgtgcagcttctagtctgggaaagtcacccaacctagcagttgtcatgtgggtaacctcaggcacctctaagcctgtcctggaagaaggaccagcagcccctccagaactctgcccaggacagcaggtgcctgctggctctgggtttggaagttggggtgggtagggggtggtaagtactatatatggctctggaaaaccagctgctacttccaaatctattgtccataatggtttctttctgaggttgcttcttggcctcagaggaccccaggggatgtttggaaatagcctctctacccttctggagcatggtttacaaaagccagctgacttctggaattgtctatggaggacagtttgggtgtaggttactgatgtctcaactgaatagcttgtgttttataagctgctgttggctattatgctgggggagtcttttttttttatattgtatttttgtatgccttttgcaaagtggtgttaactgtttttgtacaaggaaaaaaactcttggggcaatttcctgttgcaagggtctgatttattttgaaaggcaagttcacctgaaattttgtatttagttgtgattactgattgcctgattttaaaatgttgccttctgggacatcttctaataaaagatttctcaaaca

SEQ ID NO: 6 - DNA, 2613 bp, synthetic construct
  misc_feature 1..2613: note = hu N-Myc
  source 1..2613: mol_type = other DNA
aggctgtgacagtcatctgtctggacgcgctgggtggatgcggggggctcctgggaactgtgttggagccgagcaagcgctagccaggcgcaagcgcgcacagactgtagccatccgaggacacccccgcccccccggcccacccggagacacccgcgcagaatcgcctccggatcccctgcagtcggcgggagtgttggaggtcggcgccggcccccgccttccgcgccccccacgggaaggaagcacccccggtattaaaacgaacggggcggaaagaagccctcagtcgccggccgggaggcgagccgatgccgagctgctccacgtccaccatgccgggcatgatctgcaagaacccagacctcgagtttgactcgctacagccctgcttctacccggacgaagatgacttctacttcggcggccccgactcgacccccccgggggaggacatctggaagaagtttgagctgctgcccacgcccccgctgtcgcccagccgtggcttcgcggagcacagctccgagcccccgagctgggtcacggagatgctgcttgagaacgagctgtggggcagcccggccgaggaggacgcgttcggcctggggggactgggtggcctcacccccaacccggtcatcctccaggactgcatgtggagcggcttctccgcccgcgagaagctggagcgcgccgtgagcgagaagctgcagcacggccgcgggccgccaaccgccggttccaccgcccagtccccgggagccggcgccgccagccctgcgggtcgcgggcacggcggggctgcgggagccggccgcgccggggccgccctgcccgccgagctcgcccacccggccgccgagtgcgtggatcccgccgtggtcttcccctttcccgtgaacaagcgcgagccagcgcccgtgcccgcagccccggccagtgccccggcggcgggccctgcggtcgcctcgggggcgggtattgccgccccagccggggccccgggggtcgcccctccgcgcccaggcggccgccagaccagcggcggcgaccacaaggccctcagtacctccggagaggacaccctgagcgattcagatgatgaagatgatgaagaggaagatgaagaggaagaaatcgacgtggtcactgtggagaagcggcgttcctcctccaacaccaaggctgtcaccacattcaccatcactgtgcgtcccaagaacgcagccctgggtcccgggagggctcagtccagcgagctgatcctcaaacgatgccttcccatccaccagcagcacaactatgccgccccctctccctacgtggagagtgaggatgcacccccacagaagaagataaagagcgaggcgtccccacgtccgctcaagagtgtcatccccccaaaggctaagagcttgagcccccgaaactctgactcggaggacagtgagcgtcgcagaaaccacaacatcctggagcgccagcgccgcaacgaccttcggtccagctttctcacgctcagggaccacgtgccggagttggtaaagaatgagaaggccgccaaggtggtcattttgaaaaaggccactgagtatgtccactccctccaggccgaggagcaccagcttttgctggaaaaggaaaaattgcaggcaagacagcagcagttgctaaagaaaattgaacacgctcggacttgctagacgcttctcaaaactggacagtcactgccactttgcacattttgattttttttttaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttctttttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggaagttcatacctaagtactgtaataatacctcaatgtttgaggagcatgttttgtatacaaatatattgttaatctctgttatgtactgtactaattcttacactgcctgtatactttagtatgacgctgatacataactaaatttgatacttatattttcgtatgaaaatgagttgtgaaagttttgagtagatattactttatcactttttgaactaagaaacttttgtaaagaaatttactatatatatatgcctttttcctagcctgtttcttcctgttaatgtatttgttcatgtttggtgcatagaactgggtaaatgcaaagttctgtgtttaatttcttcaaaatgtatatatttagtgctgcatcttatagcactttgaaatacctcatgtttatgaaaataaatagcttaaaattaaa

SEQ ID NO: 7 - DNA, 379 bp, synthetic construct
  misc_feature 1..379: note = TRE3G promoter (from AAV TRE3G OSK shortWPRE SV40polyA)
  source 1..379: mol_type = other DNA
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 8 - DNA, 1056 bp, synthetic construct
  misc_feature 1..1056: note = mouse Oct4 (from AAV TRE3G OSK shortWPRE SV40polyA)
  source 1..1056: mol_type = other DNA
atggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaac

SEQ ID NO: 9 - DNA, 957 bp, synthetic construct
  misc_feature 1..957: note = mouse Sox2 (from AAV TRE3G OSK shortWPRE SV40polyA)
  source 1..957: mol_type = other DNA
atgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatg

SEQ ID NO: 10 - DNA, 1449 bp, synthetic construct
  misc_feature 1..1449: note = mouse Klf4 (from AAV TRE3G OSK shortWPRE SV40polyA)
  source 1..1449: mol_type = other DNA
atgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccagcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgcgacagggccttttccaggtcggaccaccttgccttacacatgaagaggcacttt

SEQ ID NO: 11 - DNA, 1320 bp, synthetic construct
  misc_feature 1..1320: note = mouse c-Myc (from AAV TRE3G cMyc shortWPRE SV40polyA)
  source 1..1320: mol_type = other DNA
atgcccctcaacgtgaacttcaccaacaggaactatgacctcgactacgactccgtacagccctatttcatctgcgacgaggaagagaatttctatcaccagcaacagcagagcgagctgcagccgcccgcgcccagtgaggatatctggaagaaattcgagctgcttcccaccccgcccctgtccccgagccgccgctccgggctctgctctccatcctatgttgcggtcgctacgtccttctccccaagggaagacgatgacggcggcggtggcaacttctccaccgccgatcagctggagatgatgaccgagttacttggaggagacatggtgaaccagagcttcatctgcgatcctgacgacgagaccttcatcaagaacatcatcatccaggactgtatgtggagcggtttctcagccgctgccaagctggtctcggagaagctggcctcctaccaggctgcgcgcaaagacagcaccagcctgagccccgcccgcgggcacagcgtctgctccacctccagcctgtacctgcaggacctcaccgccgccgcgtccgagtgcattgacccctcagtggtctttccctacccgctcaacgacagcagctcgcccaaatcctgtacctcgtccgattccacggccttctctccttcctcggactcgctgctgtcctccgagtcctccccacgggccagccctgagcccctagtgctgcatgaggagacaccgcccaccaccagcagcgactctgaagaagagcaagaagatgaggaagaaattgatgtggtgtctgtggagaagaggcaaacccctgccaagaggtcggagtcgggctcatctccatcccgaggccacagcaaacctccgcacagcccactggtcctcaagaggtgccacgtctccactcaccagcacaactacgccgcacccccctccacaaggaaggactatccagctgccaagagggccaagttggacagtggcagggtcctgaagcagatcagcaacaaccgcaagtgctccagccccaggtcctcagacacggaggaaaacgacaagaggcggacacacaacgtcttggaacgtcagaggaggaacgagctgaagcgcagcttttttgccctgcgtgaccagatccctgaattggaaaacaacgaaaaggcccccaaggtagtgatcctcaaaaaagccaccgcctacatcctgtccattcaagcagacgagcacaagctcacctctgaaaaggacttattgaggaaacgacgagaacagttgaaacacaaactcgaacagcttcgaaactctggtgcataa

SEQ ID NO: 12 - DNA, 1182 bp, synthetic construct
  misc_feature 1..1182: note = EF-1a promoter (from AAV TRE3G cMyc shortWPRE SV40polyA)
  source 1..1182: mol_type = other DNA
tcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaagccaggcctcaactcaagcacgaggcgaaggggctccttaagcgcaaggcctcgaactctcccacccacttccaacccgaagctcgggatcaagaatcacgtactgcagccaggggcgtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagc

SEQ ID NO: 13 - DNA, 747 bp, synthetic construct
  misc_feature 1..747: note = Tet-On 3G (from AAV TRE3G cMyc shortWPRE SV40polyA)
  source 1..747: mol_type = other DNA
ttacccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacat

SEQ ID NO: 14 - DNA, 361 bp, synthetic construct
  misc_feature 1..361: note = short WPRE & SV40 polyA (from AAV TRE3G OSK shortWPRE SV40polyA)
  source 1..361: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtt

SEQ ID NO: 15 - DNA, 225 bp, synthetic construct
  misc_feature 1..225: note = bGH polyA (from AAV TRE3G cMyc shortWPRE SV40polyA)
  source 1..225: mol_type = other DNA
ccatagagcccaccgcatccccagcatgcctgctattgacttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacag

SEQ ID NO: 16 - DNA, 951 bp, synthetic construct
  misc_feature 1..951: note = dnNFKBIA (h-IKB alpha sup repressor)
  source 1..951: mol_type = other DNA
atgttccaggcggccgagcgtccccaggagtgggccatggagggcccccgcgacgggctgaagaaggagcggctactggacgaccgccacgacgccggcctggacgccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggccttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgtta

SEQ ID NO: 17 - protein, 5 aa, synthetic construct
  REGION 1..5: note = furin cleavage site consensus sequence
  SITE 2: note = misc_feature - Xaa can be any naturally occurring amino acid
RXKRR

SEQ ID NO: 18 - DNA, 568 bp, synthetic construct
  misc_feature 1..568: note = IRES
  source 1..568: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacga

SEQ ID NO: 19 - DNA, 1659 bp, synthetic construct
  misc_feature 1..1659: note = hu p16 promoter
  source 1..1659: mol_type = other DNA
gcgggccacatttccaggttcccggagctgggcctagtcccgaatcctctggcacacacccacccactcaggccgcgggtccagcccgcgaggtttaggacggatccaggcagaccgcaggctccgggtcggggcaccgggtcagcgcgccggcctgaaggcggcgtcctgggctcgacttcccgcgcgcggagagccggcgagcccgcgtccgagttcctggacgagagccgagcctcgcttagaccgcgctcaggacccggctcctccgcattctccggctgcccctgtgtcctcgactcacccctcctttctgccgctccttcctttccttgccctgcttttactgttcccaaacaggaccgcttttcctgtctcccagctggaaaggaggaagggagagagtccagaaaggatcggtgatgtggaagaaaaggggaggaggggacatggagggggagaccggagagagaacgtacgccgaggagtcaggcggcgggatcaaggggagtcggggtgtctgggcgcggggcagagcgtggaggcggcagcggccaacggtcgccaagacaaccattctacgcgaggacgcggcgacaggaggggagcggccagcaggggaggggagcgcgggggaagaggaaagaggaagaagcgctcagatgctccgcggctgtcgtgaaggttaaaaccgaaaataaaaatgggctagacacaaaggactcggtgcttgtcccagccaggcgccctcggcgacgcgggcagctgggaggggaatgggcgcccggacccagctgggacccccgggtgcgactccacctacctagtccggcgccaggccgggtcgacagctccggcagcgccagcgccgcgccgtgtccagatgtcgcgtcagaggcgtgcagcggtttagtttaatttcgcttgttttccaaatctagaagaggagcggagcggcttttagttcaaaactgacattcagcctcctgattggcggatagagcaatgagatgacctcgctttcctttcttcctttttcatttttaaataatctagtttgaagaatggaagactttcgacgaggggagccaggaataaaataaggggaataggggagcggggacgcgagcagcaccagaatccgcgggagcgcggctgttcctggtagggccgtgtcaggtgacggatgtagctagggggcgagctgcctggagttgcgttccaggcgtccggcccctgggccgtcaccgcggggcgcccgcgctgagggtgggaagatggtggtgggggtgggggcgcacacagggcgggaaagtggcggtaggcgggagggagaggaacgcgggccctgagccgcccgcgcgcgcgcctccctacgggcgcctccggcagcccttcccgcgtgcgcagggctcagagccgttccgagatcttggaggtccgggtgggagtgggggtggggtgggggtgggggtgaaggtggggggcgggcgcgctcagggaaggcgggtgcgcgcctgcggggcggagatgggcagggggcggtgcgtgggtcccagtctgcagttaagggggcaggagtggcgctgctcacctctggtgccaaagggcggcgcagcggctgccgagctcggccctggaggcggcgagaacatggtg

SEQ ID NO: 20 - DNA, 900 bp, synthetic construct
  misc_feature 1..900: note = mouse p16 promoter
  source 1..900: mol_type = other DNA
ttttcctgtttcgcgggggccggggtggaggcggagaaggaggcgaggaggcagcggagaagggggcagccgggaggggggaggggaccggagaaggaggcgacgcggggcggggaaagaggaagagccggggatgctcgcgcttaaaaccgaaaataaacaacgtcctcgggctgcggcctcgtgtctgcccgaccgcgcgggggcctggccgggacacacttaccggtccgccgcggcgtgcaggccctggtcccgggtacacgcagcggctcttctctgcgcgctgttcccaaccggaggagcgcagcgtaccgctaagggttcaaaacgcccgcgcgcgtcctgattggcgggccggcccatggctggcctcgccgatcttcctattttcttctttttatagatggactcggagcaagggaaacctcgccgaggggcgtcaggcatagacgaaagtttcgggacccgaaagttaaccggagcgaaagccgatcggcgcaggagacgttgccggccggctgcgaaagggcgcagctactgctagggggcgggcggtgcgcacgcgtcccgcggcgctggctgtcaccgcgatgggtggcgagcgaagcgagcgggatccggagcgtgccctgcgcgggaggcagcgggaccccggatgcggcagggcccgcgcgcgcctccccctgggcgcctctgggaagctttcccgcgcgactggggaccgcgcgccttgggactcgggggcgcgcctgagggcggagatgggcgtggagcaaagatgggccgggggcggcgcgtgggtctcgaggtgcctcaacgccgaaggggctgggggcggcgcttctcacctcgcttgtcacagtgaggccgccgctgagggagtacagcagcgggagc

SEQ ID NO: 21 - DNA, 8483 bp, synthetic construct
  misc_feature 1..8483: note = AAV H-p16 OSK short WPRE sv40 polya
  source 1..8483: mol_type = other DNA
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagcgggccacatttccaggttcccggagctgggcctagtcccgaatcctctggcacacacccacccactcaggccgcgggtccagcccgcgaggtttaggacggatccaggcagaccgcaggctccgggtcggggcaccgggtcagcgcgccggcctgaaggcggcgtcctgggctcgacttcccgcgcgcggagagccggcgagcccgcgtccgagttcctggacgagagccgagcctcgcttagaccgcgctcaggacccggctcctccgcattctccggctgcccctgtgtcctcgactcacccctcctttctgccgctccttcctttccttgccctgcttttactgttcccaaacaggaccgcttttcctgtctcccagctggaaaggaggaagggagagagtccagaaaggatcggtgatgtggaagaaaaggggaggaggggacatggagggggagaccggagagagaacgtacgccgaggagtcaggcggcgggatcaaggggagtcggggtgtctgggcgcggggcagagcgtggaggcggcagcggccaacggtcgccaagacaaccattctacgcgaggacgcggcgacaggaggggagcggccagcaggggaggggagcgcgggggaagaggaaagaggaagaagcgctcagatgctccgcggctgtcgtgaaggttaaaaccgaaaataaaaatgggctagacacaaaggactcggtgcttgtcccagccaggcgccctcggcgacgcgggcagctgggaggggaatgggcgcccggacccagctgggacccccgggtgcgactccacctacctagtccggcgccaggccgggtcgacagctccggcagcgccagcgccgcgccgtgtccagatgtcgcgtcagaggcgtgcagcggtttagtttaatttcgcttgttttccaaatctagaagaggagcggagcggcttttagttcaaaactgacattcagcctcctgattggcggatagagcaatgagatgacctcgctttcctttcttcctttttcatttttaaataatctagtttgaagaatggaagactttcgacgaggggagccaggaataaaataaggggaataggggagcggggacgcgagcagcaccagaatccgcgggagcgcggctgttcctggtagggccgtgtcaggtgacggatgtagctagggggcgagctgcctggagttgcgttccaggcgtccggcccctgggccgtcaccgcggggcgcccgcgctgagggtgggaagatggtggtgggggtgggggcgcacacagggcgggaaagtggcggtaggcgggagggagaggaacgcgggccctgagccgcccgcgcgcgcgcctccctacgggcgcctccggcagcccttcccgcgtgcgcagggctcagagccgttccgagatcttggaggtccgggtgggagtgggggtggggtgggggtgggggtgaaggtggggggcgggcgcgctcagggaaggcgggtgcgcgcctgcggggcggagatgggcagggggcggtgcgtgggtcccagtctgcagttaagggggcaggagtggcgctgctcacctctggtgccaaagggcggcgcagcggctgccgagctcggccctggaggcggcgagaacatggtgatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaacggatccggagccacgaacttctctctgttaaagcaagcaggagatgttgaagaaaaccccgggcctatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgggatccggagccacgaacttctctctgttaaagcaagcaggagatgttgaagaaaaccccgggcctatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtttcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctg

SEQ ID NO: 22 - DNA, 6641 bp, synthetic construct
  misc_feature 1..6641: note = AAV H-p16 IKKB sup repressor-GFP short WPRE sv40 polya
  source 1..6641: mol_type = other DNA
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagcgggccacatttccaggttcccggagctgggcctagtcccgaatcctctggcacacacccacccactcaggccgcgggtccagcccgcgaggtttaggacggatccaggcagaccgcaggctccgggtcggggcaccgggtcagcgcgccggcctgaaggcggcgtcctgggctcgacttcccgcgcgcggagagccggcgagcccgcgtccgagttcctggacgagagccgagcctcgcttagaccgcgctcaggacccggctcctccgcattctccggctgcccctgtgtcctcgactcacccctcctttctgccgctccttcctttccttgccctgcttttactgttcccaaacaggaccgcttttcctgtctcccagctggaaaggaggaagggagagagtccagaaaggatcggtgatgtggaagaaaaggggaggaggggacatggagggggagaccggagagagaacgtacgccgaggagtcaggcggcgggatcaaggggagtcggggtgtctgggcgcggggcagagcgtggaggcggcagcggccaacggtcgccaagacaaccattctacgcgaggacgcggcgacaggaggggagcggccagcaggggaggggagcgcgggggaagaggaaagaggaagaagcgctcagatgctccgcggctgtcgtgaaggttaaaaccgaaaataaaaatgggctagacacaaaggactcggtgcttgtcccagccaggcgccctcggcgacgcgggcagctgggaggggaatgggcgcccggacccagctgggacccccgggtgcgactccacctacctagtccggcgccaggccgggtcgacagctccggcagcgccagcgccgcgccgtgtccagatgtcgcgtcagaggcgtgcagcggtttagtttaatttcgcttgttttccaaatctagaagaggagcggagcggcttttagttcaaaactgacattcagcctcctgattggcggatagagcaatgagatgacctcgctttcctttcttcctttttcatttttaaataatctagtttgaagaatggaagactttcgacgaggggagccaggaataaaataaggggaataggggagcggggacgcgagcagcaccagaatccgcgggagcgcggctgttcctggtagggccgtgtcaggtgacggatgtagctagggggcgagctgcctggagttgcgttccaggcgtccggcccctgggccgtcaccgcggggcgcccgcgctgagggtgggaagatggtggtgggggtgggggcgcacacagggcgggaaagtggcggtaggcgggagggagaggaacgcgggccctgagccgcccgcgcgcgcgcctccctacgggcgcctccggcagcccttcccgcgtgcgcagggctcagagccgttccgagatcttggaggtccgggtgggagtgggggtggggtgggggtgggggtgaaggtggggggcgggcgcgctcagggaaggcgggtgcgcgcctgcggggcggagatgggcagggggcggtgcgtgggtcccagtctgcagttaagggggcaggagtggcgctgctcacctctggtgccaaagggcggcgcagcggctgccgagctcggccctggaggcggcgagaacatggtgatgttccaggcggccgagcgtccccaggagtgggccatggagggcccccgcgacgggctgaagaaggagcggctactggacgaccgccacgacgccggcctggacgccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggccttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttaggatccggagagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatttgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtttcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctg

SEQ ID NO: 23 - DNA, 5888 bp, synthetic construct
  misc_feature 1..5888: note = AAV mp16 h-IKB alpha super repressor-GFP
  source 1..5888: mol_type = other DNA
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagattttcctgtttcgcgggggccggggtggaggcggagaaggaggcgaggaggcagcggagaagggggcagccgggaggggggaggggaccggagaaggaggcgacgcggggcggggaaagaggaagagccggggatgctcgcgcttaaaaccgaaaataaacaacgtcctcgggctgcggcctcgtgtctgcccgaccgcgcgggggcctggccgggacacacttaccggtccgccgcggcgtgcaggccctggtcccgggtacacgcagcggctcttctctgcgcgctgttcccaaccggaggagcgcagcgtaccgctaagggttcaaaacgcccgcgcgcgtcctgattggcgggccggcccatggctggcctcgccgatcttcctattttcttctttttatagatggactcggagcaagggaaacctcgccgaggggcgtcaggcatagacgaaagtttcgggacccgaaagttaaccggagcgaaagccgatcggcgcaggagacgttgccggccggctgcgaaagggcgcagctactgctagggggcgggcggtgcgcacgcgtcccgcggcgctggctgtcaccgcgatgggtggcgagcgaagcgagcgggatccggagcgtgccctgcgcgggaggcagcgggaccccggatgcggcagggcccgcgcgcgcctccccctgggcgcctctgggaagctttcccgcgcgactggggaccgcgcgccttgggactcgggggcgcgcctgagggcggagatgggcgtggagcaaagatgggccgggggcggcgcgtgggtctcgaggtgcctcaacgccgaaggggctgggggcggcgcttctcacctcgcttgtcacagtgaggccgccgctgagggagtacagcagcgggagcgccaccatgttccaggcggccgagcgtccccaggagtgggccatggagggcccccgcgacgggctgaagaaggagcggctactggacgaccgccacgacgccggcctggacgccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggccttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttaggatccggagagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatttgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtttcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctg

SEQ ID NO: 24 - DNA, 7772 bp, synthetic construct
  misc_feature 1..7772: note = AAV mp16-OSK
  source 1..7772: mol_type = other DNA
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagattttcctgtttcgcgggggccggggtggaggcggagaaggaggcgaggaggcagcggagaagggggcagccgggaggggggaggggaccggagaaggaggcgacgcggggcggggaaagaggaagagccggggatgctcgcgcttaaaaccgaaaataaacaacgtcctcgggctgcggcctcgtgtctgcccgaccgcgcgggggcctggccgggacacacttaccggtccgccgcggcgtgcaggccctggtcccgggtacacgcagcggctcttctctgcgcgctgttcccaaccggaggagcgcagcgtaccgctaagggttcaaaacgcccgcgcgcgtcctgattggcgggccggcccatggctggcctcgccgatcttcctattttcttctttttatagatggactcggagcaagggaaacctcgccgaggggcgtcaggcatagacgaaagtttcgggacccgaaagttaaccggagcgaaagccgatcggcgcaggagacgttgccggccggctgcgaaagggcgcagctactgctagggggcgggcggtgcgcacgcgtcccgcggcgctggctgtcaccgcgatgggtggcgagcgaagcgagcgggatccggagcgtgccctgcgcgggaggcagcgggaccccggatgcggcagggcccgcgcgcgcctccccctgggcgcctctgggaagctttcccgcgcgactggggaccgcgcgccttgggactcgggggcgcgcctgagggcggagatgggcgtggagcaaagatgggccgggggcggcgcgtgggtctcgaggtgcctcaacgccgaaggggctgggggcggcgcttctcacctcgcttgtcacagtgaggccgccgctgagggagtacagcagcgggagcgccaccatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaacgctagcggcagcggcgccacgaacttctctctgttaaagcaagcaggagatgttgaagaaaaccccgggcctgcatgcatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatggcatgcggctccggcgagggcaggggaagtcttctaacatgcggggacgtggaggaaaatcccggcccactcgagatgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccagcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgcgacagggccttttccaggtcggaccaccttgccttacacatgaagaggcacttttaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtttcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctg

SEQ ID NO: 25 - DNA, 6610 bp, synthetic construct
  misc_feature 1..6610: note = AAV mp16 CBRLuc-GFP
  source 1..6610: mol_type = other DNA
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagattttcctgtttcgcgggggccggggtggaggcggagaaggaggcgaggaggcagcggagaagggggcagccgggaggggggaggggaccggagaaggaggcgacgcggggcggggaaagaggaagagccggggatgctcgcgcttaaaaccgaaaataaacaacgtcctcgggctgcggcctcgtgtctgcccgaccgcgcgggggcctggccgggacacacttaccggtccgccgcggcgtgcaggccctggtcccgggtacacgcagcggctcttctctgcgcgctgttcccaaccggaggagcgcagcgtaccgctaagggttcaaaacgcccgcgcgcgtcctgattggcgggccggcccatggctggcctcgccgatcttcctattttcttctttttatagatggactcggagcaagggaaacctcgccgaggggcgtcaggcatagacgaaagtttcgggacccgaaagttaaccggagcgaaagccgatcggcgcaggagacgttgccggccggctgcgaaagggcgcagctactgctagggggcgggcggtgcgcacgcgtcccgcggcgctggctgtcaccgcgatgggtggcgagcgaagcgagcgggatccggagcgtgccctgcgcgggaggcagcgggaccccggatgcggcagggcccgcgcgcgcctccccctgggcgcctctgggaagctttcccgcgcgactggggaccgcgcgccttgggactcgggggcgcgcctgagggcggagatgggcgtggagcaaagatgggccgggggcggcgcgtgggtctcgaggtgcctcaacgccgaaggggctgggggcggcgcttctcacctcgcttgtcacagtgaggccgccgctgagggagtacagcagcgggagcgccaccatggatggtaaagcgtgagaaaaatgtcatctatggccctgagcctctccatcctttggaggatttgactgccggcgaaatgctgtttcgtgctctccgcaagcactctcatttgcctcaagccttggtcgatgtggtcggcgatgaatctttgagctacaaggagttttttgaggcaaccgtcttgctggctcagtccctccacaattgtggctacaagatgaacgacgtcgttagtatctgtgctgaaaacaatacccgtttcttcattccagtcatcgccgcatggtatatcggtatgatcgtggctccagtcaacgagagctacattcccgacgaactgtgtaaagtcatgggtatctctaagccacagattgtcttcaccactaagaatattctgaacaaagtcctggaagtccaaagccgcaccaactttattaagcgtatcatcatcttggacactgtggagaatattcacggttgcgaatctttgcctaatttcatctctcgctattcagacggcaacatcgcaaactttaaaccactccacttcgaccctgtggaacaagttgcagccattctgtgtagcagcggtactactggactcccaaagggagtcatgcagacccatcaaaacatttgcgtgcgtctgatccatgctctcgatccacgctacggcactcagctgattcctggtgtcaccgtcttggtctacttgcctttcttccatgctttcggctttcatattactttgggttactttatggtcggtctccgcgtgattatgttccgccgttttgatcaggaggctttcttgaaagccatccaagattatgaagtccgcagtgtcatcaacgtgcctagcgtgatcctgtttttgtctaagagcccactcgtggacaagtacgacttgtcttcactgcgtgaattgtgttgcggtgccgctccactggctaaggaggtcgctgaagtggccgccaaacgcttgaatcttccagggattcgttgtggcttcggcctcaccgaatctaccagtgcgattatccagactctcggggatgagtttaagagcggctctttgggccgtgtcactccactcatggctgctaagatcgctgatcgcgaaactggtaaggctttgggcccgaaccaagtgggcgagctgtgtatcaaaggccctatggtgagcaagggttatgtcaataacgttgaagctaccaaggaggccatcgacgacgacggctggttgcattctggtgattttggatattacgacgaagatgagcatttttacgtcgtggatcgttacaaggagctgatcaaatacaagggtagccaggttgctccagctgagttggaggagattctgttgaaaaatccatgcattcgcgatgtcgctgtggtcggcattcctgatctggaggccggcgaactgccttctgctttcgttgtcaagcagcctggtacagaaattaccgccaaagaagtgtatgattacctggctgaacgtgtgagccatactaagtacttgcgtggcggcgtgcgttttgttgactccatccctcgtaacgtaacaggcaaaattacccgcaaggagctgttgaaacaattgttggtgaaggccggcggtggatccggagagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatttgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtttcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgagtgaggccgccgctgagggagtacagcagcgggagcgccacc

SEQ ID NO: 26 - DNA, 7279 bp, synthetic construct
  misc_feature 1..7279: note = plasmid AAV TRE3G OSK shortWPRE SV40 polyA
  source 1..7279: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagcggccgcacgcgttcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacgaattcgccaccatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaacgctagcggcagcggcgccacgaacttctctctgttaaagcaagcaggagatgttgaagaaaaccccgggcctgcatgcatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatggcatgcggctccggcgagggcaggggaagtcttctaacatgcggggacgtggaggaaaatcccggcccactcgagatgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccagcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgcgacagggccttttccaggtcggaccaccttgccttacacatgaagaggcacttttaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtttcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgc

SEQ ID NO: 27 - DNA, 7462 bp, synthetic construct
  misc_feature 1..7462: note = plasmid AAV TRE3G cMyc shortWPRE SV40polyA + EF1a TetON3G
  source 1..7462: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagcggccgcacgcgccatagagcccaccgcatccccagcatgcctgctattgacttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagcggccgcttacccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacatggtggcgctagcctcgagaatttggatcggtcccgttctcttctatggaggtcaaaacagcgtggatggcgtctccaggcgatctgacggttcactaaacgagctctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaagccaggcctcaactcaagcacgaggcgaaggggctccttaagcgcaaggcctcgaactctcccacccacttccaacccgaagctcgggatcaagaatcacgtactgcagccaggggcgtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcacgcatgctcttctccacctcagtgatgacgagagcgggcgggtgagggggcgggaacgcagcgatctctgggttctacgttagtgggagtttaacgacggtccctgggattccccaaggcaggggcgagtccttttgtatgaattactcacgcgttcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacgaattcgccaccatgcccctcaacgtgaacttcaccaacaggaactatgacctcgactacgactccgtacagccctatttcatctgcgacgaggaagagaatttctatcaccagcaacagcagagcgagctgcagccgcccgcgcccagtgaggatatctggaagaaattcgagctgcttcccaccccgcccctgtccccgagccgccgctccgggctctgctctccatcctatgttgcggtcgctacgtccttctccccaagggaagacgatgacggcggcggtggcaacttctccaccgccgatcagctggagatgatgaccgagttacttggaggagacatggtgaaccagagcttcatctgcgatcctgacgacgagaccttcatcaagaacatcatcatccaggactgtatgtggagcggtttctcagccgctgccaagctggtctcggagaagctggcctcctaccaggctgcgcgcaaagacagcaccagcctgagccccgcccgcgggcacagcgtctgctccacctccagcctgtacctgcaggacctcaccgccgccgcgtccgagtgcattgacccctcagtggtctttccctacccgctcaacgacagcagctcgcccaaatcctgtacctcgtccgattccacggccttctctccttcctcggactcgctgctgtcctccgagtcctccccacgggccagccctgagcccctagtgctgcatgaggagacaccgcccaccaccagcagcgactctgaagaagagcaagaagatgaggaagaaattgatgtggtgtctgtggagaagaggcaaacccctgccaagaggtcggagtcgggctcatctccatcccgaggccacagcaaacctccgcacagcccactggtcctcaagaggtgccacgtctccactcaccagcacaactacgccgcacccccctccacaaggaaggactatccagctgccaagagggccaagttggacagtggcagggtcctgaagcagatcagcaacaaccgcaagtgctccagccccaggtcctcagacacggaggaaaacgacaagaggcggacacacaacgtcttggaacgtcagaggaggaacgagctgaagcgcagcttttttgccctgcgtgaccagatccctgaattggaaaacaacgaaaaggcccccaaggtagtgatcctcaaaaaagccaccgcctacatcctgtccattcaagcagacgagcacaagctcacctctgaaaaggacttattgaggaaacgacgagaacagttgaaacacaaactcgaacagcttcgaaactctggtgcataatctagactagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtttcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgc

SEQ ID NO: 28 - DNA, 7457 bp, synthetic construct
  misc_feature 1..7457: note = plasmid AAV-TRE3G-hMyc-Ef1a-rTta
  source 1..7457: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagcggccgcacgcgccatagagcccaccgcatccccagcatgcctgctattgacttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagcggccgcttacccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacatggtggcgctagcctcgagaatttggatcggtcccgttctcttctatggaggtcaaaacagcgtggatggcgtctccaggcgatctgacggttcactaaacgagctctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaagccaggcctcaactcaagcacgaggcgaaggggctccttaagcgcaaggcctcgaactctcccacccacttccaacccgaagctcgggatcaagaatcacgtactgcagccaggggcgtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcacgcatgctcttctccacctcagtgatgacgagagcgggcgggtgagggggcgggaacgcagcgatctctgggttctacgttagtgggagtttaacgacggtccctgggattccccaaggcaggggcgagtccttttgtatgaattactcacgcgttcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacgaattcgccaccatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaactagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtttcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgc

SEQ ID NO: 29 - DNA, 6651 bp, synthetic construct
  misc_feature 1..6651: note = plasmid AAV-CAG-CBRLuc-GFP
  source 1..6651: mol_type = other DNA
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccgggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgccaccatggtaaagcgtgagaaaaatgtcatctatggccctgagcctctccatcctttggaggatttgactgccggcgaaatgctgtttcgtgctctccgcaagcactctcatttgcctcaagccttggtcgatgtggtcggcgatgaatctttgagctacaaggagttttttgaggcaaccgtcttgctggctcagtccctccacaattgtggctacaagatgaacgacgtcgttagtatctgtgctgaaaacaatacccgtttcttcattccagtcatcgccgcatggtatatcggtatgatcgtggctccagtcaacgagagctacattcccgacgaactgtgtaaagtcatgggtatctctaagccacagattgtcttcaccactaagaatattctgaacaaagtcctggaagtccaaagccgcaccaactttattaagcgtatcatcatcttggacactgtggagaatattcacggttgcgaatctttgcctaatttcatctctcgctattcagacggcaacatcgcaaactttaaaccactccacttcgaccctgtggaacaagttgcagccattctgtgtagcagcggtactactggactcccaaagggagtcatgcagacccatcaaaacatttgcgtgcgtctgatccatgctctcgatccacgctacggcactcagctgattcctggtgtcaccgtcttggtctacttgcctttcttccatgctttcggctttcatattactttgggttactttatggtcggtctccgcgtgattatgttccgccgttttgatcaggaggctttcttgaaagccatccaagattatgaagtccgcagtgtcatcaacgtgcctagcgtgatcctgtttttgtctaagagcccactcgtggacaagtacgacttgtcttcactgcgtgaattgtgttgcggtgccgctccactggctaaggaggtcgctgaagtggccgccaaacgcttgaatcttccagggattcgttgtggcttcggcctcaccgaatctaccagtgcgattatccagactctcggggatgagtttaagagcggctctttgggccgtgtcactccactcatggctgctaagatcgctgatcgcgaaactggtaaggctttgggcccgaaccaagtgggcgagctgtgtatcaaaggccctatggtgagcaagggttatgtcaataacgttgaagctaccaaggaggccatcgacgacgacggctggttgcattctggtgattttggatattacgacgaagatgagcatttttacgtcgtggatcgttacaaggagctgatcaaatacaagggtagccaggttgctccagctgagttggaggagattctgttgaaaaatccatgcattcgcgatgtcgctgtggtcggcattcctgatctggaggccggcgaactgccttctgctttcgttgtcaagcagcctggtacagaaattaccgccaaagaagtgtatgattacctggctgaacgtgtgagccatactaagtacttgcgtggcggcgtgcgttttgttgactccatccctcgtaacgtaacaggcaaaattacccgcaaggagctgttgaaacaattgttggtgaaggccggcggtggatccggagagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatttgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtttcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgagtgaggccgccgctgagggagtacagcagcgggagcgccacc

SEQ ID NO: 30 - DNA, 15171 bp, synthetic construct
  misc_feature 1..15171: note = plasmid pLenti-TRE3G-hOSKM-Cherry-rtTa-BlastR
  source 1..15171: mol_type = other DNA
gcttaagcggtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgagggttttcccagtcacgacgttgtaaaacgacggccagtgaattcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagaattcgccaccatggcgggacacctggcttcagattttgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaacgctagcggcagcggcgccacgaacttctctctgttaaagcaagcaggagatgttgaagaaaaccccgggcctgcttgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatggcatgcggctccggcgagggcaggggaagtcttctaacatgcggggacgtggaggaaaatcccggcccactcgagatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcattttctcgagggctcgggccagtgtactaattatgctctcttgaaattggctggagatgttgagagcaacccaggtcccggcgcgccaatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaaaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaatcggtaaattcagatctaacttgtttattgcagcttataatggttacaaataaagcaatagcatcacaaatttcacaaataaagcatttttttcactgcattctagttgtggtttgtccaaactcatcaatgtatcttatcatgtctggatcctggtacctggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggggatccggcgcaacaaacttctctctgctgaaacaagccggagatgtcgaagagaatcctggaccgccaccggtcgccaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 31 - skipped

SEQ ID NO: 32 - DNA, 20 bp, synthetic construct
  misc_feature 1..20: note = Luciferase (forward)
  source 1..20: mol_type = other DNA
ctcactgagactacatcagc

SEQ ID NO: 33 - DNA, 20 bp, synthetic construct
  misc_feature 1..20: note = Luciferase (reverse)
  source 1..20: mol_type = other DNA
tccagatccacaaccttcgc

SEQ ID NO: 34 - DNA, 21 bp, synthetic construct
  misc_feature 1..21: note = TGF? (forward)
  source 1..21: mol_type = other DNA
aacccccattgctgtcccgtg

SEQ ID NO: 35 - DNA, 21 bp, synthetic construct
  misc_feature 1..21: note = TGF? (reverse)
  source 1..21: mol_type = other DNA
ggcgtatcagtgggggtcagc

SEQ ID NO: 36 - DNA, 22 bp, synthetic construct
  misc_feature 1..22: note = NGAL (forward)
  source 1..22: mol_type = other DNA
gatgttgttatccttgaggccc

SEQ ID NO: 37 - DNA, 19 bp, synthetic construct
  misc_feature 1..19: note = NGAL (reverse)
  source 1..19: mol_type = other DNA
cactgactacgagtttgcc

SEQ ID NO: 38 - DNA, 20 bp, synthetic construct
  misc_feature 1..20: note = TNF (forward)
  source 1..20: mol_type = other DNA
actgaacttcggggtgatcg

SEQ ID NO: 39 - DNA, 20 bp, synthetic construct
  misc_feature 1..20: note = TNF (reverse)
  source 1..20: mol_type = other DNA
gcttggtggtttgctacgac

SEQ ID NO: 40 - DNA, 20 bp, synthetic construct
  misc_feature 1..20: note = GFP (forward)
  source 1..20: mol_type = other DNA
ccccgaccacatgaacgacg

SEQ ID NO: 41 - DNA, 23 bp, synthetic construct
  misc_feature 1..23: note = GFP (reverse)
  source 1..23: mol_type = other DNA
gccgtcgtccttgaagaagatgg

SEQ ID NO: 42 - DNA, 22 bp, synthetic construct
  misc_feature 1..22: note = CXCL10 (forward)
  source 1..22: mol_type = other DNA
ttccgtaagctatgtgcaggta

SEQ ID NO: 43 - DNA, 22 bp, synthetic construct
  misc_feature 1..22: note = CXCL10 (reverse)
  source 1..22: mol_type = other DNA
tcaggtgaactcagaactgatg

SEQ ID NO: 44 - DNA, 20 bp, synthetic construct
  misc_feature 1..20: note = HIF (forward)
  source 1..20: mol_type = other DNA
caactgccaccactgatgaa

SEQ ID NO: 45 - DNA, 20 bp, synthetic construct
  misc_feature 1..20: note = HIF (reverse)
  source 1..20: mol_type = other DNA
tgggtagaaggtggagatgc

SEQ ID NO: 46 - DNA, 20 bp, synthetic construct
  misc_feature 1..20: note = ETR (forward)
  source 1..20: mol_type = other DNA
tctcggagagcagagacaca

SEQ ID NO: 47 - DNA, 21 bp, synthetic construct
  misc_feature 1..21: note = ETR (reverse)
  source 1..21: mol_type = other DNA
tggactttggagtttctccct

SEQ ID NO: 48 - DNA, 24 bp, synthetic construct
  misc_feature 1..24: note = GADPH (forward)
  source 1..24: mol_type = other DNA
caactccctcaagattgtcagcaa

SEQ ID NO: 49 - DNA, 20 bp, synthetic construct
  misc_feature 1..20: note = GADPH (reverse)
  source 1..20: mol_type = other DNA
ggcatggactgtggtcatga

SEQ ID NO: 50 - DNA, 20 bp, synthetic construct
  misc_feature 1..20: note = MCP1 (forward)
  source 1..20: mol_type = other DNA
ctgtctcagccagatgttaa

SEQ ID NO: 51 - DNA, 20 bp, synthetic construct
  misc_feature 1..20: note = MCP1 (reverse)
  source 1..20: mol_type = other DNA
tgggatcatcttgccagtga

(19) United States
(12) Patent Application Publication
(10) Pub. No.: US 2026/0256132 A1
(43) Pub. Date: Sep. 3, 2026

(54) USE OF REGENERATION FACTORS IN ORGAN TRANSPLANTATION

(71) Applicants: ALTOS LABS, INC., Redwood City, CA (US); HOSPITAL CLÍNIC DE BARCELONA, Barcelona (ES)

(72) Inventors: Juan Carlos IZPISUA BELMONTE, Redwood City, CA (US); Pradeep Reddy DUBBAKA VENU, Redwood City, CA (US); Yosu LUQUE, Paris (FR); Concepcion RODRIGUEZ ESTEBAN, Redwood City, CA (US); Josep Maria CAMPISTOL PLANA, Barcelona (ES)

(21) Appl. No.: 19/149,371

(22) PCT Filed: Jan. 19, 2024
(86) PCT No.: PCT/US2024/012281

§ 371 Date: Jul. 18, 2025

(30) Foreign Application Priority Data
Jan. 19, 2023 (EP) 23382042.2

(51) Int. Cl. A01N 1/126; A61K 35/22; A61K 35/407

(52) U.S. Cl. CPC A01N 1/126; A61K 35/22; A61K 35/407

(57) ABSTRACT

Provided are materials and methods for rejuvenating an ex vivo organ prior to transplantation of the organ into a subject in need thereof. The methods and materials can be used to treat, ameliorate, rejuvenate, and/or regenerate organs, e.g., kidneys and livers, that are of old age and/or characterized by organ damage due to an ischemic injury, a toxic injury, a perfusion injury, a hypertensive injury, an immune injury, a physical injury, steatosis, virus induced hepatitis, alcohol, or fibrosis not associated with any known cause. The materials and methods provided can increase the pool of available donor organs, e.g., kidneys and livers. Furthermore, the materials and methods provided can treat an organ that is then self-transplanted.

### REFERENCE TO SEQUENCE LISTING SUBMITTED ELECTRONICALLY VIA EFS-WEB

The content of the electronically submitted sequence listing (Name: 4967_023PC01_Seglisting_ST26.XML; Size: 166,421 bytes; and Date of Creation: Jan. 19, 2024), filed with the application, is incorporated herein by reference in its entirety.

### CROSS REFERENCE TO RELATED APPLICATIONS

The present application claims the priority benefit of EP Application No. 23382042.2, filed Jan. 19, 2023, which is hereby incorporated by reference in their entirety.

### BACKGROUND

At any given moment, tens of thousands of patients in countries around the world sit on waiting-lists for an organ transplant and cannot be served due to a scarcity of suitable organs. In an aging population, the potential number of available older organs rises but insufficient quality of the organs often prevents their use in transplantation. The need is particularly acute for common chronic diseases affecting organs such as kidneys and livers.

Chronic Kidney Disease (CKD) is a global health burden due to aging populations and the high and increasing prevalence of diabetes mellitus and hypertension worldwide. Aging is associated in the kidney with a decline in glomerular filtration rate, increased vascular and interstitial lesions and increased susceptibility to acute kidney injury (e.g., ischemia-reperfusion injury (Weinstein, J. R. & Anderson, S. Advances in Chronic Kidney Disease vol. 17 302-307, 2010)). Transplantation is currently the best replacement therapy for end stage kidney disease, but many patients cannot be transplanted in a timely manner due to organ scarcity. In addition, 20-30% of the organs that are collected from donors are discarded for transplantation due to insufficient quality, increasing the scarcity and delaying life-saving transplantation. The donor organ rejection rate is very high in the U.S, particularly in marginal donors (Aubert et al., JAMA Int. Med. 179(10): 1365-74, 2019). The need for kidneys, as well as other organs, cannot be met and one possibility would be to expand the donor pool to donor organs that are currently considered of insufficient quality, for example, old organs which are often considered of insufficient quality due to hypertension history or other conditions (Ojo, A. O. et al. American Journal of Transplantation vol. 4 27-37, 2004).

Chronic Liver Disease also is a global burden due to aging populations and the high and increasing prevalence of pathologies, such as, hepatitis, hepatocellular carcinoma (HCC), and liver cirrhosis worldwide. Transplantation is currently the best replacement therapy for end stage liver disease and HCC, but many patients cannot be transplanted in a timely manner due to organ scarcity. Expanding the donor pool to donor livers that are currently considered to be of insufficient quality, eg, to older donors by remediating dysfunction in old livers, could address the current donor liver shortage.

Reprogramming of somatic cells to a pluripotent state has been accomplished by overexpressing Yamanaka factors (Oct-3/4, Sox2, Klf4 and c-Myc [OSKM]). However, the reprogramming process is long and complex and full reprogramming can induce tumor development (Takahashi, K. et al. Cell 131, 861-872, 2007; Takahashi, K. & Yamanaka, S. Cell 126, 663-676, 2006; Polo, J. M. et al. Cell 151, 1617-1632, 2012; Abad, M. et al. Nature 502, 340-345, 2013). Therefore, other alternatives to revert the consequences of aging in donor organs are needed.

Partial reprogramming has emerged as an alternative to full reprogramming and a potential treatment of physical effects from aging. However, the challenges of translating reprogramming from the bench to the clinic in general, and for the kidney and organs specifically, need to be addressed. For example, viral vectors when systemically injected do not deliver the therapeutics to the kidney because most of the vectors are taken up by the liver, the infection is generally inefficient, and/or the vector dose is limited by off-target effects (Rubin, J. D. et al. Mol Diagn Ther 24, 375-396, 2020). Therefore, specific therapies that improve transplanted organ function and/or outcome, and/or increase the number of donor organs by remediating dysfunction in organs regarded as suboptimal or poor candidates for transplantation are needed to address the increasing need for donor organs and expand the donor organ pool for transplantation.

### BRIEF SUMMARY

The present disclosure provides an organ perfusate comprising a first polynucleotide encoding at least one regeneration factor and at least one vasodilator. In some aspects, the first polynucleotide encoding the at least one regeneration factor is operably linked to a promoter. In some aspects, the organ perfusate further comprises at least one tonicity agent. In some aspects, the organ perfusate further comprises at least one of a buffer, an inorganic salt, an amino acid, a substrate for metabolism, a hormone, an anti-oxidant, an anti-inflammatory agent, an anti-coagulant, or an anti-microbial agent. In some aspects, the organ perfusate further comprises human serum albumin, dextran, and an extracellular electrolyte composition.

In some aspects, the tonicity agent is selected from the group consisting of dextrose, glycerin, mannitol, potassium chloride, sodium chloride, and combinations thereof. In some aspects, the vasodilator is selected from the group consisting of carbon monoxide, an angiotensin converting enzyme (ACE) inhibitor, an angiotensin receptor blocker, a calcium channel blocker, prostacyclin, hydralazine, minoxidil, nitroglycerin, and combinations thereof.

In some aspects, the organ perfusate further comprises an oxygenation agent. In some aspects, the oxygenation agent is selected from the group consisting of a red blood cell, hemoglobin, pyridoxylated hemoglobin, a synthetic hemoglobin-based oxygen carrier, and combinations thereof. In some aspects, the synthetic hemoglobin-based oxygen carrier is a polymerized hemoglobin-based oxygen carrier, Lifor™, Aquix RS-I, Hemarina®, or Perfluorocarbon.

In some aspects, the organ perfusate further comprises a second polynucleotide encoding at least one regeneration factor operably linked to a promoter.

In some aspects, the promoter of the first polynucleotide and the second polynucleotide are inducible promoters.

In some aspects, the at least one regeneration factor encoded by the first polynucleotide is selected from the group consisting of an Oct family transcription factor, a Sox family transcription factor, a Klf family transcription factor, and a Myc family transcription factor. In some aspects, the at least one regeneration factor encoded by the second polynucleotide is selected from the group consisting of an Oct family transcription factor, a Sox family transcription factor, a Klf family transcription factor, and a Myc family transcription factor. In some aspects, the at least one regeneration factor encoded by the second polynucleotide is different from or not the same regeneration factor encoded by the first polynucleotide. In some aspects, the Oct family transcription factor is selected from the group consisting of Oct1, Oct3, Oct4, Oct6, and variants thereof. In some aspects, the Sox family transcription factor is selected from the group consisting of Sox1, Sox2, Sox3, Sox7, Sox15, Sox17, Sox18, and variants thereof. In some aspects, the Klf family transcription factor is selected from the group consisting of Kfl1, Klf4, Klf5, and variants thereof. In some aspects, the Myc family transcription factor is selected from the group consisting of c-Myc, L-Myc, N-Myc, and variants thereof.

In some aspects, the polynucleotide, the second polynucleotide, or both, are encapsulated in a nanoparticle. In some aspects, the nanoparticle is a lipid nanoparticle, a polymeric nanoparticle, a ligand-conjugated lipid nanoparticle, or a ligand-conjugated polymeric nanoparticle.

In some aspects, the polynucleotide, the second polynucleotide, or both, are present within a viral genome, a plasmid, a minicircle vector, or a transposon. In some aspects, the viral genome is selected from an AAV genome, an adenoviral genome, a retroviral genome, or a lentiviral genome.

In some aspects, the organ perfusate further comprises a regeneration factor-protein transfer domain fusion protein.

In some aspects, the organ perfusate further comprises a regeneration factor polypeptide.

In some aspects, the organ perfusate further comprises an Oct family transcription factor, a Sox family transcription factor, a Klf family transcription factor, and/or a Myc family transcription factor. In some aspects, the Oct family transcription factor is selected from the group consisting of Oct1, Oct3, Oct4, Oct6, and variants thereof. In some aspects, the Sox family transcription factor is selected from the group consisting of Sox1, Sox2, Sox3, Sox7, Sox15, Sox17, Sox18, and variants thereof. In some aspects, the Klf family transcription factor is selected from the group consisting of Kfl1, Klf4, Klf5, and variants thereof. In some aspects, the Myc family transcription factor is selected from the group consisting of c-Myc, L-Myc, N-Myc, and variants thereof.

In some aspects, the organ perfusate further comprises an enhancing agent selected from the group consisting of soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), and combinations thereof.

The present disclosure also provides a method of ex vivo rejuvenation of an organ, the method comprising: providing an organ; contacting the organ ex vivo with a composition comprising a polynucleotide encoding at least one regeneration factor operably linked to an inducible promoter; and adding to the composition a compound that induces the inducible promoter to promote expression of the at least one regeneration factor, whereby the at least one regeneration factor is expressed and the organ is rejuvenated.

In some aspects of the methods described herein, the compound is added intermittently. In some aspects, the intermittent addition comprises adding the compound once per day on two consecutive days and not adding the agent on the following 5 consecutive days. In some aspects, the intermittent addition is performed 2-10 times. In some aspects, the intermittent addition is performed 2-10 times over a period of about 1 week to about 6 weeks.

In some aspects of the methods described herein, the composition further comprises a vasodilator. In some aspects, the composition further comprises a tonicity agent. In some aspects, the composition further comprises at least one of a buffer, an inorganic salt, an amino acid, a substrate for metabolism, a hormone, an anti-oxidant, an anti-inflammatory agent, an anti-coagulant, or an anti-microbial agent.

In some aspects of the methods described herein, the organ is provided in a perfusion system selected from a Hugo-Sachs system, an Organ Assist system, an OrganOX system, a Radnoti system, an ARK Kidney system, and Aferetica PerLife® system.

In some aspects of the methods described herein, the organ is a kidney or a liver.

The present disclosure also provides a method of transplanting an organ into a subject in need thereof, the method comprising: providing an organ; contacting the organ ex vivo with a composition comprising a polynucleotide encoding at least one regeneration factor operably linked to an inducible promoter; adding to the composition a compound that induces the inducible promoter; and transplanting the organ into the subject.

In some aspects of the methods described herein, the compound is added for about 1 minute to about 24 hours to the composition. In some aspects, the compound is added intermittently to the composition. In some aspects, the intermittent addition of the compound comprises adding the compound once per day on two consecutive days and not adding the compound on the following 5 consecutive days. In some aspects, the intermittent addition of the compound is repeated 2-10 times. In some aspects, the intermittent addition is performed 2-10 times over a period of about 1 week to about 6 weeks.

The present disclosure also provides a method of transplanting an organ into a subject in need thereof, the method comprising: providing an organ; contacting the organ ex vivo with a composition comprising a polynucleotide encoding at least one regeneration factor operably linked to an inducible promoter; transplanting the organ into the subject; and administering to the subject a compound that induces the inducible promoter.

In some aspects of the methods described herein, the compound is administered for about 1 minute to about 24 hours to the subject. In some aspects, the compound is administered intermittently to the subject. In some aspects, the intermittent administration of the compound comprises administering the compound once per day on two consecutive days and not administering the compound on the following 5 consecutive days. In some aspects, the intermittent administration of the compound is repeated about 2-10 times. In some aspects, the intermittent administration of the compound is performed about 2-10 times every 3 months. In some aspects, intermittent administration of the compound is performed about 2-10 times every 6 months. In some aspects, the composition further comprises a vasodilator.

In some aspects of the methods described herein, the composition further comprises a tonicity agent. In some aspects, the composition further comprise at least one of a buffer, an inorganic salt, an amino acid, a substrate for metabolism, a hormone, an anti-oxidant, an anti-inflammatory agent, an anti-coagulant, or an anti-microbial agent.

In some aspects of the methods described herein, wherein the explanted organ is provided in a perfusion system selected from a Hugo-Sachs system, an Organ Assist system, an OrganOX system, a Radnoti system, an ARK Kidney system, and Aferetica PerLife® system.

In some aspects of the methods described herein, wherein the organ is a kidney or a liver.

In some aspects of the methods described herein, the method further comprises taking a biopsy from the organ prior to being transplanted.

In some aspects of the methods described herein, the method further comprises taking a biopsy of the organ after being transplanted.

In some aspects of the methods described herein, the method further comprises monitoring the subject who has been transplanted with the organ for organ function. In some aspects, the monitoring comprises measuring one or more of blood urea levels, serum creatinine levels, bilirubin levels, blood pH, blood bicarbonate levels, blood sodium levels, blood potassium levels, or blood lactate levels.

In some aspects of the methods described herein, the method further comprises administering an immunosuppressive medication to the subject.

The present disclosure also provides an ex vivo organ for transplantation to a subject in need thereof, the ex vivo organ comprising: (i) a perfusate; and (ii) a polynucleotide encoding at least one regeneration factor. In some aspects, the ex vivo organ is damaged by at least one of old age, an ischemic injury, a hypertensive injury, a toxic injury, a perfusion injury, an immune injury, a physical injury, steatosis, virus induced hepatitis, alcohol, or a fibrosis not associated with any known cause. In some aspects, the polynucleotide further comprises an inducible promoter operably linked to the polynucleotide encoding the at least one regeneration factor. In some aspects, the perfusate intermittently comprises a compound that induces the inducible promoter.

### DESCRIPTION OF FIGURES

FIG. 1A shows direct injection of AAV vectors into a rat kidney via clamped renal vein and luciferin expression, in the injected and the non-injected (contralateral) kidney 7 days after in situ injection. FIG. 1B shows in vivo luciferin levels in rats transplanted with a kidney perfused with a GFP-Luc AAV vector (animal on the left), a control rat (animal in the middle), and a rat systemically injected (via penile vein) with the GFP-Luc AAV vector (animal on the right).

FIG. 2A shows a schematic of allogenic transplantation of old Dark Agouti kidneys perfused with AAV OSKM vectors, into young Lewis recipient rats treated from day 7 after transplantation for 2 days per week with doxycycline, to induce OSKM expression. FIG. 2B shows H&E staining imagerey of an aged kidney perfused with GFP-AAV, following allogenic transplantation demonstrating immune injury and ischemic injury. FIG. 2C shows the percent survival rates of rats allotransplanted with aged kidneys perfused with GFP and OSKM AAV vectors up to 110 days after transplantation with doxycycline treatments stopped at day 88. FIG. 2D shows BUN levels in rats 2 weeks after transplantation with aged kidneys perfused with GFP and OSKM AAV vectors. FIG. 2E shows levels of serum creatinine (SCr) in rats up to 7 weeks after transplantation with aged kidneys perfused with GFP AAV and OSKM AAV vectors (right panel) compared to rats transplanted with kidneys perfused with GFP AAV vectors (left panel). FIG. 2F shows serum pH, HCO₃, sodium, potassium, lactate, BUN and serum creatinine levels in rats 2 weeks after transplantation with aged kidneys perfused with GFP AAV and OSKM AAV vectors. FIG. 2G shows serum pH, HCO₃, sodium, potassium, lactate, BUN and serum creatinine levels in rats 3 weeks after transplantation with aged kidneys perfused with GFP AAV and OSKM AAV vectors. FIG. 2H shows serum pH, HCO₃, sodium, potassium, lactate, BUN and serum creatinine levels in rats 4 weeks after transplantation with aged kidneys perfused with GFP AAV and OSKM AAV vectors. FIG. 2I shows serum pH, HCO₃, sodium, potassium, lactate, BUN and serum creatinine levels in rats 2-4 weeks after transplantation (1-3 weeks post-nephrectomy) with aged kidneys perfused with GFP AAV and OSKM AAV vectors. FIG. 2J shows BUN levels in rats 2-4 weeks after transplantation (1-3 weeks post-nephrectomy) with aged kidneys perfused with GFP AAV and OSKM AAV vectors. FIG. 2K shows levels of serum creatinine (Cr) in surviving rats up to 15 weeks after transplantation with aged kidneys perfused with GFP AAV and OSKM AAV vectors. FIG. 2L shows BUN levels in rats up to 14 weeks after transplantation with aged kidneys perfused with GFP AAV and OSKM AAV vectors. FIG. 2M shows potassium levels in rats up to 12 weeks after transplantation with aged kidneys perfused with GFP AAV and OSKM AAV vectors. FIG. 2N shows a representative picture of a transplanted kidney from the OSKM group (right) collected 100 days after transplantation and a normal kidney for comparison (left).

FIG. 3A shows a schematic of isogenic transplantation in an ischemic kidney transplant model in which young Lewis rat kidneys perfused with AAV OSKM vectors are transplanted into young Lewis recipient rats treated immediately after transplantation for an extended period with doxycycline for 4 days per week. FIG. 3B shows the survival rates of rats transplanted with kidneys perfused with no AAV control and OSKM AAV vectors. FIG. 3C shows the levels of serum creatinine in control rats transplanted with kidneys perfused with no AAV control (number of surviving animals (n) shown below the graph). FIG. 3D shows the levels of serum creatinine in rats transplanted with kidneys perfused with OSKM AAV (number of surviving animals (n) shown below the graph). FIG. 3E shows the levels of serum creatinine in rats transplanted with kidneys perfused with GFP AAV (square plots on graph) and OSKM AAV (circle plots on graph). FIG. 3F shows the levels of BUN of rats transplanted with kidneys perfused with no AAV control (number of surviving animals (n) shown below the graph). FIG. 3G shows the levels of BUN of rats transplanted with kidneys perfused with OSKM AAV vectors (number of surviving animals (n) shown below the graph). FIG. 3H shows the levels of BUN in rats transplanted with kidneys perfused with no AAV control (large circles) and OSKM AAV (small circles).

FIG. 4 shows a schematic of isogenic transplantation in a cardiac arrest kidney induced nephropathy model, in which young lewis rats are perfused with control (no AAV) or treatment (perfusion in the presence of AAV/DJ-OSK+AAV/DJ cMyc-rtTA).

FIG. 5A shows AST levels in rats 1 week after i.v. injection of PBS (Control), AAV-GFP/Luc control vectors (Vehicle) or AAV-OSK+AAV-cMyc-rtTA treatment vectors (Treatment). FIG. 5B shows ALT levels in rats 1 week after i.v. injection of PBS (Control), AAV-GFP/Luc control vectors (Vehicle) or AAV-OSK+AAV-cMyc-rtTA treatment vectors (Treatment). FIG. 5C shows Caspase 3 levels in rats 1 week after i.v. injection of PBS (Control), AAV-GFP/Luc control vectors (Vehicle) or AAV-OSK+AAV-cMyc-rtTA treatment vectors (Treatment). FIG. 5D shows Bilirubin levels in rats 1 week after i.v. injection of PBS (Control), AAV-GFP/Luc control vectors (Vehicle) or AAV-OSK+AAV-cMyc-rtTA treatment vectors (Treatment). FIG. 5E shows MPO levels in rats 1 week after i.v. injection of PBS (Control), AAV-GFP/Luc control vectors (Vehicle) or AAV-OSK+AAV-cMyc-rtTA treatment vectors (Treatment). FIG. 5F shows MDA levels in rats 1 week after i.v. injection of PBS (Control), AAV-GFP/Luc control vectors (Vehicle) or AAV-OSK+AAV-cMyc-rtTA treatment vectors (Treatment). FIG. 5G shows Ki67 levels in rats 1 week after i.v. injection of PBS (Control), AAV-GFP/Luc control vectors (Vehicle) or AAV-OSK+AAV-cMyc-rtTA treatment vectors (Treatment). FIG. 5H shows PCNA levels in rats 1 week after i.v. injection of PBS (Control), AAV-GFP/Luc control vectors (Vehicle) or AAV-OSK+AAV-cMyc-rtTA treatment vectors (Treatment). FIG. 5I shows HGF levels in rats 1 week after i.v. injection of PBS (Control), AAV-GFP/Luc control vectors (Vehicle) or AAV-OSK+AAV-cMyc-rtTA treatment vectors (Treatment). FIG. 5J shows TGF-β levels in rats 1 week after i.v. injection of PBS (Control), AAV-GFP/Luc control vectors (Vehicle) or AAV-OSK+AAV-cMyc-rtTA treatment vectors (Treatment). FIG. 5K shows a representative H&E image of a liver 1 week after i.v. injection of PBS (Control). FIG. 5L shows an H&E image of a liver 1 week after i.v. injection of AAV-GFP/Luc control vectors (Vehicle). FIG. 5M shows an H&E image of a liver 1 week after i.v. injection of AAV-OSK+AAV-cMyc-rtTA treatment vectors (Treatment).

FIG. 6A shows AST levels in rats 12 weeks after i.v. injection of PBS (Control), AAV-GFP/Luc control vectors (Vehicle) or AAV-OSK+AAV-cMyc-rtTA treatment vectors (Treatment). FIG. 6B shows ALT levels in rats 12 weeks after i.v. injection of PBS (Control), AAV-GFP/Luc control vectors (Vehicle) or AAV-OSK+AAV-cMyc-rtTA treatment vectors (Treatment). FIG. 6C shows Caspase 3 levels in rats 12 weeks after i.v. injection of PBS (Control), AAV-GFP/Luc control vectors (Vehicle) or AAV-OSK+AAV-cMyc-rtTA treatment vectors (Treatment). FIG. 6D shows Bilirubin levels in rats 12 weeks after i.v. injection of PBS (Control), AAV-GFP/Luc control vectors (Vehicle) or AAV-OSK+AAV-cMyc-rtTA treatment vectors (Treatment). FIG. 6E shows MPO levels in rats 12 weeks after i.v. injection of PBS (Control), AAV-GFP/Luc control vectors (Vehicle) or AAV-OSK+AAV-cMyc-rtTA treatment vectors (Treatment). FIG. 6F shows MDA levels in rats 12 weeks after i.v. injection of PBS (Control), AAV-GFP/Luc control vectors (Vehicle) or AAV-OSK+AAV-cMyc-rtTA treatment vectors (Treatment). FIG. 6G shows Ki67 levels in rats 12 weeks after i.v. injection of PBS (Control), AAV-GFP/Luc control vectors (Vehicle) or AAV-OSK+AAV-cMyc-rtTA treatment vectors (Treatment). FIG. 6H shows PCNA levels in rats 12 weeks after i.v. injection of PBS (Control), AAV-GFP/Luc control vectors (Vehicle) or AAV-OSK+AAV-cMyc-rtTA treatment vectors (Treatment). FIG. 6I shows HGF levels in rats 12 weeks after i.v. injection of PBS (Control), AAV-GFP/Luc control vectors (Vehicle) or AAV-OSK+AAV-cMyc-rtTA treatment vectors (Treatment). FIG. 6J shows TGF-β levels in rats 12 weeks after i.v. injection of PBS (Control), AAV-GFP/Luc control vectors (Vehicle) or AAV-OSK+AAV-cMyc-rtTA treatment vectors (Treatment). FIG. 6K shows a representative H&E image of a liver 12 weeks after i.v. injection of PBS (Control). FIG. 6L shows an H&E image of a liver 12 weeks after i.v. injection of AAV-GFP/Luc control vectors (Vehicle). FIG. 6M shows an H&E image of a liver 12 weeks after i.v. injection of AAV-OSK+AAV-cMyc-rtTA treatment vectors (Treatment).

FIG. 7 shows a 10× view of representative sections of hemoxylin-eosin stained OSKM-treated kidneys.

FIG. 8 shows a 10× view of representative sections of hemoxylin-eosin stained kidneys treated with the AAV-GFP/luc control.

FIG. 9A shows BUN levels in rats two weeks after transplantation in the ischemic injury model with kidneys perfused with OSKM-AAV, or no-AAV control. FIG. 9B shows BUN levels in rats three weeks after transplantation in the ischemic injury model with kidneys perfused with OSKM-AAV, or no-AAV control. FIG. 9C shows serum creatine levels in rats two weeks after transplantation in the ischemic injury model with kidneys perfused with OSKM-AAV, or no-AAV control. FIG. 9D shows serum creatine levels in rats three weeks after transplantation in the ischemic injury model with kidneys perfused with OSKM-AAV, or no-AAV control.

FIG. 10A shows the levels of BUN in rats transplanted with kidneys perfused with no-AAV control (no AAV vector)(n=the number of surviving animals). FIG. 10B shows the levels of BUN in rats transplanted with kidneys perfused with OSKM AAV vectors (n=the number of surviving animals). FIG. 10C shows the levels of serum creatinine in rats transplanted with kidneys perfused with no-AAV control (solid circles) and OSKM AAV (hollow circles).

FIG. 11 shows a rat model of segmental (70%) hepatic ischemia.

FIG. 12A shows AST levels one week after administration of PBS control, vehicle control (AAV-GFP/Luc+AAV-rtTA), or AAV-OSKM (AAV-OSK+AAV cMyc/rtTA). FIG. 12B shows ALT levels one week after administration of PBS control, vehicle control (AAV-GFP/Luc+AAV-rtTA) or AAV-OSKM (AAV-OSK+AAV cMyc/rtTA). FIG. 12C shows bilirubin levels one week after administration of PBS control, vehicle control (AAV-GFP/Luc+AAV-rtTA) or AAV-OSKM (AAV-OSK+AAV cMyc/rtTA). FIG. 12D shows caspase 3 levels one week after administration of PBS control, vehicle control (AAV-GFP/Luc+AAV-rtTA) or AAV-OSKM (AAV-OSK+AAV cMyc/rtTA). FIG. 12E shows MPO levels one week after administration of PBS control, vehicle control (AAV-GFP/Luc+AAV-rtTA) or AAV-OSKM (AAV-OSK+AAV cMyc/rtTA). FIG. 12F shows MDA levels one week after administration of PBS control, vehicle control (AAV-GFP/Luc+AAV-rtTA) or AAV-OSKM (AAV-OSK+AAV cMyc/rtTA). FIG. 12G shows PCNA levels one week after administration of PBS control, vehicle control (AAV-GFP/Luc+AAV-rtTA) or AAV-OSKM (AAV-OSK+AAV cMyc/rtTA). FIG. 12H shows ki67 levels one week after administration of PBS control, vehicle control (AAV-GFP/Luc+AAV-rtTA) or AAV-OSKM (AAV-OSK+AAV cMyc/rtTA).

FIG. 13A shows AST levels four days after administration of PBS control, vehicle control, (AAV-GFP/Luc+AAV-rtTA) or AAV-OSKM (AAV-OSK+AAV cMyc/rtTA). FIG. 13B shows ALT levels four days after administration of PBS control, vehicle control (AAV-GFP/Luc+AAV-rtTA) or AAV-OSKM (AAV-OSK+AAV cMyc/rtTA). FIG. 13C shows caspase 3 levels four days after administration of PBS control, vehicle control (AAV-GFP/Luc+AAV-rtTA) or AAV-OSKM (AAV-OSK+AAV cMyc/rtTA). FIG. 13D shows MPO levels four days after administration of PBS control, vehicle control (AAV-GFP/Luc+AAV-rtTA) or AAV-OSKM (AAV-OSK+AAV cMyc/rtTA). FIG. 13E shows MDA levels four days after administration of PBS control, vehicle control (AAV-GFP/Luc+AAV-rtTA) or AAV-OSKM (AAV-OSK+AAV cMyc/rtTA). FIG. 13F shows PCNA levels four days after administration of PBS control, vehicle control (AAV-GFP/Luc+AAV-rtTA) or AAV-OSKM (AAV-OSK+AAV cMyc/rtTA). FIG. 13G shows ki67 levels four days after administration of PBS control, vehicle control (AAV-GFP/Luc+AAV-rtTA) or AAV-OSKM (AAV-OSK+AAV cMyc/rtTA). FIG. 13H shows HGF levels four days after administration of PBS control, vehicle control (AAV-GFP/Luc+AAV-rtTA) or AAV-OSKM (AAV-OSK+AAV cMyc/rtTA). FIG. 13I shows TGF-β levels four days after administration of PBS control, vehicle control (AAV-GFP/Luc+AAV-rtTA) or AAV-OSKM (AAV-OSK+AAV cMyc/rtTA).

FIG. 14A shows AST levels two days after administration of PBS control, vehicle control, (AAV-GFP/Luc+AAV-rtTA) or AAV-OSKM (AAV-OSK+AAV cMyc/rtTA). FIG. 14B shows ALT levels two days after administration of PBS control, vehicle control (AAV-GFP/Luc+AAV-rtTA) or AAV-OSKM (AAV-OSK+AAV cMyc/rtTA). FIG. 14C shows caspase 3 levels two days after administration of PBS control, vehicle control (AAV-GFP/Luc+AAV-rtTA) or AAV-OSKM (AAV-OSK+ AAV cMyc/rtTA). FIG. 14D shows MPO levels two days after administration of PBS control, vehicle control (AAV-GFP/Luc+AAV-rtTA) or AAV-OSKM (AAV-OSK+AAV cMyc/rtTA). FIG. 14E shows MDA levels two days after administration of PBS control, vehicle control (AAV-GFP/Luc+AAV-rtTA) or AAV-OSKM (AAV-OSK+AAV cMyc/rtTA). FIG. 14F shows PCNA levels two days after administration of PBS control, vehicle control (AAV-GFP/Luc+AAV-rtTA) or AAV-OSKM (AAV-OSK+AAV cMyc/rtTA). FIG. 14G shows ki67 levels two days after administration of PBS control, vehicle control (AAV-GFP/Luc+AAV-rtTA) or AAV-OSKM (AAV-OSK+AAV cMyc/rtTA). FIG. 14H shows HGF levels two days after administration of PBS control, vehicle control (AAV-GFP/Luc+AAV-rtTA) or AAV-OSKM (AAV-OSK+AAV cMyc/rtTA). FIG. 14I shows TGF-β levels two days after administration of PBS control, vehicle control (AAV-GFP/Luc+AAV-rtTA) or AAV-OSKM (AAV-OSK+AAV cMyc/rtTA).

FIG. 15A shows levels of mean serum creatinine (SCr) changes in both control and treated groups of rats 8 days after transplantation. FIG. 15B shows levels of mean BUN changes in rats 8 days after transplantation. FIG. 15C shows individual urine protein (left panel) and urine albumin (right panel) in both control and treated groups of rats after transplantation. FIG. 15D shows flow and resistance registries during normothermic kidney perfusion.

FIG. 16A shows the expression profile of Neutrophil Gelatinase-Associated Lipocalin (NGAL) in control and OSKM treated kidneys. FIG. 16B shows the expression profile of Hypoxia-Inducible Factor (HIF) in control and OSKM treated kidneys. FIG. 16C shows the expression profile of Endothelin Receptor (ETR) in control and OSKM treated kidneys. FIG. 16D shows the expression profile of Transforming Growth Factor-ß (TGFß) in control and OSKM treated kidneys. FIG. 16E shows the expression profile of Tumor Necrosis Factor (TNF) in control and OSKM treated kidneys. FIG. 16F shows the expression profile of Monocyte Chemoattractant Protein 1 (MCP1) in control and OSKM treated kidneys. FIG. 16G shows the expression profile of C-X-C motif chemokine ligand 10 (CXCL10) in control and OSKM treated kidneys. FIG. 16H shows the expression profile of the evaluated genes in control and OSKM treated kidneys.

FIG. 17A shows the hematoxylin and eosin kidney staining of glomerular compartments at 4× showing tubular and glomerular compartments at 4×, with no AAV administration. FIG. 17B shows the hematoxylin and eosin kidney staining of glomerular compartments at 4× showing tubular and glomerular compartments at 4×, with AAV-OSKM administration. FIG. 17C shows the hematoxylin and eosin kidney staining of glomerular compartments at 4× showing tubular and glomerular compartments at ×10, with no AAV administration. FIG. 17D shows the hematoxylin and eosin kidney staining of glomerular compartments at 4× showing tubular and glomerular compartments at 4×, with AAV-OSKM administration. FIG. 17E shows the Masson's Trichrome kidney staining of tubular and glomerular compartments at 4×, with no AAV administration. FIG. 17F shows the Masson's Trichrome kidney staining of tubular and glomerular compartments 4×, with AAV-OSKM administration.

FIG. 18 shows the modified Remuzzi score analysis of OSKM-treated and control-treated samples.

### DETAILED DESCRIPTION

Provided are materials and methods for the rejuvenation of organs to improve organ function and/or organ transplantation outcome to increase the number of donor organs by rendering organs regarded as suboptimal or poor candidates for transplantation suitable for transplantation. Also provided are materials and methods for the rejuvenation of organs to improve organ function and/or organ transplantation outcome for self-transplantation (i.e., rejuvenation of a subject's organ). The materials described herein include, but are not limited to, polynucleotides, vectors, and compositions that are introduced into a donor organ ex vivo, e.g., in an organ perfusate. The materials and methods enable expression of regeneration factors in the perfused organ such that, e.g., age-related changes can be ameliorated and aged or injured organs treated to improve suitability for transplantation.

The methods and materials described herein can be used for ex vivo perfusion of a human kidney to revert changes associated with at least one of old age, an ischemic injury, a hypertensive injury, a toxic injury, a perfusion injury, an immune injury, a physical injury, steatosis, virus induced hepatitis, alcohol, or fibrosis not associated with any known cause in the kidney prior to transplanting the kidney into a human recipient. The methods and materials described herein can also be used for ex vivo perfusion of a human liver to revert changes associated with at least one of old age, an ischemic injury, a hypertensive injury, a toxic injury, a perfusion injury, an immune injury, physical injury, steatosis, virus induced hepatitis, alcohol, or fibrosis not associated with a known cause in the liver prior to transplanting the liver into a human recipient. Advantageously, the methods and materials provided herein allow a treatment of a kidney or liver by ex vivo perfusion with a perfusate comprising the materials described herein, followed by measuring the effects of the materials on the kidney or liver, e.g., by biopsy and functional assessment of the kidney or liver, and, if desired, one or more further treatments of the kidney or liver with the methods and materials as described herein prior to transplanting the kidney or liver into a recipient patient in need thereof.

Furthermore, the methods and materials as used herein allow a treatment of a kidney or liver in vivo after the kidney or liver has been perfused ex vivo with a perfusate comprising the materials described herein to regulate the effect of the materials on the kidney or liver in vivo. Measurement, e.g., by biopsy or functional assessments can be obtained from the transplanted patient and the kidney or liver can be treated, based on the results of these measurements, to enhance and/or sustain an effect of the materials provided to the kidney or liver during the ex vivo perfusion prior to transplantation.

### I. Definitions

In order that the present disclosure can be more readily understood, certain terms are first defined. Additional definitions are set forth throughout the detailed disclosure.

It is to be noted that the term “a” or “an” entity refers to one or more of that entity; for example, “a polynucleotide sequence,” is understood to represent one or more polynucleotide sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a polynucleotide molecule must be an integer. For example, “at least 18 nucleotides of a 21-nucleotide polynucleotide molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

Additionally, any reference referred to as being “incorporated herein” is to be understood as being incorporated in its entirety.

The terms “polynucleotide,” “polynucleotide,” and “oligonucleotide,” are used interchangeably in the present application. These terms refer only to the primary structure of the molecule. Thus, these terms include double- and single-stranded DNA, as well as double- and single-stranded RNA. The terms “polynucleotide,” “polynucleotide,” and “oligonucleotide,” as used herein, are defined as it is generally understood by the skilled person as a molecule comprising two or more covalently linked nucleosides. Oligonucleotide can also be referred to as oligomers. Oligonucleotides are commonly made synthetically in the laboratory by solid-phase chemical synthesis followed by purification. Polynucleotides can be made recombinantly, enzymatically, or synthetically, e.g., by solid-phase chemical synthesis followed by purification. When referring to a sequence of an oligonucleotide, polynucleotide or polynucleotide, reference is made to the sequence or order of nucleobase moieties, or modifications thereof, of the covalently linked nucleotides or nucleosides.

A “nucleotide,” as used herein, refers to a monomeric unit of an oligonucleotide or polynucleotide that comprises a nucleoside and an internucleosidic linkage. “G,” “C,” “A,” “T,” and “U” each generally stand for a naturally-occurring nucleotide that contains guanine, cytosine, adenine, thymidine, and uracil as a nucleobase, respectively. However, it will be understood that the term “nucleotide” can refer to an alternative nucleotide, as further detailed below, or a surrogate replacement moiety. An oligonucleotide or polynucleotide is not limited to naturally occurring nucleosides but can contain non-natural nucleoside and linkages as disclosed herein, e.g., to produce an oligonucleotide that is modified to enhance its stability or cell permeability. The skilled person is well aware that guanine, cytosine, adenine, and uracil can be replaced by other moieties without substantially impairing the base-pairing properties of an oligonucleotide or polynucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of oligonucleotides or polynucleotides by a nucleotide containing, for example, inosine. In another example, adenine and cytosine anywhere in the oligonucleotide or polynucleotide can be replaced with guanine and uracil, respectively to form G-U Wobble base pairing with a target RNA. Sequences containing such replacement moieties are suitable for the compositions and methods featured herein.

The terms “nucleobase” and “base,” are used herein interchangeably and include the purine (e.g. adenine and guanine) and pyrimidine (e.g. uracil, thymine, and cytosine) moiety present in nucleosides and nucleotides which form hydrogen bonds in polynucleotide hybridization. The term nucleobase also encompasses alternative nucleobases which can differ from naturally-occurring nucleobases, but are functional during polynucleotide hybridization. In this context “nucleobase” refers to both naturally occurring nucleobases such as adenine, guanine, cytosine, thymidine, uracil, xanthine, and hypoxanthine, as well as alternative nucleobases. Such variants are for example described in Hirao et al (2012) Accounts of Chemical Research vol 45 page 2055 and Bergstrom (2009) Current Protocols in Polynucleotide Chemistry Suppl. 37 1.4.1. Alternative nucleobases include modified purines or pyrimidines. For example, adenine and guanine may be substituted with other naturally-occurring purines, including but not limited to N⁶-methyladenine, N²-methylguanine, hypoxanthine, and 7-methylguanine. Cytosine, uracil, and thymine may be substituted with other naturally-occurring pyrimidines, including but not limited to 5-methylcytosine, 5-hydroxymethylcytosine, pseudouracil, and 4-thiouracil. In some aspects, an oligomer may contain thymine bases in place of uracil. Other alternative nucleobases include isocytosine, pseudoisocytosine, 5-thiozolo-cytosine, 5-propynyl-cytosine, 5-propynyl-uridine, 5-bromouridine 5-thiazolo-uridine, 2-thio-uridine, pseudouridine, 1-methylpseudouridine, 5-methoxyuridine, inosine, diaminopurine, 6-aminopurine, 2-aminopurine, 2,6-diaminopurine, 2-chloro-6-aminopurine, agmatidine, lysidine, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-aminomethylcytosine, 5-hydroxymethylcytosine, 5-hydroxybutynl-2′-deoxyuridine), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, 8-aza-7-deazaguanosine, and N4-ethylcytosine, or derivatives thereof; N²-cyclopentylguanine (cPent-G), N²-cyclopentyl-2-aminopurine (cPent-AP), and N²-propyl-2-aminopurine (Pr-AP), pseudouracil or derivatives thereof, and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)).

Certain modified or substituted nucleobases are particularly useful for increasing the binding affinity of an antisense oligonucleotide. These include 5-substituted pyrimidines, 6-azapyrimidines and N-2, N-6 and 0-6 substituted purines, including 2-aminopropyladenine, 5-propynyluracil, 5-propynylcytosine, and 5-methylcytosine.

The term “nucleoside” refers to a monomeric unit of an oligonucleotide or a polynucleotide having a nucleobase and a sugar moiety. A nucleoside can include those that are naturally-occurring as well as alternative nucleosides, such as those described herein. The nucleobase of a nucleoside can be a naturally-occurring nucleobase or an alternative nucleobase. Similarly, the sugar moiety of a nucleoside can be a naturally-occurring sugar or an alternative sugar.

The term “alternative nucleoside” refers to a nucleoside having an alternative sugar or an alternative nucleobase, such as those described herein.

A “sugar” or “sugar moiety,” includes naturally occurring sugars having a furanose ring. A sugar also includes an “alternative sugar,” defined as a structure that is capable of replacing the furanose ring of a nucleoside. In some aspects, alternative sugars are non-furanose (or 4′-substituted furanose) rings or ring systems or open systems. Alternative sugars can include sugar surrogates wherein the furanose ring has been replaced with another ring system such as, for example, a morpholino or hexitol ring system. Sugar moieties useful in the preparation of oligonucleotides having motifs include, without limitation, β-D-ribose, β-D-2′-deoxyribose, substituted sugars (such as 2′, 5′ and bis substituted sugars), 4′-S-sugars (such as 4′-S-ribose, 4′-S-2′-deoxyribose and 4′-S-2′-substituted ribose), bicyclic alternative sugars (such as the 2′-OCH₂-4′ or 2′-O—(CH₂)₂-4′ bridged ribose derived bicyclic sugars) and sugar surrogates (such as when the ribose ring has been replaced with a morpholino or a hexitol ring system). The type of heterocyclic base and internucleoside linkage used at each position is variable and is not a factor in determining the motif. In most nucleosides having an alternative sugar moiety, the heterocyclic nucleobase is generally maintained to permit hybridization. In some aspects, an oligonucleotide or polynucleotide contains nucleosides with, e.g., 2′FANA modification and nucleosides without 2′ FANA modification, which nucleosides may contain alternative sugars as describe herein.

An “internucleosidic linkage” refers to a linkage between nucleosides in an oligonucleotide or polynucleotide, which linkage can include a phosphate linkage or an alternative internucleosidic linkage. Many “alternative internucleosidic linkages” or “non-natural linkages” are known in the art, including, but not limited to, phosphate, phosphorothioate, and boronophosphate linkages. Alternative nucleosides include bicyclic nucleosides (BNAs) (e.g., locked nucleosides (LNAs) and constrained ethyl (cEt) nucleosides), peptide nucleosides (PNAs), phosphotriesters, phosphorothionates, phosphoramidates, and other variants of the phosphate backbone of native nucleoside, including those described herein.

An “alternative nucleotide,” as used herein, refers to a nucleotide having an alternative nucleoside or an alternative sugar, and an internucleoside linkage, which can include alternative nucleoside linkages.

Oligonucleotide or polynucleotides can include (i) compounds that have one or more 2′ FANA modified nucleosides, (ii) compounds that have one or more furanose moieties that are replaced by furanose derivatives or by any structure, cyclic or acyclic, that can be used as a point of covalent attachment for the base moiety, (iii) compounds that have one or more phosphodiester linkages that are either modified, as in the case of phosphoramidate or phosphorothioate linkages, or completely replaced by a suitable linking moiety as in the case of formacetal or riboacetal linkages, and/or (iv) compounds that have one or more linked furanose-phosphodiester linkage moieties replaced by any structure, cyclic or acyclic, that can be used as a point of covalent attachment for the base moiety. An oligonucleotide or polynucleotide can comprise one or more alternative nucleosides or nucleotides (e.g., including those described herein). It is also understood that oligonucleotide includes compositions lacking a sugar moiety or nucleobase but are still capable of forming a pairing with or hybridizing to a target sequence. Oligonucleotides include short polynucleotides (e.g., of 100 or fewer linked nucleosides). “Chimeric” oligonucleotides or “chimeras,” as used herein, are oligonucleotides which contain two or more chemically distinct regions, each made up of at least one monomer unit, i.e., a nucleotide or nucleoside in the case of an oligonucleotide. Chimeric oligonucleotides also include “gapmers.” For example, chimeric oligonucleotides can contain unmodified nucleosides and 2′ FANA modified nucleosides. In some aspects, the 2′ FANA modified nucleosides are located at a 5′ or a 3′ portion, or both, of an oligonucleotide. In some aspects, the 2′ FANA modified nucleosides are located throughout an oligonucleotide. In some aspects, a chimeric oligonucleotide comprises 2′ FANA modified nucleosides located at a 5′ and/or a 3′ portion and at least one unmodified nucleoside in the center of the oligonucleotide.

The term “complementary,” as used herein and unless otherwise indicated, is used to describe a first nucleotide or nucleoside sequence in relation to a second nucleotide or nucleoside sequence and refers to the ability of an oligonucleotide or polynucleotide comprising the first nucleotide or nucleoside sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can be used. The skilled person will be able to determine the set of conditions most appropriate for a test of complementarity of two sequences in accordance with the ultimate application of the hybridized nucleotides or nucleosides. Complementary sequences can include, or be formed entirely from, non-Watson-Crick base pairs and/or base pairs formed from non-natural and alternative nucleotides or nucleosides, in so far as the above requirements with respect to their ability to hybridize are fulfilled. Such non-Watson-Crick base pairs include, but are not limited to, G:U Wobble or Hoogstein base pairing. Complementary sequences between an oligonucleotide and a target sequence as described herein include base-pairing of the oligonucleotide or polynucleotide comprising a first nucleotide or nucleoside sequence to an oligonucleotide or polynucleotide comprising a second nucleotide or nucleoside sequence over the entire length of one or both nucleotide or nucleoside sequences. Such sequences can be referred to as “fully complementary” with respect to each other herein. Complementary sequences between an oligonucleotide or polynucleotide and a target sequence as described herein include base-pairing of the oligonucleotide or polynucleotide comprising a first nucleotide or nucleoside sequence to an oligonucleotide or polynucleotide comprising a second nucleotide or nucleoside sequence over less than the entire length of one or both nucleotide or nucleoside sequences. Such sequences can be referred to as “partially complementary” with respect to each other herein. In some aspects, the oligonucleotide or polynucleotide comprises a contiguous nucleotide region and can comprise further nucleotide(s) or nucleoside(s). For example, a nucleotide linker region which can be used to attach a functional group to the contiguous nucleotide sequence. The nucleotide linker region can be complementary to the target polynucleotide. In some aspects the internucleoside linkages present between the nucleotides of the contiguous nucleotide region are all phosphorothioate internucleoside linkages. In some aspects, the contiguous nucleotide region comprises one or more sugar-modified nucleosides.

The terms “linker” or “linking group,” as used herein refer to a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety to an oligonucleotide or polynucleotide. In some aspects, the conjugate, oligonucleotide conjugate, or polynucleotide conjugate can comprise a linker region which is positioned between the oligonucleotide or polynucleotide and the conjugate moiety. In some aspects, the linker between the conjugate and oligonucleotide or polynucleotide is biocleavable. Phosphodiester containing biocleavable linkers are described in more detail in WO 2014/076195 (herein incorporated by reference).

The term “promoter,” as used herein, refers to a DNA sequence recognized by the machinery of a cell, or introduced synthetic machinery, required to initiate the specific transcription of a gene. The term “promoter” is also meant to encompass those polynucleotide elements sufficient for promoter-dependent gene expression controllable for cell-type specific, tissue-specific or inducible expression by external signals or agents; such elements can be located in the 5′ or 3′ regions of the native gene. In some aspects, the promoter can be a constitutively active promoter, a cell-type specific promoter, or an inducible promoter.

The terms “operatively linked,” “operatively inserted,” “operatively positioned,” “operably linked,” “under control,” or “under transcriptional control,” as used herein mean that a promoter is in the correct location and orientation in relation to a polynucleotide to control RNA polymerase initiation and expression of a gene. The terms “operatively linked” or “operably linked” mean that a polynucleotide sequence and a regulatory sequence(s) are connected in such a way as to permit RNA expression when the appropriate molecules (e.g., transcriptional activator proteins) are bound to the regulatory sequence(s). The term “operatively inserted” or “operably inserted” means that the polynucleotide of interest introduced into a cell is positioned adjacent a polynucleotide sequence which directs transcription and translation of the introduced polynucleotide (i.e., facilitates the production of, e.g., a polypeptide encoded by a DNA of interest).

The term “inducible promoter,” as used herein, refers to a promoter that is operatively or operably linked to a polynucleotide sequence in such a way as to permit RNA expression when the appropriate molecules (e.g., transcriptional activator proteins) are bound to the promoter sequence(s). In some aspects, a transcriptional activator protein is activated when contacted with an activating agent. Examples of inducible promoters include, but are not limited to, tetracycline-inducible promoters, hormone inducible promoters, e.g. tamoxifen inducible promoters (promoter binding domain fused to an estrogen receptor), steroid-inducible promoters (promoter binding domain fused to a mutant progesterone receptor that binds RU486 but not endogenous progesterone), or coumermycine-inducible promoters. An inducible promoter may comprise a tetracycline-responsive element (TRE) (e.g., a TRE3G promoter, a TRE2 promoter, or a P tight promoter). An inducible promoter may also comprise a mifepristone-responsive promoter (e.g., comprising GAL4-upstream activating sequences), or a coumermycin/novobiocin-inducible/repressable promoter. As an example, a TRE (e.g., TRE3G) promoter may comprise a nucleic acid (e.g., engineered nucleic acid) sequence that is at least 70% (e.g., at least 75%, 80%, 85%, 90%, 95%, 98%, 99%, or 100%) identical to SEQ ID NO: 7.

The term “tissue-specific promoter,” as used herein, refers to any promoter that activates a higher transcription of a polynucleotide in a specific tissue compared to other tissues. For example, a tissue-specific promoter used in the polynucleotides described herein can specifically induce transcription in endothelial cells (e.g., Tie-2 promoter); smooth muscle cells (e.g., α-smooth muscle actin promoter, smooth muscle myosin heavy chain promoter, calponin promoter, transgelin promoter, h-caldesmon promoter, smoothelin promoter); pericytes (e.g. p75 promoter, endosialin promoter, desmin promoter); renal tubular epithelial cells (e.g., sodium-dependent phosphate cotransporter gene NPT2a promoter, sodium-potassium-2-chloride cotransporter promoter, or aquaporin 2 promoter).

The term “senescent-cell specific promoter,” as used herein, refers to a promoter that is expressed in a senescent cell but not, or in a significantly reduced manner, in a non-senescent cell. Examples of senescent-cell specific promoters include, but are not limited to, a p16 promoter, a p21 promoter, a mir146a promoter, an ATF3 promoter, GADD45b protmoer, MMP13 promoter, or BTG2 promoter.

The term “WPRE,” as used herein, refers to a Woodchuck Hepatitis Virus Post-translational Regulatory Element that is a DNA sequence that, when transcribed, creates a tertiary structure enhancing expression of genes of a viral vector.

The term “IRES,” as used herein, refers to an element that promotes direct internal ribosome entry to the initiation codon, such as ATG, of a cistron (a protein encoding region), thereby leading to the cap-independent translation of the gene. See, e.g., Jackson R J et al., Trends Biochem Sci 15(12):477-83 (199); Jackson R J and Kaminski, A. RNA 1(10):985-1000 (1995). Under translational control of an IRES translation proceeds in a cap-independent manner.

The term “termination signal sequence,” as used herein, can be any genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “polyA site,” i.e., a site on a RNA transcript to which adenine residues will be added by post-transcriptional polyadenylation.

The term “proteolytic cleavable site,” as used herein, refers to a polynucleotide encoding an amino acid sequence that can be proteolytically cleaved and includes, but is not limited to, a self-processing cleavage site and a furin cleavage site.

The term “self-processing cleavage site,” as used herein, refers to a post-translational or co-translational processing cleavage site or sequence that can be a DNA or amino acid sequence, exemplified herein by a 2A site, sequence or domain or a 2A-like site, sequence or domain. The self-processing peptide is the peptide expression product of the DNA sequence that encodes a self-processing cleavage site or sequence, which upon translation, mediates rapid intramolecular (cis) cleavage of a protein or polypeptide comprising the self-processing cleavage site to yield discrete mature protein or polypeptide products.

The term “furin cleavage site,” as used herein, refers to a polynucleotide encoding an amino acid sequence that can be cleaved by endogenous subtilisin-like proteases, such as furin and other serine proteases within the protein secretion pathway. In some aspects, the furin cleavage site includes a consensus sequence RXK(R)R of SEQ ID NO: 17.

The term “vector,” as used herein, refers to any vehicle for the cloning of and/or transfer of a polynucleotide into a host cell, such as a plasmid, phage, transposon, minicircle vector, cosmid, chromosome, artificial chromosome, virus, virion, etc. A vector can be a replicon to which another polynucleotide segment can be attached so as to bring about the replication of the attached segment. A “replicon” refers to any genetic element (e.g., plasmid, phage, cosmid, chromosome, virus) that functions as an autonomous unit of replication in vivo, i.e., is capable of replication under its own control. The term “vector” includes both viral and nonviral vehicles for introducing a polynucleotide into a cell in vitro, ex vivo or in vivo. Vectors useful for the aspects described herein include, but are not limited to, plasmids, modified eukaryotic viruses, or modified bacterial viruses. In some aspects, insertion of a polynucleotide into a suitable vector can be accomplished by ligating the appropriate polynucleotide fragments into a chosen vector that has complementary cohesive termini. Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters. In some aspects, the delivery vector is selected from the group consisting of a viral vector (e.g., an AAV vector), a plasmid, a lipid, a cationic polymer, a protein particle, a bacterial vector, and a lysosome. Some aspects of the disclosure are directed to biological vectors, which can include viruses, particularly attenuated and/or replication-deficient viruses. In some aspects, a vector can include microRNA targeting sequences to increase specificity of vector-mediated transgene expression. In some aspects, the delivery vector of the disclosure is a viral vector selected from the group consisting of an adeno-associated viral (AAV) vector, an adenoviral vector, a lentiviral vector, a retroviral vector, poxvirus vector, a baculovirus vector, a herpes viral vector, simian virus 40 (SV40), cytomegalovirus (CMV), mouse mammary tumor virus (MMTV), and Moloney murine leukemia virus.

The term “retroviral vector,” as used herein, refers to any vector that comprises or derives from components of a retroviral vector and is suitable to infect mammalian cells, preferably human cells. The term retroviral vector typically designates a retroviral particle or virion or virus comprising a payload. The terms “retroviral genome” and “retroviral vector” can be used interchangeably and refer to a retroviral nucleic acid into which nucleic acids of interest have been inserted in place of certain viral sequences to produce a virus that is replication defective. In order to package a retroviral genome or retroviral vector into a retroviral particle, a packaging cell line expressing gag, pol, and env genes but without a retroviral long terminal repeat (LTR) may be provided. Retroviral vectors have the ability to integrate their genes into a host genome, thereby transferring a large amount of foreign genetic material to the host genome.

The term “retroviral particle,” as used herein, refers to a retrovirus that comprises a capsid and a retroviral vector having at least one payload region (e.g., a polynucleotide encoding at least one regeneration factor) and at least one LTR. In some aspects, the retroviral particle is pseudo-typed by combining a retroviral vector with a heterologous viral envelope protein. In some aspects, the heterologous viral envelope protein is a naturally occurring envelope protein, e.g., from a vescular stomatitits virus. In some aspects, the heterologous viral envelope protein is an engineered viral envelope protein, e.g., to target a specific cell type. In some aspects, a retroviral vector comprises a polynucleotide encoding a regeneration factor. In some aspects, a retroviral vector comprises a polynucleotide encoding an Oct4 protein, Sox2 protein, Klf4 protein and/or a c-Myc protein. In some aspects, a retroviral vector comprises a polynucleotide comprising a polycistronic cassette encoding an Oct4 protein, Sox2 protein, Klf4 protein and a c-Myc protein. In some aspects, a retroviral vector comprises a polynucleotide comprising a polycistronic cassette encoding an Oct4 protein, Sox2 protein, and a Klf4 protein. In some aspects, a retroviral vector comprises a polynucleotide encoding a transcriptional activator and one or more of an Oct4 protein, Sox2 protein, Klf4 protein, c-Myc and/or a dnNFκBIA protein.

The term “gag genes,” as used herein, refers to the nucleic acids enoding group-specific antigen proteins that are major components of the capsid and specifically recognize, bind, and package retroviral genomic RNA into assembling virions.

The term “pol genes,” as used herein, refers to the genes that encode pol enzymes necessary for viral replication such as reverse transcriptase, protease, and integrase.

The term “env genes,” as used herein, refers to genes that encode envelope proteins that enable retroviruses to bind to target cell surface receptors and to enter target cells by membrane fusion.

The term “lentiviral vector,” as used herein, refers to any vector that comprises or derives from components of a lentiviral vector and is suitable to infect mammalian cells, preferably human cells. The term lentiviral vector typically designates a lentiviral particle or virion or virus comprising a payload. The terms “lentiviral genome” and “lentiviral vector” can be used interchangeably and refer to a lentiviral nucleic acid into which nucleic acids of interest have been inserted in place of certain viral sequences to produce a virus that is replication defective. In order to package a lentiviral genome or lentiviral vector into a lentiviral particle, a packaging cell line containing gag, pol, and env genes, and optionally, tat and rev genes, but without a long terminal repeat (LTR) may be provided. In some aspects, the packaging cell line comprises a rev gene but does not comprise a tat gene. Lentiviral vectors have the ability to integrate their genes into a host genome, thereby transferring a large amount of foreign genetic material to the host genome. However, in some aspects, the lentiviral vectors are non-integrating. In some aspect, the lentiviral vectors are self-inactivating vectors. In some aspects, the 3′ end of a U3 region in the lentiviral 3′ LTR is altered such that the vector RNA is produced from the intact 5′ LTR in a packaging cell line but cannot be regenerated in a target cell. In some aspects, a U3 region in a 5′ LTR of a lentiviral vector is partially or completely replaced with a heterologous promoter and/or enhancer. In some aspects, the lentiviral vectors are lentiviral circles that do not comprise a lentiviral capsid and are introduced as lentiviral DNAs into a cell, e.g., by electroporation or via a nanoparticle. In some aspects, the lentiviral circles do not comprise an integrase and remain episomal in a cell.

The term “lentiviral particle,” as used herein, refers to a lentivirus that comprises a capsid and a lentiviral vector having at least one payload region (e.g., a polynucleotide encoding at least one regeneration factor) and at least one LTR. In some aspects, the lentiviral particle is pseudo-typed by combining a lentiviral vector with a heterologous viral envelope glycoprotein. In some aspects, the heterologous viral envelope glycoprotein is a naturally occurring envelope glycoprotein, e.g., from a vescular stomatitits virus. In some aspects, the heterologous viral envelope glycoprotein is an engineered viral envelope glycoprotein, e.g., to target a specific cell type. In some aspects, a lentiviral vector comprises a polynucleotide encoding a regeneration factor. In some aspects, a lentiviral vector comprises a polynucleotide encoding an Oct4 protein, Sox2 protein, Klf4 protein and/or a c-Myc protein. In some aspects, a lentiviral vector comprises a polynucleotide comprising a polycistronic cassette encoding an Oct4 protein, Sox2 protein, Klf4 protein and a c-Myc protein. In some aspects, a lentiviral vector comprises a polynucleotide comprising a polycistronic cassette encoding an Oct4 protein, Sox2 protein, and a Klf4 protein. In some aspects, a lentiviral vector comprises a polynucleotide encoding a transcriptional activator and one or more of an Oct4 protein, Sox2 protein, Klf4 protein, c-Myc and/or a dnNFκBIA protein.

The term “adenoviral vector,” as used herein, refers to any vector that comprises or derives from components of an adenovirus vector and is suitable to infect mammalian cells, preferably human cells. The term adenoviral vector typically designates an adenovirus particle or virion comprising a payload. The terms “adenovirus genome” and “adenovirus vector” can be used interchangeably. The adenovirus vector can be derived from various serotypes, e.g., serotype Ad2 or serotype Ad5 or can comprises elements of more than one serotype. In some aspects, the adenoviral vector is a helper-dependent, replication defective adenoviral vector.

The term “adenovirus particle,” as used herein, refers to an adenovirus that comprises an adenovirus capsid and an adenoviral vector having at least one payload region (e.g., a polynucleotide encoding a regeneration factor) and at least one inverted terminal repeat (ITR). In some aspects, the adenovirus particle is prepared with a helper-dependent adenovirus vector comprising two ITRs, a payload and, optionally, non-coding stuffer sequences and a helper virus comprising adenovirus replication, packaging, and capsid genes. In some aspects, the adenoviral vector comprises a polynucleotide encoding a transcriptional activator. In some aspects, an adenoviral vector comprises a polynucleotide encoding an Oct4 protein, Sox2 protein, Klf4 protein and/or a c-Myc protein. In some aspects, an adenoviral vector comprises a polynucleotide comprising a polycistronic cassette encoding an Oct4 protein, Sox2 protein, Klf4 protein and a c-Myc protein. In some aspects, an adenoviral vector comprises a polynucleotide comprising a polycistronic cassette encoding an Oct4 protein, Sox2 protein, and a Klf4 protein. In some aspects, an adenoviral vector comprises a polynucleotide encoding a transcriptional activator and one or more of an Oct4 protein, Sox2 protein, Klf4 protein, c-Myc and/or a dnNFκBIA protein.

The terms “adeno-associated virus vector” or “AAV vector,” as used herein, refer to any vector that comprises or derives from components of an adeno-associated vector and is suitable to infect mammalian cells, preferably human cells. The term AAV vector typically designates an AAV-type viral particle or virion or virus comprising a payload. The terms “AAV genome” and “AAV vector” can be used interchangeably. The AAV vector can be derived from various serotypes, including combinations of serotypes (i.e., “pseudotyped” AAV) or from various genomes (e.g., single stranded or self-complementary). In addition, the AAV vector can be replication defective and/or targeted. As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV-DJ, AAVrh8, AAVrh10, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an AAV vector includes a derivative of a known AAV vector. In some aspects, an AAV vector includes a modified or an artificial AAV vector. In some aspects, the AAV vector is modified relative to the wild-type AAV serotype sequence.

The term “AAV particle,” as used herein, refers to an AAV virus that comprises an AAV capsid and an AAV vector having at least one payload region (e.g., a polynucleotide encoding a therapeutic protein or peptide) and at least one inverted terminal repeat (ITR). In some aspects, the AAV vector comprises a polynucleotide encoding a transcriptional activator. In some aspects, an AAV vector comprises a polynucleotide encoding an Oct4 protein, Sox2 protein, Klf4 protein and/or a c-Myc protein. In some aspects, the AAV vector comprises a polynucleotide comprising a polycistronic cassette encoding an Oct4 protein, Sox2 protein, Klf4 protein and a c-Myc protein. In some aspects, the AAV vector comprises a polynucleotide comprising a polycistronic cassette encoding an Oct4 protein, Sox2 protein, and a Klf4 protein. In some aspects, an AAV vector comprises a polynucleotide encoding a dnNFκBIA protein. In some aspects, an AAV vector comprises a polynucleotide encoding a transcriptional activator and one or more of an Oct4 protein, Sox2 protein, Klf4 protein, c-Myc and/or a dnNFκBIA protein.

The term “AAV rep gene,” as used herein, refers to the large open reading frame (ORF), known as the AAV replication (rep) region, of an AAV genome. This ORF encodes replication gene products Rep78, Rep 68, Rep 52, and Rep 40, which are named for their apparent molecular weights and which allow for the replication, assembly, and packaging of a complete AAV virus.

The term “AAV cap gene,” as used herein, refers to the large open reading frame (ORF), known as the AAV capsid (cap) region, of an AAV genome. This ORF encodes at least three capsid proteins: VP1, VP2, and VP3, which allow for the assembly of AAV capsids into which AAV genomes are packaged with the aid of AAV Rep proteins to generate AAV viruses.

The phrase “AAV helper functions for generating a productive AAV infection,” as used herein, refers to AAV rep and AAV cap genes provided by a source other than the polynucleotide comprising the payload region and the at least one ITR, such that AAV capsid production, AAV payload/ITR polynucleotide replication and AAV payload/ITR insertion into the assembled AAV capsid can occur. AAV helper functions can be provided by co-infection of the AAV producer cells with wild-type AAV virions, by providing one or more plasmids comprising the AAV rep and AAV cap genes to the AAV producer cell, or by infecting the AAV producer cell with a non-AAV virus carrying the AAV rep and AAV cap genes. AAV particles produced according to the methods described herein lack AAV rep and AAV cap genes and contain the AAV payload/ITR polynucleotide. When administered to a cell of a subject, the AAV virion, in the absence of AAV rep and AAV cap genes, cannot replicate or form more AAV virions in the subject's cell. Instead, the AAV virions release their pay load/ITR polynucleotide upon entry into a subject's cells and the payload gene is transcribed in the subject's cell to produce the payload protein.

The term “inhibiting,” as used herein, is used interchangeably with “reducing,” “silencing,” “downregulating,” “suppressing,” and other similar terms, and includes any level of inhibition.

The term “in vitro,” as used herein, refers to events that occur in an artificial environment, e.g., in a test tube or reaction vessel, in cell culture, in a Petri dish, etc., where the events are not preceeded by events occurring within an organism.

The term “ex vivo,” as used herein, refers to events that occur outside an organism such as, e.g., in a test tube, a reaction vessel, cell culture, perfusion device, etc., rather than within an organism, where the events are, for example, preceeded and/or followed by events occurring within an organism. For example, “ex vivo” perfusion of an organ refers to perfusion of an organ that has been removed from a subject and perfused outside of the subject's body.

The term “in vivo,” as used herein, refers to events that occur within an organism (e.g., animal, human, or cell or tissue thereof).

The term “transfection,” as used herein, refers to methods to introduce exogenous polynucleotides into a cell. Methods of transfection include, but are not limited to, chemical methods, physical treatments and cationic lipids or mixtures. The list of agents that can be transfected into a cell is large and includes, e.g., siRNA, shRNA, sense and/or anti-sense sequences, DNA encoding one or more genes and organized into an expression plasmid, e.g., a vector.

The phrases “contacting a cell with a polynucleotide,” “contacting a cell with an oligonucleotide,” “contacting a tissue with a polynucleotide,” “contacting a tissue with an oligonucleotide,” “contacting an organ with a polynucleotide,” or “contacting an organ with an oligonucleotide,” contacting a cell with a polypeptide,” “contacting a tissue with a polypeptide,” “contacting an organ with a polypeptide,” “contacting a cell with a compound,” “contacting a tissue with a compound,” or “contacting an organ with a compound,” “contacting a cell with a nanoparticle,” “contacting a tissue with a nanoparticle,” or “contacting an organ with a nanoparticle,” as used herein, includes contacting a cell or an organ by any possible means. Contacting a cell, tissue, or organ with a polynucleotide, oligonucleotide, or compound includes contacting a cell, tissue, or organ in vitro, in vivo, or ex vivo with the polynucleotide, oligonucleotide, polypeptide, nanoparticle, or compound. The contacting can be done directly or indirectly. Thus, for example, the polynucleotide, oligonucleotide, polypeptide, nanoparticle, or compound can be put into physical contact with the cell, tissue, or organ by the individual performing the method, or alternatively, the polynucleotide, oligonucleotide, polypeptide, nanoparticle, or compound can be put into a situation that will permit or cause it to subsequently come into contact with the cell, tissue, or organ.

Contacting a cell, tissue or organ in vitro or ex vivo can be done, for example, by incubating the cell, tissue, or organ with a polynucleotide, oligonucleotide, polypeptide, nanoparticle, or compound. Contacting a cell, tissue, or organ in vivo can be done, for example, by injecting a polynucleotide, oligonucleotide, polypeptide, nanoparticle, or compound into or near the cell, tissue, or organ; by injecting the polynucleotide, oligonucleotide, polypeptide, nanoparticle, or compound into the bloodstream; or by injecting the polynucleotide, oligonucleotide, polypeptide, nanoparticle, or compound into another area, e.g., the bloodstream or the subcutaneous space, such that the polynucleotide, oligonucleotide, polypeptide, nanoparticle, or compound will subsequently reach the tissue, the cell, or the organ to be contacted. For example, the polynucleotide, oligonucleotide, polypeptide, nanoparticle, or compound can contain and/or be coupled to a ligand that directs the polynucleotide, oligonucleotide, polypeptide, nanoparticle, or compound to a site of interest, e.g., a vessel of an organ. Combinations of in vitro and in vivo methods of contacting are also possible. For example, a cell, or tissue can be contacted in vitro or an organ can be contacted ex vivo with a polynucleotide, oligonucleotide, polypeptide, nanoparticle, or compound and subsequently transplanted into a subject.

In some aspect, contacting a cell, tissue, or organ with a polynucleotide, oligonucleotide, polypeptide, nanoparticle, or compound includes introducing or delivering the polynucleotide, oligonucleotide, polypeptide, nanoparticle, or compound into the cell, tissue or organ by facilitating or effecting uptake or absorption into the cell, tissue or organ. Absorption or uptake of a polynucleotide, oligonucleotide, polypeptide, nanoparticle, or compound can occur through unaided diffusive or active cellular processes, or by auxiliary agents or devices. For example, in vitro introduction into a cell, tissue, or organ includes methods known in the art such as electroporation and lipofection. Ex vivo introduction into an organ further includes methods such as perfusion. In some aspects, a cell or organ is contacted with a polynucleotide, oligonucleotide, polypeptide, nanoparticle, compound, an AAV vector, AAV capsid, retroviral vector, lentiviral vector, adenoviral vector or composition in vitro or in vivo. For example, a polynucleotide, oligonucleotide, polypeptide, nanoparticle, compound, AAV vector, AAV capsid, retroviral vector, lentiviral vector, adenoviral vector, or composition can be put into physical contact with the cell, tissue, or organ or be put into a situation that will permit or cause it to subsequently come into contact with the cell, tissue, or organ. In some aspects, contacting a cell or tissue in vitro or a tissue or organ ex vivo can be done, e.g., by incubating the cell, tissue, or organ with the polynucleotide, oligonucleotide, polypeptide, nanoparticle, compound, AAV vector, AAV capsid, retroviral vector, lentiviral vector, adenoviral vector, or composition. In some aspects, contacting an organ ex vivo can be done, e.g., by perfusing the organ with the polynucleotide, oligonucleotide, polypeptide, nanoparticle, compound, AAV vector, AAV capsid, retroviral vector, lentiviral vector, adenoviral vector, or composition. In some aspects, contacting a cell, tissue, or organ in vivo can be done, e.g., by injecting the polynucleotide, oligonucleotide, polypeptide, nanoparticle, compound, AAV vector, AAV capsid, retroviral vector, lentiviral vector, adenoviral vector, or composition of the disclosure into or near the tissue where a target cell is located, into the tissue, or into the organ, or by injecting the polynucleotide, oligonucleotide, polypeptide, nanoparticle, compound, AAV vector, AAV capsid, retroviral vector, lentiviral vector, adenoviral vector, or composition into an area, e.g., the bloodstream or the subcutaneous space, such that the polynucleotide, oligonucleotide, polypeptide, nanoparticle, compound, AAV vector, AAV capsid, retroviral vector, lentiviral vector, adenoviral vector, or composition will subsequently reach the tissue where the cell to be contacted is located, the tissue or organ is located. Further, a polynucleotide, oligonucleotide, polypeptide, nanoparticle, compound, AAV vector, AAV virus, retroviral vector, lentiviral vector, or adenoviral vector can be encapsulated and/or coupled to a ligand that directs the polynucleotide, oligonucleotide, polypeptide, nanoparticle, compound, AAV vector, AAV virus, retroviral vector, lentiviral vector, adenoviral vector to a site of interest, e.g., in the organ. Combinations of in vitro and in vivo methods of contacting are also possible. For example, a cell or organ can be contacted ex vivo with a polynucleotide, oligonucleotide, polypeptide, nanoparticle, compound, AAV vector, AAV capsid, retroviral vector, lentiviral vector, adenoviral vector, or a composition and subsequently transplanted into a subject.

In some aspects, contacting a cell or organ with a polynucleotide, oligonucleotide, polypeptide, nanoparticle, compound, AAV vector, AAV capsid, retroviral vector, lentiviral vector, adenoviral vector, or a composition as described herein includes “introducing” or “delivering” (directly or indirectly) the polynucleotide, oligonucleotide, polypeptide, nanoparticle, compound, AAV vector, AAV capsid, retroviral vector, lentiviral vector, adenoviral vector, or composition into the cell or organ by facilitating or effecting uptake or absorption into the cell, tissue, organ. Introducing a polynucleotide, oligonucleotide, polypeptide, nanoparticle, compound, AAV vector, AAV capsid, retroviral vector, lentiviral vector, adenoviral vector, or composition into a cell or an organ can be ex vivo and/or in vivo.

The term “lipid nanoparticle,” as used herein refers to a vesicle comprising a lipid layer encapsulating a pharmaceutically active molecule, such as a polynucleotide molecule, e.g., a polynucleotide or oligonucleotide. Lipid nanoparticles can have an average diameter between 10 and 1000 nanometers comprise a solid lipid core matrix and surfactants. Lipid nanoparticles, typically contain a cationic lipid, a non-cationic lipid, and a lipid that prevents aggregation of the particle (e.g., a PEG-lipid conjugate). Lipid nanoparticles are described in, for example, U.S. Pat. Nos. 6,858,225; 6,815,432; 8,158,601; and 8,058,069, the entire contents of which are hereby incorporated herein by reference. The lipids in the nanoparticles can be triglycerides, diglycerides, monoglycerides, fatty acids, steroids, or waxes, and mixtures thereof. The lipid nanoparticles can be prepared using high shear homogenization, ultrasound, colvent emulsification/evaporation, or microemulsion. In some aspects, the lipid nanoparticles comprise ionizable cationic lipids.

The term “cationic lipid,” as used herein, refers to lipid which has a cationic, or positive, charge at physiologic pH. Cationic lipids can take a variety of forms including, but not limited to, liposomes or micelles. Cationic lipids useful for certain aspects of the present disclosure are known in the art, and, generally comprise both polar and non-polar domains, bind to polyanions, such as nucleic acid molecules or negatively supercharged proteins, and are typically known to facilitate the delivery of nucleic acids into cells. Examples of useful cationic lipids include polyethylenimine, polyamidoamine (PAMAM) starburst dendrimers, Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,Ntrimethylammonium methylsulfate (DOTAP), 3-β[N—(N′,N′dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB).

The term “liposome,” as used herein, refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the oligonucleotide composition. The lipophilic material isolates the aqueous interior from an aqueous exterior, which typically does not include the oligonucleotide composition, although in some examples, it can. Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids.

The term “micelle,” as used herein refers to a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

The term “organ,” as used herein refers to any organ that can be submitted to the materials and methods as described herein and used for transplantation into a subject in need thereof.

The term “organoid,” as used herein, refers to a miniaturized and simplified version of an organ produced in three dimensional culture outside of a body from a tissue, embryonic stem cell or induced pluripotent stem cell.

The term “organ transplantation,” as used herein, refers to a procedure in which an organ is removed from an organism (termed the donor) and is transferred to a recipient organism. In some aspects, the donor and recipient are the same organism. Examples of organ transplantation include, but are not limited to, kidney transplantation, liver transplantation, lung transplantation, heart transplantation, pancreas transplantation, or corneal transplantation. The organ may be treated or modified according to methods described herein prior to transplantation. In some aspects, the organ is obtained from one organism (the donor), submitted to the materials and methods described herein, and introduced into a second organism (the recipient). In some aspects, the organ is obtained from one organism, submitted to the materials and methods described herein, and introduced back into that same organism (self-transplantation). In some aspects, two organs, e.g., two kidneys, are obtained from one organism, submitted to the materials and methods described herein, and the two organs are introduced into one recipient or one of the two organs is introduced into one recipient and the other into a different recipient. In some aspects, two organs, e.g., two kidneys, are obtained from one organism, submitted to the materials and methods described herein, and the two kidneys are introduced into the same organism or one kidney is introduced into the same organism and the other kidney is introduced into a different organism or both kidneys are introduced into different organisms. In some aspects, an organ portion is obtained from an organism, submitted to the materials and methods described herein, and introduced into a different recipient organism. In some aspects, an organ portion is obtained from an organism, submitted to the materials and methods described herein, and introduced into the same organism (self-transplantation). In some aspects, one organ, e.g., a liver is obtained from one organism, submitted to the materials and methods described herein, then split in two or more organ portions and each organ portion is introduced into a different recipient or one organ portion is introduced into the organism from which the organ originated and one or more organ portions are introduced into one or more different recipients

The term “perfusion,” as used herein, refers to a passage of fluid through a circulatory system or a lymphatic system to a tissue and/or organ. In some aspects, perfusion comprises passage of a fluid through a blood vessel. In some aspects, perfusion comprises passage of a fluid through a lymphatic vessel. In some aspects, perfusion comprises cold perfusion. In some aspects, perfusion comprises normothermic perfusion. In some aspects, the perfusion comprises normothermic machine perfusion using a perfusion machine. In some aspects, normothermic machine perfusion is at ~38° C. In some aspects, normothermic machine perfusion is at about 22° C. to about 38° C.; or at about 23° C. to about 37° C.; at about 24° C. to about 36° C.; at about 25° C. to about 35° C.; at about 26° C. to about 34° C.; at about 27° C. to about 33° C.; at about 28° C. to about 32° C.; or at about 33° C.; about 34° C.; about 35° C.; about 36° C.; or about 37° C. In some aspects, the perfusion comprises cold perfusion followed by normothermic machine perfusion. In some aspects, cold perfusion is at about 2° C. to about 21° C.; or at about 3° C. to about 20° C.; at about 4° C. to about 19° C.; at about 5° C. to about 18° C.; at about 6° C. to about 17° C.; at about 7° C. to about 16° C.; at about 8° C. to about 15° C.; at about 9° C. to about 14° C.; at about 10° C. to about 13° C.; or at about 2° C.; about 3° C.; about 4° C.; about 5° C.; or about 6° C.; about 7° C.; about 8° C.; about 9° C.; about 10° C.; about 11° C.; about 12° C.; about 13° C.; about 14° C.; about 15° C.; or about 16° C.

The terms “perfusion machine,” “perfusion system,” or “perfusion device,” as used herein are interchangeable and refer to a machine, system or device that comprises a pump, a reservoir to hold an organ or organoid, a second reservoir to hold a perfusate, and tubing to connect the reservoir holding the organ and the perfusate reservoir. In some aspects, the perfusion system further comprises tubing that connects a blood vessel or a lymphatic vessel of an organ with the perfusate reservoir. For example, a perfusion system can be a Hugo Sachs/Harvard Apparatus, Kidney Assist™ system, OrganOX system, Radnoti system, ARK Kidney system, and Aferetica PerLife® system.

The term “perfusate,” as used herein, refers to a liquid composition that runs through a blood vessel or lymphatic vessel of an organ ex vivo or otherwise permeates an organ ex vivo. A perfusate can comprise at least one of a buffer, an inorganic salt, an amino acid, a substrate for metabolism, a hormone, a vasodilator, a tonicity agent, an oxygenation agent, an anti-oxidant, an anti-inflammatory agent, an anti-coagulant, or an anti-microbial agent and at least one of a polynucleotide, oligonucleotide, compound, AAV vector, lentiviral vector, retroviral vector, or adenoviral vector.

The term “tissue repair” in the context of damaged tissue refers to restoration of tissue architecture, function following tissue damage, or a combination thereof. Tissue repair includes, but is not limited to, tissue regeneration, cell growth, and/or tissue replacement of existing tissue (reprogramming).

The term “regeneration” refers to production of new tissue or cells within a tissue. In some embodiments, the methods provided herein promote organ regeneration.

The terms “tissue regeneration” or “organ regeneration,” as used herein, refer to at least partial regeneration, replacement, restoration, or regrowth of a tissue, organ, or other body structure, or portion thereof, for example, following loss, damage, or degeneration, where said tissue regeneration but for the methods described in the present disclosure would not take place. Regeneration of an organ is associated, e.g., with an increase in size and/or cell number in the injured or diseased organ

The term “reprogramming” refers to the process of altering a cell using reprogramming factors (e.g. reversing (in whole or in part) or preventing (in whole or in part) changes in cells that are causes of dysfunction, deterioration, cell death, or aging). Reprogramming may be complete reprogramming, such that a differentiated cell (e.g., somatic cell) is reprogrammed to a pluripotent stem cell. Cellular reprogramming may be incomplete, such that a differentiated cell (e.g., somatic cell) retains its cellular identity (e.g., lineage-specific stem cell). Cellular reprogramming may be incomplete, e.g., such that a cell is rejuvenated, or takes on more youthful attributes (e.g. increased survival, reduced inflammation, or ability to divide).

The terms “dedifferentiation,” “increasing cell potency,” or “increasing developmental potency,” as used herein, refer to processes that alter or reverse the differentiation state of a differentiated cell (e.g., a somatic cell) by driving the differentiation of a cell backwards to a more undifferentiated or more primitive type of cell. For example, a cell that has an increased cell potency has more developmental plasticity (i.e., can differentiate into more cell types).

The term “enhancing agent,” as used herein, refers to an agent or combination of agents that enhance the efficiency or rate of reprogramming and/or rejuvenation and/or regeneration can include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA (e.g., MK0683, vorinostat and other hydroxamic acids), vitamin C, trichostatin (TSA), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenyl butyrate (e.g., sodium phenyl butyrate) and other short chain fatty acids, Scriptaid, Suramin Sodium, APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVPLAQ-824, CBHA (m-carboxycinnaminic acid bishydroxamic acid), JNJ16241199, Tubacin, A-161906, proxamide, oxamflatin, 3-Cl-UCHA (e.g., 6-(3-chlorophenylureido) caproic hydroxamic acid), AOE (2-amino-8-oxo-9,10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Aton Pharma, Titan Pharmaceuticals, Schering A G, Pharmion, MethylGene, and Sigma Aldrich.

The term “potency,” as used herein, refers to the sum of all developmental options accessible to a cell (i.e., the developmental potency). Cell potency is a continuum, ranging from the most plastic cell, a totipotent stem cell, which has the most developmental potency to the least plastic cell, a terminally differentiated cell, which has the least developmental potency. The continuum of cell potency includes, but is not limited to, totipotent cells, pluripotent cells, multipotent cells, oligopotent cells, unipotent cells, and terminally differentiated cells. As used herein, the term “pluripotent” refers to the ability of a cell to form all lineages of the body or soma (i.e., the embryo proper). For example, an embryonic stem cell is a type of pluripotent stem cell that is able to form cells from each of the three germs layers: the ectoderm, the mesoderm, and the endoderm. Pluripotency can be determined, in part, by assessing pluripotency characteristics of the cells. Pluripotency characteristics include, but are not limited to: (i) pluripotent stem cell morphology; (ii) the potential for unlimited self-renewal (iii) expression of pluripotent stem cell markers including, but not limited to SSEA1 (mouse only), SSEA3/4; SSEA5, TRA1-60/81; TRA1-85, TRA2-54, GCTM-2, TG343, TG30, CD9, CD29, CD133/prominin, CD140a, CD56, CD73, CD90, CD105, Oct4, Nanog, Sox2, CD30 and/or CD50; (iv) ability to differentiate to all three somatic lineages (ectoderm, mesoderm and endoderm) (v) teratoma formation consisting of the three somatic lineages; and (vi) formation of embryoid bodies consisting of cells from the three somatic lineages.

The term “regeneration factor,” or “reprogramming factor,” as used herein, refers to an agent that can revert a terminally differentiated cell into a cell of a less differentiated state, such as a multipotent or pluripotent state. Examples of regeneration factors include, but are not limited to, Oct family genes, Sox family genes, Klf family genes, Myc family genes, SALL4, NANOG, LIN28, STELLA, NOBOX, ESRRB, NR5A2, CEBPA or a STAT family genes, including, but not limited to, STAT1, STAT2, STAT3, STAT4, STAT5 (STAT5A and STAT5B), and STAT6; and a dominant negative nuclear factor kappa B IA (dnNFκBIA). In some aspects, the regeneration factors are Oct4, Sox2, Klf4, and c-Myc; Oct4, Sox2, Nanog and Lin28; Oct4, Sox2, and Klf4; or various combinations of Oct4, Sox2, klf4, nanog, ESRRB, NR5A2, CEBPA, Myc, Lin28A and Lin28B.

The terms “preventing full dedifferentiation,” “blocking full dedifferentiation,” as used herein, refer to methods that revert a cell to a less differentiated state without allowing the cell to revert to a stem cell state. The methods can comprise contacting a cell for a time period with at least one regeneration factor. The methods can comprise contacting a cell with at least one regeneration factor that does not induce reversion of a cell to a stem cell state.

The term “rejuvenating a cell,” as used herein, is meant to include preventing or reversing (in whole or in part) the cellular causes of aging without inducing a pluripotent state.

The term “Oct family gene,” as used herein, refers to the family of Octamer transcription factors and includes, but is not limited to, Oct1, Oct3, Oct4, Oct6, and variants thereof.

The term “Sox family gene,” as used herein, refers to the family of Syr-related HMG-box transcription factors and includes, but is not limited to, Sox1, Sox2, Sox3, Sox7, Sox15, Sox17, Sox18, and variants thereof.

The term “Klf family gene,” as used herein, refers to the family of Kruppel-like factor transcription factors and includes, but is not limited to, Kfl1, Klf2, Klf4, Klf5, and variants thereof.

The term “Myc family gene,” as used herein, refers to the family of Myc proto-oncogene transcription factors and includes, but is not limited to, c-Myc, L-Myc, N-Myc, and variants thereof. For example, a Myc variant can have a deletion of amino acids at positions 1 to 41 in the amino acid sequence of human c-Myc of SEQ ID NO: 4. In some aspects, a Myc variant has a deletion of amino acids at positions 1 to 64 in the amino acid sequence of human c-Myc. In some aspects, a Myc variant has a deletion of amino acids at positions 1 to 107 in the amino acid sequence of human c-Myc. In some aspects, a Myc variant has a deletion of amino acids at positions 1 to 13 and has a mutation at position 135 in the amino acid sequence of human c-Myc, wherein the mutation at position 135 is a deletion or substitution. In some aspects, the Trp at position 135 of human c-Myc is substituted with Glu or Gly. In some aspects, a L-Myc variant has at least the amino acid sequence at and after position 70 in the amino acid sequence of human L-Myc as shown in SEQ ID NO: 5. In some aspects, a L-Myc variant has at least amino acids at and after position 45 in the amino acid sequence of human L-Myc as shown in SEQ ID NO: 5. In some aspects, a L-Myc variant has at least amino acids at and after position 22 in the amino acid sequence of human L-Myc as shown in SEQ ID NO: 5. In some aspects, a L-Myc variant has a mutation at position 321 in the amino acid sequence of human L-Myc as shown in SEQ ID NO: 5. In some aspects, the mutation at position 321 in SEQ ID NO: 5 is a substitution or deletion. In some aspects, a Val at position 321 in SEQ ID NO: 5 is substituted with Asp. In some aspects, the Myc is human N-Myc of SEQ ID NO: 6.

The term “polypeptide variant,” as used herein, typically exhibits at least about 70%, 75%, 80%, 85%, 90%, 91%, 92%, 93%, 94%, 95%, 96%, 97%, 98%, or 99% or more identity, along its length, to a polypeptide sequences set forth herein. In a particular aspect, the variant or combination of variants employed retain the ability to induce pluripotency as described herein. A polypeptide variant may differ from a naturally occurring polypeptide in one or more substitutions, deletions, additions and/or insertions. Such variants may be naturally occurring or may be synthetically generated, for example, by modifying one or more of the above polypeptide sequences and evaluating their effects.

The term “percent (%) sequence identity,” as used herein, with respect to a reference polynucleotide or polypeptide sequence is defined as the percentage of polynucleotides or amino acids in a candidate sequence that are identical to the polynucleotides or amino acids in the reference polynucleotide or polypeptide sequence, after aligning the sequences and introducing gaps, if necessary, to achieve the maximum percent sequence identity. Alignment for purposes of determining percent polynucleotide or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values can be generated using the sequence comparison computer program BLAST. Those skilled in the art can determine appropriate parameters for aligning sequences, including any algorithms needed to achieve maximal alignment over the full length of the sequences being compared. For example, percent sequence identity values can be generated using the sequence comparison computer program BLAST. As an illustration, the percent sequence identity of a given polynucleotide or amino acid sequence, A, to, with, or against a given polynucleotide or amino acid sequence, B, (which can alternatively be phrased as a given polynucleotide or amino acid sequence, A that has a certain percent sequence identity to, with, or against a given polynucleotide or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of polynucleotides in B. It will be appreciated that where the length of polynucleotide or amino acid sequence A is not equal to the length of polynucleotide or amino acid sequence B, the percent sequence identity of A to B will not equal the percent sequence identity of B to A.

The term “amino acid substitution” in a variant, as used herein, refers to the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, praline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Within a particular group, certain substitutions may be of particular interest, e.g., replacements of leucine by isoleucine (or vice versa), serine by threonine (or vice versa), or alanine by glycine (or vice versa). Of course non-conservative substitutions are often compatible with retaining function as well. In some aspects, a substitution or deletion does not alter or delete an amino acid important for activity. Insertions or deletions may range in size from about 1 to 20 amino acids, e.g., 1 to 10 amino acids. In some instances, larger domains may be removed without substantially affecting function. In certain embodiments of the disclosure the sequence of a variant can be obtained by making no more than a total of 5, 10, 15, or 20 amino acid additions, deletions, or substitutions to the sequence of a naturally occurring enzyme. In some aspects, no more than 1%, 5%, 10%, or 20% of the amino acids in a polypeptide are insertions, deletions, or substitutions relative to the original polypeptide. Guidance in determining which amino acid residues may be replaced, added, or deleted without eliminating or substantially reducing activities of interest, may be obtained by comparing the sequence of the particular polypeptide with that of homologous polypeptides (e.g., from other organisms) and minimizing the number of amino acid sequence changes made in regions of high homology (conserved regions) or by replacing amino acids with those found in homologous sequences since amino acid residues that are conserved among various species are more likely to be important for activity than amino acids that are not conserved. In some aspects, a variant of a polypeptide comprises a heterologous polypeptide portion. The heterologous portion often has a sequence that is not present in or homologous to the original polypeptide. A heterologous portion may be, e.g., between 5 and about 5,000 amino acids long, or longer. Often it is between 5 and about 1,000 amino acids long. In some aspects, a heterologous portion comprises a sequence that is found in a different polypeptide, e.g., a functional domain. In some aspects, a heterologous portion comprises a sequence useful for purifying, expressing, solubilizing, and/or detecting the polypeptide. In some aspects, a heterologous portion comprises a polypeptide “tag”, e.g., an affinity tag or epitope tag. For example, the tag can be an affinity tag (e.g., HA, TAP, Myc, 6×His, Flag, GST), fluorescent or luminescent protein (e.g., EGFP, ECFP, EYFP, Cerulean, DsRed, mCherry), or a solubility-enhancing tag (e.g., a SUMO tag, NUS A tag, SNUT tag, or a monomeric mutant of the Ocr protein of bacteriophage T7). See, e.g., Esposito D and Chatterjee D K. Curr Opin Biotechnol.; 17(4):353-8 (2006). In some aspects, a tag can serve multiple functions. A tag is often relatively small, e.g., ranging from a few amino acids up to about 100 amino acids long. In some aspects, a tag is more than 100 amino acids long, e.g., up to about 500 amino acids long, or more. In some aspects, a polypeptide has a tag located at the N- or C terminus, e.g., as an N- or C-terminal fusion. The polypeptide could comprise multiple tags. In some aspects, a 6×His tag and a NUS tag are present, e.g., at the N-terminus. In some aspects, a tag is cleavable, so that it can be removed from the polypeptide, e.g., by a protease. In some aspects, this is achieved by including a sequence encoding a protease cleavage site between the sequence encoding the portion homologous to the original polypeptide and the tag. Exemplary proteases include, e.g., thrombin, TEV protease, Factor Xa, PreScission protease, etc. In some aspects, a “self-cleaving” tag is used. Sequences encoding a tag can be located 5′ or 3′ with respect to a polynucleotide encoding the polypeptide (or both). In some aspects, a tag or other heterologous sequence is separated from the rest of the polypeptide by a polypeptide linker. For example, a linker can be a short polypeptide (e.g., 15-25 amino acids). Often a linker is composed of small amino acid residues such as serine, glycine, and/or alanine. A heterologous domain could comprise a transmembrane domain, a secretion signal domain, etc.

The term “level,” as used herein refers to a level or activity of a protein or mRNA encoding one or more proteins (e.g., a regeneration factor), optionally as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein can be expressed in mass/vol (e.g., g/dL, mg/mL, g/mL, or ng/mL) or percentage relative to total protein or mRNA in a sample.

The phrase “determining the level of a protein,” as used herein refers to the detection of a protein, or an mRNA encoding the protein, by methods known in the art either directly or indirectly. “Directly determining” means performing a process, e.g., performing an assay or test on a sample or analyzing a sample to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure RNA levels include, but are not limited to, transcriptome sequencing, bulk RNA sequencing, and single cell RNA sequencing.

The term “transcriptomic profile,” as used herein, refers to the set of all RNA molecules in one cell or a population of cells. It is sometimes used to refer to all RNAs, or just mRNA, depending on the particular experimental setting. It differs from the exome in that it includes only those RNA molecules found in a specified cell population, and usually includes the amount or concentration of each RNA molecule in addition to the molecular identities. Methods of obtaining a transcriptomic profile include DNA microarrays and next generation sequencing technologies such as RNA-Seq. Transcription can also be studied at the level of individual cells by single-cell transcriptomics. One approach for inferring transcriptome sequences maps sequence reads onto a reference genome, either of the organism itself (whose transcriptome is being studied) or of a closely related species. Another approach, de novo transcriptome assembly, uses software to infer transcripts directly from short sequence reads.

The term “tissue damage,” “cell damage”, and “organ damage” as used herein, refers to any type of damage or injury to cells, a tissue, or an organ. The term encompasses, in various aspects, damage due to aging, damage due to disease, damage due to physical trauma or surgery, damage caused by exposure to deleterious substance(s), and other disruptions in the structure and/or functionality of cells, a tissue, or an organ.

The term “subject,” as used herein refers to any organism (and may be used interchangeably with subject herein) to which a material or method described herein can be administered, e.g., for experimental, diagnostic, prophylactic, and/or therapeutic purposes. Exemplary organisms include mammals, e.g., humans, non-human primates, rodents (e.g., mouse, rat, rabbit), ungulates (e.g., ovine, bovine, equine, caprine species), canines, and felines. Often, a subject is an individual to whom a compound is to be delivered, e.g., for experimental, diagnostic, and/or therapeutic purposes or from whom a sample is obtained or on whom a diagnostic procedure is performed (e.g., a sample or procedure that will be used to assess tissue damage and/or to assess the effect of a compound described in the disclosure).

The term “donor,” as used herein, refers to a subject from whom an organ is obtained by surgery and includes a mammal e.g., a human, a non-human primate, a rodent (e.g., mouse, rat, rabbit), an ungulate (e.g., ovine, bovine, equine, caprine species), a canine, and a feline.

The term “recipient,” as used herein, refers to a subject to whom an organ is transferred by surgery and includes a mammal e.g., a human, a non-human primate, a rodent (e.g., mouse, rat, rabbit), an ungulate (e.g., ovine, bovine, equine, caprine species), a canine, and a feline.

The term “treat”, “treating”, “therapy”, “therapeutic” and similar terms in regard to a subject refer to both therapeutic treatment and prophylactic or preventative measures wherein the object is to prevent or slow down (lessen) an undesired physiological, e.g., an age-related, condition, disorder, or disease, or obtain beneficial or desired clinical results. In some aspects, an age-related condition includes signs and/or symptoms associated with natural aging. In some aspects, treating reduces or lessens the symptoms associated with, e.g., an age-related disease or disorder. In some aspects, the treating results in a beneficial or desired clinical result. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. In some aspects, treatment includes eliciting a clinically significant response without excessive levels of side effects. In some aspects, treatment includes prolonging survival as compared to expected survival if not receiving treatment. As used herein, the term “amelioration” or “ameliorating” refers to a lessening of severity of at least one indicator of a condition or disease. As used herein, the term “preventing” or “prevention” refers to delaying or forestalling the onset, development or progression of a condition or disease for a period of time, including weeks, months, or years. Ameliorating the disease or disorder includes slowing the course of the disease or disorder or reducing the severity of later-developing an age-related disease or disorder. The “prophylactically effective amount” can vary depending on the characteristics of the agent, composition, how the agent is administered, the degree of risk of disease, and the history, age, weight, family history, genetic makeup, the types of preceding or concomitant treatments, if any, and other individual characteristics of the patient to be treated. In some aspects, treatment is provided to an explanted organ such as to regenerate and/or rejuvenate an aged organ, regenerate and/or rejuvenate an injured organ, and/or reverse (in whole or in part) the physical changes associated with at least one of old age, an ischemic injury, a hypertensive injury, a toxic injury, a perfusion injury, an immune injury; a physical injury, steatosis, virus induced hepatitis, alcohol, or a fibrosis not associated with any known cause.

The terms “effective amount,” “therapeutically effective amount,” and a “sufficient amount” of, e.g., a polynucleotide, oligonucleotide, polypeptide, vector, agent, nanoparticle or composition as described herein refer to a quantity sufficient to, when administered to a subject, a tissue, or an organ, effect beneficial or desired results, including clinical results. As such, an “effective amount” or synonym thereto depends on the context in which it is being applied. In some aspects, a therapeutically effective amount of an agent (e.g., a polynucleotide, a vector, a nanoparticle, or a composition as described herein) is an amount that results in a beneficial or desired result in a subject or an ex vivo tissue or organ as compared to a control. The amount of a given agent (e.g., a polynucleotide, a vector, a nanoparticle, or a composition as described herein) will vary depending upon various factors, such as the given agent, the pharmaceutical formulation, the route of administration, the type of disease or disorder, the organ, the identity of the subject, the organ donor and the organ recipient (e.g., age, sex, and/or weight) being treated, and the like. For example, in the context of treating an ex vivo tissue or organ, it is an amount of the agent that is sufficient to achieve a desired response in the tissue or organ as compared to the response obtained without administration of the agent. A desired response may, e.g., be a rejuvenation of an aged organ or a reversal of changes in an organ that occurred as a consequence of at least one of old age, an ischemic injury, a hypertensive injury, a toxic injury, a perfusion injury, an immune injury, a physical injury, steatosis, virus induced hepatitis, alcohol, or a fibrosis not associated with any known cause.

The term “prophylactically effective amount,” as used herein, refers to an amount of a polynucleotide, a vector, a nanoparticle, or a composition as described herein that, when administered to a subject, tissue or organ, is sufficient to prevent or ameliorate a disease or an injury in the subject or organ. Ameliorating a disease or an injury includes slowing the course of a disease or an injury-induced process or reducing the severity of a later-developing disease or injury-induced disease. The prophylactically effective amount can vary depending on the therapeutic agent used and how the therapeutic agent is administered, the degree of risk of disease or injury, the history, age, weight, family history, genetic makeup, types of preceding or concomitant treatments, if any, of a subject, including an organ donor and an organ recipient and other individual characteristics of the subject, organ donor and organ recipient. A prophylactically effective amount can refer to, for example, an amount of the agent that reduces the level and/or activity of an ischemic injury, a hypertensive injury, a toxic injury, a perfusion injury, an immune injury, a physical injury, steatosis, virus induced hepatitis, alcohol, or fibrosis not associated with any known cause in an organ and can refer to a quantity sufficient to, when administered to a subject, including a human, a tissue, or an organ, can delay the onset of one or more of the signs and symptoms of schemia or injury described herein by at least 120 days, for example, at least 6 months, at least 12 months, at least 2 years, at least 3 years, at least 4 years, at least 5 years, at least 10 years or more, when compared with the predicted onset.

The terms “intermittent addition,” or “intermittent administration,” as used herein, refer to the repeated addition of a polynucleotide, polypeptide, vector, or nanoparticle to a composition, where each addition is followed by a time period of no addition; or to the repeated administration of a polynucleotide, polypeptide, vector, or nanoparticle to a subject, where each administration is followed by a time period of no administration.

The term “gene therapy,” as used herein, refers to the insertion of polynucleotide sequences (e.g., a polynucleotide comprising a promoter operably linked to a polynucleotide encoding a therapeutic molecule as disclosed herein) into an individual's cells, tissues and/or an organ to treat, reduce the symptoms of, or reduce the likelihood of a disease or aging-related condition. Gene therapy also includes insertion of a transgene that is inhibitory in nature, i.e., that inhibit, decrease or reduce expression, activity or function of an endogenous gene or protein, such as an undesirable (e.g., injury inducing) or aberrant (e.g., pathogenic) gene or protein. Such transgenes can be exogenous. An exogenous molecule or sequence is understood to be molecule or sequence not normally occurring in the cell, tissue, organ and/or individual to be treated.

The term “biopsy,” as used herein refers to a material or procedure to obtain such material from a tissue or organ of a subject, e.g., by fine needle aspiration. A biopsy typically contains a number of cell types of a tissue or organ and can provide information on the morphology, histopathology, and functionality of a tissue or organ.

The term “pharmaceutical composition,” as used herein, refers to a composition comprising a compound or vector described herein, e.g., a polynucleotide, oligonucleotide, AAV vector, lentiviral vector, retroviral vector, or adenoviral vector as described herein, formulated with a pharmaceutically acceptable excipient, that can be manufactured or sold with the approval of a governmental regulatory agency as part of a therapeutic regimen for the treatment of a disease or an organ prior to transplantation.

The term “pharmaceutically acceptable excipient,” as used herein, refers to any ingredient other than the compound or vector described herein (for example, a vehicle capable of suspending or dissolving the active compound or vector) and having the properties of being substantially nontoxic and non-inflammatory in a patient or organ.

### II. Polynucleotides

Provided are polynucleotides for expression of regeneration factors. In some aspects, the polynucleotides comprise polynucleotide sequences encoding at least one regeneration factor. In some aspects, the polynucleotides comprise polynucleotide sequences encoding at least one transcriptional activator. In some aspects, the polynucleotides comprise polynucleotide sequences encoding at least one regeneration factor and at least one transcriptional activator.

In some aspects, the polynucleotides are non-integrating RNAs. In some aspects, the polynucleotides are non-integrating DNAs. In some aspects, the polynucleotides are integrating RNAs. In some aspects, the polynucleotides are integrating DNAs.

In some aspects, the polynucleotides are present in a vector. In some aspects, the polynucleotides are present in a plasmid. In some aspects, the polynucleotides are present in a viral vector. In some aspects, the polynucleotides are present in an AAV vector, a lentiviral vector, a retroviral vector, or an adenoviral vector.

In some aspects, the polynucleotides comprise at least one inducible promoter. In some aspects, the polynucleotides comprise at least one constitutive promoter. In some aspects, the polynucleotides comprise at least one cell-type specific promoter.

In some aspects, the non-integrating RNAs encode at least one regeneration factor that rejuvenates a cell while retaining the cell in a differentiated state. In some aspects, the non-integrating DNAs encode at least one regeneration factor that rejuvenates a cell while retaining the cell in a differentiated state. In some aspects, the integrating RNAs encode at least one regeneration factor operably linked to an inducible promoter and contacting a cell with an inducing compound rejuvenates the cell while retaining the cell in a differentiated state. In some aspects, the integrating DNAs encode at least one regeneration factor operably linked to an inducible promoter and contacting a cell with an inducing compound rejuvenates the cell while retaining the cell in a differentiated state.

In some aspects, the polynucleotides comprise transposon elements.

In some aspects, the polynucleotides comprise a polynucleotide sequence encoding at least one transcriptional transactivator.

In some aspects, the polynucleotides comprise an inducible promoter operably linked to a polynucleotide sequence encoding at least one regeneration factor and a polynucleotide sequence encoding at least one transcriptional transactivator.

In some aspects, the inducible promoter is a tetracycline-inducible promoter; a hormone inducible promoter; a steroid-inducible promoter; a cumate-inducible promoter, or a coumermycin-inducible promoter. In some aspects, the hormone inducible promoter is a tamoxifen inducible promoter comprising a transactivator domain fused to an estrogen receptor. In some aspects, the steroid-inducible promoter is a progesterone promoter comprising a transactivator domain fused to a mutant progesterone receptor that binds RU486 but not endogenous progesterone. In some aspects, the coumermycin-inducible promoter is a hybrid promoter comprising a transactivator domain that binds the hybrid promoter after being homodimerized by coumermycin. In some aspects, the cumate-inducible promoter comprises an operator site (CuO) downstream of a constitutive promoter and a repressor protein (CymR) bound to the CuO site, wherein addition of cumate relieves repression; or a chimeric transactivator protein formed by fusion of a CymR with an activation domain, which chimeric transactivation protein binds CuO upstream of a constitutive promoter and cumate abrogates DNA binding of the chimeric transactivator thereby terminating transcriptional activation (see, e.g., Mullick et al., BMC Biotechnology 6:43, 2006).

In some aspects, the tetracycline-inducible promoter comprises multiple copies of a tet operator sequence that can bind a reverse tetracycline-controlled transactivator protein (rtTA). In some aspects, the tetracycline-inducible promoter comprises multiple copies of a tet operator sequence that can bind a tTA-M2, rtTA3 or rtTA4 transactivator protein.

In some aspects, the inducible promoter of the polynucleotide is bound by a transactivator protein in the presence of an inducing compound. In some aspects, the tetratcycline-inducible promoter of the polynucleotide is bound by an rTA protein in the presence of tetracycline. In some aspects, the tetracycline-inducible promoter is operably linked to at least one regeneration factor. In some aspects, the tetracycline-inducible promoter is operably linked to a polycistronic cassette comprising a polynucleotide encoding at least two regeneration factors. In some aspects, the tetracycline-inducible promoter is operably linked to a polycistronic cassette comprising a polynucleotide encoding Oct4 and Sox2. In some aspects, the polycistronic cassette further comprises a polynucleotide encoding Klf4. In some aspects, the polycistronic cassette further comprises a polynucleotide encoding c-Myc. In some aspects, the polynucleotide further comprises a polynucleotide encoding a transcriptional transactivator. In some aspects, the polynucleotide further comprises a polynucleotide encoding rtTA. In some aspects, a polynucleotide comprises a polynucleotide encoding rtTA operably linked to an inducible promoter. In some aspects, a polynucleotide comprises a polynucleotide encoding rtTA operably linked to a constitutive promoter. In some aspects, a polynucleotide comprises a polynucleotide encoding rtTA operably linked to a cell-type specific promoter.

In some aspects, a polynucleotide comprises at least one constitutive promoter, e.g., CPI, CMV, EF1-alpha, SV40, PGK1, Ubc, human beta actin, CAG, CamKIIa, TEF1, GDS, CaMV35S, Grp78, Grp94, Hsp70, EGFR, H1, and U6 promoter.

In some aspects, a polynucleotide comprises a polynucleotide sequence encoding at least one regeneration factor operably linked to a constitutive promoter and a loxP sequence framing the polynucleotide sequence encoding the at least one regeneration factor. In some aspects, a polynucleotide comprises more than one constitutive promoter and more than one expression cassette, optionally, being framed by loxP sequences.

In some aspects, the polynucleotide comprises an EF1-alpha promoter operably linked to a polynucleotide encoding a mutant reverse tetracycline-controlled transactivator rTA-M2. In some aspects, the polynucleotide comprises an EF1-alpha promoter operably linked to a polynucleotide encoding a mutant reverse tetracycline-controlled transactivator rTA-M2 and an inducible promoter operably linked to a c-Myc gene. In some aspects, the EF1-alpha promoter comprises a polynucleotide sequence of SEQ ID NO: 12.

In some aspects, the polynucleotide comprises a TetO7 tetracycline operator promoter operably linked to a polycistronic cassette comprising polynucleotide sequences encoding an Oct4 gene, a Sox2, gene and/or a Klf4 gene. In some aspects, the polynucleotide further comprises a TetO7 tetracycline operator promoter operably linked to a c-Myc gene.

In some aspects, the polynucleotide comprises a polynucleotide sequence of SEQ ID NO: 26. In some aspects, the polynucleotide comprises a polynucleotide sequence of SEQ ID NO: 27. In some aspects, the polynucleotide comprises a binding site for an estrogen receptor binding transcription factor element operably linked to a c-Myc gene. In some aspects, the polynucleotide comprises a binding site for an estrogen receptor binding transcription factor element operably linked to a polycistronic cassette comprising polynucleotide sequences encoding an Oct4 gene, a Sox2, gene and/or a Klf4 gene. In some aspects, the polynucleotide comprises a binding site for a chimeric regulator protein that is responsive to RU486 but not endogenous progestins operably linked to a c-Myc gene. In some aspects, the polynucleotide comprises a binding site for a chimeric regulator protein that is responsive to RU486 but not endogenous progestins operably linked to a polycistronic cassette comprising polynucleotide sequences encoding an Oct4 gene, a Sox2, gene and/or a Klf4 gene.

In some aspects, the polynucleotides comprise a promoter that is active in senescent cells. In some aspects, the promoter is a promoter of a cyclin-dependent kinase inhibitor 2A (CDKN2A)/p16 gene. In some aspects, the promoter is a p21 promoter. In some aspects, the promoter is a mir146A promoter. In some aspects, the promoter comprises a polynucleotide sequence that is at least 85%, 86%, 87%, 88%, 89%, 90%, 91%, 92%, 93%, 94%, 95%, 96%, 97%, 98%, or 99%, identical to the polynucleotide sequence of SEQ ID NO: 19. In some aspects, the promoter comprises the polynucleotide sequence of SEQ ID NO: 19. In some aspects, the promoter comprises a polynucleotide sequence that is at least 85%, 86%, 87%, 88%, 89%, 90%, 91%, 92%, 93%, 94%, 95%, 96%, 97%, 98%, or 99%, identical to the polynucleotide sequence of SEQ ID NO: 20. In some aspects, the promoter comprises the polynucleotide sequence of SEQ ID NO: 20.

In some aspects, the promoter comprises a polynucleotide sequence that is at least 85%, 86%, 87%, 88%, 89%, 90%, 91%, 92%, 93%, 94%, 95%, 96%, 97%, 98%, or 99%, identical to the polynucleotide sequence of SEQ ID NO: 7. In some aspects, the promoter comprises the polynucleotide sequence of SEQ ID NO: 7. In some aspects, the polynucleotide comprises a polynucleotide encoding a transactivator that is at least 85%, 86%, 87%, 88%, 89%, 90%, 91%, 92%, 93%, 94%, 95%, 96%, 97%, 98%, or 99%, identical to a polynucleotide sequence of SEQ ID NO: 12. In some aspects the polynucleotide comprises a polynucleotide sequence of SEQ ID NO: 12.

In some aspects, the polynucleotide comprises a polynucleotide sequence encoding an Oct4 protein. In some aspects, the Oct4 protein is a human Oct4 protein. In some aspects, the polynucleotide comprises a polynucleotide sequence that is at least 85%, 86%, 87%, 88%, 89%, 90%, 91%, 92%, 93%, 94%, 95%, 96%, 97%, 98%, or 99%, identical to the human Oct4 sequence of SEQ ID NO: 1. In some aspects, the polynucleotide comprises the polynucleotide sequence of SEQ ID NO: 1. In some aspects, the Oct4 protein is a mouse Oct4 protein. In some aspects, the polynucleotide comprises a polynucleotide sequence that is at least 85%, 86%, 87%, 88%, 89%, 90%, 91%, 92%, 93%, 94%, 95%, 96%, 97%, 98%, or 99%, identical to the human Oct4 sequence of SEQ ID NO: 8. In some aspects, the polynucleotide comprises the polynucleotide sequence of SEQ ID NO: 8.

In some aspects, the polynucleotide comprises a polynucleotide sequence encoding a Sox2 protein. In some aspects, the Sox2 protein is a human Sox2 protein. In some aspects, the polynucleotide comprises a polynucleotide sequence that is at least 85%, 86%, 87%, 88%, 89%, 90%, 91%, 92%, 93%, 94%, 95%, 96%, 97%, 98%, or 99%, identical to the human Sox2 sequence of SEQ ID NO: 2. In some aspects, the polynucleotide comprises the polynucleotide sequence of SEQ ID NO: 2. In some aspects, the Sox2 protein is a mouse Sox2 protein. In some aspects, the polynucleotide comprises a polynucleotide sequence that is at least 85%, 86%, 87%, 88%, 89%, 90%, 91%, 92%, 93%, 94%, 95%, 96%, 97%, 98%, or 99%, identical to the human Sox2 sequence of SEQ ID NO: 9. In some aspects, the polynucleotide comprises the polynucleotide sequence of SEQ ID NO: 9.

In some aspects, the polynucleotide comprises a polynucleotide sequence encoding a Klf4 protein. In some aspects, the Klf4 protein is a human Klf4 protein. In some aspects, the polynucleotide comprises a polynucleotide sequence that is at least 85%, 86%, 87%, 88%, 89%, 90%, 91%, 92%, 93%, 94%, 95%, 96%, 97%, 98%, or 99%, identical to the human Klf4 sequence of SEQ ID NO: 3. In some aspects, the polynucleotide comprises the polynucleotide sequence of SEQ ID NO: 3. In some aspects, the Klf4 protein is a mouse Klf4 protein. In some aspects, the polynucleotide comprises a polynucleotide sequence that is at least 85%, 86%, 87%, 88%, 89%, 90%, 91%, 92%, 93%, 94%, 95%, 96%, 97%, 98%, or 99%, identical to the human Klf4 sequence of SEQ ID NO: 10. In some aspects, the polynucleotide comprises the polynucleotide sequence of SEQ ID NO: 10.

In some aspects, the polynucleotide comprises a polynucleotide sequence encoding a c-Myc protein. In some aspects, the c-Myc protein is a human c-Myc protein. In some aspects, the polynucleotide comprises a polynucleotide sequence that is at least 85%, 86%, 87%, 88%, 89%, 90%, 91%, 92%, 93%, 94%, 95%, 96%, 97%, 98%, or 99%, identical to the human c-Myc sequence of SEQ ID NO: 4. In some aspects, the polynucleotide comprises the polynucleotide sequence of SEQ ID NO: 4. In some aspects, the c-Myc protein is a mouse c-Myc protein. In some aspects, the polynucleotide comprises a polynucleotide sequence that is at least 85%, 86%, 87%, 88%, 89%, 90%, 91%, 92%, 93%, 94%, 95%, 96%, 97%, 98%, or 99%, identical to the human c-Myc sequence of SEQ ID NO: 11. In some aspects, the polynucleotide comprises the polynucleotide sequence of SEQ ID NO: 11.

In some aspects, the polynucleotide comprises a polynucleotide sequence encoding a human L-Myc protein of SEQ ID NO: 5. In some aspects, the polynucleotide comprises a polynucleotide sequence encoding a rodent L-Myc protein. In some aspects, the polynucleotide comprises a polynucleotide sequence encoding a human N-Myc protein of SEQ ID NO: 6. In some aspects, the polynucleotide comprises a polynucleotide sequence encoding a rodent N-Myc protein.

In some aspects, a polynucleotide comprises a polynucleotide sequence encoding an Oct4 protein and/or a polynucleotide sequence encoding a Sox2 protein and/or a polynucleotide sequence encoding a Klf4 protein all of which are operably linked to an inducible promoter. In some aspects, a polynucleotide comprises a polynucleotide sequence encoding an Oct4 protein and/or a polynucleotide sequence encoding a Sox2 protein and/or a polynucleotide sequence encoding a Klf4 protein and/or a c-Myc protein all of which are operably linked to an inducible promoter. In some aspects, a polynucleotide comprises a polycistronic cassette comprising a polynucleotide sequence encoding an Oct4 protein, a Sox2 protein, and a Klf4 protein. In some aspects, a polynucleotide comprises a polycistronic cassette comprising a polynucleotide sequence encoding an Oct4 protein, a Sox2 protein, a Klf4 protein, and a c-Myc protein. In some aspects, one polynucleotide comprises a polynucleotide sequence encoding an Oct4 protein and/or a polynucleotide sequence encoding a Sox2 protein and/or a polynucleotide sequence encoding a Klf4 protein all of which are operably linked to an inducible promoter and a second polynucleotide comprises a polynucleotide encoding a c-Myc protein operably linked to an inducible promoter.

In some aspects, the polynucleotide further comprises a proteolytic cleavable site. In some aspects, the proteolytic cleavable site is a self-processing cleavage site or a furin cleavage site. In some aspects, the self-processing cleavage site is a P2A, E2A, F2A, or T2A peptide. In some aspects, the furin cleavage site comprise the consensus sequence RXK(R)R of SEQ ID NO: 17. Thus, the polypeptide produced when the polynucleotide is transcribed in a cell can be cleaved by proteases in the cell to release the Oct4, Sox2, and Klf4 proteins.

In some aspects, a polynucleotide comprises a polynucleotide sequence encoding an Oct4 protein, a polynucleotide sequence encoding a Sox2 protein and a polynucleotide sequence encoding a Klf4 protein ordered in a 5′-3′ direction on the polynucleotide such that a desired amount of each of Oct4, Sox2, and Klf4 is produced in a cell transduced with the polynucleotide. The amount of Oc4, Sox2, and Klf4 protein required in a specific cell type to induce partial reprogramming may vary and a polynucleotide can be chosen that provides an optimal ratio of Oct4, Sox2, and Klf4 protein levels for partial reprogramming of the respective cell type. In some aspects, the level of protein produced from a polynucleotide is highest the closest a protein encoding polynucleotide sequence is located to the promoter sequence. In some aspects, the Oct4, Sox2, and Klf4 are located in 5′ to 3′ order from the promoter sequence. In some aspects, the Oct4, Sox2, Klf4, and c-Myc are located in 5′ to 3′ order from the promoter sequence.

In some aspects, the polynucleotide comprises an inducible promoter, a polynucleotide sequence encoding an Oct4 protein, a proteolytic cleavable site, and a polynucleotide sequence encoding a Sox2 protein.

In some aspects, the polynucleotide comprises an inducible promoter, a polynucleotide sequence encoding an Oct4 protein, a proteolytic cleavable site, and a polynucleotide sequence encoding a Klf4 protein.

In some aspects, the polynucleotide comprises an inducible promoter, a polynucleotide sequence encoding a Sox2 protein, a proteolytic cleavable site, and a polynucleotide sequence encoding a Klf4 protein.

In some aspects, the polynucleotide comprises an inducible promoter, a polynucleotide sequence encoding an Oct4 protein, a first proteolytic cleavable site, a polynucleotide sequence encoding a Sox2 protein, a second proteolytic cleavable site, and a polynucleotide sequence encoding a Klf4 protein.

In some aspects, the polynucleotide comprises an inducible promoter, a polynucleotide sequence encoding an Oct4 protein, a first proteolytic cleavable site, a polynucleotide sequence encoding a Klf4 protein, a second proteolytic cleavable site, and a polynucleotide sequence encoding a Sox2 protein in 5′-3′ direction.

In some aspects, the polynucleotide comprises an inducible promoter, a polynucleotide sequence encoding a Sox2 protein, a first proteolytic cleavable site, a polynucleotide sequence encoding an Oct4 protein, a second proteolytic cleavable site, and a polynucleotide sequence encoding a Klf4 protein in 5′-3′ direction.

In some aspects, the polynucleotide comprises an inducible promoter, a polynucleotide sequence encoding a Sox2 protein, a first proteolytic cleavable site, a polynucleotide sequence encoding a Ktlf4 protein, a second proteolytic cleavable site, and a polynucleotide sequence encoding an Oct4 protein in 5′-3′ direction.

In some aspects, the polynucleotide comprises an inducible promoter, a polynucleotide sequence encoding a Klf4 protein, a first proteolytic cleavable site, a polynucleotide sequence encoding an Oct4 protein, a second proteolytic cleavable site, and a polynucleotide sequence encoding a Sox2 protein in 5′-3′ direction.

In some aspects, the polynucleotide comprises an inducible promoter, a polynucleotide sequence encoding a Klf4 protein, a first proteolytic cleavable site, a polynucleotide sequence encoding a Sox2 protein, a second proteolytic cleavable site, and a polynucleotide sequence encoding an Oct4 protein in 5′-3′ direction.

In some aspects, the inducible promoter is a tetracycline-inducible or an RU486-inducible promoter.

In some aspects, the first proteolytic site is a P2A peptide, an E2A peptide, a F2A peptide, a T2A peptide or a RXK(R)R consensus sequence of SEQ ID NO: 17.

In some aspects, the second proteolytic site is a P2A peptide, an E2A peptide, a F2A peptide, a T2A peptide or a RXK(R)R consensus sequence of SEQ ID NO: 17.

In some aspects, the polynucleotide comprises a second promoter.

In some aspects, the polynucleotide comprises a first inducible promoter, a polynucleotide sequence encoding an Oct4 protein, a second inducible or non-inducible promoter, a polynucleotide sequence encoding a Sox2 protein, a proteolytic cleavable site, and a polynucleotide sequence encoding a Klf4 protein in 5′-3′ direction.

In some aspects, the polynucleotide comprises a first inducible promoter, a polynucleotide sequence encoding an Oct4 protein, a second inducible or non-inducible promoter, a polynucleotide sequence encoding a Klf4 protein, a proteolytic cleavable site, and a polynucleotide sequence encoding a Sox2 protein in 5′-3′ direction.

In some aspects, the polynucleotide comprises a first inducible promoter, a polynucleotide sequence encoding an Oct4 protein, a proteolytic cleavable site, a polynucleotide sequence encoding a Sox2 protein, a second inducible or non-inducible promoter, and a polynucleotide sequence encoding a Klf4 protein in 5′-3′ direction.

In some aspects, the polynucleotide comprises a first inducible promoter, a polynucleotide sequence encoding a Sox2 protein, a proteolytic cleavable site, a polynucleotide sequence encoding an Oct4 protein, a second inducible or non-inducible promoter, and a polynucleotide sequence encoding a Klf4 protein in 5′-3′ direction.

In some aspects, the polynucleotide comprises a first inducible promoter, a polynucleotide sequence encoding an Oct4 protein, a proteolytic cleavable site, a polynucleotide sequence encoding a Klf4 protein, a second inducible or non-inducible promoter, and a polynucleotide sequence encoding a Sox2 protein in 5′-3′ direction.

In some aspects, the polynucleotide comprises a first inducible promoter, a polynucleotide sequence encoding a Klf4 protein, a proteolytic cleavable site, a polynucleotide sequence encoding an Oct4 protein, a second inducible or non-inducible promoter, and a polynucleotide sequence encoding a Sox2 protein in 5′-3′ direction.

In some aspects, the first promoter is inducible or non-inducible and the second promoter is inducible.

In some aspects, the first and the second inducible promoter are induced by the same inducing compound. In some aspects, the first and the second inducible promoter are induced by different inducing compounds. For example, the first inducible promoter can be a promoter that is induced by tetracycline and the second inducible promoter can be a promoter that is induced by coumermycin. In some aspects, the polynucleotide comprises a first inducible promoter operably linked to a polynucleotide encoding at least one of Oct4, Sox2, and/or Klf4. In some aspects, the polynucleotide comprises a first inducible promoter that is a tetracycline-inducible promoter and is operably linked to a polynucleotide encoding at least one of Oct4, Sox2, and/or Klf4. In some aspects, the polynucleotide further comprises a second inducible promoter operably linked to c-Myc. In some aspects, the polynucleotide further comprises a second inducible promoter that is a coumermycin-inducible promoter operably linked to c-Myc. In some aspects, the first inducible promoter that is a tetracycline-inducible promoter and is operably linked to a polynucleotide encoding at least one of Oct4, Sox2, and/or Klf4 and the second inducible promoter that is a coumermycin-inducible promoter operably linked to c-Myc are present on different polynucleotides.

In some aspects, the first and the second promoter initiate transcription in the same direction. In some aspects, the first and the second promoter initiate transcription in different directions.

In some aspects, the polynucleotide further comprises a polyadenylation signal sequence. In some aspects, the polyadenylation signal sequence is an SV40 polyadenylation signal sequence, a human growth hormone polyadenylation signal sequence, or a bovine growth hormone polyadenylation signal sequence.

In some aspects, the polynucleotide further comprises a WPRE sequence.

In some aspects, the polynucleotide further comprises an IRES.

In some aspects, the polynucleotide comprises an inducible promoter, a polynucleotide sequence encoding an Oct4 protein, a first IRES, a polynucleotide sequence encoding a Sox2 protein, a second IRES, and a polynucleotide sequence encoding a Klf4 protein in 5′-3′ direction.

In some aspects, the polynucleotide comprises an inducible promoter, a polynucleotide sequence encoding an Oct4 protein, a first IRES, a polynucleotide sequence encoding a Klf4 protein, a second IRES, and a polynucleotide sequence encoding a Sox2 protein in 5′-3′ direction.

In some aspects, the polynucleotide comprises an inducible promoter, a polynucleotide sequence encoding a Sox2 protein, a first IRES, a polynucleotide sequence encoding an Oct4 protein, a second IRES, and a polynucleotide sequence encoding a Klf4 protein in 5′-3′ direction.

In some aspects, the polynucleotide comprises an inducible promoter, a polynucleotide sequence encoding a Sox2 protein, a first IRES, a polynucleotide sequence encoding a Klf4 protein, a second IRES, and a polynucleotide sequence encoding an Oct4 protein in 5′-3′ direction.

In some aspects, the polynucleotide comprises an inducible promoter, a polynucleotide sequence encoding a Klf4 protein, a first IRES, a polynucleotide sequence encoding an Oct4 protein, a second IRES, and a polynucleotide sequence encoding a Sox2 protein in 5′-3′ direction.

In some aspects, the polynucleotide comprises an inducible promoter, a polynucleotide sequence encoding a Klf4 protein, a first IRES, a polynucleotide sequence encoding a Sox2 protein, a second IRES, and a polynucleotide sequence encoding an Oct4 protein in 5′-3′ direction.

In some aspects, the polynucleotide comprises an inducible promoter, a polynucleotide sequence encoding an Oct4 protein, a second inducible or non-inducible promoter, a polynucleotide sequence encoding a Sox2 protein, an IRES, and a polynucleotide sequence encoding a Klf4 protein in 5′-3′ direction.

In some aspects, the polynucleotide comprises a inducible promoter, a polynucleotide sequence encoding an Oct4 protein, a second inducible or non-inducible promoter, a polynucleotide sequence encoding a Klf4 protein, an IRES, and a polynucleotide sequence encoding a Sox2 protein in 5′-3′ direction.

In some aspects, the polynucleotide comprises a first inducible promoter, a polynucleotide sequence encoding an Oct4 protein, an IRES, a polynucleotide sequence encoding a Sox2 protein, a second inducible or non-inducible promoter, and a polynucleotide sequence encoding a Klf4 protein in 5′-3′ direction.

In some aspects, the polynucleotide comprises a first inducible promoter, a polynucleotide sequence encoding a Sox2 protein, an IRES, a polynucleotide sequence encoding an Oct4 protein, a second inducible or non-inducible promoter, and a polynucleotide sequence encoding a Klf4 protein in 5′-3′ direction.

In some aspects, the polynucleotide comprises a first inducible promoter, a polynucleotide sequence encoding an Oct4 protein, an IRES, a polynucleotide sequence encoding a Klf4 protein, a second inducible or non-inducible promoter, and a polynucleotide sequence encoding a Sox2 protein in 5′-3′ direction.

In some aspects, the polynucleotide comprises a first inducible promoter, a polynucleotide sequence encoding a Klf4 protein, an IRES, a polynucleotide sequence encoding an Oct4 protein, a second inducible or non-inducible promoter, and a polynucleotide sequence encoding a Sox2 protein in 5′-3′ direction.

In some aspects, the polynucleotide comprises an inducible promoter operably linked to a polynucleotide encoding a c-Myc protein. In some aspects, the polynucleotide comprises a non-inducible promoter operably linked to a polynucleotide encoding a transactivator. In some aspects, the polynucleotide comprises an inducible promoter operably linked to a polynucleotide encoding a c-Myc protein and a non-inducible promoter operably linked to a polynucleotide encoding a transactivator.

In some aspects, the polynucleotide further comprises a polynucleotide sequence encoding a marker protein. In some aspects, the marker protein is a β-galactosidase protein, a green fluorescent protein, a red fluorescent protein, a yellow fluorescent protein, cyan fluorescent protein, or a blue fluorescent protein, a tdTomato protein, or a mCherry protein.

In some aspects, the polynucleotide further comprises an intron sequence. In some aspects, the intron sequence comprises a CMV intronic sequence, a β-actin intronic sequence, a SV40 enhancer sequence, or a combination thereof.

In some aspects, provided is a polynucleotide that encodes a Cas9 fusion protein (CRISPR activator) and a guide RNA sequence targeting a promoter or enhancer of the endogenous locus of at least one of Oct4, Sox2, and Klf4. In some aspects, the polynucleotide encodes a Cas9 fusion protein and a guide RNA sequence targeting a promoter or enhancer of the endogenous locus of Oct4, Sox2, Klf4 and, optionally, c-Myc.

In some aspects, the polynucleotide as described herein is an RNA. In some aspects, the polynucleotide as described herein is a DNA. In some aspects, the polynucleotide is present within a vector. In some aspects, the polynucleotide is present within an integrating vector. In some aspects, the polynucleotide is present within a non-integrating vector. In some aspects, the polynucleotide is present within an AAV vector, an adenoviral vector, a lentiviral vector, or a retroviral vector. In some aspects, the polynucleotide is a non-integrating RNA.

### III. Vectors, Cells

Also provided are vectors comprising a polynucleotide as described herein.

In some aspects, the vectors are viral vectors. In some aspects, the vectors are non-viral vectors. In some aspects, the vectors are lipids. In some aspects, the vectors are polymers.

In some aspects, the viral vectors are adeno-associated viral (AAV) vectors, adenoviral vectors, lentiviral vectors, or retroviral vectors. In some aspects, the AAV vector is a AAV1, AAV2, AAV3, AAV4, AAV5, AAV6, AAV7, AAV8, AAVRH8, AAVrh9, AAV9, AAVrh10, AAV10, AAVRH10, AAV11, AAV12, or AAV-DJ vector.

In some aspects, an AAV vector with broad target spectrum is chosen to transduce various cell types. In some aspects, the AAV vector is an AAV-DJ vector.

In some aspects, an AAV vector with a select target cell or select target cells is chosen. The target specificity of different AAV vectors is known in the art (see, e.g, AAV Production Protocol, Genemedi Biotech, Inc. 2018.).

In some aspects, an AAV vector is modified to target a select cell type or select cell types. For example, an AAV cap sequence can be modified to remove a cell-targeting epitope from the capsid and introduce an alternative cell-targeting sequence in the capsid. AAV cap sequences modified in this manner are known in the art.

In some aspects, the viral vectors are retroviral vectors and comprise a polynucleotide as described herein. In some aspects, the retroviral vectors are self-inactivating retroviral vectors. In some aspects, the retroviral vectors are pseudo-typed retroviral vectors. In some aspects, the retroviral vectors are modified to target a specific cell type or cell types. In some aspects, a retroviral vector comprises a polynucleotide encoding an Oct4 protein, Sox2 protein, Klf4 protein and/or a c-Myc protein. In some aspects, a retroviral vector comprises a polynucleotide encoding an Oct4 protein, Sox2 protein, Klf4 protein and a c-Myc protein. In some aspects, a retroviral vector comprises a polynucleotide comprising a polycistronic cassette encoding an Oct4 protein, Sox2 protein, Klf4 protein and/or a c-Myc protein. In some aspects, a retroviral vector comprises a polynucleotide encoding an Oct4 protein, Sox2 protein, and a Klf4 protein. In some aspects, a retroviral vector comprises a polynucleotide comprising a polycistronic cassette encoding an Oct4 protein, Sox2 protein, and a Klf4 protein. In some aspects, a retroviral vector comprises a polynucleotide encoding a transcriptional activator and one or more of an Oct4 protein, Sox2 protein, Klf4 protein, c-Myc and/or a dnNFκBIA protein.

In some aspects, the viral vectors are lentiviral vectors and comprise a polynucleotide as described herein. In some aspects, the lentiviral vectors are self-inactivating lentiviral vectors and comprise at least one deletion in an enhancer/promoter unit in the U3 region of the 3′ LTR. In some aspects, the lentiviral vectors are pseudo-typed lentiviral vectors. In some aspects, the lentiviral vectors are pseudotyped with a vesicular stomatitis virus glycoprotein (VSV G). In some aspects, the lentiviral vectors are pseudo-typed with glycoproteins from retroviridae, rhabdoviridae, arenaviridae, flaviviridae, paramyxoviridae, baculoviridae, filoviridae or combinations thereof. In some aspects, the lentiviral vectors are modified with target cell specific binding moieties to target specific cells or cell types. In some aspects, the lentiviral vectors comprise a tat-independent heterologous promoter that replaces a portion of the 5′LTR. In some aspects, the lentiviral vectors comprise an inactivating mutation in an integrase gene such that the lentiviral vectors remain episomal in a transduced cell.

In some aspects, a lentiviral vector comprises a polynucleotide encoding an Oct4 protein, a Sox2 protein, a Klf4 protein and/or a c-Myc protein. In some aspects, a lentiviral vector comprises a polynucleotide encoding an Oct4 protein, a Sox2 protein, a Klf4 protein, and a c-Myc protein. In some aspects, a lentiviral vector comprises a polynucleotide comprising a polycistronic cassette encoding an Oct4 protein, a Sox2 protein, a Klf4 protein and/or a c-Myc protein. In some aspects, a lentiviral vector comprises a polynucleotide encoding an Oct4 protein, a Sox2 protein, and a Klf4 protein. In some aspects, a lentiviral vector comprises a polynucleotide comprising a polycistronic cassette encoding an Oct4 protein, a Sox2 protein, and a Klf4 protein. In some aspects, a lentiviral vector comprises a polynucleotide encoding a transcriptional activator and one or more of an Oct4 protein, a Sox2 protein, a Klf4 protein, a c-Myc and/or a dnNFκBIA protein. In some aspects, the non-viral vector is a plasmid DNA, a RNA, a cationic polymer, a lipid, a lipopolymer or a chemical derivative thereof.

In some aspects, the cationic polymer is present in an amount sufficient to produce a ratio of amine nitrogen in the cationic polymer to phosphate in the DNA plasmid vector or RNA vector from about 0.1:1 to about 100:1.

In some aspects, the cationic polymer is present in an amount sufficient to produce a ratio of amine nitrogen in the cationic polymer to phosphate in the DNA plasmid vector or RNA vector from about 0.1:1 to about 10:1.

In some aspects, the cationic polymer is present in an amount sufficient to produce a ratio of amine nitrogen in the cationic polymer to phosphate in the DNA plasmid vector or RNA vector from about 0.1:1 to about 5:1.

In some aspects, the non-viral vector comprises about 0.5 mg/ml to about 5.0 mg/ml polynucleotide complexed with the cationic polymer. In some aspects, the cationic polymer is a poly(ethylenimine) (PEI) polymer, poly-L-lysine, poly(L-arginine) (PLA), polyallylamine (PAH), polyamidoamine, diethylaminoethyl dextrans, chitosan, poly(dimethyl-aminoethyl methacrylates) polyvinylamine homo- or copolymer, a poly(vinylbenzyl-tri-Cl-C4-alkylammonium salt), a polymer of an aliphatic or araliphatic dihalide and an aliphatic N,N,N′,N′-tetra-Cl-C4-alkyl-alkylenediamine, a poly(vinylpyridin) or poly(vinylpyridinium salt), a poly(N,Ndiallyl-N,N-di-Cl-C4-alkyl-ammoniumhalide), a homo- or copolymer of a quaternized diel-C4-alkyl-aminoethyl acrylate or methacrylate, POLYQUAD™, a polyaminoamide and the like; or derivatives thereof.

Further provided are cells comprising a polynucleotide as described herein or a vector as described herein. In some aspects, the cells can be bacterial cells, yeast cells, fungal cells, insect cells, or mammalian cells.

In some aspects, the cells comprising a polynucleotide as described herein can be used to manufacture a viral vector. In some aspects, the cells are insect cells comprising a polynucleotide as described herein and further viral vector production components and are used to prepare a viral vector as described herein. In some aspects, the cells are mammalian cells comprising a polynucleotide as described herein and further viral vector production components and are used to prepare a viral vector as described herein.

In some aspects, the cells are insect cells comprising a polynucleotide as described herein and AAV viral vector production components and are used to prepare AAV vectors.

In some aspects, the cells are mammalian cells comprising a polynucleotide as described herein and AAV viral vector production components and are used to prepare AAV vectors. In some aspects, the AAV vector production components comprise polynucleotides encoding Rep proteins and Cap proteins and an AAV vector comprising a polynucleotide that encodes, e.g, at least one regeneration factor and comprises at least one ITR. In some aspects, the mammalian cells, upon expression of the Rep and Cap proteins enable the packaging of the AAV vector encoding the at least one regeneration factor into an AAV capsid.

In some aspects, the cells are mammalian cells comprising a polynucleotide as described herein and retroviral vector production components and are used to prepare retroviral vectors. In some aspects, the retroviral vector production components comprise polynucleotides encoding gag, pol, and env proteins, and a retroviral vector comprising a polynucleotide that encodes, e.g., at least one regeneration factor and comprises a LTR. In some aspects, the mammalian cells, upon expression of gag, pol, and env proteins enable the packaging of the retroviral vector encoding the at least one regeneration factor into a retroviral capsid.

In some aspects, the cells are mammalian cells comprising a polynucleotide as described herein and lentiviral vector production components and are used to prepare lentiviral vectors. In some aspects, the lentiviral vector production components comprise polynucleotides encoding gag, pol, and env proteins and, optionally, tat and rev proteins, and a retroviral vector comprising a polynucleotide that encodes, e.g., at least one regeneration factor and comprises a LTR. In some aspects, the mammalian cells, upon expression of gag, pol, and env proteins, and, optionally, tat and rev proteins, enable the packaging of the lentiviral vector encoding the at least one regeneration factor into a lentiviral capsid.

In some aspects, provided are cells comprising a polynucleotide or vector or nanoparticle as described herein, which cells are present in an organ. In some aspects, the cells comprising a polynucleotide as described herein are present in an organ that has been exposed to an ischemic injury, a hypertensive injury, a toxic injury, a perfusion injury, an immune injury, a physical injury, steatosis, virus induced hepatitis, alcohol, or fibrosis not associated with any known cause.

In some aspects, the cells comprising a polynucleotide as described herein can be used to treat a subject. In some aspects, the cells comprising a polynucleotide as described herein can be administered to a subject in need of reversing senescence, wherein the cells after being administered express the proteins encoded by the polynucleotides described herein and the expressed proteins reverse cellular processes associated with a senescent phenotype. In some aspects, the cells comprising a polynucleotide as described herein after being administered to a subject replicate. In some aspects, the cells comprising a polynucleotide as described herein after being administered to a subject do not replicate. In some aspects, the cells comprising a polynucleotide as described herein can be hematopoietic progenitor cells. In some aspects, the cells comprising a polynucleotide as described herein can be hematopoietic stem cells. In some aspects, the cells comprising a polynucleotide as described herein can be muscle stem cells. In some aspects, the cells comprising a polynucleotide as described herein can be adipose stem cells.

### IV. Nanoparticles

Further provided are lipid particles, e.g., lipid nanoparticles comprising a polynucleotide as described herein. In some aspects, the lipid nanoparticles are of a size of about 10 nm to about 1000 nm. In some aspects, the lipid nanoparticles comprise a solid lipid core matrix and a surfactant. In some aspects, the lipid nanoparticles comprise cationic lipids. In some aspects, the lipid nanoparticles comprise cationic lipids, non-cationic lipids and, optionally, lipids that prevent aggregation.

In some aspects, the lipid nanoparticles comprise amphiphilic lipids arranged in at least one bilayer with an aqueous interior comprising a polynucleotide encoding a regeneration factor. In some aspects, the polynucleotide of the lipid nanoparticles is a DNA, a RNA or a mixture of both. In some aspects, the lipid nanoparticles comprise more than one polynucleotide and each polynucleotide encodes a regeneration factor. In some aspects, the lipid nanoparticles comprise a RNA encoding Oct4. In some aspects, the lipid nanoparticles comprise a RNA encoding Sox2. In some aspects, the lipid nanoparticles comprise a RNA encoding Klf4. In some aspects, the lipid nanoparticles comprise a RNA encoding c-Myc. In some aspects, the lipid nanoparticles comprise a RNA encoding Oct4, a RNA encoding Sox2, a RNA encoding Klf4, and a RNA encoding c-Myc. In some aspects, the lipid nanoparticles comprise a RNA encoding Oct4, a RNA encoding Sox2, and a RNA encoding Klf4. In some aspects, the lipid nanoparticles comprise a RNA encoding Oct4, Sox2, Klf4, and c-Myc. In some aspects, the lipid nanoparticles comprise a RNA encoding Oct4, Sox2, and Klf4. In some aspects, the lipid nanoparticles comprise a RNA comprising a polycistronic cassette encoding Oct4, Sox2, Klf4, and c-Myc. In some aspects, the lipid nanoparticles comprise a RNA comprising a polycistronic cassette encoding Oct4, Sox2, and Klf4.

In some aspects, the lipid nanoparticles comprise an additional agent. In some aspects, the lipid nanoparticles comprise an enhancing agent as described herein.

### V. Compositions, Perfusates, and Kits

Also provided are compositions comprising polynucleotides, vectors, and/or nanoparticles as described herein. In some aspects, provided are perfusates comprising polynucleotides, vectors, and/or nanoparticles as described herein. In some aspects, the perfusates or compositions are liquid solutions comprising polynucleotides, vectors, and/or nanoparticles as described herein and inorganic salts.

In some aspects, a perfusate or composition comprises a phosphate bicarbonate buffered solution.

In some aspects, a perfusate or composition comprises at least one of sodium chloride, calcium chloride, potassium chloride, magnesium chloride, sodium bicarbonate, mono-potassium phosphate, or sodium phosphate dibasic. In some aspects, a perfusate or composition comprises sodium chloride, calcium chloride, potassium chloride, magnesium chloride, sodium bicarbonate, mono-potassium phosphate, and sodium phosphate dibasic.

In some aspects, a perfusate or composition comprises a dextran/albumin solution. In some aspects, a perfusate or composition comprises calcium gluconate.

In some aspects, a perfusate or composition comprises at least one vasodilator. In some aspects, the vasodilator is carbon monoxide, an angiotensin converting enzyme (ACE) inhibitor, an angiotensin receptor blocker, a calcium channel blocker, prostacyclin, hydralazine, minoxidil, and nitroglycerin. In some aspects, a perfusate or composition comprises a vasodilator that is not carbon monoxide.

In some aspects, a perfusate or composition comprises at least one tonicity agent. In some aspects, the tonicity agent is dextrose, glycerin, mannitol, potassium chloride, or sodium chloride.

In some aspects, a perfusate or composition comprises at least one of a buffer, an inorganic salt, a substrate for metabolism, a growth factor, a hormone, an anti-oxidant, an anti-inflammatory agent, an immunosuppressive agent, an anti-coagulant, or an anti-microbial agent. In some aspects, the substrate for metabolism is an amino acid or glucose. In some aspects, a perfusate or composition comprises insulin.

In some aspects, the growth factor is at least one of fibroblast growth factor (FGF), insulin-like growth factor (IGF), transforming growth factor beta (TGF-P), epiregulin, epidermal growth factor (“EGF”), endothelial cell growth factor (“ECGF”), nerve growth factor (“NGF”), leukemia inhibitory factor (“LIF”), bone morphogenetic protein-4 (“BMP-4”), hepatocyte growth factor (“HGF”), vascular endothelial growth factor-A (“VEGF-A”), and cholecystokinin octapeptide.

In some aspects, the immunosuppressive agent includes, but is not limited to, steroidal (e.g., prednisone) or non-steroidal (e.g., sirolimus (Rapamune, Wyeth-Ayerst Canada), tacrolimus (Prograf, Fujisawa Canada), and anti-IL2R daclizumab (Zenapax, Roche Canada). In some aspects, the immunosuppressant agent is 15-deoxyspergualin, cyclosporin, methotrexate, rapamycin, Rapamune (sirolimus/rapamycin), FK506, Lisofylline (LSF), mycophenolate mofetil, anti-thymocyte globulin, belatacept, or everolimus.

In some aspects, an anti-microbial agent is benzalkonium chloride, benzethonium chloride, benzyl alcohol, cetylpyridinium chloride, chlorobutanol, phenol, phenylethyl alcohol, phenylmercuric nitrate, thimersol; an antibiotic including, but not limited to, amoxicillin, penicillin, sulfa drugs, cephalosporins, erythromycin, streptomycin, gentamicin, tetracycline, chlarithromycin, ciproflozacin, azithromycin; an antifungal including, but not limited to, myconazole and terconazole; and combinations of thereof.

In some aspects, an antioxidant includes, but is not limited to, molecules having thiol groups such as reduced glutathione (GSH) or its precursors, glutathione or glutathione analogs, glutathione monoester, and N-acetylcysteine; superoxide dismutase, catalase, vitamin E, Trolox, lipoic acid, lazaroids, butylated hvdroxyanisole (BHA), vitamin K and the like; and combinations thereof.

In some aspects, a perfusate or composition comprises an oxygenation agent. In some aspects, the oxygenation agent is a plasma free, leukocyte-depleted red blood cell, hemoglobin, pyridoxylated hemoglobin, a synthetic hemoglobin-based oxygen carrier including, but not limited to, a polymerized hemoglobin-based oxygen carrier; or an acellular oxygen-carrying medium including, but not limited to, Lifor™, Aqix® RS-I, or STEEN Solution™. In some aspects, the oxygenation agent is HEMOXYCarrier®, Hemopure, Hemarina®, or Perfluorocarbon.

In some aspects, a perfusate or composition comprises at least one of human serum albumin, dextran and an extracellular electrolyte composition. In some aspects, a perfusate or composition comprises human serum albumin, dextran and an extracellular electrolyte composition.

In some aspects, a perfusate or composition comprises a dextran/albumin solution, packed red blood cells, calcium gluconate, heparin, cefozolin, verapamil, amino acids, glucose, aninsluin, and Ringer's lactate.

In some aspects, a perfusate or composition comprises packed red blood cells and colloid solution (e.g., Gelafuncin, B Braun), cefuroxime, heparin, sodium bicarbonate, calcium gluconate, insulin, sodium taurocholate, prostacyclin, amino acids, and glucose.

In some aspects, a perfusate or composition comprises a polynucleotide as described herein and a delivery agent or a vector as described herein.

In some aspects, the delivery agent is a viral vector. In some aspects, the delivery agent is a retroviral vector. In some aspects, the delivery agent is a lentiviral vector. In some aspects, the delivery agent is an AAV vector. In some aspects, the delivery agent is AAV-DJ vector. In some aspects, the delivery agent is a nanoparticle. In some aspects, the delivery agent is a lipid nanoparticle. In some aspects, the delivery agent is a polymeric nanoparticle. In some aspects, the lipid or polymeric nanoparticle are conjugated to a ligand moiety to target the lipid or polymeric nanoparticle to a specific cell, tissue or organ. In some aspects, the delivery agent is a cationic polymer. In some aspects, the delivery agent is a lipid, a lipopolymer or a chemical derivative thereof.

In some aspects, the cationic polymer of the composition is present in an amount sufficient to produce a ratio of amine nitrogen in the cationic polymer to phosphate in the DNA plasmid or RNA from about 0.1:1 to about 100:1.

In some aspects, the cationic polymer of the composition is present in an amount sufficient to produce a ratio of amine nitrogen in the cationic polymer to phosphate in the DNA plasmid or RNA from about 0.1:1 to about 10:1.

In some aspects, the cationic polymer of the composition is present in an amount sufficient to produce a ratio of amine nitrogen in the cationic polymer to phosphate in the DNA plasmid or RNA from about 0.1:1 to about 5:1.

In some aspects, the polynucleotide as described herein is present at about 0.5 mg/ml to about 5.0 mg/ml complexed with the cationic polymer of the composition. In some aspects, the cationic polymer of the composition is a poly(ethylenimine) (PEI) polymer, poly-L-lysine, polyamidoamine, diethylaminoethyl dextrans, chitosan, poly(dimethyl-aminoethyl methacrylates) or a derivative thereof.

Further provided are pharmaceutical compositions comprising polynucleotides, vectors, and/or nanoparticles as described herein. In some aspects, the pharmaceutical compositions comprise a pharmaceutically acceptable carrier or excipient.

Some examples of materials that can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Additional non-limiting examples of agents suitable for compositions described herein include: PEG conjugated polynucleotides, phospholipid conjugated polynucleotides, polynucleotides containing lipophilic moieties, phosphorothioates, P-glycoprotein inhibitors (such as Pluronic P85) which can enhance entry of drugs into various tissues; biodegradable polymers, such as poly (DL-lactide-coglycolide) microspheres for sustained release delivery after transplantation.

In some aspects, the compositions further comprise wetting agents, emulsifiers and lubricants, release agents, coating agents, preservatives and antioxidants.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In some aspects, a composition of the disclosure comprises an excipient selected from cyclodextrins, celluloses, liposomes, micelle forming agents, e.g., bile acids, and polymeric carriers, e.g., polyesters and polyanhydrides; and a polynucleotide described herein. In some aspects, a composition renders orally bioavailable a polynucleotide described herein.

Methods of preparing these compositions, perfusates, or pharmaceutical compositions include a step of bringing into association a polynucleotide as described herein with a carrier and, optionally, one or more accessory ingredients. In general, the compositions perfusates, or pharmaceutical compositions are prepared by uniformly and intimately bringing into association a polynucleotide with liquid carriers, or finely divided solid carriers, or both.

Liquid dosage forms of the compositions, perfusates, or pharmaceutical compositions as described herein include pharmaceutically acceptable emulsions, microemulsions, solutions, and suspensions. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to a polynucleotide or vector, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for parenteral administration or administration in a perfusate may comprise one or more polynucleotides, vectors, or nanoparticles as described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the compositions isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These pharmaceutical compositions may also contain adjuvants such as preservatives, wetting agents, emulsifying agents and dispersing agents. Prevention of the action of microorganisms upon the polynucleotides, vectors, or nanoparticles described herein may be ensured by the inclusion of various antibacterial and antifungal agents, for example, paraben, chlorobutanol, phenol sorbic acid, and the like.

Depot forms may be made by forming microencapsulated matrices of the polynucleotides or vectors in biodegradable polymers such as polylactide-polyglycolide. Depending on the ratio of polynucleotide or vector to polymer, and the nature of the particular polymer employed, the rate of polynucleotide or vector release can be controlled. Examples of other biodegradable polymers include poly(orthoesters) and poly(anhydrides). Formulations may also prepared by entrapping the polynucleotide or vector in liposomes or microemulsions that are compatible with the organ tissues.

Regardless of the route of administration selected, the polynucleotide, vector or nanoparticle described herein, and/or the pharmaceutical compositions of the present disclosure, may be formulated into pharmaceutically-acceptable dosage forms by conventional methods known to those of skill in the art. Actual dosage levels of the polynucleotide or vector in the pharmaceutical compositions may be varied so as to obtain an amount of the polynucleotide or vector which is effective to achieve the desired therapeutic response for a particular organ or patient, composition, and mode of administration, without being unacceptably toxic to the organ or patient.

For example, the dosage level of a polynucleotide, vector or nanoparticle in a perfusate will depend upon a variety of factors including the activity of the particular polynucleotide, vector, or nanoparticle employed, the type or organ perfused, the method of perfusion, the time of perfusion, and/or other drugs, compounds and/or materials used in combination with the particular polynucleotide, vector, or nanoparticle employed and the age, sex, weight, condition, general health and prior medical history of a subject from which the organ was retrieved and the age, sex, weight, condition, general health and prior medical history of a subject that receives the organ.

In some aspects, the polynucleotides or vectors described herein are administered to cells and/or organs by a variety of methods including, but not limited to, iontophoresis, incorporation into vehicles, such as liposomes, hydrogels, cyclodextrins, biodegradable nanocapsules, and bioadhesive microspheres. In some aspects, microemulsification technology may be utilized to improve bioavailability of polynucleotides or vectors as described herein and enhance bioavailability of the polynucleotide or vector by directing absorption to the lymphatic system instead of the circulatory system, e.g., of an explanted organ.

In some aspects, the formulations contain micelles formed from a polynucleotide or vector described herein and at least one amphiphilic carrier, in which the micelles have an average diameter of less than about 100 nm. In some aspects, micelles having an average diameter less than about 50 nm are used with polynucleotides or vectors described herein. In some aspects, micelles having an average diameter less than about 30 nm, or even less than about 20 nm are used.

While all suitable amphiphilic carriers are contemplated, the presently preferred carriers are generally those that have Generally-Recognized-as-Safe (GRAS) status. Examples of amphiphilic carriers include saturated and monounsaturated polyethyleneglycolyzed fatty acid glycerides, such as those obtained from fully or partially hydrogenated various vegetable oils. Such oils may advantageously consist of tri-, di-, and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers may be useful, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, and the like.

Hydrophilic polymers suitable for use with polynucleotides or vectors described herein are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. In some aspects, polymers have a molecular weight of from about 100 or 120 daltons up to about 5,000 or 10,000 daltons, or from about 300 daltons to about 5,000 daltons. In some aspects, the polymer is polyethyleneglycol having a molecular weight of from about 100 to about 5,000 daltons, or having a molecular weight of from about 300 to about 5,000 daltons. In some aspects, the polymer is polyethyleneglycol of 750 daltons (PEG(750)). Polymers may also be defined by the number of monomers therein; in some aspects, polymers of at least about three monomers, such PEG polymers consisting of three monomers (approximately 150 daltons) are used.

Other hydrophilic polymers which may be suitable for use in the present disclosure include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In some aspects, a composition of the present disclosure comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

In some aspects, a composition of the present disclosure comprises a cationic polymer. In some aspects, the cationic polymer comprises a polyethyleneimine (PEI) backbone. In some aspects, the PEI backbone is linked to a lipid or polyethylene glycol. In some aspects, the cationic polymer comprises a cationic dextran, a cationic chitosan, a cationic gelatin, a cationic cellulose, or a cationic cyclodextrin.

In some aspects, a composition of the present disclosure comprises a liposome. Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 m in diameter; large unilamellar vesicles (LUVS) are typically larger than 0.05 m. Oligolamellar large vesicles and multilamellar vesicles have multiple, usually concentric, membrane layers and are typically larger than 0.1 m. Liposomes with several nonconcentric membranes, i.e., several smaller vesicles contained within a larger vesicle, are termed multivesicular vesicles.

In some aspects, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment. Polymer-derivatized lipids such as PEG lipids may act to inhibit micelle/membrane fusion. Active agents, such as polynucleotides or vectors, contained within liposomes described herein are in solubilized form. Aggregates of surfactant and active agent (such as emulsions or micelles containing the polynucleotide or vector) may be entrapped within the interior space of liposomes according to the present disclosure.

Liposomes according to the present disclosure may be prepared by any of a variety of techniques that are known in the art. See, e.g., U.S. Pat. No. 4,235,871; Published PCT applications WO 96/14057; New RRC, Liposomes. A practical approach, IRL Press, Oxford (1990), pages 33-104; Lasic D D, Liposomes from physics to applications, Elsevier Science Publishers BV, Amsterdam, 1993 For example, liposomes described herein may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In some aspects, an active agent is first dispersed by sonication in a lysophosphatidylcholine or other low CMC surfactant (including polymer grafted lipids). The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In some aspects, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. In some aspects, reagents such as DharmaFECT® and Lipofectamine® may be utilized to introduce polynucleotides or vectors into cells.

The release characteristics of a formulation of the present disclosure depend on the encapsulating material, the concentration of encapsulated polynucleotides or vectors, and the presence of release modifiers. For example, release can be manipulated to be pH dependent, for example, using a pH sensitive coating that releases only at a low pH or a higher pH. A coating can be used to prevent release from occurring until after an organ comprising the formulation has been transplanted into a subject. For example, multiple coatings or mixtures of coatings can be used to encapsulate the polynucletoides or vecotrs as described herein to be used in a perfusate or composition such that the encapsulated polynucleotides or vectors are taken up by clels of a perfused irgan but the polynucletoides or vectors are only released in the presence of a low pH that occurs, e.g., with a metabolic acidosis when the function of a transplanted kidney deteriorates.

Release can also be manipulated by inclusion of salts or pore forming agents, which can increase water uptake or release of a composition by diffusion from an encapsulation. Excipients which modify the solubility of the composition can also be used to control the release rate. Agents which enhance degradation of the matrix or release from the encapsulating matrix can also be incorporated. The agents can be added to the composition, added as a separate phase (i.e., as particulates), or can be co-dissolved in the polymer phase depending on the composition. In some aspects, the amount is between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®.

In some aspects, encapsulating particles are coated a cell adhesive polymer. Examples include polymers with free carboxyl groups, such as chitosan, celluloses, and especially polyacrylates (as used herein, polyacrylates refers to polymers including acrylate groups and modified acrylate groups such as cyanoacrylates and methacrylates).

In some aspects, a polynucleotide or vector may be formulated to be contained within, or, adapted to be released by a surgical or medical device or implant. Examples of medical devices include a vascular stent implanted prior to transplantation of an organ, e.g., a stent implanted in an organ artery or an organ ureter to maintain patency. In some aspects, an implant may be coated or otherwise treated with a polynucleotide or vector. For example, hydrogels, or other polymers, such as biocompatible and/or biodegradable polymers, may be used to coat an implant with the compositions as described herein (e.g., the composition may be adapted for use with a medical device by using a hydrogel or other polymer). Polymers and copolymers for coating medical devices with an agent are well-known in the art.

### VI. Organs and Perfusion Systems

Provided herewith are organs and methods of preparing them for transplantation. In some aspects, the organs are explanted from a donor. In some aspects, an organ is explanted from one subject (the donor), submitted to the materials and methods as described herein, and transplanted into another subject (the recipient). In some aspects, an organ is explanted from one subject (the donor), submitted to the materials and methods as described herein, and transplanted into the same subject (the donor and the recipient are one subject). In some aspects, the donors are human subjects. In some aspects, the recipients are human subjects in need of an organ transplant. In some aspects, the organs are organoids generated in vitro. In some aspects, the organoids are rejuvenated using the methods described herein before being transplanted into a recipient. In some aspects, the organoids comprises vasculature, including blood and lymphatic vessels, and are perfused through tubing connected to the blood and/or lymphatic vessels with a perfusate as described herein.

In some aspects, the organs are submitted to perfusion prior to, during, and/or after being submitted to the materials and methods as described herein. In some aspects, the organs are submitted to cold perfusion prior to, during, and/or after being submitted to the materials and methods as described herein. In some aspects, the organs are submitted to normothermic perfusion prior to, during, and/or after being submitted to the materials and methods as described herein. In some aspects, the organs are submitted to cold perfusion prior to and normothermic perfusion during and/or after being submitted to the materials and methods as described herein. In some aspects, the organs are submitted to cold perfusion prior to and during and normothermic perfusion after being submitted to the materials and methods as described herein In some aspects, the cold perfusion and/or normothermic perfusion is performed using a perfusion system.

In some aspects, the perfusion system is a normothermic machine perfusion system. In some aspects, the perfusion system is a hypothermic (cold) machine perfusion system.

In some aspects, a perfusion system is used in the methods described herein. In some aspects, the perfusion system is a Hugo Sachs/Harvard Apparatus, Kidney Assist™ system, OrganOX system, Radnoti system, ARK Kidney system, or Aferetica PerLife® system.

In some aspects, the organ is operably connected to the perfusion system such that a perfusate moved by the perfusion system enters the organ through a blood vessel of the organ. In some aspects, the organ is operably connected to the perfusion system such that a perfusate moved by the perfusion system enters the organ through an artery of the organ. In some aspects, the organ is operably connected to the perfusion system such that a perfusate moved by the perfusion system enters the organ through a vein of the organ.

In some aspects, the organ is a kidney and is operably connected to the perfusion system such that a perfusate moved by the perfusion system enters the organ through a ureter of the organ.

In some aspects, the organ is operably connected to the perfusion system through a perfusate bath whereby the organ is contained in the perfusate bath and the perfusion system moves the perfusate such that the perfusate enters or permeates the organ through a blood vessel, a ureter, or a lymphatic vessel.

In some aspects, the organ is perfused with a perfusate that comprises at least one of albumin, dextran and an extracellular electrolyte composition. In some aspects, the perfusate comprises human serum albumin, dextran and an extracellular electrolyte composition.

In some aspects, the organ is perfused with a perfusate that comprises a polynucleotide, vector, and/or nanoparticle described herein and at least one inorganic salt. In some aspects, the organ is perfused with a perfusate or composition comprises a phosphate bicarbonate buffered solution.

In some aspects, the organ is perfused with a perfusate that comprises at least one of sodium chloride, calcium chloride, potassium chloride, magnesium chloride, sodium bicarbonate, mono-potassium phosphate, or sodium phosphate dibasic. In some aspects, the organ is perfused with a perfusate that comprises sodium chloride, calcium chloride, potassium chloride, magnesium chloride, sodium bicarbonate, mono-potassium phosphate, and sodium phosphate dibasic.

In some aspects, the organ is perfused with a perfusate that comprises at least one vasodilator. In some aspects, the organ is perfused with a perfusate that comprises at least one tonicity agent. In some aspects, the organ is perfused with a perfusate that comprises least one of a buffer, an inorganic salt, an amino acid, a nutrient, a cytokine, a growth factor, a hormone, an anti-oxidant, an anti-inflammatory agent, an immunosuppressive agent, an anti-coagulant, or an anti-microbial agent.

In some aspects, the organ is perfused with a pefusate comprising an oxygenation agent that is a plasma free, leukocyte-depleted red blood cell, hemoglobin, a synthetic hemoglobin-based oxygen carrier, an acellular oxygen-carrying medium, a polymerized hemoglobin-based oxygen carrier, or a pyridoxylated hemoglobin. In some aspects, the organ is perfused with a perfusate comprising an acellular oxygen-carrying medium selected from the group consisting of Lifor™, Aqix® RS-I, STEEN Solution™ Hemarina®, and Perfluorocarbon.

In some aspects, the organ is perfused with a perfusate that comprises a tonicity agent comprising dextrose, glycerin, mannitol, potassium chloride, or sodium chloride.

In some aspects, the organ is perfused with a perfusate that comprises a vasodilator that is carbon monoxide, an angiotensin converting enzyme (ACE) inhibitor, an angiotensin receptor blocker, a calcium channel blocker, prostacyclin, hydralazine, minoxidil, or nitroglycerin. In some aspects, the organ is perfused with a perfusate that comprises a carbon monoxide releasing molecule (e.g., CORM-3, CORM-A1, see e.g., Csongradi et al. Curr Pharm Biotechnol. 13: 819-26, 2012). In some aspects, the organ is perfused with a perfusate into which carbon monoxide has been added. In some aspects, the organ is perfused with a perfusate comprising between about 20 ppm and 250 ppm of carbon monoxide. In some aspects, the perfusate does not comprise carbon monoxide as a vasodilator.

In some aspects, the organ is perfused with a perfusate that comprises a polynucleotide, a polypeptide, a lipid nanoparticle, a polymeric nanoparticle, or a ligand-conjugated lipid or polymeric nanoparticle as described herein. In some aspects, the organ is perfused with a perfusate that comprises a regeneration factor-protein transfer domain fusion protein. In some aspects, the organ is perfused with a perfusate that comprises a viral vector comprising a polynucleotide encoding at least one regeneration factor as described herein. In some aspects, the organ is perfused with a perfusate that comprises an AAV vector, an adenoviral vector, a retroviral vector, or a lentiviral vector. In some aspects, the organ is perfused with a perfusate that comprises a regeneration factor polypeptide.

In some aspects, the organ is a kidney and is perfused with a perfusate that comprises at least one of human serum albumin, dextran and an extracellular electrolyte composition. In some aspects, a kidney is perfused with a perfusate that comprises at least one of human serum albumin, dextran and an extracellular electrolyte composition.

In some aspects, the organ is a kidney and the perfusion system used to prefuse the kidney is a normothermic perfusion system. In some aspects, the organ is a kidney and the perfusion system is a Hugo Sachs/Harvard Apparatus, Kidney Assist™ system, OrganOX system, Radnoti system, ARK Kidney system, or Aferetica PerLife® system. In some aspects, the kidney is perfused using a normothermic perfusion system with a perfusate comprising a dextran/albumin solution, packed red blood cells, calcium gluconate, heparin, an antibiotic, vasodilator, amino acids, glucose, insulin, and Ringer's lactate.

In some aspects, the organ is a liver and the perfusion system used to perfuse the liver is a normothermic perfusion system. In some aspects, the organ is a liver and the perfusion system is a Hugo Sachs/Harvard Apparatus, Kidney Assist™ system, OrganOX system, Radnoti system, ARK Kidney system, or Aferetica PerLife® system. In some aspects, the liver is perfused using a normothermic perfusion system with a perfusate comprising packed red blood cells, a colloid solution, sodium bicarbonate, calcium gluconate, heparin, an antibiotic, a vasodilator, amino acids, glucose, and insulin, and sodium taurocholate in isotonic saline solution.

In some aspects, the organ is a kidney and is perfused with a perfusate that comprises a polynucleotide, polypeptide, vector, and/or nanoparticle described herein. In some aspects, the kidney is characterized by at least one of old age, an ischemic injury, a hypertensive injury, a toxic injury, a perfusion injury, oan immune injury, a physical injury, steatosis, virus induced hepatitis, alcohol, or a fibrosis not associated with any known cause prior to being exposed to a perfusate described herein. In some aspects, an aged or injured kidney is contacted with a perfusate for different periods of time and/or is contaced with a perfusate comprising different components depending on the age and/or kind and extent of injury. In some aspects, an aged or injured kidney may be exposed for several days or weeks to a perfusate prior to being transplanted to a recipient. In some aspects, an aged or injured kidney might be assessed by diagnostic testing and/or biopsy before and/or after being exposed to a perfusate as described herein to determine whether signs of age or injury including, but not limited to, inflammatory cell infiltrates, tubular injury, interstitial edema, or fibrotic changes are reduced after the exposure to the perfusate compared to prior to exposure to the perfusate. In some aspects, an aged or injured kidney may be exposed for one or more additional time periods to the perfusate. In some aspects, the composition of the perfusate may be adjusted according to the age or injury present in a kidney prior to perfusion. In some aspects, the composition of the perfusate may be adjusted according to the injury remaining in the kidney after a first perfusion period to perfuse the kidney for a second or more perfusion periods.

In some aspects, the kidney is perfused with a perfusate described herein at a temperature of about 2° C. to about 38° C.; or about 2, 3, 4, 5, 6, 7, 8, 9, 10, 11, 12, 13, 14, 15, 16, 17, 18, 19, 20, 21, 22° C.; or about 23, 24, 25, 26, 27, 28, 29, 30, 31, 32, 33, 34, 35, 36, 37, or 38° C.

In some aspects, the organ is a liver and is perfused with a perfusate that comprises a polynucleotide, polypeptide, vector, and/or nanoparticle described herein. In some aspects, the liver is characterized by old age, an ischemic injury, hypertensive injury, toxic injury, perfusion injury, immune injury, a physical injury, steatosis, virus induced hepatitis, alcohol, or a fibrosis not associated with any known cause prior to being exposed to a perfusate described herein. In some aspects, an aged or injured liver is contacted with a perfusate for different periods of time and/or is contaced with a perfusate comprising different components depending on the age and/or kind and/or extent of injury. In some aspects, an aged or injured liver may be exposed for several days or weeks to a perfusate prior to being transplanted to a recipient. In some aspects, an aged or injured liver might be assessed by diagnostic testing and/or biopsy before and/or after being exposed to a perfusate as described herein to determine whether signs of age or injury including, but not limited to, inflammatory cell infiltrates, tubular injury, interstitial edema, or fibrotic changes are reduced after the exposure to the perfusate compared to prior to exposure to the perfusate. In some aspects, an aged or injured liver may be exposed for one or more additional time periods to the perfusate. In some aspects, the composition of the perfusate may be adjusted according to the age or injury present in the liver prior to perfusion. In some aspects, the composition of the perfusate may be adjusted according to the injury remaining in the liver after a first perfusion period to perfuse the liver for a second perfusion period.

In some aspects, the liver is perfused with a perfusate described herein at a temperature of about 2° C. to about 38° C.; or about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C.; or about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., or about 38° C.

### VII. Methods

Provided are methods of regulating (e.g., inducing) partial cellular reprogramming, cellular rejuvenation, cellular regeneration, tissue repair, tissue regeneration, tissue rejuvenation, tissue partial reprogramming, organ regeneration, organ rejuvenation, organ partial reprogramming or any combination thereof comprising contacting a cell, a tissue or an organ with a polynucleotide, oligonucleotide, polypeptide, compound, vector, or a nanoparticle as described herein. In some aspects, the methods comprise contacting a cell, tissue, or organ ex vivo. In some aspects, the methods comprise contacting a cell, tissue or organ with a polynucleotide, e.g., engineered polynucleotide encoding at least one regeneration factor.

In some aspects, the methods comprise regeneration of an organ. In some aspects, the methods comprise rejuvenation of an organ comprising contacting an organ ex vivo with a polynucleotide, oligonucleotide, polypeptide, compound, vector, or a nanoparticle as described herein, wherein the polynucleotide, oligonucleotide, polypeptide, compound, vector, or nanoparticle comprises or encodes at least one regeneration factor, wherein the at least one regeneration factor is expressed in or present in the organ and the organ is rejuvenated. In some aspects, the methods comprise rejuvenation of an organ that is characterized by at least one of old age, an ischemic injury, a hypertensive injury, a toxic injury, a perfusion injury, an immune injury, a physical injury, steatosis, virus induced hepatitis, alcohol, or fibrosis not associated with any known cause.

In some aspects, the methods comprise transplanting into a subject in need thereof (a recipient) an organ that comprises a polynucleotide, oligonucleotide, polypeptide, compound, vector or nanoparticle as described herein.

In some aspects, the methods comprise contacting an organ ex vivo with a polynucleotide comprising a polynucleotide encoding an Oct4. In some aspects, the methods comprise contacting an organ ex vivo with a polynucleotide comprising a polynucleotide encoding a Sox2. In some aspects, the methods comprise contacting an organ ex vivo with a polynucleotide comprising a polynucleotide encoding a Klf4. In some aspects, the methods comprise contacting an organ ex vivo with a polynucleotide comprising a polynucleotide encoding a c-Myc.

In some aspects, the methods comprise contacting an organ with a first polynucleotide encoding an Oct4. In some aspects, the methods further comprise contacting an organ with a second polynucleotide encoding Sox2. In some aspects, the methods further comprise contacting an organ with a third polynucleotide encoding a Klf4. In some aspects, the methods further comprise contacting an organ with a fourth polynucleotide encoding a c-Myc.

In some aspects, the methods comprise contacting an organ with a polynucleotide encoding Oct4, Sox2, and Klf4. In some aspects, the polynucleotide further encodes c-Myc. In some aspects, the methods comprise contacting an organ with a polynucleotide comprising a polycistronic cassette comprising polynucleotides encoding Oct4, Sox2, and Klf4. In some aspects, the methods further comprise contacting the organ with a polynucleotide encoding a c-Myc.

In some aspects, the methods comprise contacting an organ with a polynucleotide comprising a first inducible promoter operably linked to a polynucleotide encoding at least one of Oct4, Sox2, and/or Klf4. In some aspects, the methods comprise contacting an organ with a polynucleotide comprising a first inducible promoter that is a tetracycline-inducible promoter and is operably linked to a polynucleotide encoding at least one of Oct4, Sox2, and/or Klf4. In some aspects, the methods further comprise contacting an organ with a polynucleotide comprising a second inducible promoter operably linked to c-Myc. In some aspects, the methods further comprise contacting the organ with an inducing compound such that the inducible promoter promotes expression of at least one of Oct4, Sox2, Kl4 and/or c-Myc.

In some aspects, the methods comprise contacting an organ with a polynucleotide comprising a first inducible promoter that is a tetracycline-inducible promoter and is operably linked to a polynucleotide encoding at least one of Oct4, Sox2, and/or Klf4. In some aspects, the methods further comprise contacting the organ with a second polynucleotide comprising a second inducible promoter that is a coumermycin-inducible promoter operably linked to c-Myc. In some aspects, the methods comprise contacting an organ with a polynucleotide comprising a first inducible promoter that is a tetracycline-inducible promoter and is operably linked to a polynucleotide encoding at least one of Oct4, Sox2, and/or Klf4 and the polynucleotide further comprises a second inducible promoter that is a coumermycin-inducible promoter and is operably linked to c-Myc.

In some aspects, the organ is contacted with a polynucleotide encoding at least one regeneration factor for about 1 minute to about 24 hours; about 1 minute; about 2 minutes; about 5 minutes; about 10 minutes; about 15 minutes; about 20 minutes; about 25 minutes; about 30 minutes; about 35 minutes; about 40 minutes; about 45 minutes; about 50 minutes; about 55 minutes; about 60 minutes; about 70 minutes; about 80 minutes; about 90 minutes; about 2 hours; about 3 hours; about 4 hours; about 5 hours; about 6 hours; about 7 hours; about 8 hours; about 9 hours; about 10 hours; about 12 hours; about 14 hours; about 16 hours; about 18 hours; about 20 hours; about 22 hours; about 24 hours; about 36 hours; about 48 hours; about 60 hours; about 72 hours; about 84 hours; about 96 hours; about 108 hours; about 120 hours; about 132 hours; about 144 hours; about 156 hours; or about 168 hours or any time period thereinbetween. In some aspects, the organ is contacted with a polynucleotide encoding at least one regeneration factor for about 1 minute to about 72 hours followed by a period of no contact and another period of being contacted with the polynucleotide for about 1 minute to about 72 hours. In some aspects, the organ is contacted with a polynucleotide as described herein repeatedly with periods of no contact in between. For example, in some aspects, the organ is contacted with a polynucleotide encoding at least one regeneration factor for about 1 minute to about 12 hours followed by a period of 12 hours of no contact, followed by another period of being contacted with the polynucleotide for about 1 minute to about 12 hours, followed by another period of 12 hours of no contact, followed by another period of being contacted. In some aspects, the periods of contacting with the polynucleotide and the no contact periods are of different time length. In some aspects, the periods of contacting with the polynucleotide are of different lengths and the no contact periods are of the same time length. In some aspects, the periods of contacting with the polynucleotide are of different lengths and the no contact periods are of different time length. In some aspects, the function of the organ is measured after being contacted with a polynucleotide and the time period of no contact and the time period of a subsequent contacting are based on the measurements of organ function.

In some aspects, the contacting occurs immediately after an organ is provided; or about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 80 minutes, about 90 minutes, about 2 hours, about 3 hours, about 4 hours, about 5 hours, about 6 hours, about 7 hours, about 8 hours, about 9 hours, about 10 hours, about 12 hours, about 14 hours, about 16 hours, about 18 hours, about 20 hours, about 22 hours, about 1 day, about 2 days, about 3 days, about 4 days, about 5 days, about 6 days, about 7 days, about 8 days, about 9 days, about 10 days, about 11 days, about 12 days, about 13 days, about 14 days; or about 3 weeks, about 4 weeks, about 5 weeks, about 6 weeks, about 7 weeks, about 8 weeks, or about 9 weeks after the organ is provided.

In some aspects, an interval between a step of contacting the organ with a polynucleotide comprising at least one regeneration factor operably linked to an inducible promoter and a step of contacting the organ with a compound that activates the inducible promoter can be 0 minutes or between about 10 minutes and about 9 weeks or more; for example, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 80 minutes, about 90 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 2 days, about 3 days, about 4 days, about 5 days, about 6 days, about 7 days, about 8 days, about 9 days, about 10 days, about 11 days, about 12 days, about 13 days, about 14 days; or about 3 weeks, 4 weeks, 5 weeks, 6 weeks, 7 weeks, 8 weeks, or 9 weeks.

In some aspects, the organ is contacted with a compound intermittently for about 2-10 times, about 2 times, about 3 times, about 4 times, about 5 times, about 6 times, about 7 times, about 8 times, about 9 times, or about 10 times.

In some aspects, the organ is contacted with a compound intermittently for about 2-10 times over a period of about 1 week to about 6 weeks, about 2-10 times over a period of about 1 week to about 5 weeks, about 2-10 times over a period of about 1 week to about 4 weeks, about 2-10 times over a period of about 1 week to about 3 weeks, about 2-10 times over a period of about 1 week to about 2 weeks, about 2-10 times over a period of about 2 weeks to about 6 weeks, about 2-10 times over a period of about 3 weeks to about 6 weeks, about 2-10 times over a period of about 4 weeks to about 6 weeks, about 2-10 times over a period of about 5 weeks to about 6 weeks, or any combination therein; or for about 2 times over a period of about 1 week to about 6 weeks, about 3 times over a period of about 1 week to about 6 weeks, about 4 times over a period of about 1 week to about 6 weeks, about 5 times over a period of about 1 week to about 6 weeks, about 6 times over a period of about 1 week to about 6 weeks, about 7 times over a period of about 1 week to about 6 weeks, about 8 times over a period of about 1 week to about 6 weeks, about 9 times over a period of about 1 week to about 6 weeks, about 10 times over a period of about 1 week to about 6 weeks, or about 2, 3, 4, 5, 6, 7, 8, 9, or 10 times over any period within about 1 week to about 6 weeks.

In some aspects, an organ is contacted with a perfusate without any polynucleotide, polypeptide, vector, or nanoparticle for a period of time followed by being contacted with a perfusate comprising a polynucleotide, polypeptide, vector, or nanoparticle as described herein.

In some aspects, the methods further comprise contacting an organ that comprises a polynucleotide, a nanoparticle comprising a polynucleotide, or a vector comprising at least one regeneration factor polynucleotide operably linked to an inducible promoter with an inducing compound.

In some aspects, the step of contacting the organ with a compound that induces an inducible promoter present in the organ as described herein comprises administering to a subject that has been transplated with the organ a compound that induces the inducible promoter.

In some aspects, the methods comprise contacting the organ that comprises a polynucleotide, a nanoparticle comprising a polynucleotide, or a vector as described herein with an inducing compound prior to transplanting the organ into a recipient. In some aspects, the methods comprise contacting the organ that comprises a polynucleotide, a nanoparticle comprising a polynucleotide, or a vector as described herein with an inducing compound after transplanting the organ into a recipient, wherein the compound is administered to the recipient.

In some aspects, the methods comprise contacting the organ after transplantation into a recipient with an inducing compound for about 1 minute to about 24 hours; about 1 minute; about 2 minutes; about 5 minutes; about 10 minutes; about 15 minutes; about 20 minutes; about 25 minutes; about 30 minutes; about 35 minutes; about 40 minutes; about 45 minutes; about 50 minutes; about 55 minutes; about 60 minutes; about 70 minutes; about 80 minutes; about 90 minutes; about 2 hours; about 3 hours; about 4 hours; about 5 hours; about 6 hours; about 7 hours; about 8 hours; about 9 hours; about 10 hours; about 12 hours; about 14 hours; about 16 hours; about 18 hours; about 20 hours; about 22 hours; about 24 hours; about 36 hours; about 48 hours; about 60 hours; about 72 hours; Abou 84 hours; about 96 hours; about 108 hours; about 120 hours; about 132 hours; about 144 hours; about 156 hours; or about 168 hours or any time period thereinbetween. In some aspects, the methods comprise contacting the organ after transplantation into a recipient with an inducing compound more than once for a time period as described above.

In some aspects, the methods comprise contacting the organ after transplantation into a recipient for a time period as described above followed by no contact. In some aspects, the methods comprise contacting the organ after transplantation into a recipient for a time period as described above followed by a biopsy of the organ and measurements of organ function, optionally, followed by contacting the organ for another time period based on the results of the biopsy and organ function measurements. In some aspects, the organ biopsy and functional measurements are repeated on a regular basis, e.g., every month, every 2 months, every 3 months, every 4 months, every 5 months, or every 6 months, and based on the results of each biopsy and functional measurement, the organ is contacted, or not, with a polynucleotide as described herein for the time periods as described herein.

In some aspects, the organ is contacted with a compound that activates the inducible promoter for the described times on 1 day of a week and the organ is not contacted with the inducing compound again for 6 days of said week. In some aspects, the organ is contacted with an inducing compound for the described times on 2 days of a week and the organ is not contacted with the inducing compound again for 5 days of said week. In some aspects, the organ is contacted with an inducing compound for the described times on 3 days of a week and the organ is not contacted with the inducing compound again for 4 days of said week. In some aspects, the organ is contacted with an inducing compound for the described times on 4 days of a week and the organ is not contacted with the inducing compound again for 3 days of said week. In some aspects, the organ is contacted with an inducing compound for the described times on 5 days of a week and the organ is not contacted with the inducing compound again for 2 days of said week. In some aspects, the organ is contacted with an inducing compound for the described times on 6 days of a week and the organ is not contacted with the inducing compound again for 1 day of said week.

In some aspects, the days of the week on which the organ is contacted with an inducing compound can be consecutive or non-consecutive. For example, the organ can be contacted with an inducing compound for the described times on days 1, 3, and 5 of a week and not contacted with the inducing agent on days 2, 4, 6, and 7 of said week.

In some aspects, the methods further comprise contacting the organ with a first inducing compound that activates a first inducible promoter. In some aspects, the first inducible promoter is a tetracycline-inducible promoter and the first inducing compound is tetracycline. In some aspects, the methods further comprise contacting the organ with a second inducing compound that activates a second inducible promoter. In some aspects, the second inducible promoter is a coumermycin-inducible promoter and the second inducing compound is coumermycin. In some aspects, the methods further comprise contacting the organ with the first inducing compound for a different time period than contacting the organ with the second inducing compound. For example, in some aspects, the methods comprise contacting an organ with tetracycline for 2 days per week for about 1-10 weeks and contacting the organ with coumermycin for 1 day per week for about 1-2 weeks, 1-3 weeks, 1-4 weeks, 1-5 weeks, 1-6 weeks, 1-7 weeks, 1-8 weeks, or 1-9 weeks. In some aspects, the methods comprise contacting the organ with tetracycline for 2 days per week for 1-10 weeks and with coumermycin for 2 days per week for about 1-2 weeks, 1-3 weeks, 1-4 weeks, 1-5 weeks, 1-6 weeks, 1-7 weeks, 1-8 weeks, or 1-9 weeks. In some aspects, methods comprise contacting the organ with tetracycline for 2 days per week and with coumermycin for 1 day per week, wherein the organ is contacted with coumermycin on a day that the organ is also contacted with tetracycline. In some aspects, the methods comprise contacting the organ with tetracycline for 2 days per week and with coumermycin for 1 day per week, wherein the organ is contacted with coumermycin on a day that the organ is not contacted with tetracycline.

In some aspects, the methods comprise contacting an organ with tetracycline for 4 days per week for about 1-10 weeks and contacting the organ with coumermycin for 1-4 days per week for about 1-10 weeks. In some aspects, methods comprise contacting the organ with tetracycline for 4 days per week and with coumermycin for 1-4 days per week, wherein the organ is contacted with coumermycin on a day that the organ is also contacted with tetracycline. In some aspects, the methods comprise contacting the organ with tetracycline for 4 days per week and with coumermycin for 1-3 day per week, wherein the organ is contacted with coumermycin on a day that the organ is not contacted with tetracycline.

In some aspects, upon each occurrence of contacting, the organ is contacted with the first inducing compound for a longer period of time than with the second inducing compound. In some aspects, the organ is contacted with the first and the second inducing compound for the same amount of time. In some aspects, the organ is contacted with the first inducing compound more than once while the organ is contacted with the second inducing compound once. In some aspects, the organ is contacted with the first inducing compound once while the organ is contacted with the second inducing agent more than once.

In some aspects, the organ is contacted with a polynucleotide encoding at least one of Oct4, Sox2, and/or Klf4 operably linked to a first inducible promoter and a second polynucleotide encoding c-Myc operably linked to a second inducible promoter. In some aspects, the methods comprise contacting the organ with a composition that comprises a first inducing compound for a first period of time and adding to the composition a second inducing compound for a second period of time. In some aspects, the first and second period of time are of the same length. In some aspects, the first inducing compound is added to the composition at the same time as the second inducing compound. In some aspects, the first inducing compound is added at a different time than the second inducing compound. In some aspects, the first and/or the second inducing compound are added to the composition, e.g., for 2, 3, or 4 days per week every week for 2 month. In some aspects, the first and/or the second inducing compound are added to the composition, e.g., for 2, 3, or 4 days per week every week for 1 month. In some aspects, the first and/or the second inducing compound are added to the composition, e.g., for 2, 3, or 4 days per week every other week for 2 months. In some aspects, the first and/or the second inducing compound are added to the composition, e.g., for 2, 3, or 4 days per week every other week for 1 month.

In some aspects, the methods comprise administering to a subject that has been transplanted with an organ that comprises a polynucleotide, vector, or nanoparticle as described herein a compound that induces an inducible promoter present in the polynucleotide or vector and further comprise administering to the subject an additional agent, e.g., an immunosuppressive agent. In some aspects, the immunosuppressive agent includes, but is not limited to, a steroidal (e.g., prednisone), non-steroidal (e.g., sirolimus (Rapamune, Wyeth-Ayerst Canada), tacrolimus (Prograf, Fujisawa Canada), an anti-IL2R antibody, e.g., daclizumab (Zenapax, Roche Canada), 15-deoxyspergualin, cyclosporin, methotrexate, rapamycin, Rapamune (sirolimus/rapamycin), FK506, Lisofylline (LSF), or mycophenolate mofetil, anti-thymocyte globulin, belatacept, or everolimus.

In some aspects, the methods comprise administering to a subject that has been transplanted with an organ that comprises a polypeptide as described herein an immunosuppressive agent, such as, a steroidal (e.g., prednisone), non-steroidal (e.g., sirolimus (Rapamune, Wyeth-Ayerst Canada), tacrolimus (Prograf, Fujisawa Canada), an anti-IL2R antibody, e.g., daclizumab (Zenapax, Roche Canada), 15-deoxyspergualin, cyclosporin, methotrexate, rapamycin, Rapamune (sirolimus/rapamycin), FK506, Lisofylline (LSF), or mycophenolate mofetil, anti-thymocyte globulin, belatacept, or everolimus.

In some aspects, the methods comprise contacting an organ that has previously been contacted with a polynucleotide encoding at least one regeneration factor operably linked to an inducible promoter with an inducing compound.

In some aspects, the methods comprise administering an inducing compound to a recipient that has been transplanted with an organ that has previously been contacted ex vivo with a polynucleotide encoding at least one regeneration factor operably linked to an inducible promoter. In some aspects, the methods comprise administering an inducing compound to a recipient that has been transplanted with an organ that comprises an exogenous polynucleotide encoding at least one regeneration factor operably linked to an inducible promoter. In some aspects, the recipient that has been transplanted with an organ that has previously been contacted with a polynucleotide encoding at least one of Oct4, Sox2, and/or Klf4 operably linked to a first inducible promoter and a second polynucleotide encoding c-Myc operably linked to a second inducible promoter. In some aspects, the methods comprise administering a first inducing compound to the recipient for a first period of time and administering a second inducing compound to recipient for a second period of time. In some aspects, the first and second period of time are of the same length. In some aspects, the first inducing compound is administered at the same time as the second inducing compound. In some aspects, the first inducing compound is administered at a different time than the second inducing compound. In some aspects, the first and/or the second inducing compound are administered, e.g., for 2, 3, or 4 days per week for 1-10 weeks every 3 months. In some aspects, the first and/or the second inducing compound are administered, e.g., for 2, 3, or 4 days per week for 1-10 weeks every 4 months. In some aspects, the first and/or the second inducing compound are administered, e.g., for 2, 3, or 4 days per week for 1-10 weeks every 6 months. In some aspects, the first and/or the second inducing compound are administered, e.g., for 2, 3, or 4 days per week for 1-10 weeks every 8 months. In some aspects, the first and/or the second inducing compound are administered, e.g., for 2, 3, or 4 days per week for 1-10 weeks every 10 months. In some aspects, the first and/or the second inducing compound are administered, e.g., for 2, 3, or 4 days per week for 1-10 weeks every 12 months.

In some aspects, for each administration of a first and a second compound to a recipient, the first inducing compound is administered for a longer period of time than the second inducing compound. In some aspects, the first and the second inducing compound are administered to the recipient for the same amount of time. In some aspects, the first inducing compound is administered for a shorter period of time than the second inducing compound. In some aspects, the first inducing compound is administered to the recipient more than once while the second inducing compound is administered once. In some aspects, the first inducing compound is administered to the recipient once while the second inducing compound is administered more than once.

In some aspects, the methods comprise (i) contacting an organ with a polynucleotide comprising a first inducible promoter operably linked to a polynucleotide encoding at least one of Oct4, Sox2, or Klf4; (ii) contacting the organ with an inducing agent that induces activity of the first promoter and results in expression of the at least one of Oct4, Sox2, or Klf4; (iii) contacting the organ with a polynucleotide comprising a second inducible promoter operably linked to a polynucleotide encoding c-Myc; and (iv) contacting the organ with a second inducing agent that induces activity of the second promoter and results in expression of c-Myc. In some aspects, step (ii) of the described method is performed more than once prior to the performance of step (iii). In some aspects, step (ii) of the described method is performed once prior to the performance of step (iii). In some aspects, step (ii) is performed more than once while step (iv) is only performed once. In some aspects, step (ii) is performed between two and 100 times while step (iv) is only performed between one and 10 times. In some aspects, step (iv) is performed once for every 2-5 times of performance of step (ii). In some aspects, step (iv) is performed once for every 1-5, 6-10, 11-15, 16-20, 21-25, 26-30, 31-35, 36-40, 41-45, or 46-50 times of performance of step (ii).

In some aspects, steps (ii) and (iv) are performed multiple times during a 1-month period and not performed during the following consecutive 3-month period. In some aspects, steps (ii) and (iv) are performed multiple times during a 1-month period and not performed during the following consecutive 6-month period. In some aspects, steps (ii) and (iv) are performed multiple times during a 1-month period and not performed during the following consecutive 9-month period. In some aspects, steps (ii) and (iv) are performed multiple times during a 1-month period and not performed during the following consecutive 12-month period.

In some aspects, the methods comprise (i) contacting an organ with a polynucleotide comprising a first inducible promoter operably linked to a polynucleotide encoding at least one of Oct4, Sox2, or Klf4 and further contacting the organ with a polynucleotide comprising a second inducible promoter operably linked to a polynucleotide encoding c-Myc; (ii) contacting the organ with an inducing compound that induces activity of the first promoter; and (iii) contacting the organ with a second inducing compound that induces activity of the second promoter. In some aspects, step (ii) of the described method is performed more than once while step (iii) is performed once. In some aspects, step (ii) is performed between 2 and 100 times while step (iii) is performed between 1 and 10 times. In some aspects, step (iii) is performed once for every 2-5 times of performance of step (ii). In some aspects, step (iv) is performed once for every 1-5, 6-10, 11-15, 16-20, 21-25, 26-30, 31-35, 36-40, 41-45, or 46-50 times of performance of step (ii).

In some aspects, steps (ii) and (iii) are performed multiple times during a 1-month period and not performed during the following consecutive 3-month period. In some aspects, steps (ii) and (iii) are performed multiple times during a 1-month period and not performed during the following consecutive 6-month period. In some aspects, steps (ii) and (iii) are performed multiple times during a 1-month period and not performed during the following consecutive 9-month period. In some aspects, steps (ii) and (iii) are performed multiple times during a 1-month period and not performed during the following consecutive 12-month period.

In some aspects, an organ contacted ex vivo with a composition as described herein is an organ characterized by at least one of old age, an ischemic injury, a hypertensive injury, a toxic injury, a perfusion injury, an immune injury, a physical injury, steatosis, virus induced hepatitis, alcohol, or a fibrosis not associated with any known cause.

In some aspects, the organ is an aged organ. In some aspects, the organ is 70 years old or older (ie the organ is removed from a subject that is 70 years old or older). In some aspects, the organ is over the age of 60. In some aspects, the organ is 70-79 years old. In some aspects, the organ is 60-69 years old. In some aspects, the organ is 50-59 years old. In some aspects, the organ is 40-49 years old. In some aspects, the organ is under age 60. In some aspects, the organ is 50 years old or younger. In some aspects, the organ is between age 6 and age 50.

In some aspects, the organ is a kidney or liver.

In some aspects, the kidney is 70 years old or older. In some aspects, the kidney is over the age of 60; is 70-79 years old; 60-69 years old; 50-59 years old; or 40-49 years old. In some aspects, the kidney is under age 60. In some aspects, the kidney is 50 years old or younger. In some aspects, the kidney is between age 6 and age 50.

In some aspects, the kidney is from a donor of old age. In some aspects, the kidney is from a donor that is 70-79 years old; 60-69 years old; 50-59 years old; or 40-49 years old. In some aspects, the kidney is from a donor having an age-related disease or condition. In some aspects, the age-related disease or condition is a history of hypertension. In some aspects, the age-related disease or condition is a history of atherosclerosis. In some aspects, the age-related disease or condition is a history of a toxic kidney injury. In some aspects, the age-related disease or condition is a history of a renal immune injury. In some aspects, the age-related disease or condition is a history of a renal perfusion injury. In some aspects, the age-related disease or condition is fibrosis. In some aspects, the kidney is from a donor having serum creatinine greater than or equal to 1.5 mg/dl. In some aspects, the kidney is from a donor having any two of the following: a history of high blood pressure, a creatinine greater than or equal to 1.5 mg/dl or death resulting from a stroke. In some aspects, the kidney is from a donor under age 50. In some aspects, the kidney is from a donor under age 50, wherein the donor suffered brain death (e.g., from traumatic injuries or other causes, such as a stroke). In some aspects, the donor is from a non-heart-beating donor (donation after cardiac death, DCD). In some aspects, the donor is from a brain-dead donor (donation after brain death, DBD). In some aspects, the kidney is from a DCD donor who deceased of an uncontrolled circulatory death, also known as category Maastricht II, cardiac arrest with unsuccessful resuscitation. In some aspects, the kidney is from a donor having had controlled circulatory death, also known as category Maastricht III, cardiac arrest happening in the hospital in a controlled manner.

In some aspects, the kidney is from a living donor. In some aspects, the kidney is from a deceased donor.

In some aspects, the liver is 70 years old or older. In some aspects, the liver is over the age of 60; is 70-79 years old; 60-69 years old; 50-59 years old; or 40-49 years old. In some aspects the kidney is from a donor having an age-related disease or condition. In some aspects, the age-related disease or condition is steatosis. In some aspects, the age-related disease or condition is hepatic fibrosis.

In some aspects, the liver is characterized by steatosis. In some aspects, the liver is from a non-heart beating donor (donation after cardiac death, DCD). In some aspects, the liver is from a brain-dead donor (donation after brain death, DBD). In some aspects, the liver is a split liver.

In some aspects, the liver is from a living donor. In some aspects, the liver is from a deceased donor.

Organ function may be characterized pre- and post-transplantation using assays and tests that are known in the art. In some aspects, the organ is characterized by at least one of old age, an ischemic injury, a hypertensive injury, a toxic injury, a perfusion injury, an immune injury, a physical injury, steatosis, virus induced hepatitis, alcohol, or a fibrosis not associated with any known cause. In some aspects, an aged or injured organ is assessed by diagnostic testing and/or biopsy before and/or after being exposed to a perfusate as described herein to determine whether signs of age or injury including, but not limited to, inflammatory cell infiltrates, tubular injury, interstitial edema, or fibrotic changes are reduced after the exposure to the perfusate compared to prior to exposure to the perfusate. In some aspects, an aged or injured organ may be exposed for one or more additional time periods to the perfusate. In some aspects, the composition of the perfusate may be adjusted according to the age or injury present in an organ prior to perfusion. In some aspects, the composition of the perfusate may be adjusted according to the injury remaining in the organ after a first perfusion period to perfuse the organ for a second or more perfusion periods.

In some aspects, kidney function is characterized by testing one or more of the following: BUN, pH, bicarbonate, sodium, potassium, serum creatinine (eg, eGFR 1 week/1 month/3 months post-transplant), protein to creatinine ratio (urine), serum KIM1, serum Klotho, CXCL10/9 (eg, measured in the urine) and/or blood pressure.

In some aspects, kidney function is characgerized in recipient by detecting and/or measuring urine production.

In some aspects, kidney histology is characterized, eg, by testing one or more of the following: Banff score (Masson Trichrome), tubular necrosis score, proliferation (eg, by assaying Ki67, PCNA), apoptosis (eg by TUNEL assay) and/or immune infiltrates (eg by assaying CD68, CD3 and/or CD19).

In some aspects, the liver is evaluated for hepatic damage, inflammation, regeneration and/or tissue repair. In some aspects, liver function is evaluated/characterized by one or more of the following AST, ALT, bilirubin, MPO level, and/or MDA levels. In some aspects, liver histology is characterized, eg, by testing one or more of the following: Ki67, PCNA, HGF and/or TGF-beta; TUNEL and/or caspase 3 assay. H&E staining may be performed to evacuate hepatic damage.

In some aspects, the liver is characterized by (evaluated for) one or more of the following: steatosis score (eg Steatosis, lobular inflammation and hepatocellular ballooning) grade 0 (healthy, <5%), grade 1 (mild, 5%-33%), grade 2 (moderate, 34%-66%), and grade 3 (severe, >66%), necrosis score, and/or for presence of immune infiltrates (eg assaying for CD68, CD3 and/or CD19).

In some aspects, liver function is evaluated by assaying Alkaline phosphatase (ALP), Albumin and total protein, Bilirubin, Gamma-glutamyltransferase (GGT), L-lactate dehydrogenase (LD) and/or Prothrombin time (PT).

In some aspects, the organ (e.g., a biopsy of the organ) is characterized using an epigenetic clock. In further embodiments, the DNA methylation age of the organ is determined using the Horvath epigenetic clock. The Horvath epigenetic clock can be used as an age estimation method based on DNA methylation at CpG dinucleotide motifs in the DNA.

In some aspects, a kidney function is characterized in the recipient by testing one or more of the following: BUN, serum creatinine, serum pH, bicarbonate, sodium, potassium, or lactate levels. In some aspects, a kidney contacted with a polynucleotide described herein exhibits improved BUN, serum creatinine, serum pH, bicarbonate, sodium, potassium, and/or lactate levels compared to prior to being contacted with the polynucleotide.

In some aspects, transplanted livers are evaluated for hepatic damage, inflammation and regeneration/tissue reparation. In some aspects, liver function is characterized in the recipient by testing one or more of the following: widely standardized hepatic lesion parameters (AST, ALT and bilirubin), apoptotic parameters (caspase 3), hepatic cell proliferation (Ki67/PCNA, HGF and TGF-0), hepatic inflammation (MPO activity for neutrophil accumulation, MDA levels for oxidative stress), and/or liver histology (H&E).

In some aspects, a liver contacted with a polynucleotide described herein exhibits reduced AST, ALT, bilirubin, caspase 3 levels, increased hepatic cell proliferation markers Ki67/PCNA, HGF and TGF-β, reduced MPO activity, reduced MDA levels, and reduced histological signs of liver damage compared to prior to being contacted with the polynucleotide.

In some aspects, the methods of rejuvenating aged cells, tissues or organs as described herein restore or improve at least one functionality of the aged cells, tissues or organs, e.g., a mitochondrial function, a proteolytic activity, a heterochromatin level, a histone methylation, a nuclear lamina polypeptide, a cytokine level.

In some aspects, the contacting of a cell, tissue, or organ with a polynucleotide as described herein leads to a change in an aggregate methylation status of a collections of individual methylation sites in the cell, a cell of the tissue or the organ, which aggregate methylation status measures chronological age (DNA methylation clock).

In some aspects, the contacting of a cell, tissue, or an organ with a polynucleotide as described herein leads to an increase in one or more nuclear and/or epigenetic markers compared to a reference value. In some aspects, the contacting of a cell, tissue, or organ with a polynucleotide as described herein leads to an increased expression of HP1gamma. In some aspects, the contacting of a cell, tissue, or organ with a polynucleotide as described herein leads to an increase in H3K9me3. In some aspects, the contacting of a cell, tissue or organ with a polynucleotide as described herein leads to an increased expression of lamina support protein LAP2alpha. In some aspects, the contacting of a cell, tissue or organ with a polynucleotide as described herein leads to an increased gene expression of SIRT1 protein.

In some aspects, a cell, tissue or organ contacted with a polynucleotide described herein exhibits improved mitochondria health and function compared to a cell, tissue or organ not contacted with a polynucleotide described herein. In some aspects, improved mitochondria health and function is measured as a change in mitochondria membrane potential, reactive oxygen species (ROS), or a combination thereof. For example, mitochondria membrane potential can be measured using Tetramethylrhodamine, Methyl Ester, Perchlorate (Thermo), a dye that is sequestered by mitochondria based on their membrane potential as described in PCT Publication WO2019/178296. Mitochondria ROS can be measured using MitoSOX, a fluorogenic dye that is oxidized by superoxides in the mitochondria as described in PCT Publication WO2019/178296.

In some aspects, provided are methods for preparing an organ for transplantation. In some aspects, the methods comprise contacting an organ prior to transplantation with a composition comprising a polynucleotide, vector or nanoparticle described herein. In some aspects, the composition is a perfusate as described herein. In some aspects, the methods comprise contacting an organ prior to transplantation with a perfusate comprising a polynucleotide, vector, or nanoparticle described herein, wherein the organ is rejuvenated. In some aspects, the methods comprise contacting an organ characterized by at least one of old age, an ischemic injury, a hypertensive injury, a toxic injury, a perfusion injury, an immune injury, a physical injury, steatosis, virus induced hepatitis, alcohol, or a fibrosis not associated with any known cause prior to transplantation with a perfusate comprising a polynucleotide, vector or nanoparticle described herein, at least one of old age, an ischemic injury, a hypertensive injury, a toxic injury, a perfusion injury, an immune injury, a physical injury, steatosis, virus induced hepatitis, alcohol, or a fibrosis not associated with any known cause is treated, ameliorated, prevented and/or reversed in the organ. In some aspects, the methods comprise contacting an organ prior to transplantation with a perfusate comprising a polynucleotide, vector or nanoparticle described herein, at least one of old age, an ischemic injury, a hypertensive injury, a toxic injury, a perfusion injury, an immune injury, a physical injury, steatosis, virus induced hepatitis, alcohol, or a fibrosis not associated with any known cause is ameliorated, prevented and/or reversed in the organ.

In some aspects, the methods comprise contacting an organ with a polynucleotide, vector or nanoparticle described herein. In some aspects, the contacting is performed by submerging an organ in a perfusate solution that comprises the polynucleotide, vector or nanoparticle as described herein. In some aspects, the contacting is performed by perfusing the organ with a perfusate solution that comprises the polynucleotide, vector or nanoparticle as described herein. In some aspects, the perfusing is accomplished by connecting a tubing to a blood vessel of an organ. In some aspects, the perfusing is accomplished by connecting a tubing to a lymphatic vessel of an organ.

In some aspects, provided are methods of preparing an organoid for transplantation. In some aspects, the organoid is generated in vitro from pluripotent stem cells. In some aspects, the organoid is contacted with a polynucleotide described herein. In some aspects, the organoid contains vasculature. In some aspects, the contacting is performed by submerging the organoid in a perfusate solution that comprises the polynucleotide, vector or nanoparticle as described herein. In some aspects, the contacting is performed by connecting a vessel of the organoid to a tubing and perfusing the organoid with a perfusate solution that comprises the polynucleotide, vector or nanoparticle as described herein. In some aspects, the perfusing is accomplished by connecting a tubing to a blood vessel of the organoid. In some aspects, the perfusing is accomplished by connecting a tubing to a lymphatic vessel of the organoid.

In some aspects, an organ or organoid is contacted with a transfection agent. In some aspects, the transfection agent comprises lipofectamine, LT-1, dextran, calcium phosphate, polybrene. In some aspects, an organ or organoid is transfected using electroporation, microinjection or a combination thereof.

In some aspects, an organ or organoid is contacted with a composition that comprises a combination therapy.

In some aspects, the composition comprises a polynucleotide, polypeptide, vector or nanoparticle as described herein and an enhancing agent. In some aspects, the composition comprises a polynucleotide, polypeptide, vector or nanoparticle as described herein and one or more of a soluble Wnt, Wnt conditioned media, BIX-01294, PD0325901, a DNA methyltransferase inhibitor, a histone deacetylase inhibitor, valproic acid, 5′-azacytidine, dexamethasone, suberoylanilide, hydroxamic acid (SAHA (e.g., MK0683, vorinostat and other hydroxamic acids), vitamin C, trichostatin (TSA), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenyl butyrate (e.g., sodium phenyl butyrate) or other short chain fatty acids, Scriptaid, Suramin Sodium, APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVPLAQ-824, CBHA (m-carboxycinnaminic acid bishydroxamic acid), JNJ16241199, Tubacin, A-161906, proxamide, oxamflatin, 3-Cl-UCHA (e.g., 6-(3-chlorophenylureido) caproic hydroxamic acid), AOE (2-amino-8-oxo-9,10-epoxydecanoic acid), CHAP31, or CHAP 50. In some aspects, the composition comprises a polynucleotide, polypeptide, vector or nanoparticle as described herein and a dominant negative form of an HDACs (e.g., catalytically inactive forms), an siRNA inhibitor of an HDACs, or an antibody that specifically binds to an HDAC.

### VIII. Kits

In some aspects, provided are kits that comprise a composition or perfusate as described herein comprising a polynucleotide, vector, and/or nanoparticle and one or more pharmaceutically acceptable carriers, excipients, and/or vehicles. In some aspects, kits are provided that comprise: a first composition or perfusate comprising a polynucleotide, vector and/or nanoparticle as described herein and one or more pharmaceutically acceptable carriers, excipients, and/or vehicles, and, optionally, a second composition or perfusate comprising a second polynucleotide, vector, and/or nanoparticle as described herein and one or more pharmaceutically acceptable carriers, excipients and/or vehicles. In some aspects, the kit further comprises an inducing compound. In some aspects, the kit comprises a first inducing compound and a second inducing compound.

In some aspects, the kit comprises a composition or perfusate as described herein packaged in a manner that facilitates their use to practice methods of the present disclosure.

In some aspects, the kit further comprises instructions on how the composition or perfusate are to be administered and tools for the preparation of and administration of the pharmaceutical composition or perfusate in a perfusion system as described herein.

### EXAMPLES

### Example 1. Methods and Materials

### Animals

Isogenic and allogenic kidney transplantation models were performed. For the isogenic renal transplantation model, 3-month-old male Lewis (LEW, Janvier Labs, Le Genest-Saint-Isle, France) rats were recipients of 3-month-old male LEW grafts (Lewin, E. et al. Scand J Urol vol. 27, 1993). For allogeneic kidney transplantations, male LEW rats were recipients of 9-month-old male Dark agouti (DA, Janvier Labs, Le Genest-Saint-Isle, France) grafts. DA and LEW strains differ partially at major histocompatibility complexes and various non-MHC loci, conferring a weaker histocompatibility combination and driving graft rejection. Systolic pressure, proteinuria and diuresis were monitored to assess the progression of hypertensive nephropathy.

### Kidney Normothermic Perfusion and AAV Administration

For kidney normothermic perfusion, a Hugo Sachs/Harvard Apparatus device was used. Briefly, this system consists in a water-jacketed and circular chamber with a glass lid assembly. The kidney was connected through the cava vein and the aorta using two cannulae for organ perfusion. The system includes different support components attached to the cannulae to maintain the organ in the most physiological conditions: a membrane oxygenator, reservoirs, cannula line holders, perfusate, gas and water lines, bubble traps and manifolds for water, gas and perfusate control. The perfusate was pre-warmed at 37° C. and oxygenated during 30 minutes before organ connection. Perfusate was composed of Gey's solution (Sigma) and 1% Penicillin/Streptomycin (Sigma). After organ connection, a pressure-controlled perfusion was performed, increasing progressively the pressure during 5 minutes to reach a mean pressure of 70 mmHg. When the target pressure was reached, AAV was administered in the perfusate and perfusion was performed for 1 hour after AAV administration.

### Human Kidney Perfusion

Human kidneys, e.g., those that will not be used for transplantation due to, e.g., ischemic, toxic, perfusion or other injury, or lack of suitability due to age, are perfused ex vivo with polynucleotides, vectors and/or nanoparticles, as described herein using a perfusion system. The perfusate for human kidney perfusion contained: 215 mL of dextran/albumin solution (Steen Solution, XVIVO Perfusion AB, Goteborg, Sweden); 400 mL of packed red blood cells; 2 mL of calcium gluconate 10%; 1300 U/L heparin; 400 mg of cefazolin; Verapamil (vasodilator); Amino acids, glucose, and insulin; and Ringer's lactate to compensate urine loss

Human kidneys will be connected to the perfusion system and perfused for at least one hour prior to addition of polynucleotides, vectors, and/or nanoparticles as described herein.

Human kidney function will then be assessed in kidney biopsies prior to and after perfusion by determining Banff score (Masson Trichrome); Tubular necrosis score; measuring proliferation markers (Ki67, PCNA); apoptosis markers (TUNEL); immune infiltrates (CD68, CD3, and/or CD19); and by single nucleus RNA sequencing (snRNAseq) and methylome analysis.

### Human Liver Perfusion

Human livers will be perfused via an Organox Metra protocol. The perfusion system will be primed with 3 units of packed red blood cells and colloid solution (Gelafundin, B Braun 500 mL).

Before connection of the liver, the blood-based perfusion was supplemented with: Cefuroxime 750 mg (antibiotic); Heparin 10,000 I.U. (anticoagulant to prevent thrombosis in the circuit); Sodium bicarbonate (buffer to adjust the pH of the infusion before the liver is placed in the device); Calcium gluconate 10% 10 ml (to correct the binding of citrate to calcium); and after the human liver is connected and during the liver perfusion the following components will be added to the perfusatel; Insulin (200 I.U.) (to control the level of perfused glucose); Heparin (25,000 I.U.) (to maintain anticoagulation); 2% solution of sodium taurocholate in isotonic saline solution (to compensate for the loss of bile salts); Flolan® 0.5 mg Prostacyclin (vasodilator to optimize microperfusion); and parenteral nutrition solution comprising amino acids and glucose for liver maintenance.

Once the priming of the circuit was completed and the infusions were connected, the perfusion liquid will circulate automatically, oxygenated and heated to 37° C., and the human liver will be connected to the perfusion system.

The rate of infusion of glucose and amino acids (parenteral nutrition) is variable and glucose levels were entered manually every 4 hours.

As an alternative, the human liver perfusion system can use non-RBC oxygen carriers including HEMOXYCarrier® Hemopure, Hemarina®, or Perfluorocarbon.

The human livers were monitored by measuring AST/ALT, ALP, albumin, bilirubin, prothrombin time, L-lactate dehydrogenase, bilirubin, and Gamma-glutamyltransferase in the perfusate. Biopsies of human livers taken prior to and at the end of perfusion can be examined using SnRNASeq, Methylome analysis and histologies will be performed to assess proliferation markers (Ki67, PCNA), apoptosis markers (e.g., TUNEL), steatosis scores (steatosis, lobular inflammation and hepatocellular ballooning) with grade 0 (healthy, <5%), grade 1 (mild, 5%-33%), grade 2 (moderate, 34%-66%), and grade 3 (severe, >66%), necrosis score, and immune infiltrates (CD68, CD3, and/or CD19).

Tissues were lysed and homogenized and total RNA was extracted using Maxwell® RSC Instrument (Promega) with the Maxwell® RSC miRNA from Tissue (Promega), according to the supplier's protocol. RNA purity and concentration were assessed using a NanoDrop™ Eight Spectrophotometer (ThermoFisher). cDNA was synthesized from the RNA template using cDNA synthesis kit from Invitrogen as per manufacturer's instructions. The resulting cDNA was diluted and this was used to determine expression levels of GFP, NGAL, MCP1, CXCL10, HIF, ETR, TFGβ and TNF. GADPH was used as the housekeeping gene. Primers used in cDNA analysis are shown in Table 2.

Real-Time qPCR was performed using the corresponding primers for the genes of interest on a 384-well plate on a QuantStudio 7 device (ThermoFisher) with SYBR Green PCR Master Mix reagent (Appliedbiosystems). Samples were run in triplicate in 10 μl reactions, and mRNA expression of the target genes was normalized to GADPH mRNA and expressed as relative gene expression to the control group.

### Adeno-Associated Viruses (AAVs)

Adeno-associated viruses (AAVs) prepared by Vector Biolabs were used to deliver DNA encoding the critical reprogramming genes under a regulatable promoter. This allowed efficient delivery and regulated expression of the reprogramming gene products. The reprogramming factors (Oct4, Sox2, Klf4 and cMyc) were cloned under a Tet-ON promoter, which is activated by the transactivator, rtTA, in the presence of doxycycline (AAV TRE3G SK shortWPRE SV40 polyA of SEQ ID NO: 26; AAV TRE3G cMyc shortWPRE SV40polyA+EF1a TetON3G of SEQ ID NO: 27; AAV-TRE3G-hMyc-EF1a-rTta of SEQ ID NO: 28; plasmid AAV-CAG-CBRLuc-GFP of SEQ ID NO: 29). Sequences of polynucleotides and their components are provided in Table 2.

To leverage its broad cellular tropism, AAV of the DJ serotype capsid was used (Grimm, D. et al. J Virol 82, 5887-5911, 2008). After AAV-mediated delivery of the programming factors to the organ, gene expression was activated by adding commercially available Doxycycline (Dox). From 2 weeks after transplantation until the end of the study, rats were weighed and placed in metabolic cages for 24-hour urine and tail-vein-blood collection weekly. Blood urea nitrogen (BUN), urine and serum creatinine (Crea) were determined.

### Luc Fluorescence Analysis.

7 days after the graft perfusion and kidney transplantation, rats were anesthetized and injected intraperitoneally with 1 ml Luciferin (15 mg/mL). 30 minutes later, luminescence images were captured with IVIS Bioluminiscence imaging system using standard approaches.

### Statistical Analysis

Statistical analysis was performed using GraphPad Prism 5 statistical software (GraphPad Software Inc.). Univariate analysis using the log-rank test (Kaplan-Meier curves) was conducted to assess rat survival (time from kidney transplantation to death). The Mann Whitney test was used to compare two groups. A value <0.05 was considered significant. Values are given as mean±standard deviation.

We chose to analyse kidney chronic lesions 6 weeks after transplantation using a validated score used to assess kidneys from human donors (Remuzzi et al. J Am Soc Nephrol 10: 2591-2598, 1999) in kidney transplantation (Table Z), with modifications as described below. This global score evaluates glomerular sclerosis, arterial narrowing, tubular necrosis, tubular atrophy and interstitial fibrosis.

### Modified Remuzzi Score Analysis

A modified Remuzzi score was used to analyse kidney chronic lesions 6 weeks after transplantation from human donors (Remuzzi et al. J Am Soc Nephrol 10: 2591-2598, 1999) in kidney transplantation, with modifications as described below. This global score evaluates glomerular sclerosis, arterial narrowing, tubular necrosis, tubular atrophy and interstitial fibrosis.

### Remuzzi Score (Rs) (Remuzzi et al, Supra)

Glomerular Global Sclerosis (G) is scored as
   0:none globally sclerosed
   1: <20% global glomerulosclerosis
   2: 20 to 50% global glomerulosclerosis
   3: >50% global glomerulosclerosis
Tubular atrophy (T) is scored as:
   0: absent
   1: <20% of tubuli affected
   2: 20 to 50% of tubuli affected
   3 >50% of tubuli affected
Interstitial Fibrosis (I) is scored as:
   0: absent
   1: <20% of renal tissue replaced by fibrous connective tissue
   2: 20 to 50% of renal tissue replaced by fibrous connective tissue
   3: >50% of renal tissue replaced by fibrous connective tissue
Arterial and arteriolar narrowing (A) is scored as:
   0: absent
   1: Increased wall thickness but to a degree that is less than the diameter of the lumen
   2: Wall thickness that is equal or slightly greater of the diameter of the lumen
   3: Wall thickness that far exceeds the diameter of the lumen with extreme luminal narrowing or occlusion

Modifications to the Remuzzi score did not include glomerular sclerosis factor (ischemic injury does not affect the glomerular compartment and so all glomeruli were normal in all the samples), and arterial/arteriolar narrowing (arteries were not observed in some slides). Additionally, acute tubular necrosis (ATN) was calculated by four different grades: none (0), low (1), moderate (2), high (4).

### Example 2. AAV Targeting to the KidneyGFP/luc

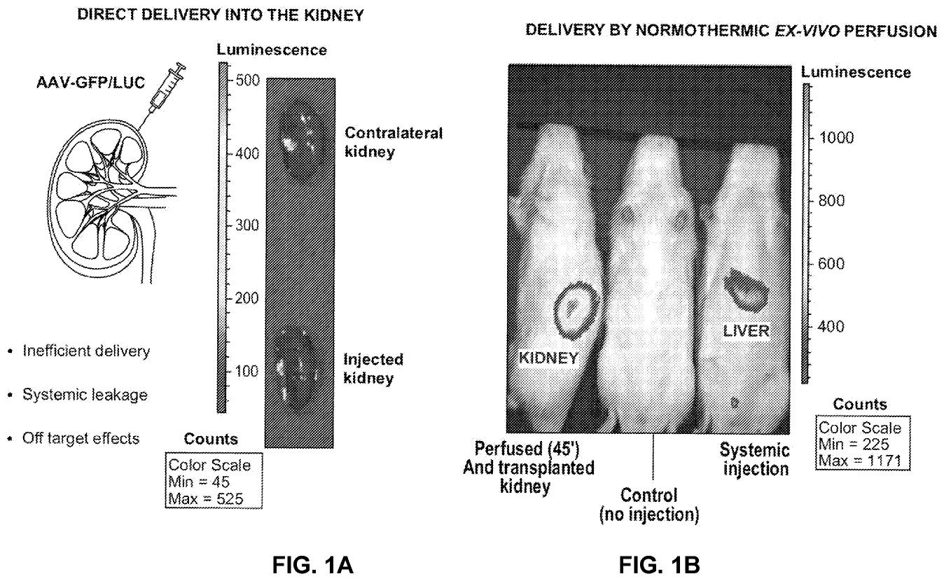

AAV vectors were injected into a clamped rat renal vein and weak luciferin expression was detected in both the injected kidney and the non-injected contralateral kidney 7 days after in situ injection (see FIG. 1A), indicating systematic leakage of the AAV, which could lead to undesired effects. The weak signal observed in the kidneys resulted from a low transduction efficiency of the AAV vectors in kidney.

Two normothermic machine perfusion (NMP) time periods were then tested with the AAV-GFP/Luc vector: at times of 45 minutes and 90 minutes. The virus titer injected was approximately 1×10¹². The total distribution volume in the perfusion system was estimated to be about 60-70 ml. Rats were transplanted with a kidney perfused ex vivo via a normothermic machine perfusion (NMP) with GFP/luc AAV, for 45 minutes as described within the methods and materials above. Luciferin was only detected in the perfused kidney (see FIG. 1B, animal on the left). By contrast, within intravenously (iv) injected rats (via penile vein), luciferin was localized to the liver and not observed within the kidneys (see FIG. 1B, rat on the right). Therefore, ex vivo normothermic machine perfusion (NMP) of rat kidney with GFP/luc AAV showed specific delivery to the perfused kidney, and an apparent absence of “leakage” to other organs and tissues.

Following 90 minutes of perfusion (the flow rate was set to approximately 4 ml/min), the perfused left kidney exhibited in vivo luciferin expression, while no luciferin expression was observed in the lungs or the contralateral kidney (right kidney, i.e., not perfused). Minimal luciferin level observed in the liver.

### Example 3. OSKM Treatment Improves Kidney Function and Rat Survival in an Aging and Allotransplantation Model

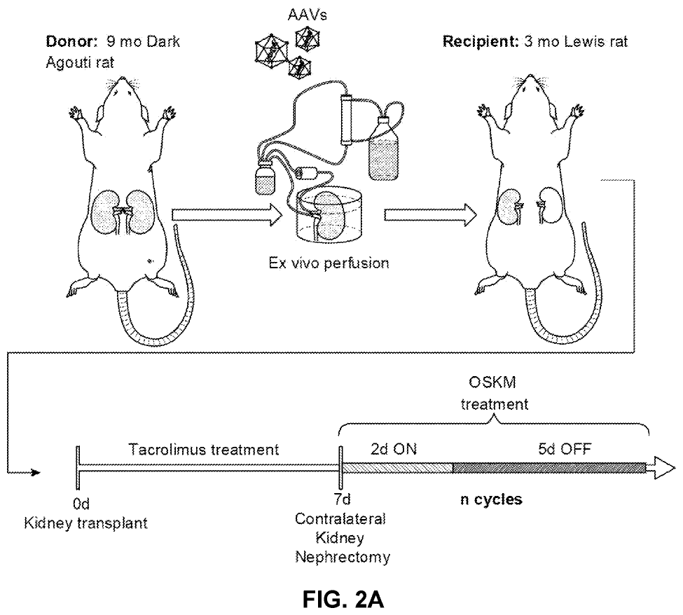

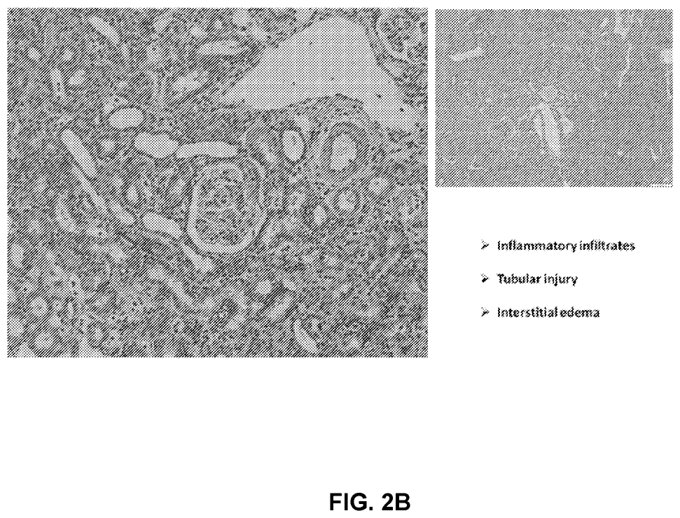

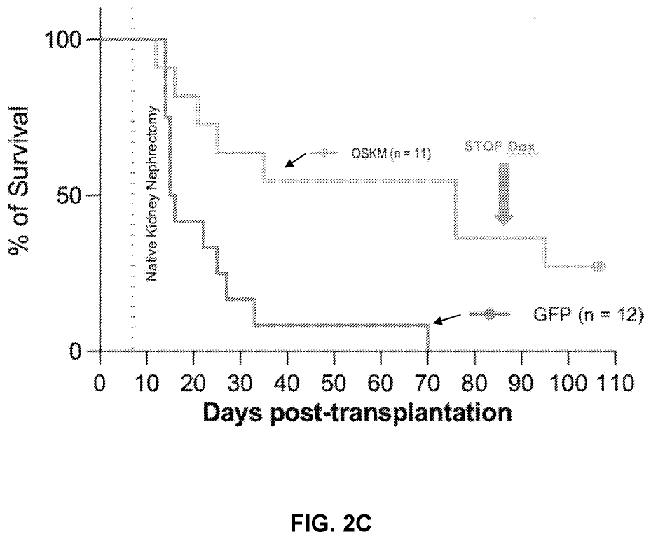

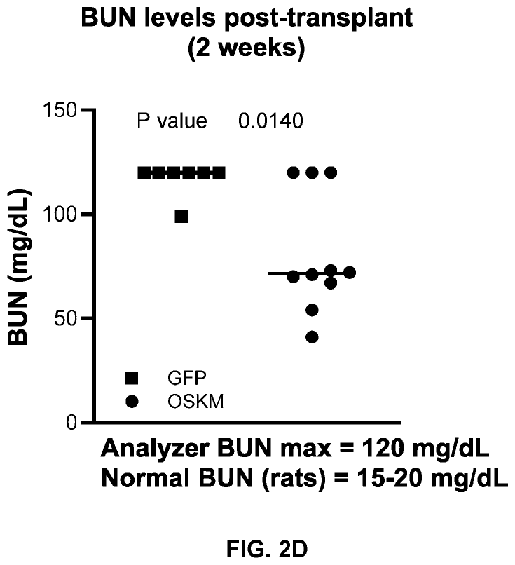

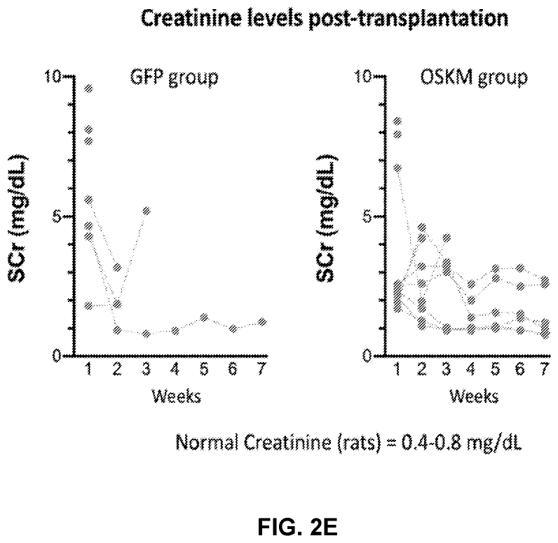

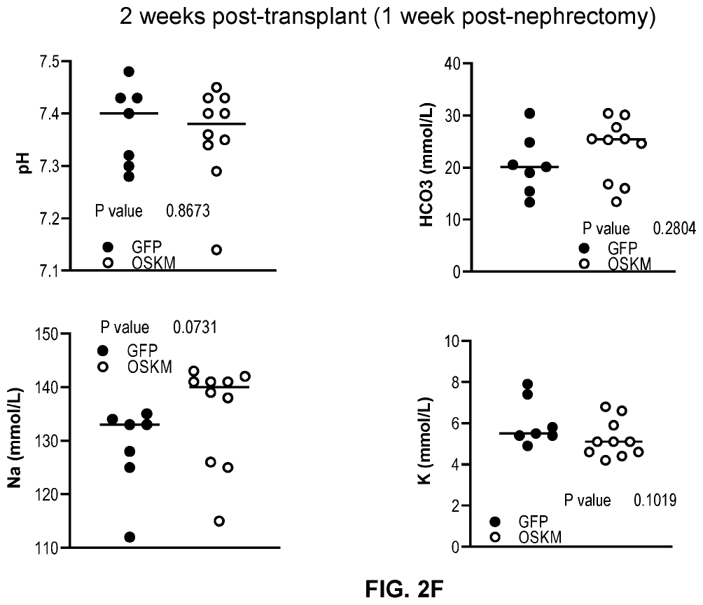

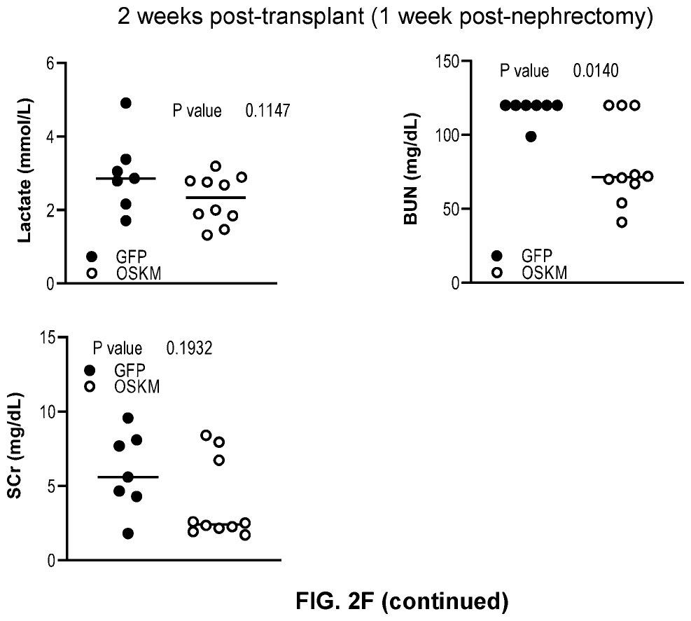

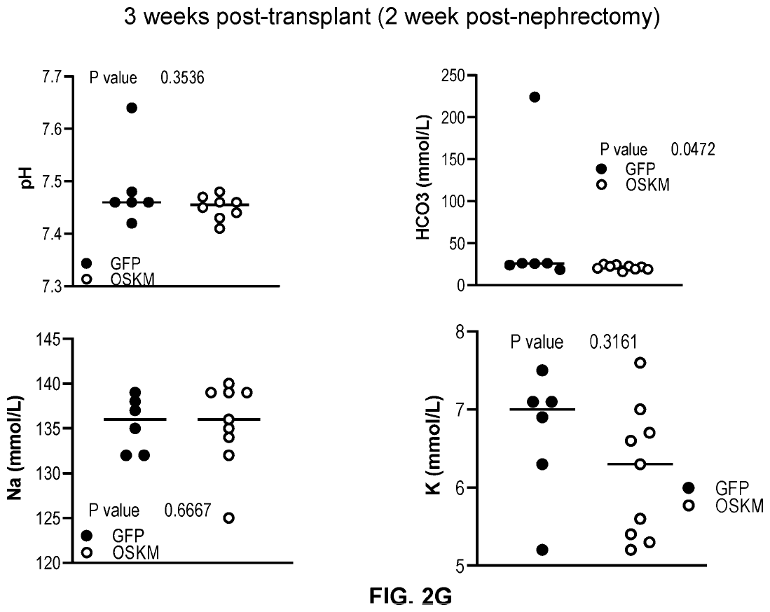

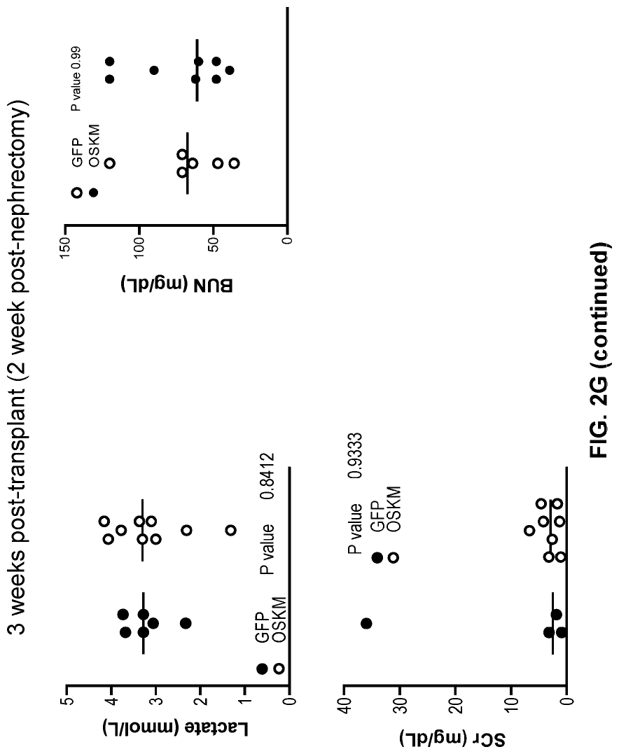

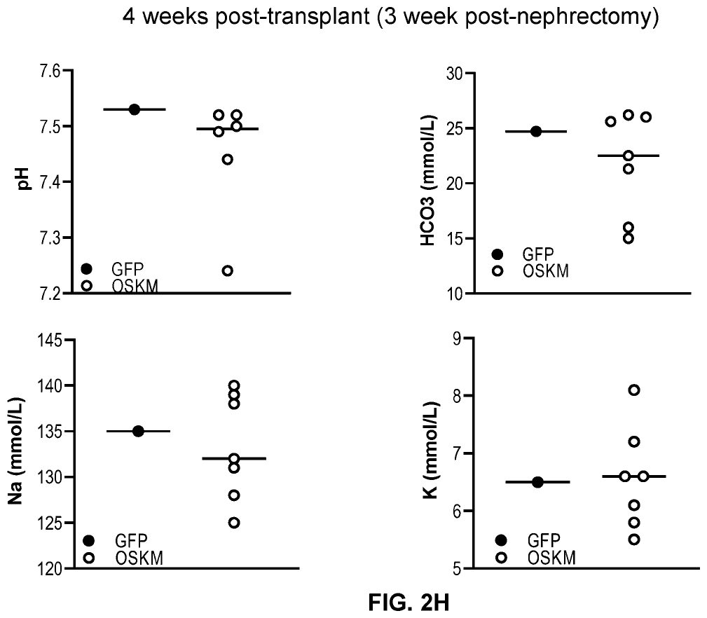

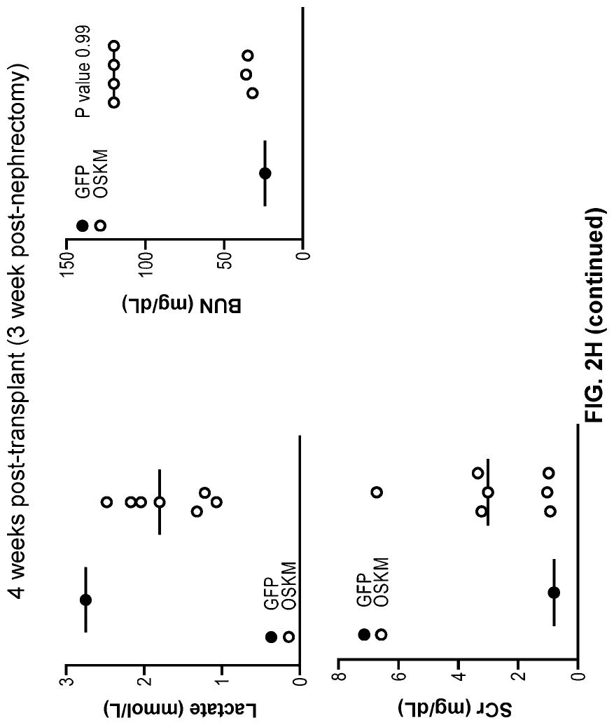

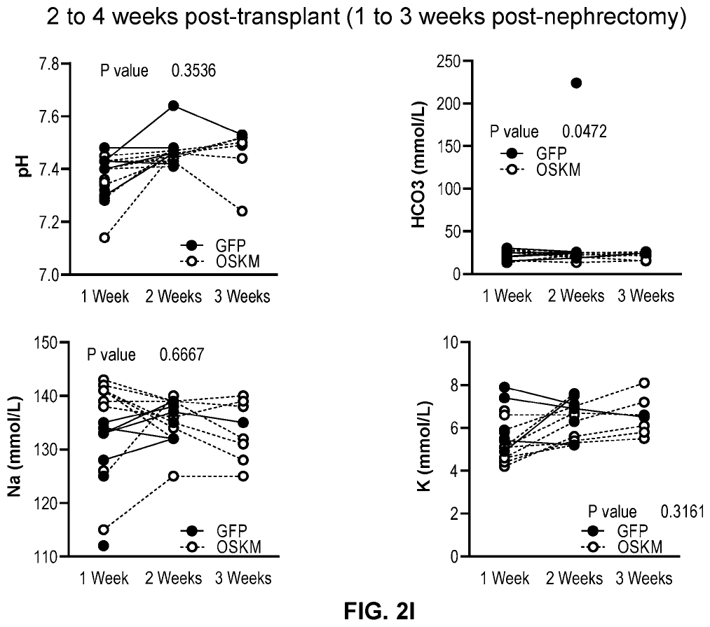

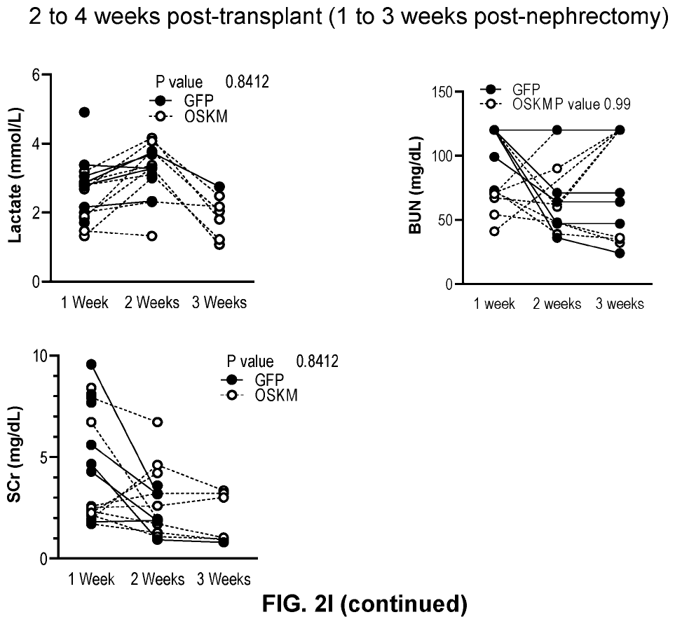

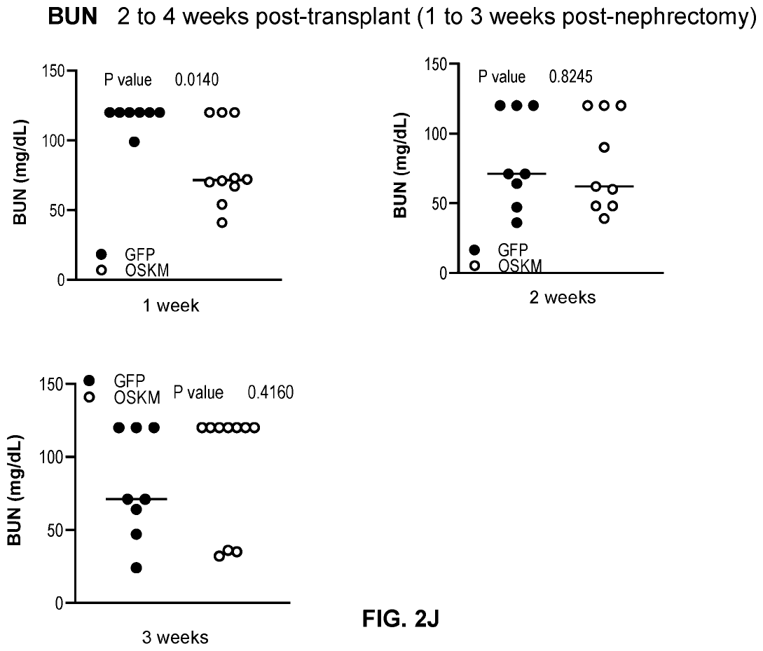

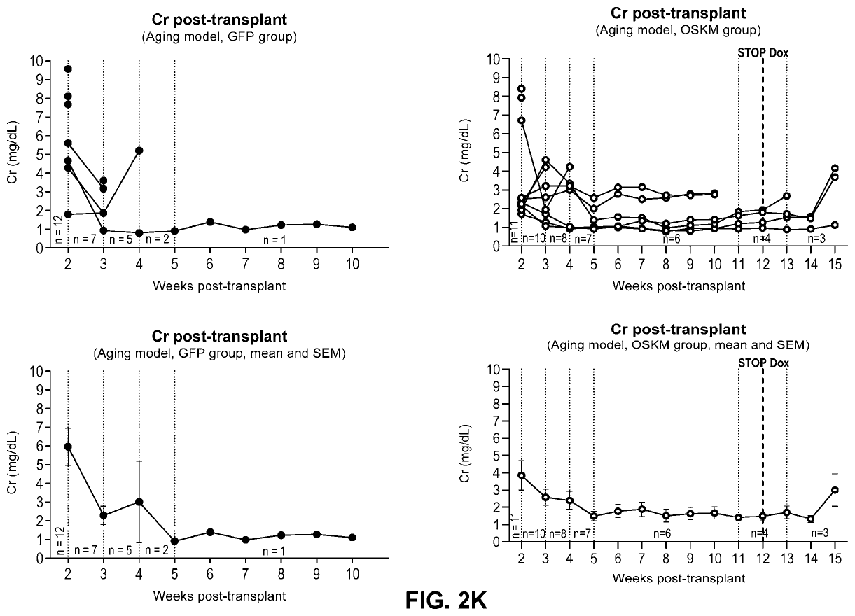

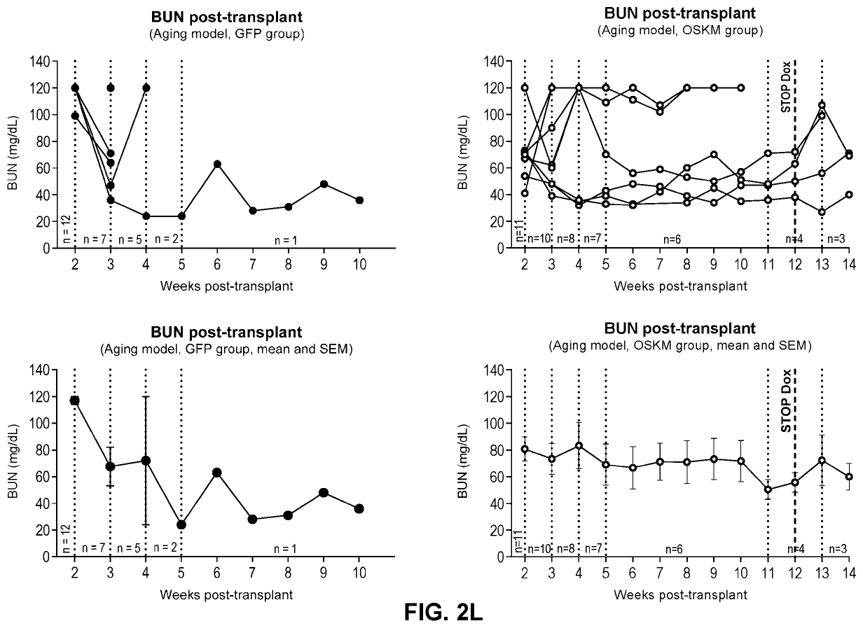

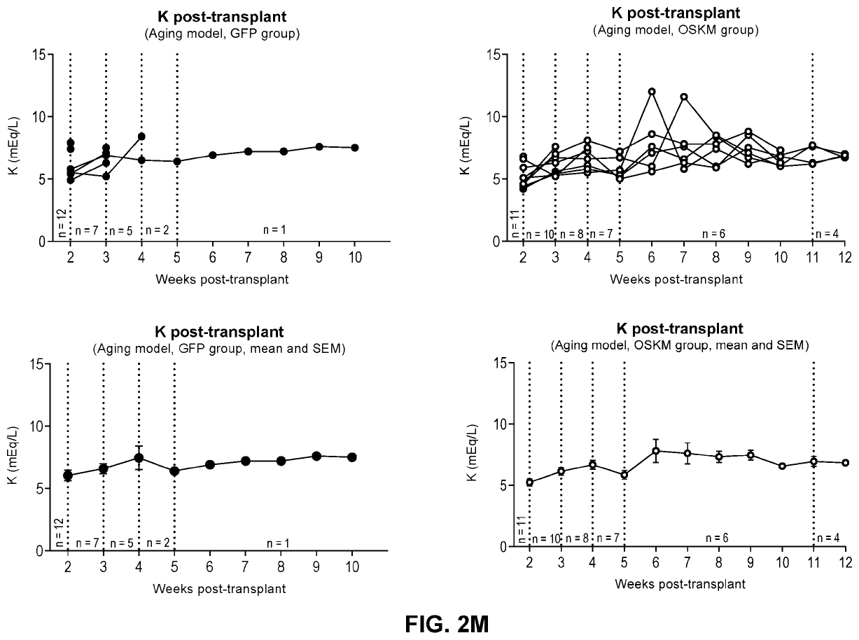

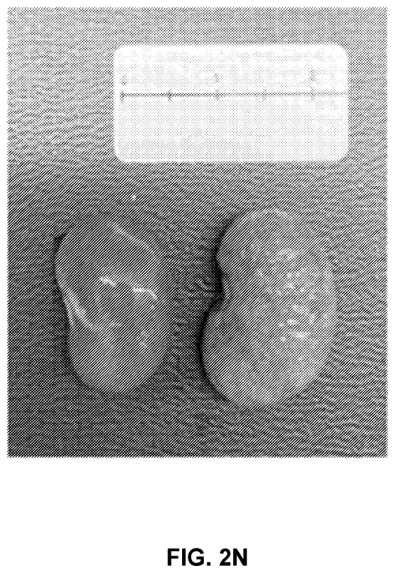

In the kidney, aging is associated with a decline in glomerular filtration rate, increased vascular and interstitial lesions and increased susceptibility to acute injury (e.g., ischemia-reperfusion injury) (Stenvinkel, P. & Larsson, T. E. Am. J. Kidney Dis. 62, 339-351, 2013; Anderson, S. & Brenner, B. M. Am. J. Med. 80, 435-442, 1986; and Baylis, C. & Corman, B., J. Am. Soc. Nephrol. JASN 9, 699-709, 1998). A clinically relevant model of kidney allotransplantation from aging donors was used to test the effect of ex vivo AAV-delivered Yamanaka factors (Oct-3/4, Sox2, Klf4 and c-Myc [OSKM]) on kidney transplant function and kidney function—dependent rat survival (see FIG. 2A). Within this model, ischemic injury occurs from the organ donation and the normothermic perfusion, along with an immune injury resulting from the different genetic backgrounds of the donor (Dark agouti strain) and the recipient (Lewis strain).

Donor kidneys from aged (9-month-old) Dark agouti rats (DA rats) underwent NMP normothermic perfusion machine for approximately 1 hour, with inducible AAV-OSK and AAV c-Myc/rtTA (each vector was approximately 1×10¹²; or total titer 2×10¹²). Anti-rejection medication, tacrolimus, was then administered for 5 days, following the kidney transplant to prevent early graft rejection. One week post-transplantation, the contralateral kidney was removed. Starting one week after transplantation, OSKM expression was driven by rtTA activation via doxycycline (Dox) supplementation in drinking water, 2 days a week. Vehicle controls were perfused with the same titer of AAV GFP/luc (1×10¹²). Dox administration stopped at 87 days post-transplantation within the AAV OSKM group (after 80 days of Dox treatment).

Histological analysis demonstrated inflammatory infiltrates, tubular injury, and interstitial edema in kidneys from aged donors transplanted with a kidney perfused with AAV-GFP (see FIG. 2B). It is known from prio research that inflammatory infiltrates re due mostly to alloimmune injury, and can also result from ischemic insult. Tubular injury occurs more prominently in ischemic injury, but can also result from alloimmune injury. Interstitial edema can be observed in both alloimmune and ischemic injuries.

The survival rate of the OSKM-treated group was significantly higher compared to the control group (see FIG. 2C). All animals in the control group died by day 70, or earlier. Dox treatment was discontinued at day 87 post-transplantation. One rat died one week post discontinuation (this rat was observed to have degraded kidney function), and the other three rats survived for three more weeks at which point, the animals were studied for analysis (see FIG. 2C). Kidney function was in the 3 surviving rats, as exhibited by stable serum creatinine levels for the 3 weeks after stopping dox (see FIG. 2K). Notably, serum creatinine levels in the OSKM-treated group were lower, and maintained the levels during the entire study period compared to the control group treated with AAV-GFP/luc. The function of the kidney was significantly increased in rats transplanted with a kidney perfused with OSKM in comparison to the control group treated with AAV-GFP/luc, exhibiting lower Blood Urea Nitrogen (BUN) levels (i.e., indicating better function) 2 weeks post kidney transplantation (see FIGS. 2D and 2F), stable BUN levels, pH, bicarbonate, sodium, potassium, and lactate levels from 3 weeks to 12 weeks post transplantation (see FIGS. 2G, 2H, 2I, 2J, 2L, and 2M), and specifically stable BUN levels in surviving rats 3 weeks after discontinuation of dox (see FIG. 2L). The BUN max level was noted to be 120 mg/dL; while normal BUN levels are noted to range from 15-20 mg/DL. 100 days post-transplantation, the transplanted kidneys were collected from the surviving OSKM treated rats (no control rat were alive at this timepoint). No evidence of tumor was found within the kidneys (see FIG. 2N). Macroscopically, the kidneys of these OSKM-treated animals were enlarged and with an irregular surface; these features are usually observed in the allotransplantation model due to alloimmune injury. Overall, the results indicated that partial reprogramming with OSKM can improve kidney function following ischemic and immune injury.

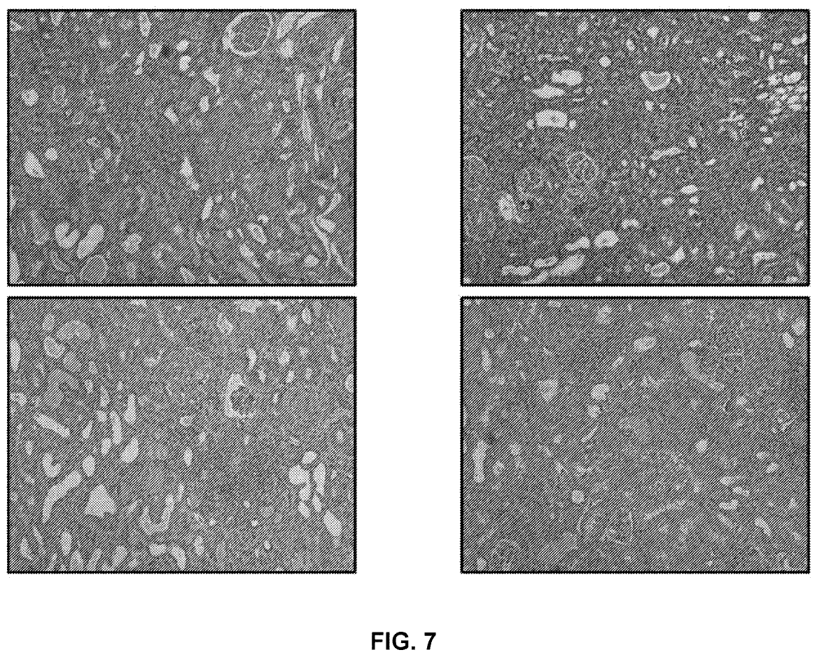

OSKM-treated kidneys were then sectioned and subjected to hematoxylin-eosin staining (see FIG. 7). Immune infiltrates, vascular lesions and tubular injury were observed in the sections above, consistent with alloimmune injury (transplant rejection) (see FIG. 7). The OSKM-treated mice did not receive anti-rejection medicine throughout the majority of the study period. The treated kidneys showed no signs of tumor or malignancy (see FIG. 7).

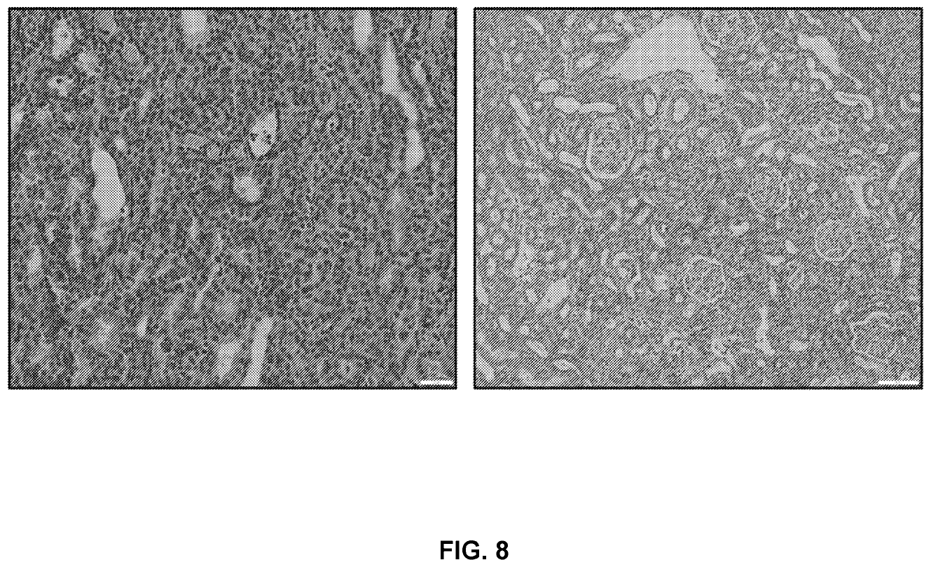

To evaluate levels of immune infiltrate in kidneys within the AAV-GFP/luc control group, kidneys were collected from two pre-mortem rats (i.e., mice showing signs of imminent death) and studied in the AAV-GFP/luc group 15 and 16 days post-transplant. The hematoxylin-eosin (H&E) stained sections showed alloimmune rejection features with an extensive immune infiltrate, as expected given that the mice were not treated with anti-rejection medicine (except as noted in the early stages of the protocol) (see FIG. 8). These results demonstrated that level of immune rejection (immune infiltrate) was lower than expected in the OSKM-treated mice. Because the OSKM-treated mice also survived longer and demonstrated improved kidney function, the results indicated that OSKM treatment had a beneficial effect in preventing allograft rejection.

### Example 4. OSKM Treatment Ameliorates Ischemic Injury in an Isogenic Kidney Transplantation Model

Ischemia-reperfusion injury is the main cause of acute kidney injury and a constant feature in kidney transplantation procedures. Ischemia-reperfusion injury (IRI) is a tissue injury that occurs when the blood supply to organs is interrupted and then blood supply is restored. IRI is usually associated with a robust inflammatory and oxidative stress response to hypoxia and reperfusion (upon, e.g., kidney transplantation), which disturbs organ function. Organ ischemia can result in tissue damage by decreasing levels of the energy-storage molecule ATP. The alterations result in accumulation of harmful reactive oxygen species, damage to mitochondrial organelles, and trigger an inflammatory response when oxygenated blood flow is restored to the transplanted organ. These symptoms are particularly acute in the higher-risk donor organs that form an increasing proportion of current organ transplants.

Organ retrieval, transport and transplantation of kidney grafts causes IRI. Cold ischemia results in IRI, and can occur during organ transplantation when the organ is cooled with a cold perfusion solution following procurement and subsequent transport. Warm ischemia also contributes to IRI, and can occur, e.g., in death by cardiovascular criteria (DCD) donors, where donor hypoxia may persist between the time of DCD and the time of organ procurement.

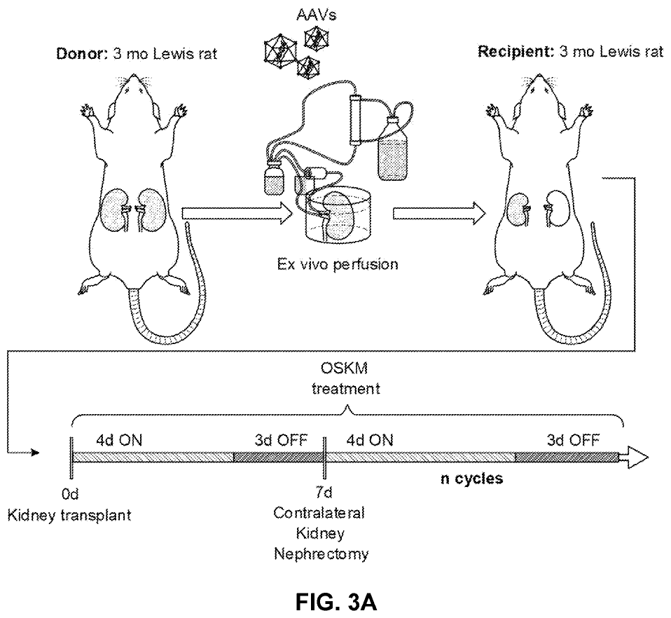

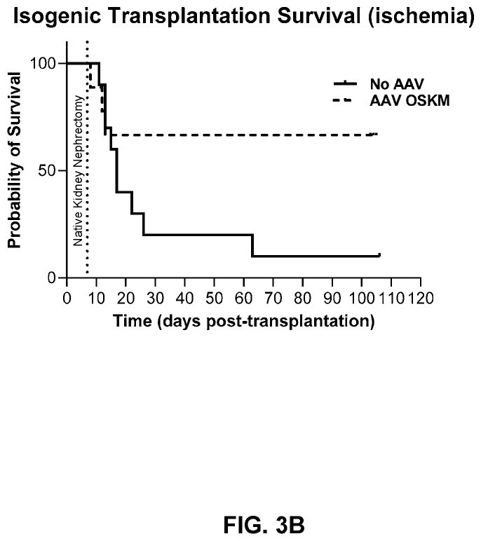

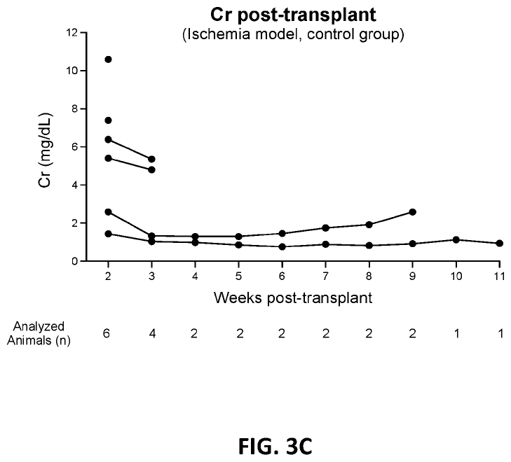

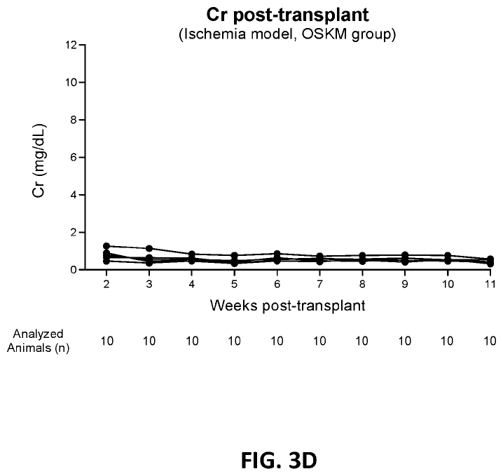

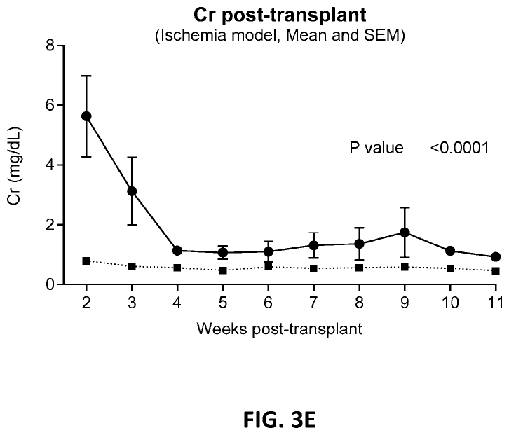

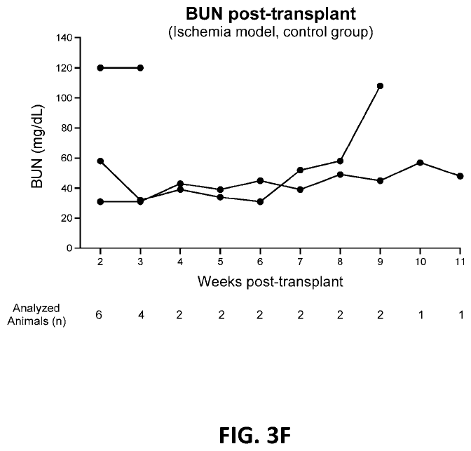

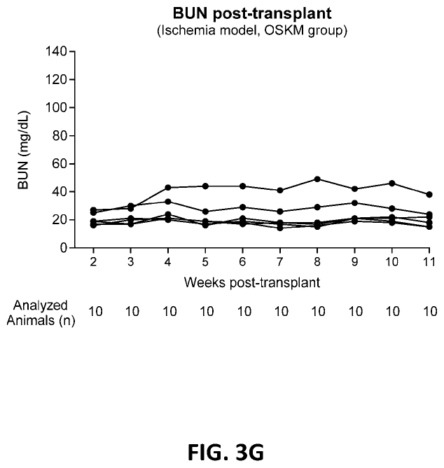

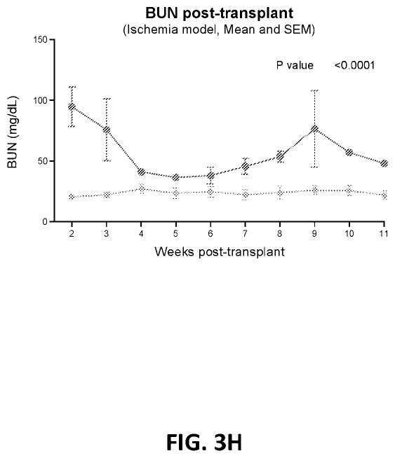

To understand whether OSKM can overcome ischemic injuries, kidney perfusion with OSKM was performed in an isogenic transplantation model (LEW to LEW, which lacks alloimmune injury) (see FIG. 3A). As shown in FIG. 3A, donor kidneys from 3-month-old endured NMP for approximately 1 hour, with inducible AAV-OSK and AAV c-Myc/rtTA (each vector 1×10¹²; total titer 2×10¹²). One week after transplantation, the contralateral (right) kidney was then removed. Starting one week after transplantation, OSKM expression was driven by rtTA activation by doxycycline (Dox), which was then added in drinking water, approximately 4 days a week. Control kidneys were then perfused, however no AAV was administered. Dox administration was stopped 105 days post-transplantation in the AAV OSKM group (after 56 days of Dox treatment). Organs were collected at day 105 post-transplant for analysis.

In this ischemic model, organ donation surgery and normothermic perfusion (1 hour) was associated with an ischemic injury, ischemic reperfusion lesions, in the ex vivo perfused kidney despite continuous oxygenation of the perfusate. The perfusate in this model, Gey's balanced solution, lacks an oxygen carrier, which is believed to result in reduced oxygen transport fto the cells. 10 of the 11 animals in the control group died by day 63 or earlier. In comparison to the OSKM-treated group, 3 animals died in the first 15 days, however 6 animals survived to the end of the study (approximately 125 days). Kidney function was stable in the 6 surviving rats, evident by stable serum creatinine and Blood Urea Nitrogen (BUN) levels noted through the end of the study.

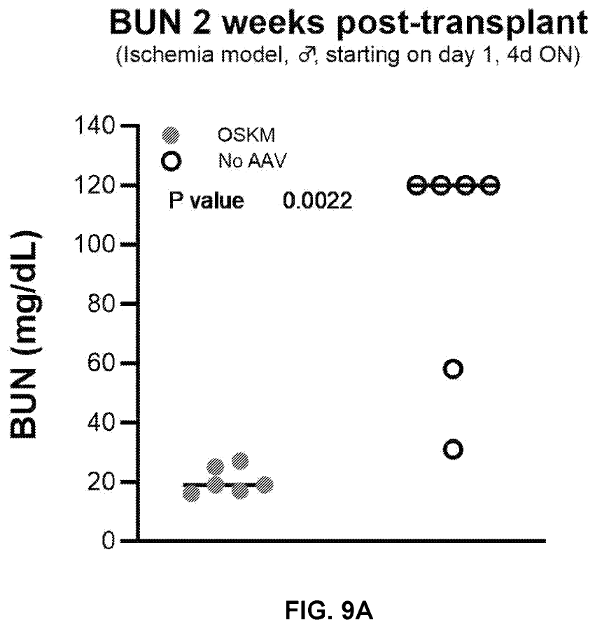

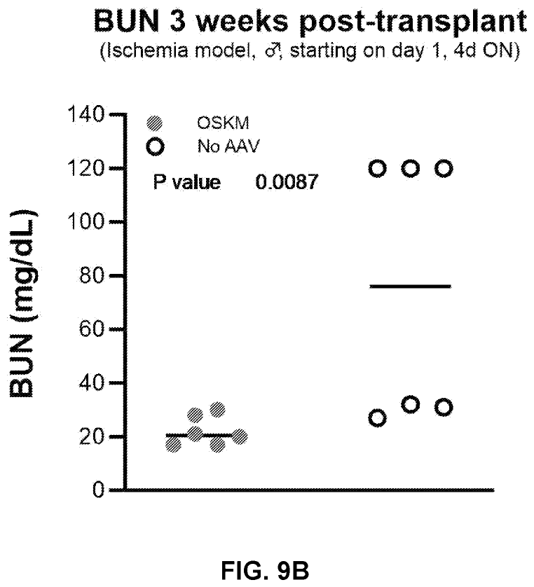

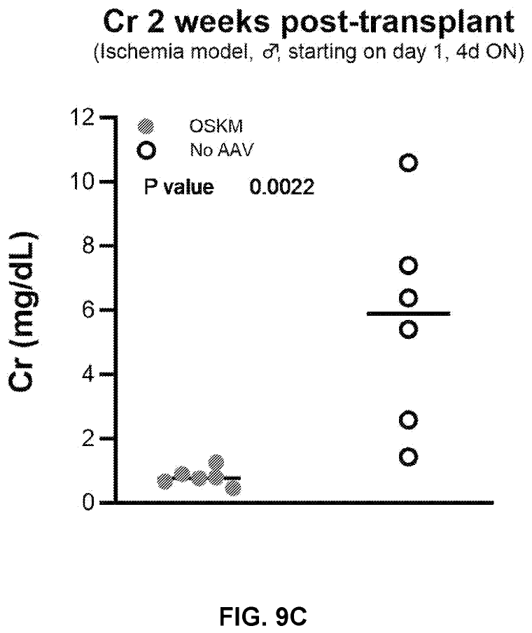

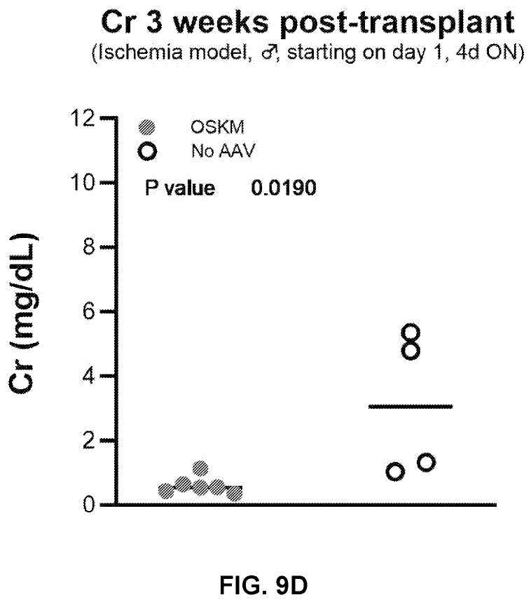

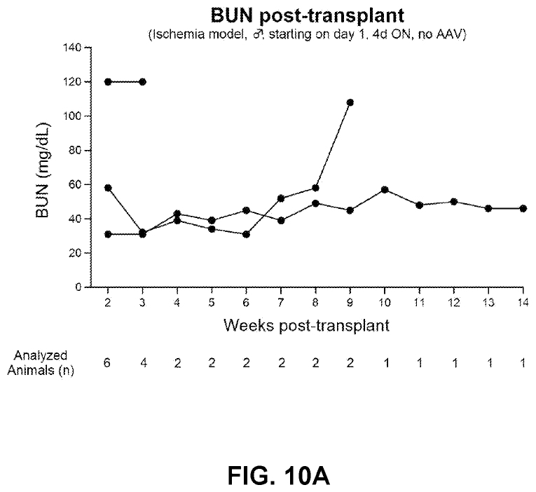

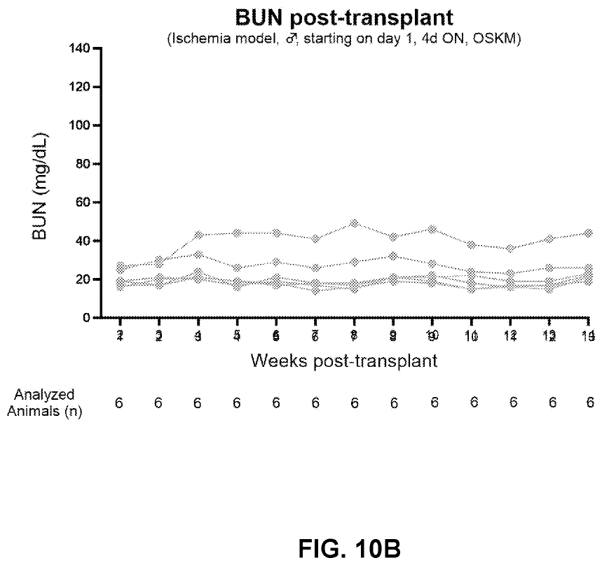

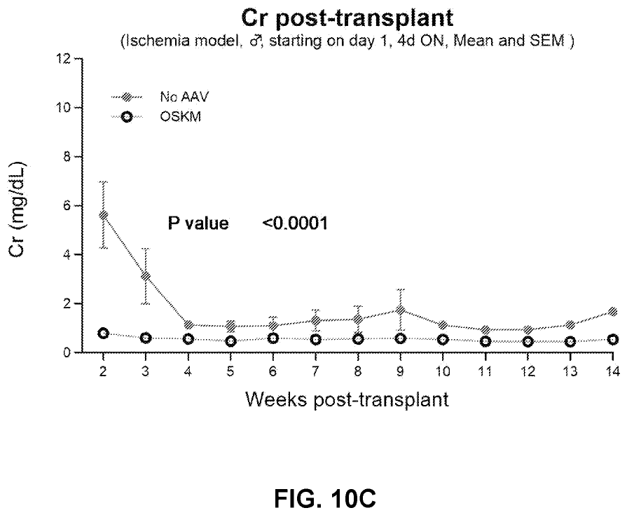

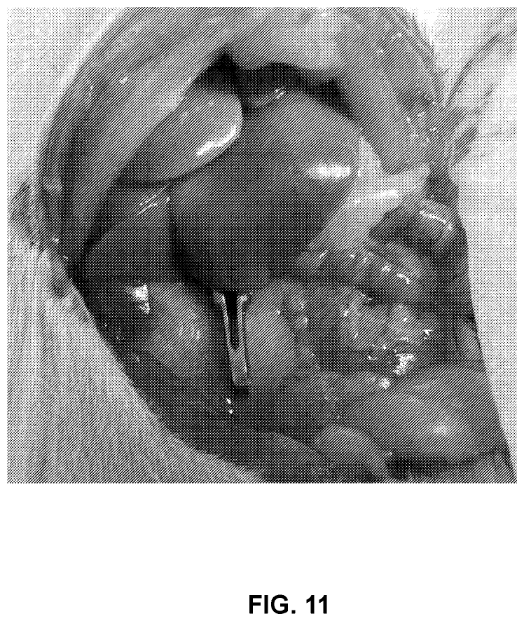

Ex vivo delivery of OSKM dramatically improve rat survival in the isogenic kidney injury model, as the survival rate in the OSKM treated animals is >60% 100 days following kidney transplantation (see FIG. 3B). Importantly, the levels of serum creatinine were lower in the OSKM treated group (see FIG. 3D, FIGS. 9C-9D, and FIG. 10C) compared to the controls (see FIG. 3C, FIGS. 9C-9D-D, and FIG. 10C). This difference is statistically significant (see FIG. 3E, FIG. 9C-9D, and FIG. 10C). BUN levels were also lower (desired effect) in the OSKM treated group (FIG. 3G, FIGS. 9A-9B, FIG. 10A) compared to the control group (see FIG. 3F, FIGS. 9A-9B, FIG. 10B). This difference was statistically significant (see FIG. 3H, FIGS. 9A-9B, FIG. 10A, and FIG. 10B). Harvested kidneys from the OSKM group were examined macroscopically and no tumors or teratomas were observed in the OSKM-treated kidneys.

Overall, the results indicated that partial reprogramming with OSKM ameliorated ischemic injury to the transplanted kidneys. The results also demonstrated preservation of the function of transplanted kidneys following isogenic transplantation in the ischemic ex vivo transplantation model.

### Example 5. OSKM Treatment in Rat Livers—Short Term Safety Studies

AAV-OSKM infection and safety were evaluated up to 1 week in vivo in Wistar rats (220-250 g) with healthy livers via administration of the AAV-OSKM vectors (intravenously through the penile vein). Within the control groups, rats were administered PBS via penic route, and on day 6 after PBS administration, doxycycline was then administered in the drinking water (1 mg/mL diluted in the drinking water). On day 7, rats were sacrificed and livers, blood, and kidneys were then retrieved (kidneys served as controls in this model). Within vehicle control groups, vehicle (AAV-GFP/Luc, 1×10¹² per rat) was administered by penic route and on day 6 after vehicle administration, doxycycline was administered in the drinking water (1 mg/mL diluted in the drinking water). On day 7, the rats were sacrificed and livers, blood, and kidneys (as controls) were retrieved.

Within the treatment groups, AAV-OSK+AAV-cMyc-rtTA vectors (1×10¹² per rat, of each vector) were administered by penile route and on day 6 after vector administration, doxycycline was administered in the drinking water (1 mg/mL diluted in the drinking water). On day 7, rats were sacrificed and livers, blood, and kidneys were retrieved (kidneys served as controls). Blood and tissue samples were then collected for examination. Livers were evaluated for hepatic damage, inflammation and regeneration/tissue reparation. Widely standardized hepatic lesion parameters (e.g., AST, ALT and bilirubin), apoptotic parameters (e.g., caspase 3), and hepatic cell proliferation (e.g., Ki67/PCNA, HGF and TGF-β) were analyzed. Regarding hepatic inflammation: neutrophil accumulation was evaluated by Myeloperoxidase (MPO) activity, and oxidative stress by Malondialdehyde (MDA) levels. Histological analyses (H &E) was utilized to evaluate hepatic damage (10×). Data are expressed as means±standard error and were statistically analysed via Kruskal-Wallis test, followed by post hoc Dunn's Multiple Comparison Test, if required. P<0.05 was considered significant.

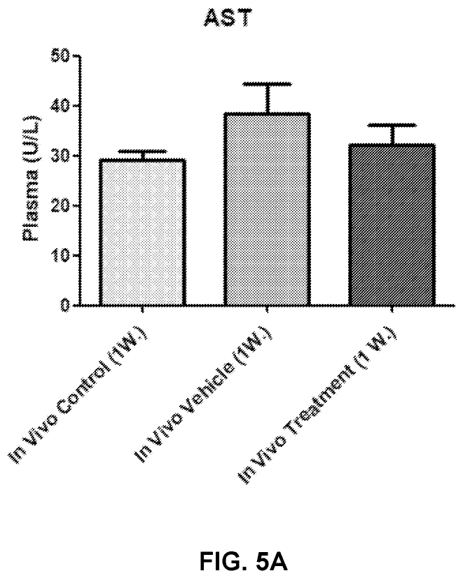

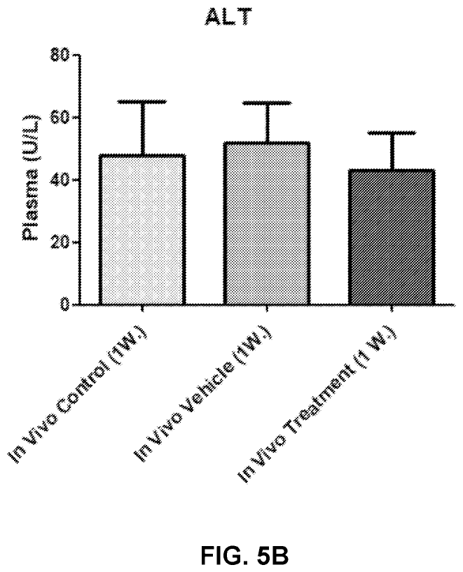

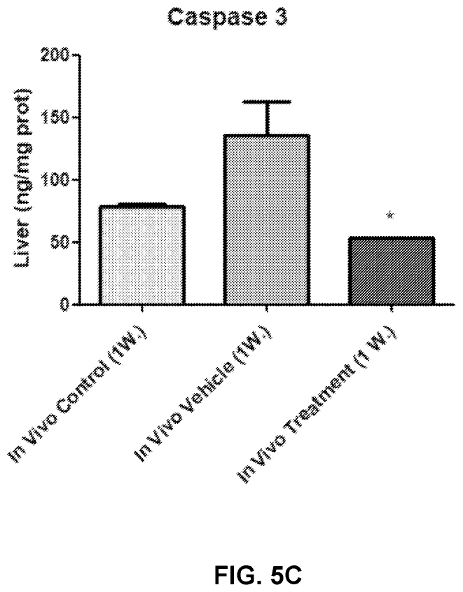

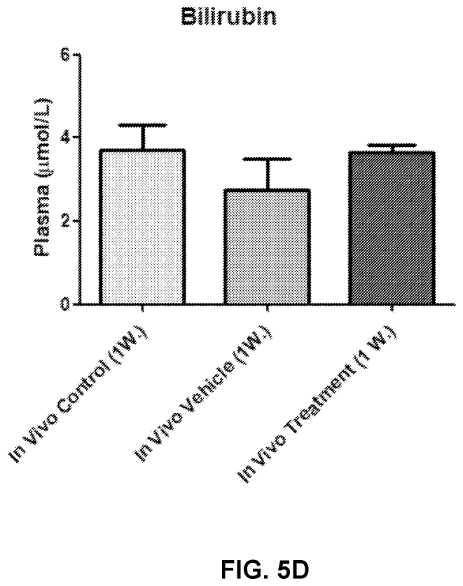

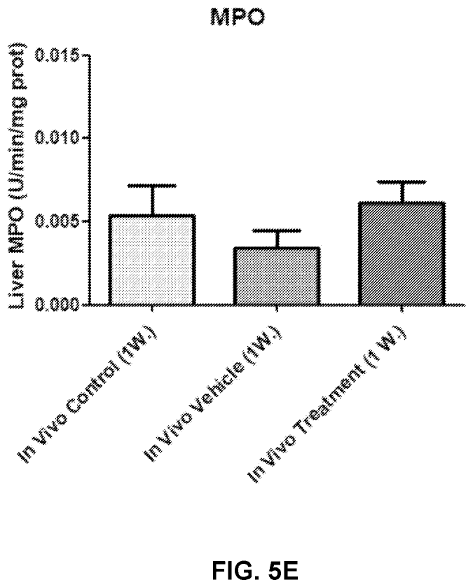

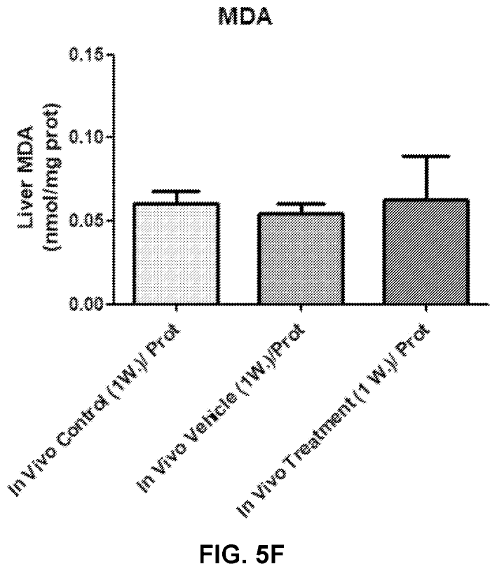

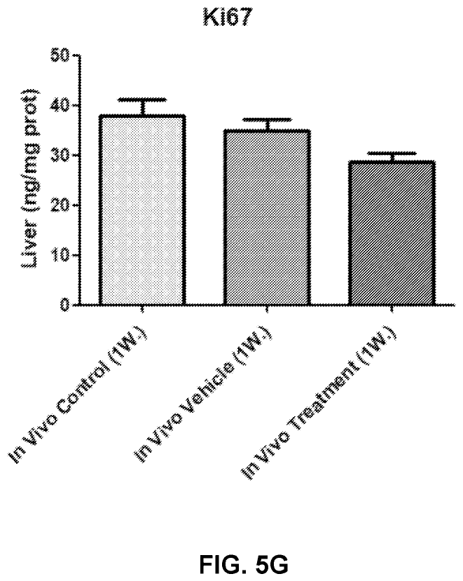

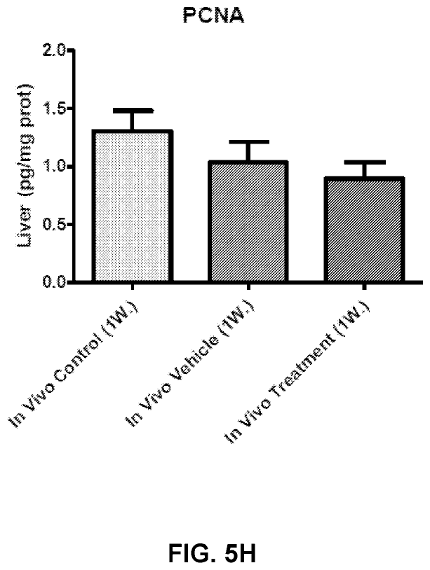

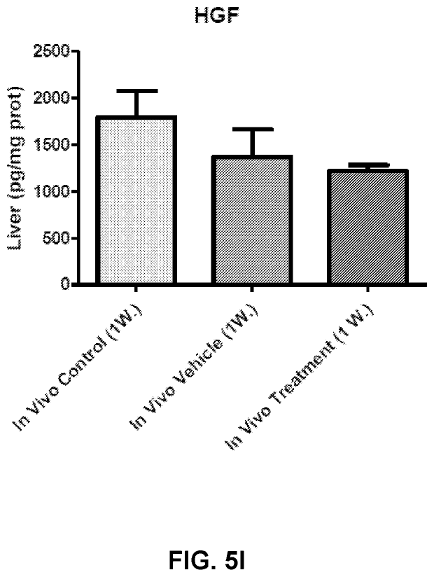

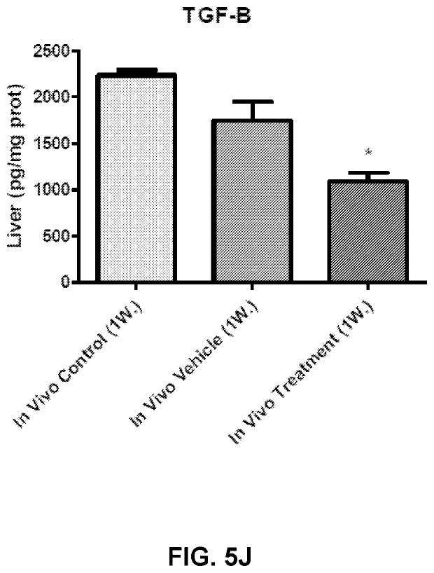

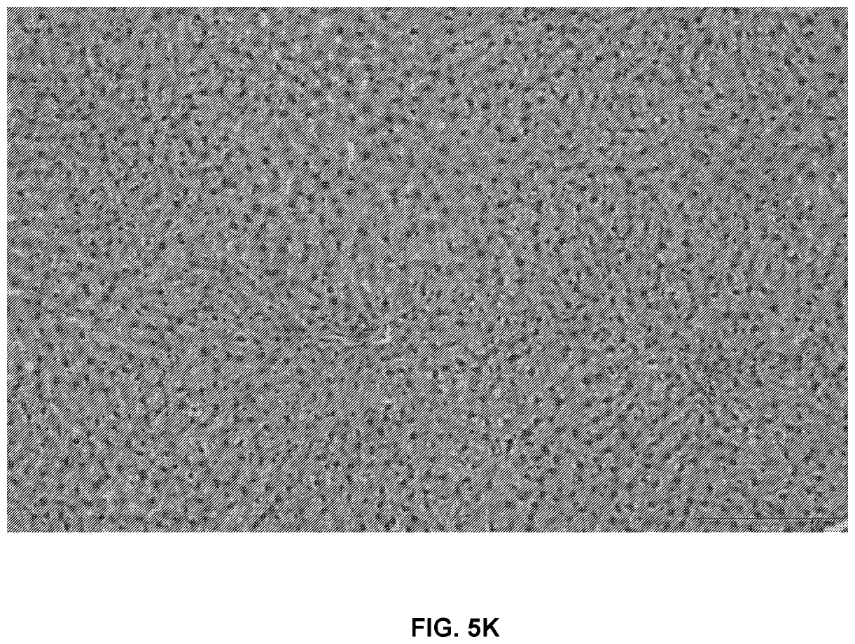

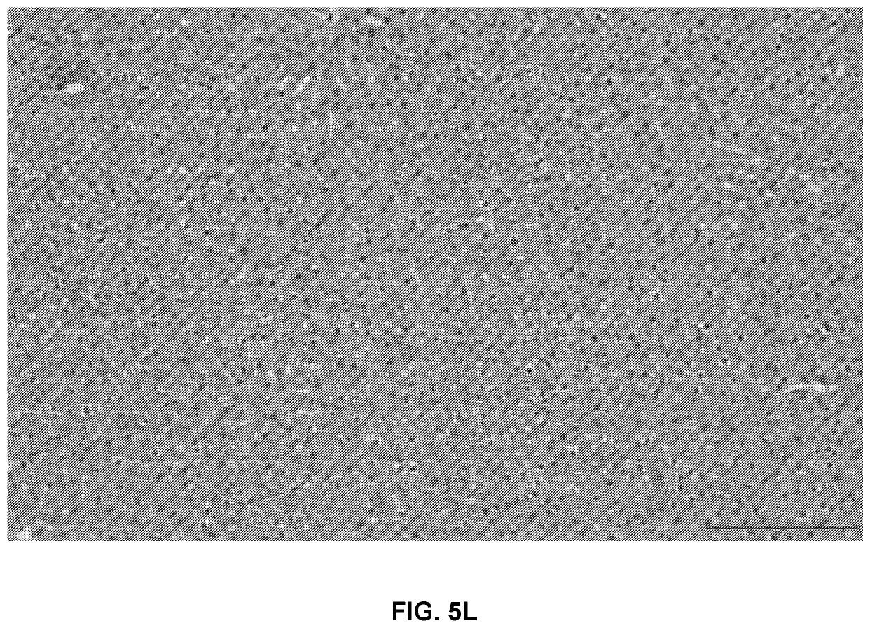

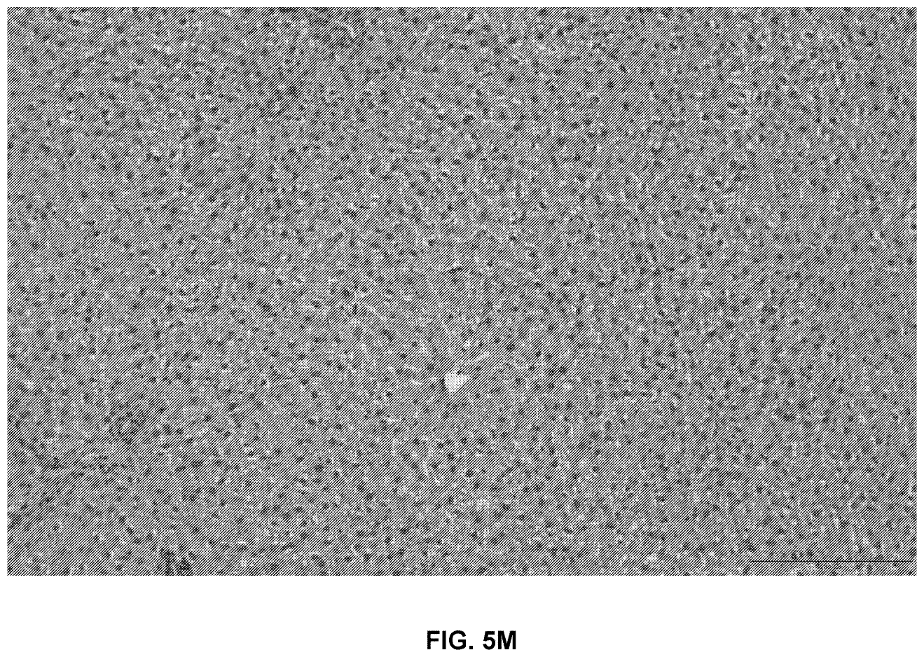

The results for the biochemical parameters of hepatic damage and functionality (AST, ALT, bilirubin) demonstrated that the levels of AST, ALT and bilirubin were similar in all of the groups of the study (see FIGS. 5A, 5B, and 5D). A reduction in caspase 3 levels was observed in the AAV-OSKM treatment group when compared with the control groups (see FIG. 5C).

The parameters of hepatic inflammation (neutrophil accumulation and oxidative stress), as determined by levels of MPO and MDA, were similar in all groups (see FIGS. 5E and 5F), indicating the lack/absence of hepatic inflammation when AAV-OSKM were administered (intravenously). In addition, the parameters of hepatic proliferation/hepatic tissue reparation, as determined by levels of Ki67, PCNA, and HGF were similar in all groups (see FIGS. 5G, 5H, and 5I); whereas a reduction in TGFβ (a potent pro-proliferation and fibrogenic molecule was observed in the AAV-OSKM treatment group when compared to the control groups (see FIG. 5J).

No apparent foci of necrosis were observed in any of the groups evaluated (see FIGS. 5K, 5L, and 5M), consistent with the biochemical measurements.

These results demonstrated that short-term in vivo treatment was safe, as determined by measuring parameters of hepatic damage and functionality, hepatic inflammation and hepatic reparation/regeneration.

### Example 6. OSKM Treatment in Rat Livers—Long Term Safety Studies

The safety of AAV-OSKM infection was evaluated up to 12 weeks following intravenous administration of AAV-OSKM in Wistar rats (220-250 g) with healthy livers. In the control groups, rats were administered PBS by penile (interchangeably termed penic) route and starting on day 6 after PBS administration doxycycline was administered for 24 hours in the drinking water (1 mg/mL diluted in the drinking water). The oral doxycycline administration for 1 day/week continued for 12 weeks. The rats were then sacrificed after 12 weeks (on Dox treatment) and liver, blood, pancreas, kidneys, adipose tissue, skin, spleen, hear, lung intestine and muscle were retrieved. In vehicle control groups, the vehicle (AAV-GFP/Luc, 1×10¹² per rat) was administered by penic route and starting on day 6 after vehicle administration, doxycycline was administered for 24 hours in the drinking water (1 mg/mL diluted in the drinking water) and oral doxycycline administration for 1 day/week was continued for 12 weeks, at which time the rats were sacrificed and liver, blood, pancreas, kidneys, adipose tissue, skin, spleen, heart, lung intestine and muscle were retrieved.

In the treatment groups, AAV-OSK+AAV-cMyc vectors (1×10¹² per rat, of each vector) were administered by the penile route and starting on day 6 after vector administration, doxycycline was administered for 24 hours in the drinking water (1 mg/mL diluted in the drinking water). The oral doxycycline administration for 1 day/week was continued for 12 weeks. The rats were sacrificed after 12 weeks and liver, blood, pancreas, kidneys, adipose tissue, skin, spleen, heart, lung intestine and muscle were retrieved.

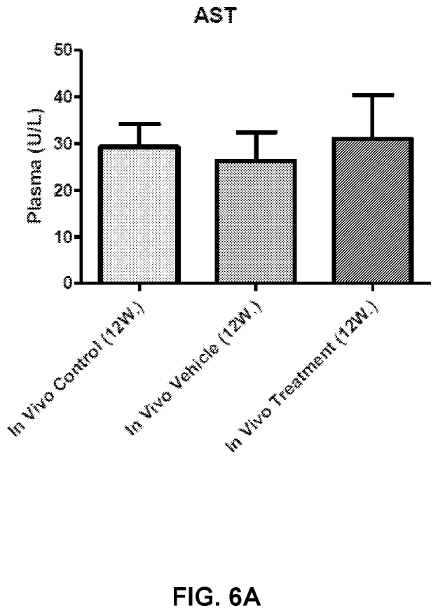

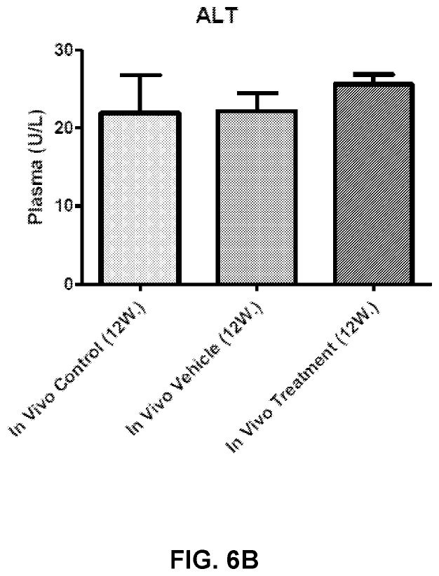

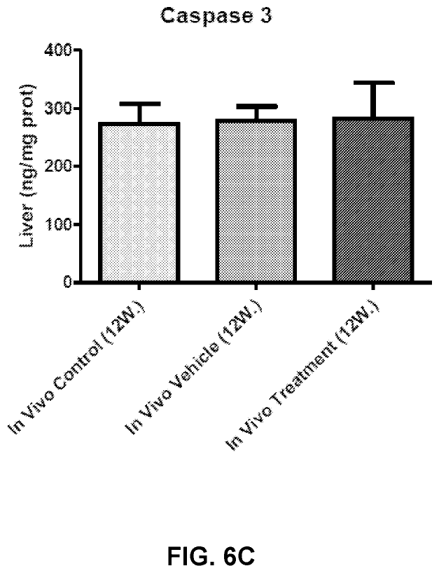

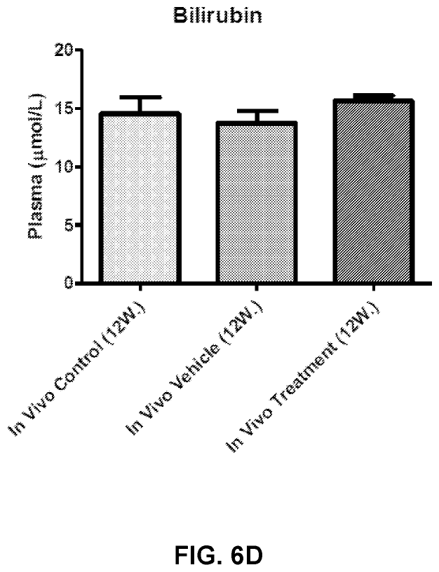

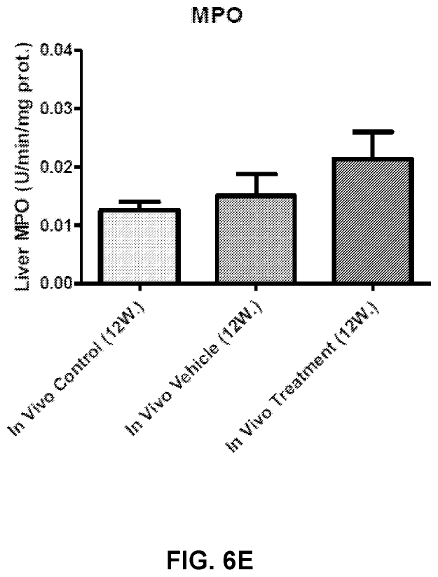

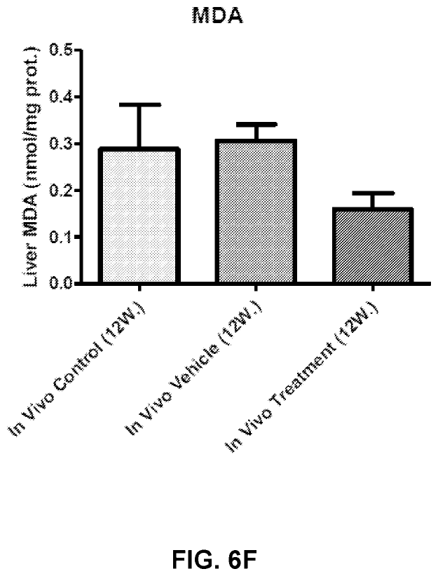

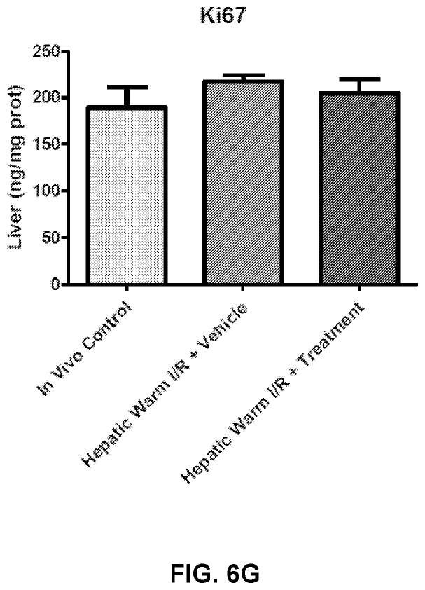

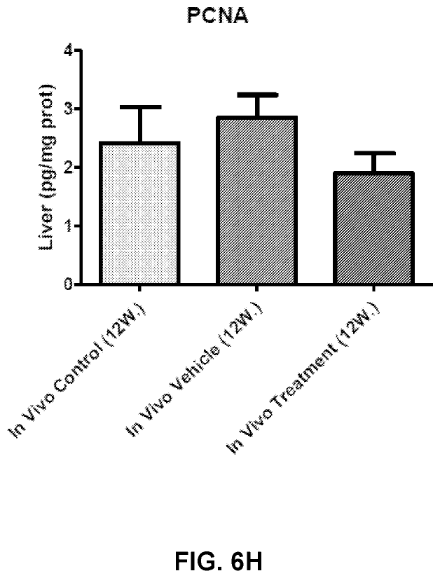

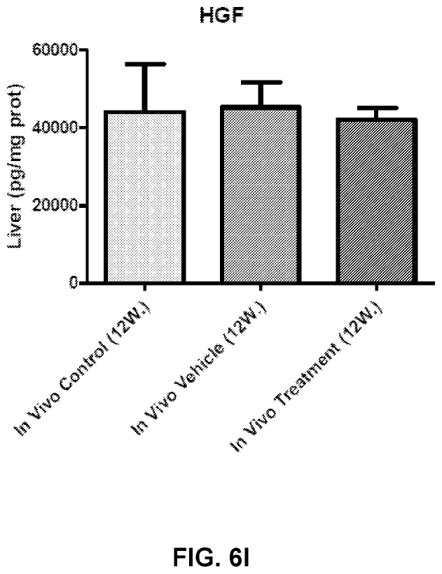

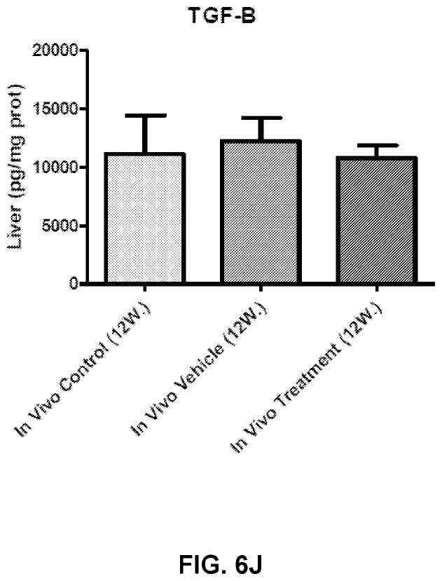

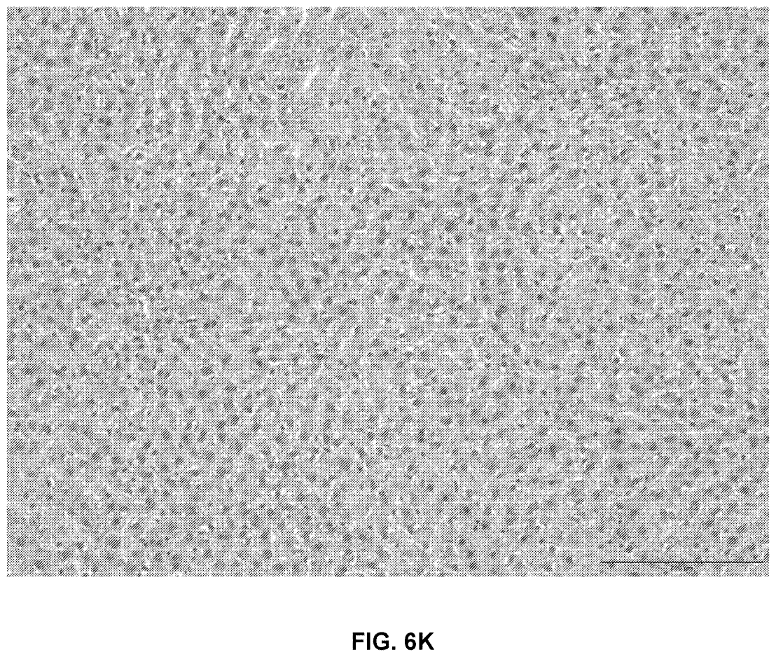

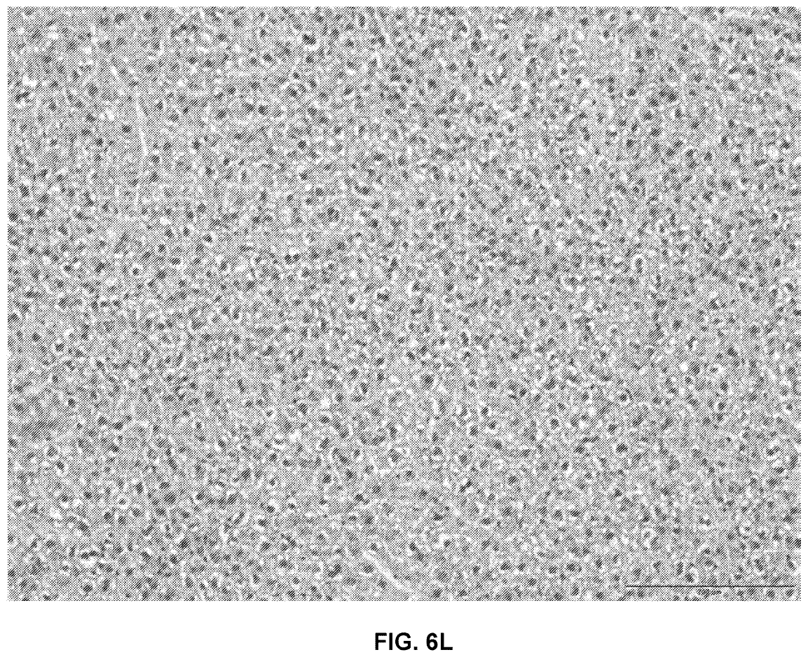

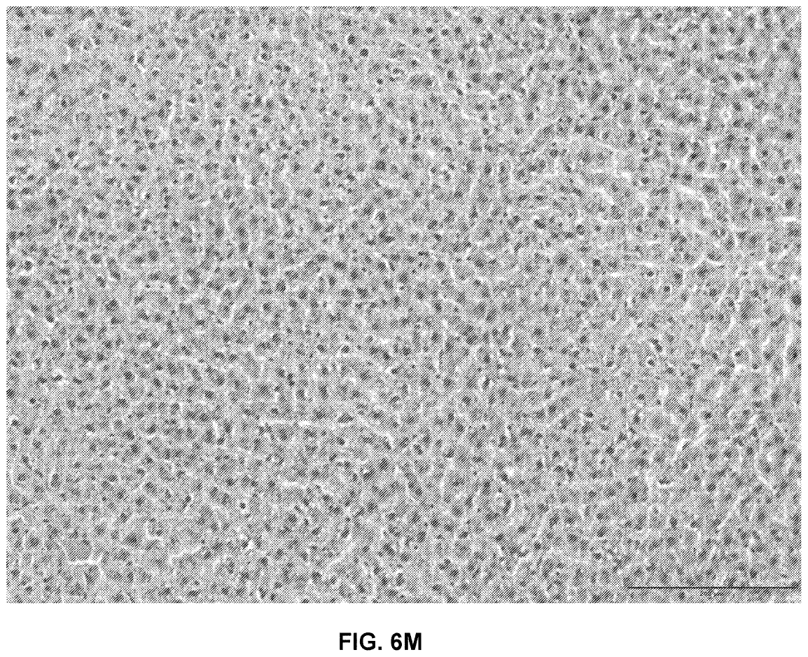

The biochemical parameters of hepatic damage and functionality (AST, ALT, bilirubin), the levels of AST, ALT and bilirubin, were similar in all of the groups (see FIGS. 6A, 6B, and 6D). As were the levels of caspase 3 (see FIG. 6C). The parameters of hepatic inflammation (neutrophil accumulation-measured by MPO and oxidative stress-measured by MPO, (see FIGS. 6E and 6F), as well as those of hepatic proliferation/hepatic tissue reparation (levels of Ki67, PCNA, HGF and TGFβ, (see FIGS. 6G, 6H, 6I and 6J)) exhibited no differences between the groups. Liver histologies in control and AAV-OSKM treated rats did not show any liver damage at 12 weeks (see FIGS. 6K, 6L, and 6M). The data demonstrate that AAV-OSKM intravenous administration and cyclic weekly Dox activation for 12 weeks is safe.

### Example 7. Effect of Pre-Injury Liver Reprogramming Delivered by AAV in Rats in Warm Hepatic Ischemia-Reperfusion Injury, an Acute, Clinically Relevant Liver Injury Model

The effects of pre-injury OSKM reprogramming were evaluated in a rat model of segmental (70%) hepatic ischemia (also termed warm hepatic ischemia reperfusion injury). This model is generally described in Peralta et al., J Hepatology 59, 1094-1106 (2013).

An acute warm hepatic ischemia-reperfusion injury was induced by clamping the major vessels to the liver for one hour, then releasing the clamp for varying periods of time. This generated a warm hepatic ischemia reperfusion (I/R) injury, modeling the I/R injury observed during hepatic resection and liver transplant surgeries. Warm hepatic ischemia reperfusion injury occurs with hepatic surgical resection because the major vessels to the liver are clamped during the surgery to avoid the blood loss during hepatic resections (i.e., hepatectomies), which are surgsurgeries to remove part or all of the liver. Acute warm ischemia reperfusion injury is also associated with the anhepatic phase (the time from the physical removal of the liver from the recipient to recirculation of the graft) during liver transplantation. The remaining liver following surgical resection is then capable of regenerating, to replace the removed portions, however warm ischemia-reperfusion negatively affects the regeneration of the remaining liver following hepatic resection which can lead to major post-operative complications. Warm ischemia/reperfusion injury associated with liver transplantation can result in negative postoperative outcomes.

PBS (control), vehicle control (5×10¹¹ AAV-GFP/Luc+5×10¹¹ AAV-rtTA) and OSKM AAV (AAV-OSK+AAV-cMyc vectors (5×10¹¹ AAV-OSK+5×10¹¹ AAV cMyc/rtTA) were administered to rats via the penile route, which delivered AAV or control to the liver, and the animals were treated with doxycycline after varying time periods (1 day, 3 days or 5 days) to induce expression of OSKM, as described below.

To generate the warm hepatic ischemia reperfusion injury, animals were anesthetized with isoflurane. After anesthetizing the animals, a midline laparotomy was performed and the hepatic artery and portal vein to the left and median liver lobes were occluded for 60 minutes. Reperfusion was initiated by removal of the occlusion clamp. After four hours of reperfusion, animals were sacrificed and organs/tissues collected for analysis.

Hepatic lesion parameters (AST, ALT and bilirubin), apoptotic parameters (caspase 3), and hepatic cell proliferation (Ki67/PCNA, HGF and TGF-β) were analyzed. Regarding hepatic inflammation: neutrophil accumulation was evaluated by MPO activity and oxidative stress by MDA levels. Data are expressed as means±standard error and were statistically analysed via Kruskal-Wallis test, followed by post hoc Dunn's Multiple Comparison Test, if required. P<0.05 was considered significant.

1 week, four day and 2 day protocols were tested.

In the one week protocol, PBS control, vehicle control (AAV-GFP/Luc+AAV-rtTA) or AAV-OSKM (AAV-OSK+AAV cMyc/rtTA) were administered. On day 6, after administration, doxycycline was added into the drinking water of the rats (1 mg/mL diluted in the drinking water). On day 7 after administration, hepatic I/R was carried out as described above. After 4 hours of reperfusion, the rats were sacrificed and organ/tissues retrieved.

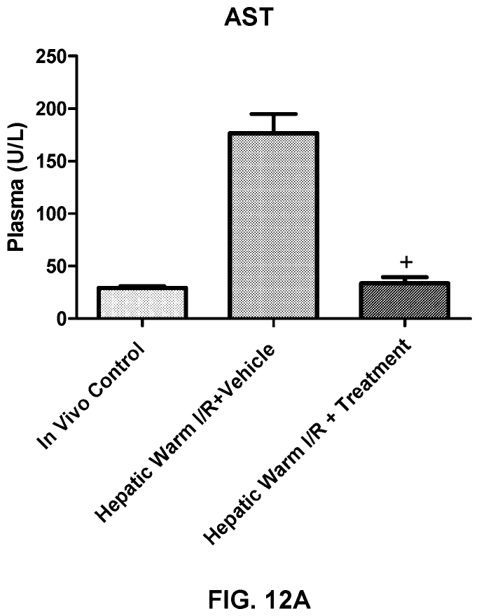

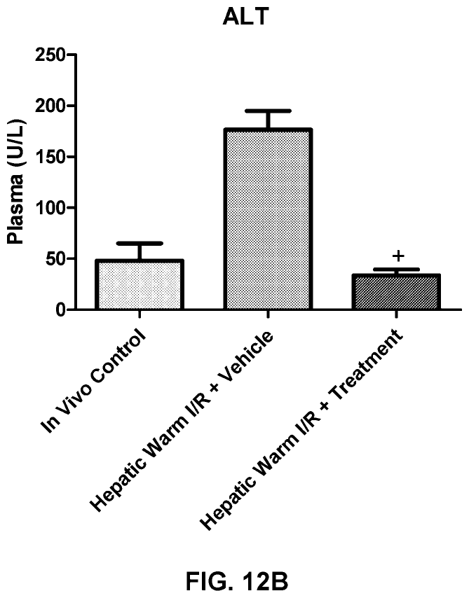

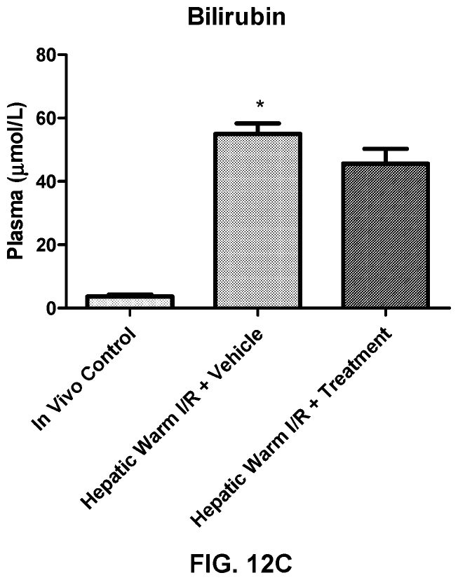

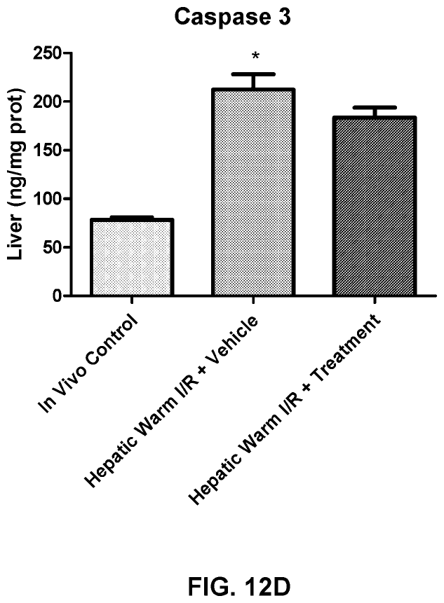

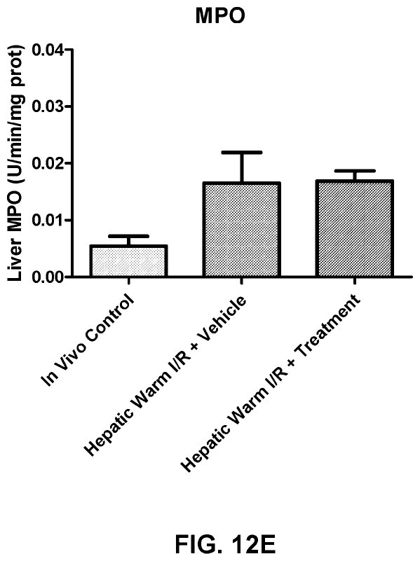

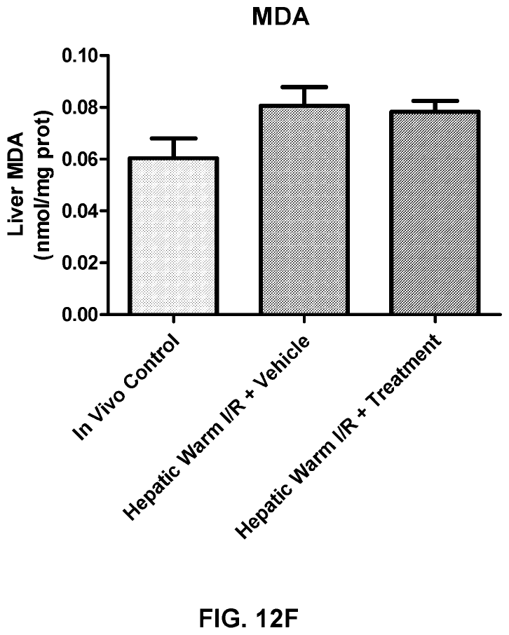

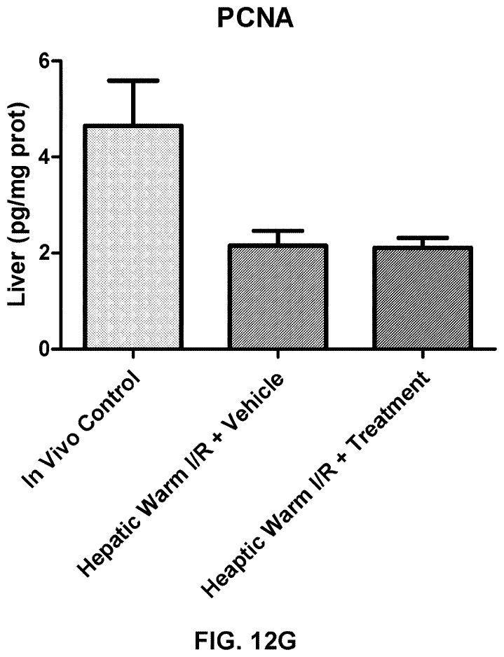

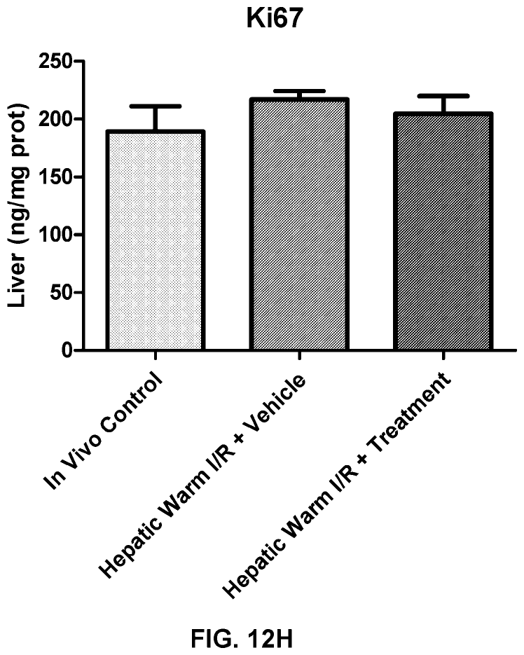

Intravenous administration of OSKM (AAV-OSK+AAV-cMyc+AAV-rtTa) 1 week before the induction of warm partial hepatic ischemia reperfusion resulted in a statistically significant reduction in the levels of biochemical markers of hepatic damage and functionality: transaminases, namely AST and ALT, when compared with the results obtained in the in vivo control vehicle group+hepatic I/R effect of OSKM. However, no differences in the levels of bilirubin were observed in both groups. It is possible that more time is required after injury to detect changes in BUN measurements, since such differences are sually observed at longer reperfusion times. apoptotic parameters (caspase 3), hepatic cell proliferation (Ki67/PCNA, HGF), neutrophil accumulation (MPO activity) and oxidative stress (MDA levels). Significant differences were not observed for any of these measurements, suggesting that the beneficial effects observed were not due to changes in oxidative stress, neutrophil accumulation and/or cell proliferation parameters (see FIGS. 12A-12H).

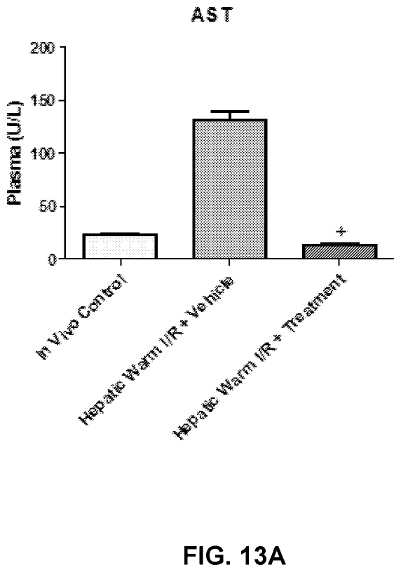

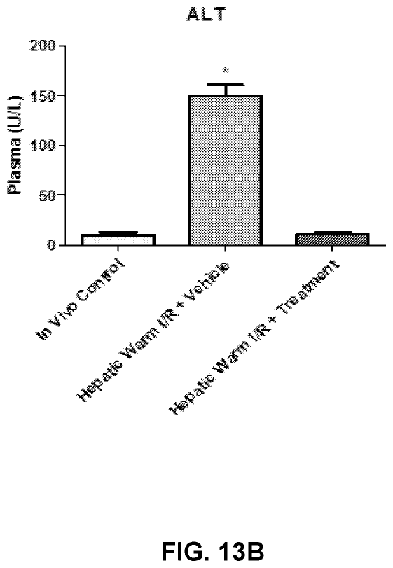

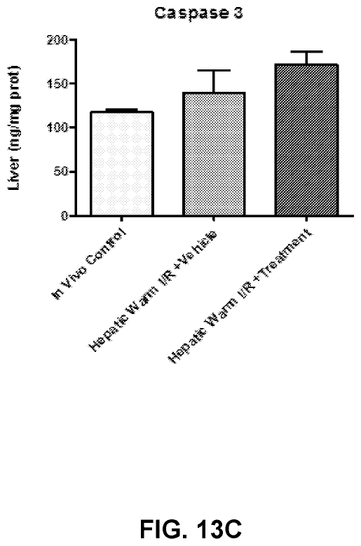

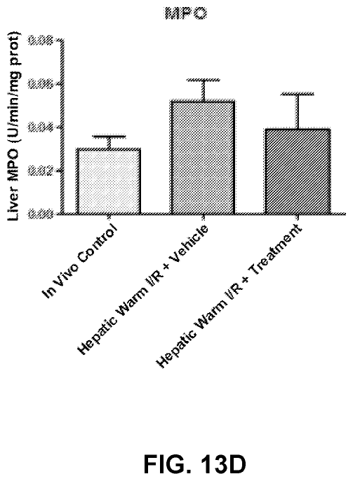

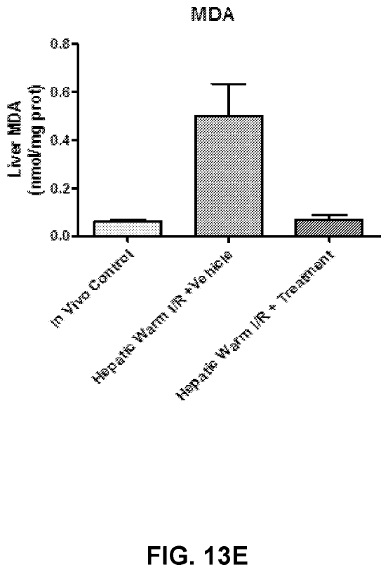

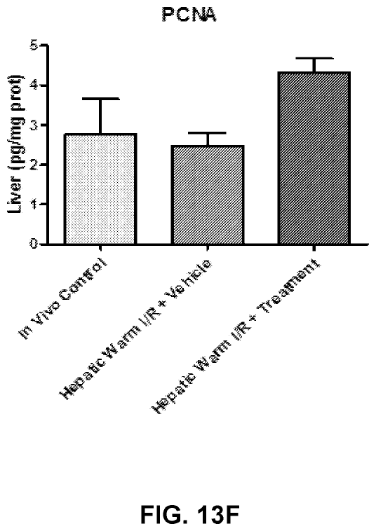

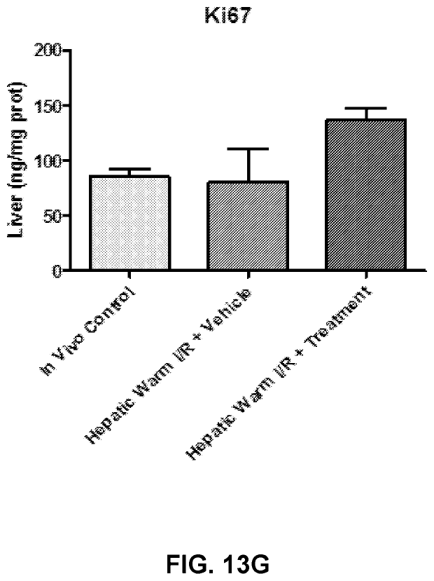

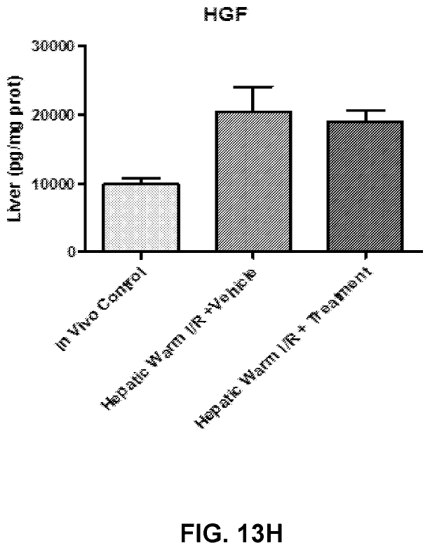

In the four days protocol, PBS control, vehicle control (AAV-GFP/Luc+AAV-rtTA) or AAV-OSKM (AAV-OSK+AAV cMyc/rtTA) were administered as described above. On day 3 after administration, doxycycline was added into the drinking water of the rats (1 mg/mL diluted in the drinking water). On day 4 after administration, hepatic I/R was carried out as described above. After 4 hours of reperfusion, the rats were sacrificed and organ/tissues retrieved. The treatment AAV-OSK+AAV-cMyc+AAV-rtTa in liver submitted to hepatic I/R induced a reduction in the values of transaminases (evaluated by ALT and AST (see FIGS. 13A-13B)), oxidative stress (measured by MDA (see FIG. 13E)). No effect was seen on Caspase 3, MPO, PCNA, Ki67, HGF, and TGFB levels (see FIGS. 13C-13D and 13F-13I, respectively).

In the two day protocol, PBS control, vehicle control (AAV-GFP/Luc+AAV-rtTA) or AAV-OSKM (AAV-OSK+AAV cMyc/rtTA) were administered as described above. On day 1 after administration, doxycycline was added into the drinking water (1 mg/mL diluted in the drinking water). On day 2 after administration, hepatic I/R was conducted as described above. After 4 hours of reperfusion, the rats were sacrificed and organ/tissues were retrieved.

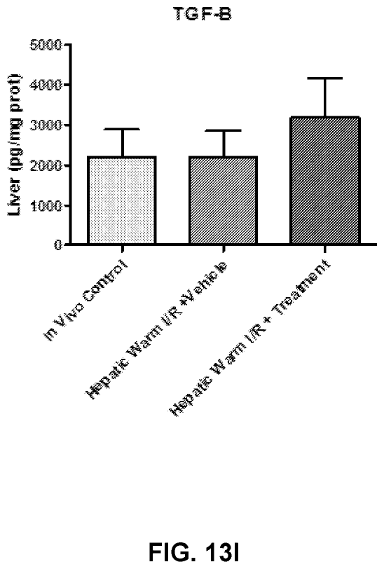

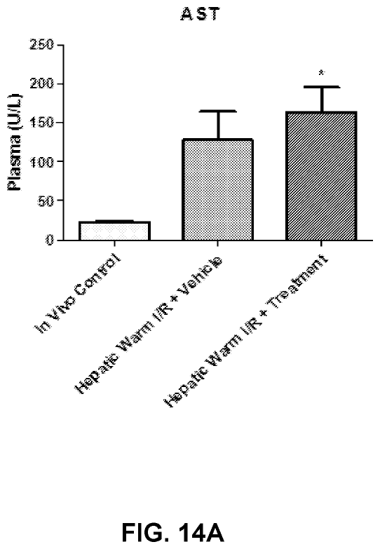

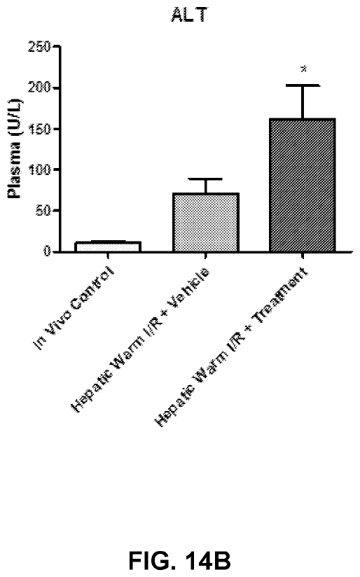

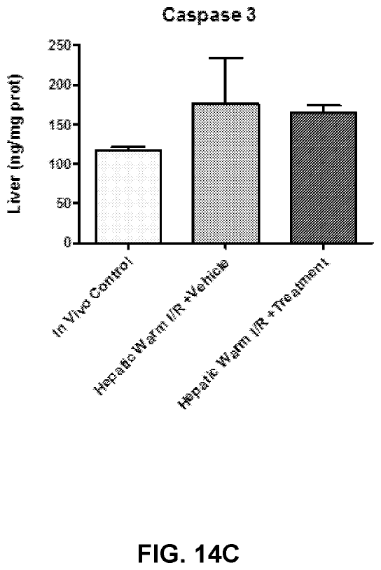

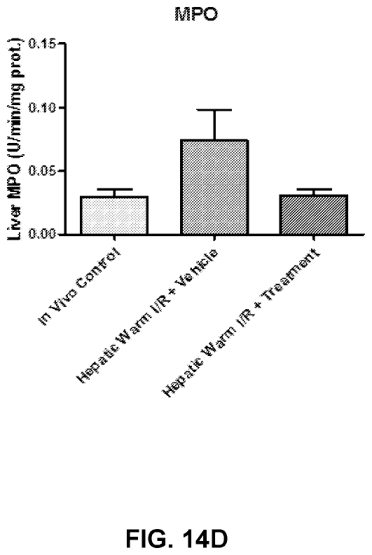

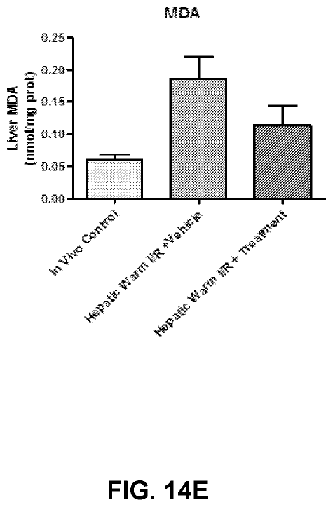

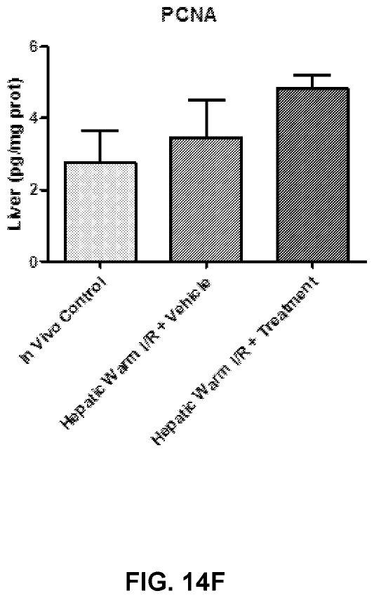

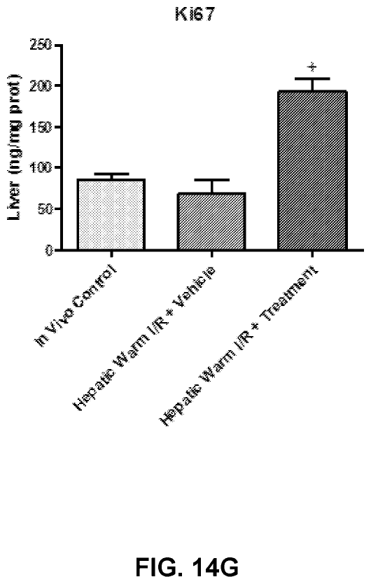

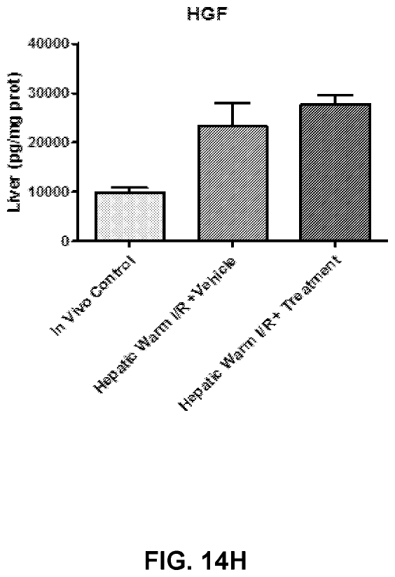

AST and ALT were not reduced compared to controls (PBS and vehicle) in OSKM-AAV treated group as seen in the 4 days (see FIGS. 13A and 13B) and 1 week protocols (see FIGS. 12A and 12B). A significant elevation in ALT levels (see FIG. 14A) and AST (see FIG. 14B) were seen in rats with warm ischemia compared to controls without warm ischemia. Caspase 3, MPO, MDA, PCNA, ki67, HGF, and TGFb did not show significant differences between control and treatment arms (see FIGS. 14C-14I, respectively).

Thus, a minimal amount of 3 days between AAV injection and OSKM activation by doxycycline is needed to observe a protective effect in liver injury markers in the warm-ischemia liver injury model. By contrast, when OSKM is activated only 2 days after AAV injection, the beneficial effect is not observed.

Therefore, treatment with OSKM prior to warm hepatic ischemia reperfusion acute liver injury resulted in beneficial effects, specifically when the AAV injection was 4 days, or one week prior to the injury; when assessed by liver injury markers following four hours of reperfusion. In contrast, treatment with OSKM if the AAV is only injected 2 days prior to the injury, did not show striking effect (as more than two days is needed for the expression of OSKM from the AAV vectors).

### Example 8. OSKM Treatment Ameliorates Ischemic Injury in a Death after Cardiac Arrest Kidney Transplantation Model

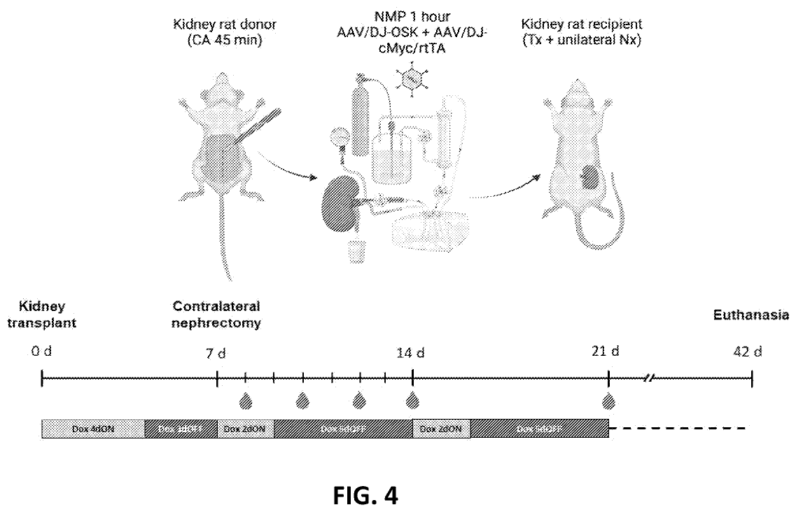

Ischemia-reperfusion injury is the main cause of acute kidney injury for kidneys in vivo and during organ transplantation. Depending on ischemic injury severity, tubular lesions and kidney function impairment can resolve after some days (classically 3 weeks in humans), or lead to chronic tubulo-interstitial lesions and fibrosis (Chawla et al. “Acute kidney injury and chronic kidney disease as interconnected syndromes”, NEJM 371(1):58-66 (2014)). In order to closely mimic cardiac death-derived ischemia, an animal (rat) model of kidney transplant following warm kidney ischemia due to cardiac arrest (45 min), was studied (see FIG. 4).

First, the rat was subjected to a cardiac arrest, which resulted in an immediate discontinuation of kidney arterial blood flow. Cardiac arrest was caused by clamping the donor ascendent aorta for 45 minutes before kidney procurement. 1 hour of kidney graft perfusion was performed with either control perfusion (without AAV), or treatment (perfusion in the presence of AAV/DJ-OSK+AAV/DJ cMyc-rtTA).

Renal transplants were performed with an end-to-side anastomosis of the aortic stump of the donor kidney and recipient's aorta, and between the recipient inferior vena cava and donor renal vein, respectively, using 9-0 running nylon sutures. The mean time for anastomosis was 24.8±3.3 minutes. Uretero-ureterostomy was performed with an end-to-end interrupted sutures technique using 11-0 nylon sutures. Ipsilateral native kidney was removed at the time of engraftment (kidney transplant). Donor kidneys were flushed with sterile physiologic saline at 4° C. after systemic application of sodium heparin (1000 UI, Rovi). The nephrectomy of the non-perfused kidney was performed 7 days after kidney transplantation to avoid early mortality due to acute kidney injury in the ischemic kidney.

1 mg/mL doxycycline (Sigma-Aldrich) was then administered in the drinking water of the recipient rats during 4 days in the first week after the procedure, starting the day of the transplantation. From the second week after transplantation and until sacrifice, doxycycline (dox) was administered in weekly cycles of 2 days a week of dox, followed by 5 days without dox. Blood tests to measure Blood Urea Nitrogen (BUN) and Serum Creatinine (SCr) were performed every-other-day after contralateral nephrectomy (i.e., 1 week post-transplantation) until day 14 post-transplantation with an EPOC Blood Analysis System (Siemens®). Afterwards, blood and urine analysis (proteinuria and microalbuminuria) were performed weekly until animal sacrifice.

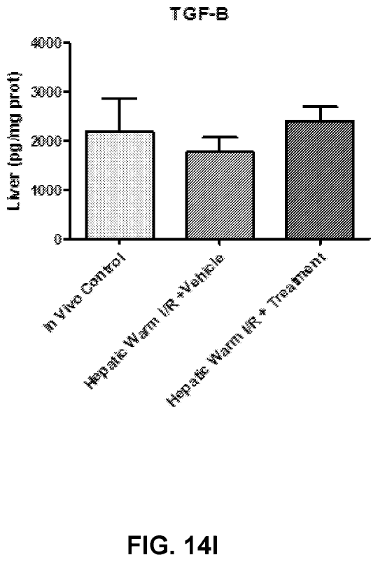

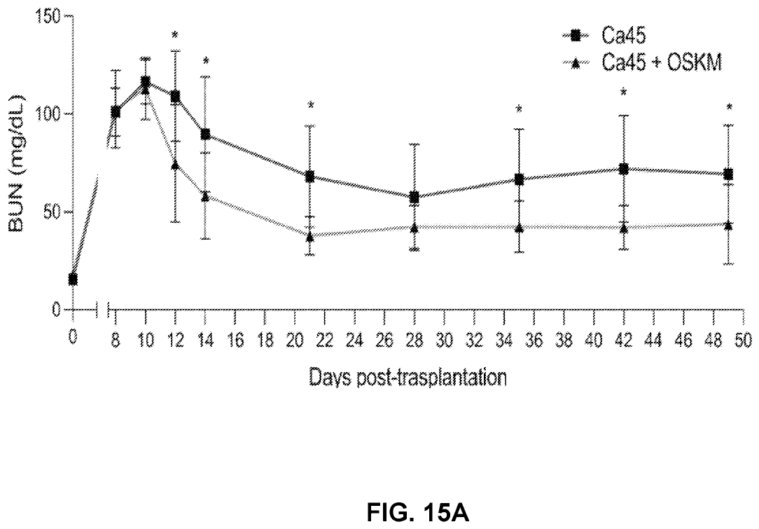

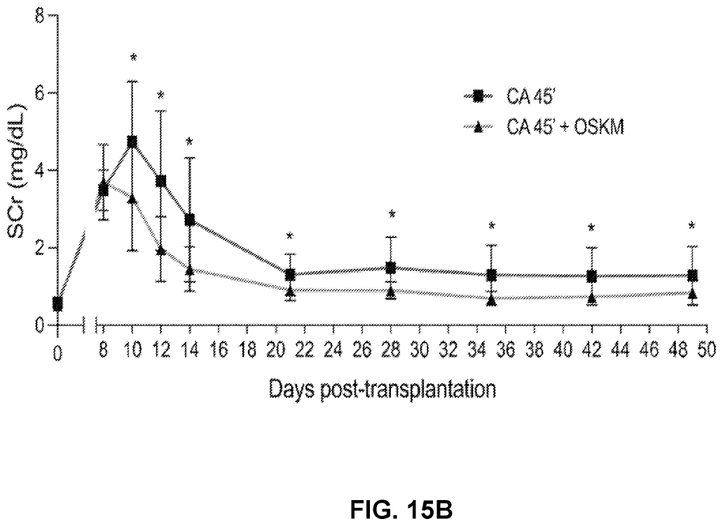

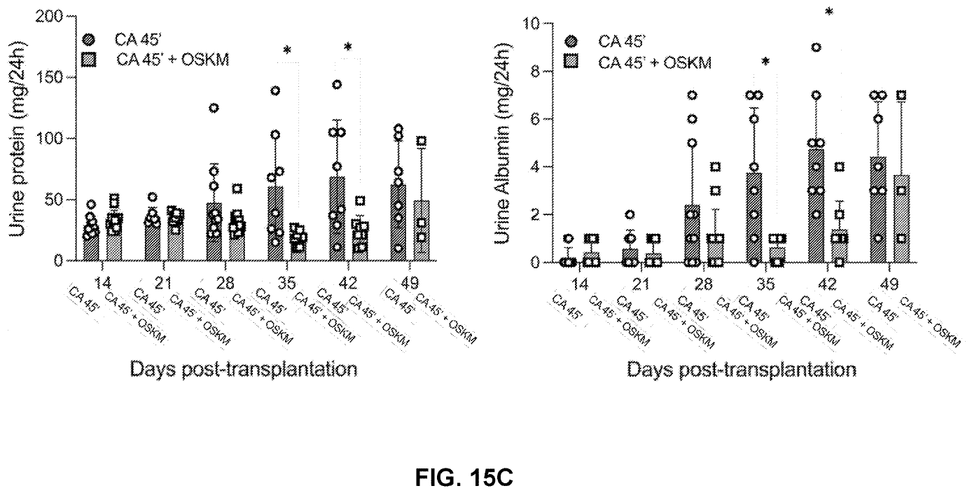

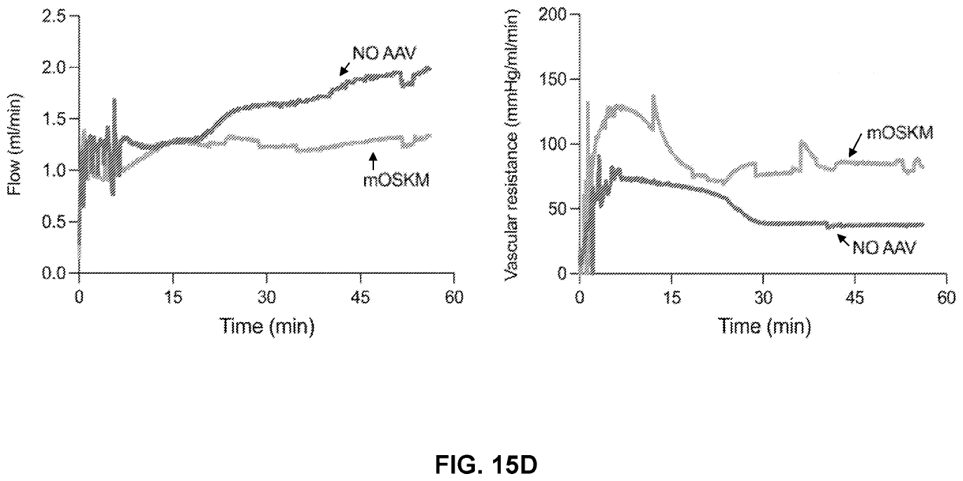

Eight days after transplantation (i.e., one day after contralateral nephrectomy), serum creatine (SCr) was noted to be similar between both control and treated groups (0.59±0.16 vs 0.52±0.03 mg/dL for the control and the treated group, respectively, P=0.12) (see FIG. 15A).

However, SCr started to decrease 10 days after transplantation in the OSKM group, versus control group of the first kidney graft, wherein improvement was not observed until 12 days post-transplantation. The worst kidney function was attained at 10 days (see FIG. 15A). Remarkably, despite kidney graft improvement, SCr values were permanently higher during the follow up compared to the OSKM group (FIG. 1A). These results suggest that OSKM treatment ameliorates kidney repair after an ischemic injury, reduces ischemia-induced chronic lesions and increases long-term kidney function.

An increase in BUN levels was observed for both groups at 8 and 10 days post-transplantation (90.56±33.01 and 106.73±33.85 vs 102.50±19.79 and 112.91±15.89 mg/dL at 8 and 10 days post-transplantation for the control and treated groups, respectively, P=0.26) (see FIG. 15B). After that timepoint, BUN levels decreased for both groups. This decrease occurred earlier for those treated animals (109.10±36.57 mg/dL vs 74.72±30.10 mg/dL for the control and the treated group, respectively, P=0.01). This accelerated improvement observed in the treated group led to better kidney function throughout the time course of the experiment, compared to the control group (see Table 1).

TABLE 1
Scores for modified Remuzzi score
criteria and total Remuzzi score.

| | Control | mOSKM |
|---|---|---|
| Tubular atrophy | 1.70 + 0.67 | 1.14 + 0.38 |
| Intersticial fibrosis | 2.1 + 0.32 | 1.43 + 0.53 |
| ATN grade | 2.4 + 0.84 | 1.29 + 0.49 |
| Total Score | 6.2 + .140 | 3.86 + 1.21 |

Similar kinetics were observed for total urine protein and albumin: urine protein levels were comparable for both groups until 28 days after transplantation, in which urine protein levels displayed increases for the control group, and markedly, a significant increase from 35 days post-transplantation for both total urine protein (57.89±41.68 vs 30.76±9.86 mg/24 h for the control and the treated group, respectively, P=0.02) and urine albumin (7.22±10.72 vs 0.63±0.52 mg/24 h for the control and the treated group, respectively, P=0.01). These differences persisted during the follow-up (see FIG. 15C). These results demonstrate that OSKM treatment ameliorated kidney repair after an ischemic injury, reduces ischemia-induced chronic lesions and increases long-term kidney function.

Regarding the hemodynamic monitoring of perfused kidneys during normothermic perfusion, we observed an initial increase in vascular resistance that progressively decreased during the perfusion in both groups, although in OSKM-treated kidneys, this resistance was higher all along the perfusion time compared to the non-treated ones. A similar scenario was observed for kidney flow during perfusion, with a progressive increase over time (see FIG. 15D). Increased vascular resistance may represent vessel vasoconstriction due to kidney injury and can be variable between rats. Higher vascular resistance can represent higher ischemic injuries in OSKM group, however the differences are not significantly different. in OSKM group, however these differences are not significantly different.

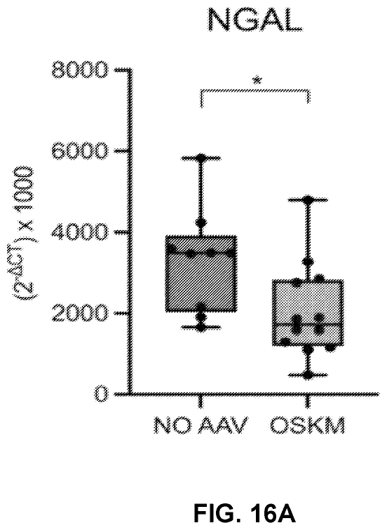

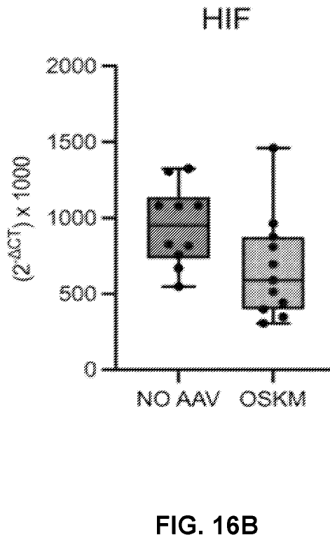

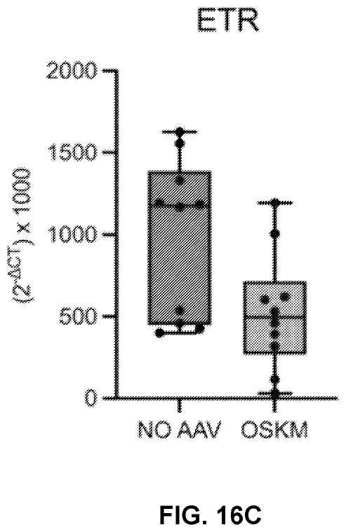

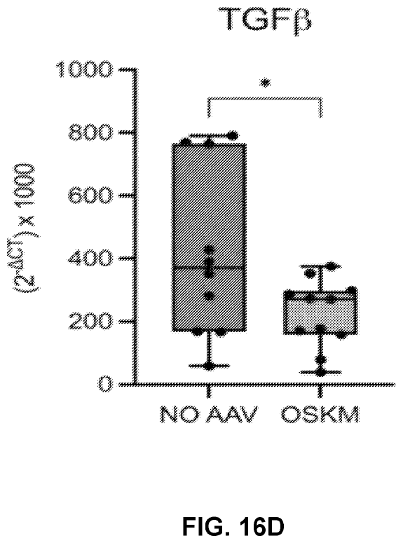

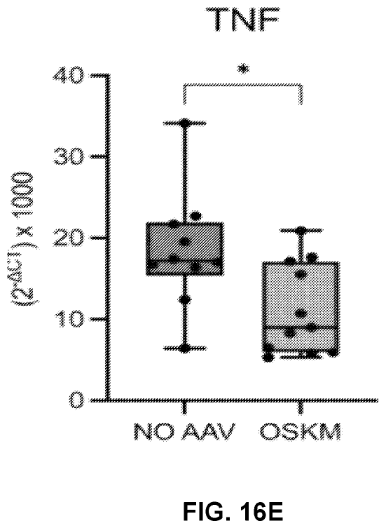

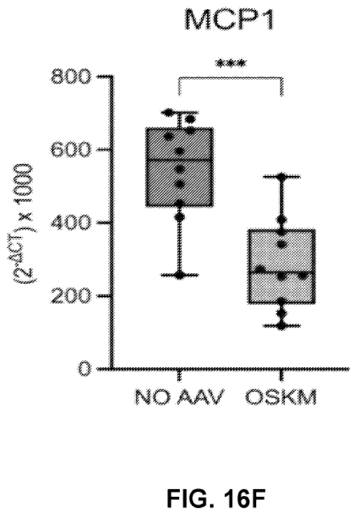

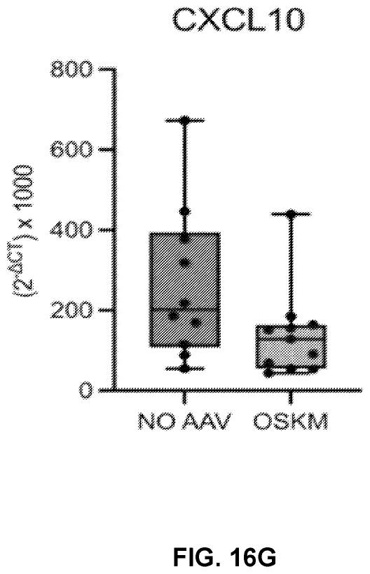

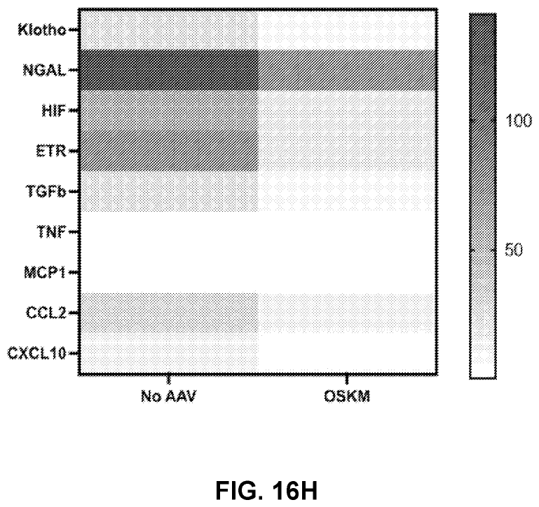

Gene expression analysis of kidney tissue at 6 weeks after transplantation revealed a significant increased expression of the kidney injury marker NGAL in non-treated rats when compared to the OSKM group (see FIG. 16A). Expression of HIF and ETR (related to kidney ischemic injury) tends to be higher in the control group (see FIGS. 16B and 16C). Expression of inflammatory markers TGFβ and TNF, as well as chemotactic-related genes MCP1 and CXCL10 which are markers for tissue inflammation was also higher in the control group compared to the treated one (see FIGS. 16D-16G). FIG. 1H summarizes the expression profile induced by OSKM treatment, showing that 6 weeks after kidney transplantation, genes related to ischemia (HIF, ETR), tubular injury (NGAL) or inflammation (CCL2, CXCL10) are higher in control kidneys compared to kidneys treated with OSKM treated kidneys. These transcriptomic data suggest that OSKM treatment ameliorated ischemia-induced chronic tubular lesions and inflammation.

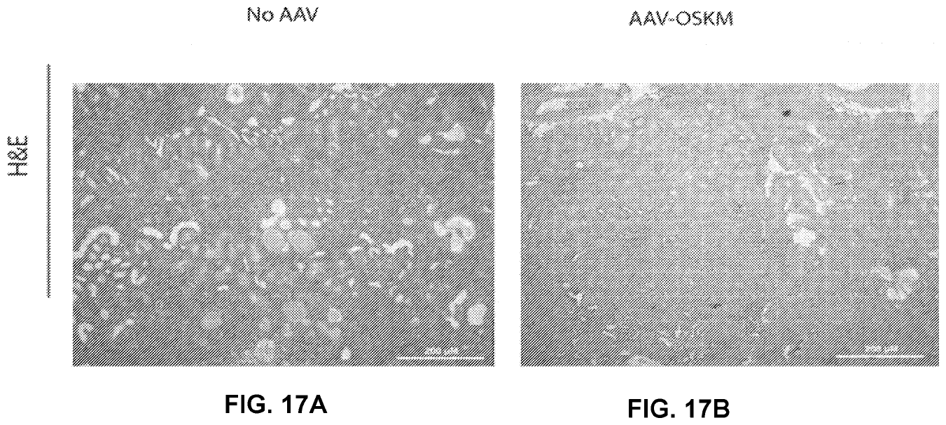

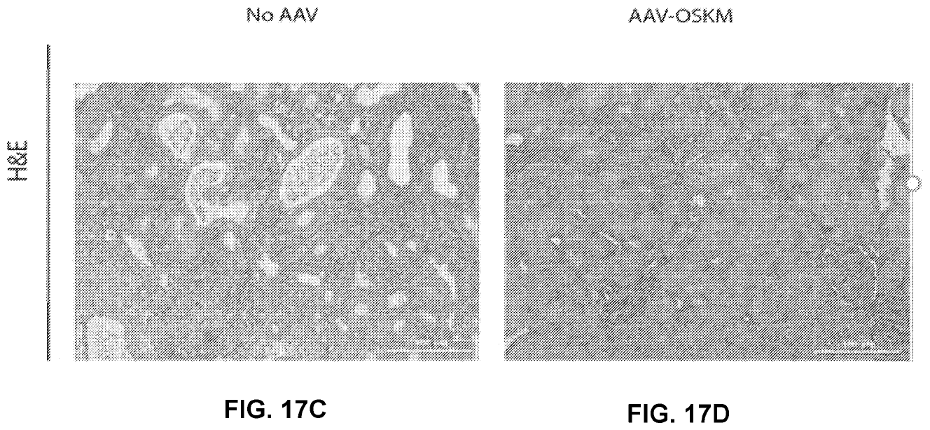

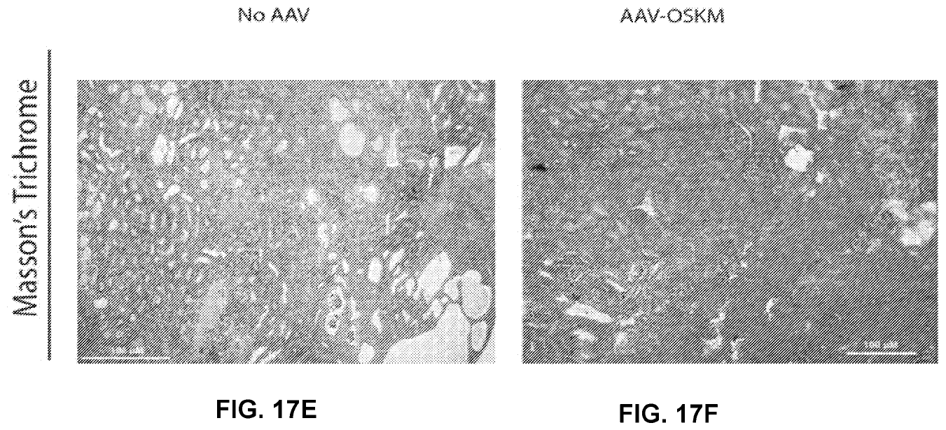

Rats were euthanized at 6 weeks post-transplantation and tissues were collected for analysis. The organ tissues were fixed in formaldehyde (Sigma) for 24 hours and then embedded in paraffin. 3-mm sections were stained with Hematoxylin-Eosin (H&E) (see FIGS. 17A-17D) and Masson's Trichrome (see FIGS. 17E-17F). Histology images were acquired using a Leica DMI6000 B inverted microscope (Leica Microsystems, Germany).

The kidney histology of both groups were assessed, and important signs of acute tubular necrosis in both control and OSKM-treated groups were observed, with dilated kidney tubules and plain tubular epithelium. These findings were significantly more marked in the non-treated (control) group (see FIGS. 17A-17D). The control group exhibited important chronicity findings, with extensive areas of severe interstitial and tubular atrophy in the kidney parenchyma (see FIG. 17E), while in the AAV treated-group, only a mild tubular atrophy and interstitial fibrosis was identified (see FIG. 3F). In the control group, intima proliferation and arteriolar obliteration, as well as a marked inflammatory infiltrate, were also observed. Glomerular sclerosis was not significant in any of the study groups. Global histological analyses of the kidneys 6 weeks after transplantation showed that OSKM treatment reduces tubular injury and kidney fibrosis induced by ischemia.

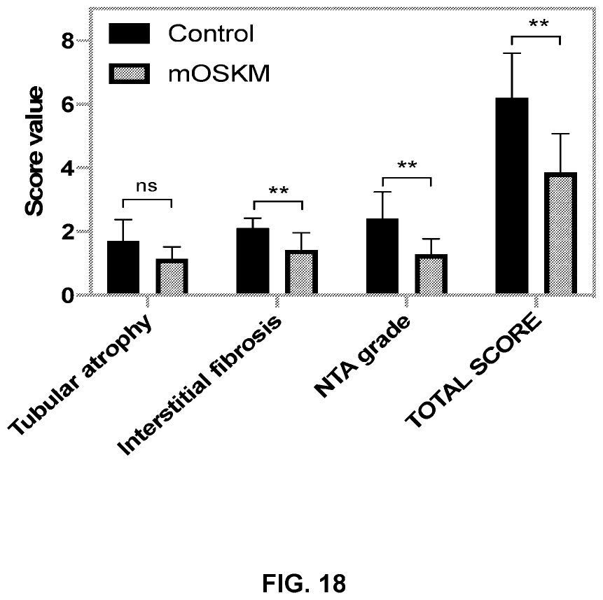

To confirm these results, a modified Remuzzi score was used to analyse the kidneys in order to globally assess tubular lesions (i.e., tubular atrophy and tubular necrosis) and kidney fibrosis. The Remuzzi score has been validated in human kidneys from donors to assess chronic and acute lesions. (Remuzzi et al. J Am Soc Nephrol 10: 2591-2598, 1999). Notably, 6 weeks after transplantation incidence and/or degree of chronic long-term histological lesions (tubular atrophy, tubular necrosis and kidney fibrosis) were significantly lower in OSKM-treated kidneys this compared to controls (see FIG. 18).

Taken together, the results of this study demonstrated that OSKM delivered during ex-vivo normothermic perfusion to an ischemic kidney and activated after transplantation ameliorates tubular injury, improves post-transplant kidney function and results in decreased chronic long-term histological lesions.

TABLE 2
Sequences

| SEQ ID NO: | Description |
|---|---|
| SEQ ID NO: 1 | Synthetic sequence hu Oct4 |
| SEQ ID NO: 2 | Synthetic Sequence hu Sox2 |
| SEQ ID NO: 3 | hu KLF4 |
| SEQ ID NO: 4 | hMyc gene (from AAV-TRE3G-hMcy-Ef1a-rTta) |
| SEQ ID NO: 5 | hu L-Myc |
| SEQ ID NO: 6 | hu N-Myc |
| SEQ ID NO: 7 | TRE3G promoter (from AAV TRE3G OSK shortWPRE SV40polyA) |
| SEQ ID NO: 8 | mouse Oct4 (from AAV TRE3G OSK shortWPRE SV40polyA) |
| SEQ ID NO: 9 | mouse Sox2 (from AAV TRE3G OSK shortWPRE SV40polyA) |
| SEQ ID NO: 10 | mouse Klf4 (from AAV TRE3G OSK shortWPRE SV40polyA) |
| SEQ ID NO: 11 | mouse c-Myc (from AAV TRE3G cMyc shortWPRE SV40polyA) |
| SEQ ID NO: 12 | EF-1a promoter (from AAV TRE3G cMyc shortWPRE SV40polyA) |
| SEQ ID NO: 13 | Tet-On 3G (from AAV TRE3G cMyc shortWPRE SV40polyA) |
| SEQ ID NO: 14 | short WPRE & SV40 polyA (from AAV TRE3G OSK shortWPRE |
| | SV40polyA) |
| SEQ ID NO: 15 | bGH polyA (from AAV TRE3G cMyc shortWPRE SV40polyA) |
| SEQ ID NO: 16 | dnNFKBIA (h-IKB alpha sup repressor) |
| SEQ ID NO: 17 | furin cleavage site consensus sequence |
| SEQ ID NO: 18 | IRES |
| SEQ ID NO: 19 | hu p16 promoter |
| SEQ ID NO: 20 | mouse p16 promoter |
| SEQ ID NO: 21 | AAV H-p16 OSK short WPRE sv40 polya |
| SEQ ID NO: 22 | AAV H-p16 IKKB sup repressor-GFP short WPRE sv40 polya |
| SEQ ID NO: 23 | AAV mp16 h-IKB alpha super repressor-GFP |
| SEQ ID NO: 24 | AAV mp16-OSK |
| SEQ ID NO: 25 | AAV mp16 CBRLuc-GFP |
| SEQ ID NO: 26 | plasmid AAV TRE3G OSK shortWPRE SV40 polyA |
| SEQ ID NO: 27 | plasmid AAV TRE3G cMyc shortWPRE SV40polyA + EF1a |
| | TetON3G |
| SEQ ID NO: 28 | plasmid AAV-TRE3G-hMyc-Ef1a-rTta |
| SEQ ID NO: 29 | plasmid AAV-CAG-CBRLuc-GFP |
| SEQ ID NO: 30 | plasmid pLenti-TRE3G-hOSKM-Cherry-rtTa-BlastR |
| SEQ ID NO: 31 | furin cleavage site consensus sequence |
| SEQ ID NO: 32 | Luciferase (forward) |
| SEQ ID NO: 33 | Luciferase (reverse) |
| SEQ ID NO: 34 | TGFβ (forward) |
| SEQ ID NO: 35 | TGFβ (reverse) |
| SEQ ID NO: 36 | NGAL (forward) |
| SEQ ID NO: 37 | NGAL (reverse) |
| SEQ ID NO: 38 | TNF (forward) |
| SEQ ID NO: 39 | TNF (reverse) |
| SEQ ID NO: 40 | GFP (forward) |
| SEQ ID NO: 41 | GFP (reverse) |
| SEQ ID NO: 42 | CXCL10 (forward) |
| SEQ ID NO: 43 | CXCL10 (reverse) |
| SEQ ID NO: 44 | HIF (forward) |
| SEQ ID NO: 45 | HIF (reverse) |
| SEQ ID NO: 46 | ETR (forward) |
| SEQ ID NO: 47 | ETR (reverse) |
| SEQ ID NO: 48 | GADPH (forward) |
| SEQ ID NO: 49 | GADPH (reverse) |
| SEQ ID NO: 50 | MCP1 (forward) |
| SEQ ID NO: 51 | MCP1 (reverse) |

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present conception has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed. The foregoing description of the specific embodiments aspects will so fully reveal the general nature of the invention conception that others can, by applying knowledge within the skill of the art, readily modify and/or adapt for various applications such specific embodiments aspects, without undue experimentation, without departing from the general concept of the presently described. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

What is claimed is:

1. An organ perfusate comprising a first polynucleotide encoding at least one regeneration factor and at least one vasodilator.

2. The organ perfusate of claim 1, wherein the first polynucleotide encoding the at least one regeneration factor is operably linked to a promoter.

3. The organ perfusate of claim 1 or 2, further comprising at least one tonicity agent.

4. The organ perfusate of any one of claims 1-3, further comprising at least one of a buffer, an inorganic salt, an amino acid, a substrate for metabolism, a hormone, an anti-oxidant, an anti-inflammatory agent, an anti-coagulant, or an anti-microbial agent.

5. The organ perfusate of any one of claims 1-3, further comprising human serum albumin, dextran, and an extracellular electrolyte composition.

6. The organ perfusate of any one of claims 3-5, wherein the tonicity agent is selected from the group consisting of dextrose, glycerin, mannitol, potassium chloride, sodium chloride, and combinations thereof.

7. The organ perfusate of any one of claims 1-6, wherein the vasodilator is selected from the group consisting of carbon monoxide, an angiotensin converting enzyme (ACE) inhibitor, an angiotensin receptor blocker, a calcium channel blocker, prostacyclin, hydralazine, minoxidil, nitroglycerin, and combinations thereof.

8. The organ perfusate of any one of claims 1-7, further comprising an oxygenation agent.

9. The organ perfusate of claim 8, wherein the oxygenation agent is selected from the group consisting of a red blood cell, hemoglobin, pyridoxylated hemoglobin, a synthetic hemoglobin-based oxygen carrier, and combinations thereof

10. The organ perfusate of claim 9, wherein the synthetic hemoglobin-based oxygen carrier is a polymerized hemoglobin-based oxygen carrier, Lifor™, Aquix RS-I, Hemarina®, or Perfluorocarbon.

11. The organ perfusate of any one of claims 1-10, further comprising a second polynucleotide encoding at least one regeneration factor operably linked to a promoter.

12. The organ perfusate of any one of claims 1-11, wherein the promoter of the first polynucleotide and the second polynucleotide are inducible promoters.

13. The organ perfusate of any one of claims 1-12, wherein the at least one regeneration factor encoded by the first polynucleotide is selected from the group consisting of an Oct family transcription factor, a Sox family transcription factor, a Klf family transcription factor, and a Myc family transcription factor.

14. The organ perfusate of any one of claims 11-13, wherein the at least one regeneration factor encoded by the second polynucleotide is selected from the group consisting of an Oct family transcription factor, a Sox family transcription factor, a Klf family transcription factor, and a Myc family transcription factor.

15. The organ perfusate of claim 13 or 14, wherein the Oct family transcription factor is selected from the group consisting of Oct1, Oct3, Oct4, Oct6, and variants thereof.

16. The organ perfusate of claim 13 or 14, wherein the Sox family transcription factor is selected from the group consisting of Sox1, Sox2, Sox3, Sox7, Sox15, Sox17, Sox18, and variants thereof.

17. The organ perfusate of claim 13 or 14, wherein the Klf family transcription factor is selected from the group consisting of Kfl1, Klf4, Klf5, and variants thereof.

18. The organ perfusate of claim 13 or 14, wherein the Myc family transcription factor is selected from the group consisting of c-Myc, L-Myc, N-Myc, and variants thereof.

19. The organ perfusate of any one of claims 1-18, wherein the first polynucleotide, the second polynucleotide, or both, are encapsulated in a nanoparticle.

20. The organ perfusate of claim 19, wherein the nanoparticle is a lipid nanoparticle, a polymeric nanoparticle, a ligand-conjugated lipid nanoparticle, or a ligand-conjugated polymeric nanoparticle.

21. The organ perfusate of any one of claims 1-20, wherein the first polynucleotide, the second polynucleotide, or both, are present within a viral genome, a plasmid, a minicircle vector, or a transposon.

22. The organ perfusate of claim 21, wherein the viral genome is selected from an AAV genome, an adenoviral genome, a retroviral genome, or a lentiviral genome.

23. The organ perfusate of any one of claims 1-22, wherein the perfusate further comprises a regeneration factor-protein transfer domain fusion protein.

24. The organ perfusate of any one of claims 1-23, wherein the perfusate further comprises a regeneration factor polypeptide.

25. The organ perfusate of any one of claims 1-24, wherein the perfusate further comprises an Oct family transcription factor, a Sox family transcription factor, a Klf family transcription factor, and/or a Myc family transcription factor.

26. The organ perfusate of claim 25, wherein the Oct family transcription factor is selected from the group consisting of Oct1, Oct3, Oct4, Oct6, and variants thereof.

27. The organ perfusate of claim 25 or 26, wherein the Sox family transcription factor is selected from the group consisting of Sox1, Sox2, Sox3, Sox7, Sox15, Sox17, Sox18, and variants thereof.

28. The organ perfusate of any one of claims 25-27, wherein the Klf family transcription factor is selected from the group consisting of Kfl1, Klf4, Klf5, and variants thereof

29. The organ perfusate of any one of claims 25-28, wherein the Myc family transcription factor is selected from the group consisting of c-Myc, L-Myc, N-Myc, and variants thereof.

30. The organ perfusate of any one of claims 1-29, further comprising an enhancing agent selected from the group consisting of soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), and combinations thereof.

31. A method of ex vivo rejuvenation of an organ, the method comprising:
(a) providing an organ;
(b) contacting the organ ex vivo with a composition comprising a polynucleotide encoding at least one regeneration factor operably linked to an inducible promoter; and
(c) adding to the composition a compound that induces the inducible promoter to promote expression of the at least one regeneration factor, whereby the at least one regeneration factor is expressed and the organ is rejuvenated.

32. The method of claim 31, wherein the compound is added intermittently.

33. The method of claim 32, wherein the intermittent addition comprises adding the compound once per day on two consecutive days and not adding the agent on the following 5 consecutive days.

34. The method of claim 31 or 32, wherein the intermittent addition is performed 2-10 times.

35. The method of claim 34, wherein the intermittent addition is performed 2-10 times over a period of about 1 week to about 6 weeks.

36. The method of any one of claims 31-35, wherein the composition further comprises a vasodilator.

37. The method of any one of claims 31-36, wherein the composition further comprises a tonicity agent.

38. The method of any one of claims 31-37, wherein the composition further comprises at least one of a buffer, an inorganic salt, an amino acid, a substrate for metabolism, a hormone, an anti-oxidant, an anti-inflammatory agent, an anti-coagulant, or an anti-microbial agent.

39. The method of any one of claims 31-38, wherein the organ is provided in a perfusion system selected from a Hugo-Sachs system, an Organ Assist system, an OrganOX system, a Radnoti system, an ARK Kidney system, and Aferetica PerLife® system.

40. The method of any one of claims 31-39, wherein the organ is a kidney or a liver.

41. A method of transplanting an organ into a subject in need thereof, the method comprising:
(a) providing an organ;
(b) contacting the organ ex vivo with a composition comprising a polynucleotide encoding at least one regeneration factor operably linked to an inducible promoter;
(c) adding to the composition a compound that induces the inducible promoter; and
(d) transplanting the organ into the subject.

42. The method of claim 41, wherein the compound is added for about 1 minute to about 24 hours to the composition.

43. The method of claim 41 or 42, wherein the compound is added intermittently to the composition.

44. The method of claim 43, wherein the intermittent addition of the compound comprises adding the compound once per day on two consecutive days and not adding the compound on the following 5 consecutive days.

45. The method of 44, wherein the intermittent addition of the compound is repeated 2-10 times.

46. The method of claim 45, wherein the intermittent addition is performed 2-10 times over a period of about 1 week to about 6 weeks.

47. A method of transplanting an organ into a subject in need thereof, the method comprising:
(a) providing an organ;
(b) contacting the organ ex vivo with a composition comprising a polynucleotide encoding at least one regeneration factor operably linked to an inducible promoter;
(c) transplanting the organ into the subject; and
(d) administering to the subject a compound that induces the inducible promoter.

48. The method of claim 47, wherein the compound is administered for about 1 minute to about 24 hours to the subject.

49. The method of claim 47 or 48, wherein the compound is administered intermittently to the subject.

50. The method of claim 49, wherein the intermittent administration of the compound comprises administering the compound once per day on two consecutive days and not administering the compound on the following 5 consecutive days.

51. The method of claim 49 or 50, wherein the intermittent administration of the compound is repeated about 2-10 times.

52. The method of claim 51, wherein the intermittent administration of the compound is performed about 2-10 times every 3 months.

53. The method of claim 52, wherein the intermittent administration of the compound is performed about 2-10 times every 6 months.

54. The method of any one of claims 31-53, wherein the composition further comprises a vasodilator.

55. The method of any one of claims 31-54, wherein the composition further comprises a tonicity agent.

56. The method of any one of claims 31-55, further comprising at least one of a buffer, an inorganic salt, an amino acid, a substrate for metabolism, a hormone, an anti-oxidant, an anti-inflammatory agent, an anti-coagulant, or an anti-microbial agent.

57. The method of any one of claims 31-56, wherein the explanted organ is provided in a perfusion system selected from a Hugo-Sachs system, an Organ Assist system, an OrganOX system, a Radnoti system, an ARK Kidney system, and Aferetica PerLife® system.

58. The method of any one of claims 31-57, wherein the organ is a kidney or a liver.

59. The method of any one of claims 31-58, wherein the method further comprises taking a biopsy from the organ prior to being transplanted.

60. The method of any one of claims 31-58, wherein the method further comprises taking a biopsy of the organ after being transplanted.

61. The method of any one of claims 31-60, further comprising monitoring the subject who has been transplanted with the organ for organ function.

62. The method of claim 61, wherein the monitoring comprises measuring one or more of blood urea levels, serum creatinine levels, bilirubin levels, blood pH, blood bicarbonate levels, blood sodium levels, blood potassium levels, or blood lactate levels.

63. The method of any one of claims 31-62, further comprising administering an immunosuppressive medication to the subject.

64. An ex vivo organ for transplantation to a subject in need thereof, the ex vivo organ comprising: (i) a perfusate; and (ii) a polynucleotide encoding at least one regeneration factor.

65. The ex vivo organ of claim 64, wherein the ex vivo organ is damaged by at least one of old age, an ischemic injury, a hypertensive injury, a toxic injury, a perfusion injury, an immune injury, a physical injury, steatosis, virus induced hepatitis, alcohol, or a fibrosis not associated with any known cause.

66. The ex vivo organ of claim 64 or 65, wherein the polynucleotide further comprises an inducible promoter operably linked to the polynucleotide encoding the at least one regeneration factor.

67. The ex vivo organ of any one of claims 64-66, wherein the perfusate intermittently comprises a compound that induces the inducible promoter.
